SEQUENCE LISTING (140 sequences)

SEQ ID NO: 1 - protein, 717 aa, unidentified
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 2 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 3 - protein, 717 aa, synthetic construct
MIKPTVSQFLTPGFKLIRNHSRTAGHKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 4 - protein, 717 aa, synthetic construct
MIKPTVSQFLTPGFKLIRNHSRTAGKKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 5 - protein, 752 aa, synthetic construct
MAPKKKRKVGIHGVPAAMIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKEF

SEQ ID NO: 6 - protein, 752 aa, synthetic construct
MAPKKKRKVGIHGVPAAMIKPTVSQFLTPGFKLIRNHSRTAGHKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKEF

SEQ ID NO: 7 - protein, 752 aa, synthetic construct
MAPKKKRKVGIHGVPAAMIKPTVSQFLTPGFKLIRNHSRTAGKKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKEF

SEQ ID NO: 8 - protein, 734 aa, synthetic construct
MAPKKKRKVGIHGVPAAMIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 9 - protein, 734 aa, synthetic construct
MAPKKKRKVGIHGVPAAMIKPTVSQFLTPGFKLIRNHSRTAGHKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 10 - protein, 734 aa, synthetic construct
MAPKKKRKVGIHGVPAAMIKPTVSQFLTPGFKLIRNHSRTAGKKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 11 - protein, 735 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKEF

SEQ ID NO: 12 - protein, 735 aa, synthetic construct
MIKPTVSQFLTPGFKLIRNHSRTAGHKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKEF

SEQ ID NO: 13 - protein, 735 aa, synthetic construct
MIKPTVSQFLTPGFKLIRNHSRTAGKKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKEF

SEQ ID NO: 14-16 - skipped (3 entries)

SEQ ID NO: 17 - protein, 8 aa, synthetic construct
  VARIANT 3: note = Any amino acid
  VARIANT 4: note = W, F, or Y
  VARIANT 5: note = Any amino acid
  VARIANT 6: note = Any amino acid
KRXXXXAF

SEQ ID NO: 18 - skipped

SEQ ID NO: 19 - protein, 6 aa, synthetic construct
  VARIANT 5: note = K or R
  VARIANT 6: note = W, F, or Y
LGKRXX

SEQ ID NO: 20 - skipped

SEQ ID NO: 21 - protein, 16 aa, synthetic construct
APKKKRKVGIHGVPAA

SEQ ID NO: 22 - protein, 18 aa, synthetic construct
KRPAATKKAGQAKKKKEF

SEQ ID NO: 23 - protein, 22 aa, synthetic construct
DYKDHDGDYKDHDIDYKDDDDK

SEQ ID NO: 24 - RNA, 20 nt, synthetic construct
  source 1..20: mol_type = other RNA
attgctccttacgaggagac

SEQ ID NO: 25 - RNA, 18 nt, synthetic construct
  source 1..18: mol_type = other RNA
atataagtggaggcgtcg

SEQ ID NO: 26 - RNA, 18 nt, synthetic construct
  source 1..18: mol_type = other RNA
gccttaacaagctgctct

SEQ ID NO: 27 - RNA, 20 nt, synthetic construct
  source 1..20: mol_type = other RNA
ctcacgtcatccagcagaga

SEQ ID NO: 28 - RNA, 38 nt, synthetic construct
  source 1..38: mol_type = other RNA
attgctccttacgaggagacatataagtggaggcgtcg

SEQ ID NO: 29 - RNA, 38 nt, synthetic construct
  source 1..38: mol_type = other RNA
attgctccttacgaggagacgccttaacaagctgctct

SEQ ID NO: 30 - RNA, 40 nt, synthetic construct
  source 1..40: mol_type = other RNA
attgctccttacgaggagacctcacgtcatccagcagaga

SEQ ID NO: 31 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQRPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 32 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQRPSPNKSIYCYQSVSPKPFITSKYRNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 33 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDRVPYKEAAGLNLIIKNAVNTYKGVQVKVDNKNKNNLAKINRKNEIAKLNGEQEISFEEIKAFDDKGYLLRRPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 34 - RNA, 53 nt, synthetic construct
  source 1..53: mol_type = other RNA
  modified_base 51: mod_base = OTHER; note = 2'-O-Methyl modified nucleotide
  modified_base 52: mod_base = OTHER; note = 2'-O-Methyl modified nucleotide
  modified_base 53: mod_base = OTHER; note = 2'-O-Methyl modified nucleotide
ctttcaagactaatagattgctccttacgaggagacgagctggtggcgtaggc

SEQ ID NO: 35 - RNA, 53 nt, synthetic construct
  source 1..53: mol_type = other RNA
  modified_base 51: mod_base = OTHER; note = 2'-O-Methyl modified nucleotide
  modified_base 52: mod_base = OTHER; note = 2'-O-Methyl modified nucleotide
  modified_base 53: mod_base = OTHER; note = 2'-O-Methyl modified nucleotide
ctttcaagactaatagattgctccttacgaggagacgagctgatggcgtaggc

SEQ ID NO: 36 - RNA, 20 nt, synthetic construct
  source 1..20: mol_type = other RNA
gtagttggagctggtggcgt

SEQ ID NO: 37 - RNA, 20 nt, synthetic construct
  source 1..20: mol_type = other RNA
gtagttggagctgatggcgt

SEQ ID NO: 38 - RNA, 36 nt, synthetic construct
  source 1..36: mol_type = other RNA
ctttcaagactaatagattgctccttacgaggagac

SEQ ID NO: 39 - RNA, 17 nt, synthetic construct
  source 1..17: mol_type = other RNA
gagctggtggcgtaggc

SEQ ID NO: 40 - RNA, 17 nt, synthetic construct
  source 1..17: mol_type = other RNA
gagctgatggcgtaggc

SEQ ID NO: 41 - RNA, 20 nt, synthetic construct
  source 1..20: mol_type = other RNA
gagagagggactttcccgtt

SEQ ID NO: 42 - RNA, 56 nt, synthetic construct
  source 1..56: mol_type = other RNA
ctttcaagactaatagattgctccttacgaggagacgagagagggactttcccgtt

SEQ ID NO: 43 - protein, 585 aa, synthetic construct
MLGRALINKYGFLIHPRRFVHLNDKSLDGTFILPSKKNHMYDVPTNDPSGILNASDIDRINNLPFFDNTSPTKETNTKEGALLSEKLASVKELFGEDPENPSFINYRFPRGLENPYFDIQVNQLKKKRLSVTQLCTTQNWCELRNFYDFYSQNLSNQLLNLKFQVQKGKKIHKSLEDETHPELNQYKSFTHNFLALTKLSMDIDNDMDALLDNWFNSINRLVSLFTKGDGHAREIVCHGFINLEDGKLVEHLLNSDSKTKENVIISGVIDHLTLRNRHNHQVQKGAAHLDTEYQSWGNILTNLLSNLKELKSNNEIVISDIKTRSVPKIPSIESVIESSKLQTMYYKFFFSHLSQDMTQTYHSFLINAQRRGLDVDAPINPTKILTFILTNPLFANDVKNLLYGLPINHSAFDNDAKGSNTFDMTAFNDLLDRGPTSFNVPIEQDEDSSESTKCVSLRDYGHFYTKWKTPLTLKYFAARLSQIYFIVGNLVSNDLMIEYYYHNDNFHNIIFPYDPLKLGTHAHDSAMVWFGGRDMHPIEPTQKNFNTYCKFCDYRHVCSWKNKNELKLIDLGKELKKIILESSMK

SEQ ID NO: 44 - protein, 702 aa, synthetic construct
MGIQGLLPQLKPIQNPVSLRRYEGEVLAIDGYAWLHRAACSCAYELAMGKPTDKYLQFFIKRFSLLKTFKVEPYLVFDGDAIPVKKSTESKRRDKRKENKAIAERLWACGEKKNAMDYFQKCVDITPEMAKCIICYCKLNGIRYIVAPFEADSQMVYLEQKNIVQGIISEDSDLLVFGCRRLITKLNDYGECLEICRDNFIKLPKKFPLGSLTNEEIITMVCLSGCDYTNGIPKVGLITAMKLVRRFNTIERIILSIQREGKLMIPDTYINEYEAAVLAFQFQRVFCPIRKKIVSLNEIPLYLKDTESKRKRLYACIGFVIHRETQKKQIVHFDDDIDHHLHLKIAQGDLNPYDFHQPLANREHKLQLASKSNIEFGKTNTTNSEAKVKPIESFFQKMTKLDHNPKVANNIHSLRQAEDKLTMAIKRRKLSNANVVQETLKDTRSKFFNKPSMTVVENFKEKGDSIQDFKEDTNSQSLEEPVSESQLSTQIPSSFITTNLEDDDNLSEEVSEVVSDIEEDRKNSEGKTIGNEIYNTDDDGDGDTSEDYSETAESRVPTSSTTSFPGSSQRSISGCTKVLQKFRYSSSFSGVNANRQPLFPRHVNQKSRGMVYVNQNRDDDCDDNDGKNQITQRPSLRKSLIGARSQRIVIDMKSVDERKSFNSSPILHEESKKRDIETTKSSQARPAVRSISLLSQFVYKGK

SEQ ID NO: 45 - protein, 291 aa, synthetic construct
MSKSWGKFIEEEEAEMASRRNLMIVDGTNLGFRFKHNNSKKPFASSYVSTIQSLAKSYSARTTIVLGDKGKSVFRLEHLPEYKGNRDEKYAQRTEEEKALDEQFFEYLKDAFELCKTTFPTFTIRGVEADDMAAYIVKLIGHLYDHVWLISTDGDWDTLLTDKVSRFSFTTRREYHLRDMYEHHNVDDVEQFISLKAIMGDLGDNIRGVEGIGAKRGYNIIREFGNVLDIIDQLPLPGKQKYIQNLNASEELLFRNLILVDLPTYCVDAIAAVGQDVLDKFTKDILEIAEQ

SEQ ID NO: 46 - protein, 319 aa, synthetic construct
MTAPILLLDGASMWFRSYFGVPSSIKAPDGRPVNAVRGFIDAISTLVTREKPRRLVVCRDDDWRPQWRVDLIPSYKAHRVAEPEPDGVPDIEEVPDDLTPQVNMILELLDAFGIPTAGAAGFEADDVLGTLSAREERDPVVVVSGDRDLLQLVRDEPAPQVRVLYLGRGLAKATKWGPAEVAEQYGVPLDRAGTAYAELALLRGDPSDGLPGVAGIGEKTAASLLAKHGSLQNILDAAHDPKSGLSKAHRTKLLGAVDYIAAAETVVRVATDAPVTFSTPTDTLPLAAGDPARVAELAAAYGVSSSISRLQTALDQLPD

SEQ ID NO: 47 - protein, 332 aa, synthetic construct
MIDKAYEELIKMKEKIMRDSYTIHKELSESVESILNELWINYSPESVEHSKKLLAIDGGMWVKETRQGVIFIVNAKAIVFEGINEINSEGKVLVHIFSPGNYAKERIELLMQLLELQLALKLVENVDYVLLDGSFSKKLGRHKSELKVDLLDDIVSIDKILSLEEKDEDNMLRFLIAENQLVLSELVSRYKDKLLFISKNSKSSDLFKQAYSDITILELFTQNCGYSKILEKKIDENYILSRKASKLLSGLNYYFTNLRLEPSERLFRLDFFNADKIFEYLKVLKPVSLKGYPYPLIKVHKDVRVGKEDRERIYSILEMKRKDISWWPSQFY

SEQ ID NO: 48 - protein, 575 aa, synthetic construct
MKKLIKRREIPIGNSVSNHPLLDRLYRARHIQNTKELDRTLKSMLNPNQLYGIEQAVNLLVEAYQPQQKIVIVGDFDADGATSTALSVLALRQLGFSDVDYLVPNRFEQGYGLSIPVAEMAIEKGVQLLMTVDNGVSSFDGVAFLKEKGIRVLVTDHHLPPETLPPADAIVNPNLSQCGFPSKSLAGVGVAFYLMLAVRAKFRELGIFTAETQPNFTDLLDLVALGTIADVVPLDQNNRILAYQGLMRIRARHCRLGIIALAEVANRNVEQFTSSDLGFCIGPRLNAAGRLDNMSIGVELLLANEMSKARELALDLDQLNQTRKEIEAGMKLEAIKICQNLTALFKELPYGITLYQPDWHQGVLGIVSSRIKDQYHRPVIAFAQDSEGILKGSARSIEGLHMRDVLERIHSQHPNMILKFGGHAMAAGLSIREEHFADFQHIFNQTVADWLDEEHLQGVIWTDGELNSNEFNLETAELIKSVGTWGQGFPEPCFDGEFKILDQRAIGQNKNHLKMLLEPKQGGVLLDAIAFNINTRLYPDLSIKQARLAYKLEINEFRGNRSLQLLVDYIEPIDE

SEQ ID NO: 49 - protein, 475 aa, synthetic construct
MMNDGKQQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIV

SEQ ID NO: 50 - protein, 661 aa, synthetic construct
MSRKSIVQIRRSEVKRKRSSTASSTSEGKTLHKNTHTSSKRQRTLTEFNIPTSSNLPVRSSSYSFSRFSCSTSNKNTEPVIINDDDHNSICLEDTAKVEITIDTDEEELVSLHDNEVSAIENRTEDRIVTELEEQVNVKVSTEVIQCPICLENLSHLELYERETHCDTCIGSDPSNMGTPKKNIRSFISNPSSPAKTKRDIATSKKPTRVKLVLPSFKIIKFNNGHEIVVDGFNYKASETISQYFLSHFHSDHYIGLKKSWNNPDENPIKKTLYCSKITAILVNLKFKIPMDEIQILPMNKRFWITDTISVVTLDANHCPGAIIMLFQEFLANSYDKPIRQILHTGDFRSNAKMIETIQKWLAETANETIDQVYLDTTYMTMGYNFPSQHSVCETVADFTLRLIKHGKNKTFGDSQRNLFHFQRKKTLTTHRYRVLFLVGTYTIGKEKLAIKICEFLKTKLFVMPNSVKFSMMLTVLQNNENQNDMWDESLLTSNLHESSVHLVPIRVLKSQETIEAYLKSLKELETDYVKDIEDVVGFIPTGWSHNFGLKYQKKNDDDENEMSGNTEYCLELMKNDRDNDDENGFEISSILRQYKKYNKFQVFNVPYSEHSSFNDLVKFGCKLKCSEVIPTVNLNNLWKVRYMTNWFQCWENVRKTRAAK

SEQ ID NO: 51 - protein, 260 aa, synthetic construct
MFDIGVNLTSSQFVKDRDDVVTRALAAGVSGMLLTGTNLHESQQAQKLAQRYACCWSTAGVHPHDSSQWQSETEDAIVALARQPDVVAIGECGLDFNRNFSTPQEQERAFQAQLRIAAELQMPVFMHCRDAHARFLALLEPWLDKLPGAVLHCFTGTREEMQECIDRGLYIGITGWVCDERRGLELRELLPFIPAEKLLIETDAPYLLPRDLTPKPASRRNEPAHLAHILARVAHWRGEDPQWLAATTDANVKTLFGIAF

SEQ ID NO: 52 - protein, 314 aa, synthetic construct
MGSQALPPGPMQTLIFFDMEATGLPFSQPKVTELCLLAVHRCALESPPTSQGPPPTVPPPPRVVDKLSLCVAPGKACSPAASEITGLSTAVLAAHGRQCFDDNLANLLLAFLRRQPQPWCLVAHNGDRYDFPLLQAELAMLGLTSALDGAFCVDSITALKALERASSPSEHGPRKSYSLGSIYTRLYGQSPPDSHTAEGDVLALLSICQWRPQALLRWVDAHARPFGTIRPMYGVTASARTKPRPSAVTTTAHLATTRNTSPSLGESRGTKDLPPVKDPGALSREGLLAPLGLLAILTLAVATLYGLSLATPGE

SEQ ID NO: 53 - DNA, 86 bp, unidentified
  source 1..86: mol_type = unassigned DNA
gttcggtcgctgcgtcacggtccaatctctctccctgaaagggcaaaagtcaaaggaaaagaatctcagagcactacaaattcttc

SEQ ID NO: 54 - protein, 26 aa, synthetic construct
GSGGSPAGSPTSTEEGTSESATPGSG

SEQ ID NO: 55 - RNA, 17 nt, synthetic construct
  source 1..17: mol_type = other RNA
gagcaacggcggaaggt

SEQ ID NO: 56 - RNA, 56 nt, synthetic construct
  source 1..56: mol_type = other RNA
ctttcaagactaatagattgctccttacgaggagactattaaatactcgtattgct

SEQ ID NO: 57-59 - skipped (3 entries)

SEQ ID NO: 60 - protein, 20 aa, synthetic construct
  VARIANT 4: note = Any amino acid
  VARIANT 8: note = Any amino acid
  VARIANT 11: note = Any amino acid
  VARIANT 15: note = Any amino acid
  VARIANT 19: note = Any amino acid
GLFXALLXLLXSLWXLLLXA

SEQ ID NO: 61-66 - skipped (6 entries)

SEQ ID NO: 67 - protein, 20 aa, synthetic construct
  VARIANT 4: note = Any amino acid
  VARIANT 8: note = Any amino acid
  VARIANT 11: note = Any amino acid
  VARIANT 15: note = Any amino acid
  VARIANT 19: note = Any amino acid
GLFXALLXLLXSLWXLLLXA

SEQ ID NO: 68-77 - skipped (10 entries)

SEQ ID NO: 78 - DNA, 86 bp, unidentified
  source 1..86: mol_type = unassigned DNA
taaacatcacgagactctaagaaaaggaaactgaaaacgggaaagtccctctctctaacctggcactgcgtcgctggcttggagac

SEQ ID NO: 79 - DNA, 91 bp, unidentified
  source 1..91: mol_type = unassigned DNA
cttcttaaacatcacgagactctaagaaaaggaaactgaaaacgggaaagtccctctctctaacctggcactgcgtcgctggcttgagaca

SEQ ID NO: 80 - DNA, 85 bp, unidentified
  source 1..85: mol_type = unassigned DNA
taaacatcacgagactctaagaaaaggaaactgaaaacgggaaagtccctctctctaacctggcactgcgtcgctggcttggaga

SEQ ID NO: 81 - DNA, 85 bp, unidentified
  source 1..85: mol_type = unassigned DNA
ttaaacatcacgagactctaagaaaaggaaactgaaaacgggaaagtccctctctctaacctggcactgcgtcgctggcttggag

SEQ ID NO: 82 - DNA, 85 bp, unidentified
  source 1..85: mol_type = unassigned DNA
aacatcacgagactctaagaaaaggaaactgaaaacgggaaagtccctctctctaacctggcactgcgtcgctggcttggagaca

SEQ ID NO: 83 - DNA, 85 bp, unidentified
  source 1..85: mol_type = unassigned DNA
acatcacgagactctaagaaaaggaaactgaaaacgggaaagtccctctctctaacctggcactgcgtcgctggcttggagacag

SEQ ID NO: 84 - DNA, 91 bp, unidentified
  source 1..91: mol_type = unassigned DNA
catgccttcttaaacatcacgagactctaagaaaaggaaactgaaaacgggaaagtccctctctctaacctggcactgcgtcgctggcttg

SEQ ID NO: 85 - DNA, 98 bp, unidentified
  source 1..98: mol_type = unassigned DNA
tctagtgcatgccttcttaaacatcacgagactctaagaaaaggaaactgaaaacgggaaagtccctctctctaacctggcactgcgtcgctggcttg

SEQ ID NO: 86 - protein, 5 aa, synthetic construct
GSGGS

SEQ ID NO: 87 - protein, 6 aa, synthetic construct
GGSGGS

SEQ ID NO: 88 - protein, 4 aa, synthetic construct
GGGS

SEQ ID NO: 89 - protein, 4 aa, synthetic construct
GGSG

SEQ ID NO: 90 - protein, 5 aa, synthetic construct
GGSGG

SEQ ID NO: 91 - protein, 5 aa, synthetic construct
GSGSG

SEQ ID NO: 92 - protein, 5 aa, synthetic construct
GSGGG

SEQ ID NO: 93 - protein, 5 aa, synthetic construct
GGGSG

SEQ ID NO: 94 - protein, 5 aa, synthetic construct
GSSSG

SEQ ID NO: 95 - protein, 6 aa, synthetic construct
HHHHHH

SEQ ID NO: 96 - DNA, 20 bp, synthetic construct
  source 1..20: mol_type = other DNA
  misc_difference 1..20: note = This sequence may encompass 5-20 nucleotides
aaaaaaaaaaaaaaaaaaaa

SEQ ID NO: 97 - DNA, 20 bp, synthetic construct
  source 1..20: mol_type = other DNA
  misc_difference 1..20: note = This sequence may encompass 5-20 nucleotides
tttttttttttttttttttt

SEQ ID NO: 98 - DNA, 20 bp, synthetic construct
  source 1..20: mol_type = other DNA
  misc_difference 1..20: note = This sequence may encompass 5-20 nucleotides
cccccccccccccccccccc

SEQ ID NO: 99 - DNA, 20 bp, synthetic construct
  source 1..20: mol_type = other DNA
  misc_difference 1..20: note = This sequence may encompass 5-20 nucleotides
gggggggggggggggggggg

SEQ ID NO: 100 - protein, 717 aa, synthetic construct
MIKPRVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 101 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 102 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGRGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 103 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPERHDKLAPSYTVVLREAV

SEQ ID NO: 104 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 105 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAQGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 106 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKKNKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 107 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVRPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 108 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITGQSMPKPTCERSGDAKKPVRARKAKAPEFHDKLAPSYTVVLREAV

SEQ ID NO: 109 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITARSMPKPTCERSGDAKKPVRARKAKAPEFHDKLAPSYTVVLREAV

SEQ ID NO: 110 - protein, 717 aa, synthetic construct
MIKPTVSQFLTPGFKLIRNHSRTAGKKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGRGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 111 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKKNKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPERHDKLAPSYTVVLREAV

SEQ ID NO: 112 - protein, 717 aa, synthetic construct
MIKPRVSQFLTPGFKLIRNHSRTAGKKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 113 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQQIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 114 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSRDAKKPVRARKAKAPEFHDKLAPSYTVVLREAV

SEQ ID NO: 115 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAKSMPKPTCERSGDAKKPVRARKAKAPEFHDKLAPSYTVVLREAV

SEQ ID NO: 116 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAREFHDKLAPSYTVVLREAV

SEQ ID NO: 117 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYRFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 118 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKRTCERSGDAKKPVRARKAKAPEFHDKLAPSYTVVLREAV

SEQ ID NO: 119 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 120 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVRPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 121 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCRRSGDAKKPVRARKAKAPEFHDKLAPSYTVVLREAV

SEQ ID NO: 122 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 123 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDKKNRNGEKFNCLKCGIELNADIDVATENLATVAITAQSMPKPTCERSGDAKKPVRARKAKAPEFHDKLAPSYTVVLREAV

SEQ ID NO: 124 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLKKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 125 - protein, 10 aa, synthetic construct
  VARIANT 1..10: note = This sequence may encompass 1-10 residues
GGGGGGGGGG

SEQ ID NO: 126 - RNA, 41 nt, synthetic construct
  source 1..41: mol_type = other RNA
atagattgctccttacgaggagacgtatttgtgaagtctta

SEQ ID NO: 127 - RNA, 41 nt, synthetic construct
  source 1..41: mol_type = other RNA
atagattgctccttacgaggagactatttgtgaagtcttac

SEQ ID NO: 128 - RNA, 41 nt, synthetic construct
  source 1..41: mol_type = other RNA
atagattgctccttacgaggagacctttgcactttccttag

SEQ ID NO: 129 - RNA, 41 nt, synthetic construct
  source 1..41: mol_type = other RNA
atagattgctccttacgaggagacagtcactctaagttact

SEQ ID NO: 130 - RNA, 41 nt, synthetic construct
  source 1..41: mol_type = other RNA
atagattgctccttacgaggagacaataaagcatagtgcaa

SEQ ID NO: 131 - RNA, 41 nt, synthetic construct
  source 1..41: mol_type = other RNA
atagattgctccttacgaggagacaaataaagcatagtgca

SEQ ID NO: 132 - RNA, 41 nt, synthetic construct
  source 1..41: mol_type = other RNA
atagattgctccttacgaggagactgagatcaacagcacag

SEQ ID NO: 133 - RNA, 41 nt, synthetic construct
  source 1..41: mol_type = other RNA
atagattgctccttacgaggagacatgagatcaacagcaca

SEQ ID NO: 134 - RNA, 41 nt, synthetic construct
  source 1..41: mol_type = other RNA
atagattgctccttacgaggagaccattttagtctgtcttc

SEQ ID NO: 135 - RNA, 41 nt, synthetic construct
  source 1..41: mol_type = other RNA
atagattgctccttacgaggagacaaaggcagaagaaataa

SEQ ID NO: 136 - RNA, 41 nt, synthetic construct
  source 1..41: mol_type = other RNA
atagattgctccttacgaggagacggctctgattcctacag

SEQ ID NO: 137 - RNA, 41 nt, synthetic construct
  source 1..41: mol_type = other RNA
atagattgctccttacgaggagacctgtaggaatcagagcc

SEQ ID NO: 138 - RNA, 41 nt, synthetic construct
  source 1..41: mol_type = other RNA
atagattgctccttacgaggagacgacttagattatgcatt

SEQ ID NO: 139 - RNA, 41 nt, synthetic construct
  source 1..41: mol_type = other RNA
atagattgctccttacgaggagacttttcttcagtatttaa

SEQ ID NO: 140 - RNA, 41 nt, synthetic construct
  source 1..41: mol_type = other RNA
atagattgctccttacgaggagactcttcagtatttaacaa

(19) United States
(12) Patent Application Publication
(10) Pub. No.: US 2025/0145974 A1
(43) Pub. Date: May 8, 2025

(54) ENGINEERED CAS-PHI PROTEINS AND USES THEREOF

(71) Applicant: Mammoth Biosciences, Inc., Brisbane, CA (US)

(72) Inventors: Wiputra Jaya HARTONO, San Francisco, CA (US); Pei-Qi LIU, Oakland, CA (US); Lucas Benjamin HARRINGTON, San Francisco, CA (US); Aaron DELOUGHERY, San Francisco, CA (US); William Douglass WRIGHT, Fairfield, CA (US); Stepan TYMOSHENKO, Sacramento, CA (US); Benjamin Julius RAUCH, San Francisco, CA (US)

(21) Appl. No.: 18/939,374

(22) Filed: Nov. 6, 2024

Related U.S. Application Data
(63) Continuation of application No. PCT/US2023/066746, filed on May 8, 2023.
(60) Provisional application No. 63/339,936, filed on May 9, 2022.
(60) Provisional application No. 63/391,588, filed on Jul. 22, 2022.
(60) Provisional application No. 63/374,428, filed on Sep. 2, 2022.
(60) Provisional application No. 63/482,725, filed on Feb. 1, 2023.

(51) Int. Cl. C12N 9/22; C12N 9/10; C12N 15/11; C12N 15/88

(52) U.S. Cl. CPC C12N 9/22; C12N 9/1007; C12N 15/11; C12N 15/88; C07K 2319/00; C12N 2310/20

(57) ABSTRACT

Provided herein are compositions, systems, and methods comprising engineered effector proteins and uses thereof. These effector proteins may be characterized as CRISPR-associated (Cas) proteins. Various compositions, systems, and methods of the present disclosure may leverage the activities of these effector proteins for the modification, detection, and engineering of nucleic acids.

### CROSS REFERENCE TO RELATED APPLICATIONS

This application is a continuation of International Patent Application No. PCT/US2023/066746, filed May 8, 2023, which claims the benefit of U.S. Provisional Application No. 63/339,936, filed May 9, 2022, U.S. Provisional Application No. 63/391,588, filed Jul. 22, 2022, U.S. Provisional Application No. 63/374,428, filed Sep. 2, 2022, and U.S. Provisional Application No. 63/482,725, filed Feb. 1, 2023, the disclosures of each of which are incorporated herein by reference in their entirety.

### SEQUENCE LISTING

The instant application contains a Sequence Listing, which has been submitted via Patent Center. The Sequence Listing titled 203477-756301US_SL.xml, which was created on Oct. 28, 2024 and is 153,024 bytes in size, is hereby incorporated by reference in its entirety.

### FIELD

The present disclosure relates generally to variant polypeptides, compositions of variant polypeptides and guide nucleic acids, systems and methods of using such polypeptides and compositions, including detecting and modifying target nucleic acids.

### BACKGROUND

Clustered Regularly Interspaced Short Palindromic Repeats (CRISPR) and associated proteins (Cas proteins), sometimes referred to as a CRISPR/Cas system, were first identified in certain bacterial species and are now understood to form part of a prokaryotic acquired immune system. CRISPR/Cas systems provide immunity in bacteria and archaea against viruses and plasmids by targeting the nucleic acids of the viruses and plasmids in a sequence-specific manner. Native systems contain a CRISPR array, which includes direct repeats flanking short spacer sequences that, in part, guide Cas proteins to their targets. The discovery of CRISPR/Cas systems has revolutionized the field of genomic manipulation and engineering, and therapeutic applications of these systems are being explored. While the programmable nature of these systems has promising implications in the field of genome engineering, there remains a need to explore alternative strategies and components to leverage the CRISPR-Cas system in ways that are sufficient for in vitro detection and effective for in vivo genome engineering. Variant polypeptides (e.g., effector proteins), guide nucleic acids, compositions, systems, and methods described herein may satisfy this need and provides related advantages.

### SUMMARY

The present disclosure provides for variant polypeptides, compositions, methods and systems comprising the same, in some instances guide nucleic acids, and uses thereof. Compositions, systems, and methods disclosed herein leverage nucleic acid modifying activities (e.g., cis cleavage activity) of these polypeptides and guide nucleic acids for the modification and detection of target nucleic acids. Accordingly, in one aspect, provided herein is a composition comprising a variant polypeptide and a guide nucleic acid for the modification of a target nucleic acid. In another aspect, provided herein are compositions comprising a variant polypeptide and a guide nucleic acid for the treatment of a disease or disorder associated with a target nucleic acid.

### Certain Embodiments

Provided herein are compositions comprising an engineered polypeptide or a nucleic acid encoding the engineered polypeptide, wherein the engineered polypeptide comprises one or more amino acid alterations of one or more residues relative to SEQ ID NO: 1, wherein the one or more amino acid alterations are at one or more positions selected from any one of the positions set forth in TABLE 1; and wherein the engineered polypeptide comprises an amino acid sequence that is at least 90% identical to the amino acid sequence of SEQ ID NO: 1. In some embodiments, the amino acid sequence of the engineered polypeptide is at least 99% identical to the amino acid sequence of SEQ ID NO: 1. In some embodiments, the one or more positions are selected from positions: 2, 5, 15, 18, 20, 21, 26, 30, 33, 34, 35, 37, 38, 41, 43, 54, 79, 92, 99, 108, 109, 110, 111, 113, 114, 116, 118, 119, 121, 132, 135, 138, 139, 149, 180, 182, 183, 184, 186, 189, 196, 198, 200, 203, 205, 206, 207, 208, 209, 220, 223, 258, 281, 348, 355, 406, 435, 471, 521, 568, 579, 612, 638, 701, 707, or any combination thereof, relative to SEQ ID NO: 1. In some embodiments, the one or more positions are selected from positions: 5, 26, 121, 198, 223, 258, 471, 579, 701, or any combination thereof, relative to SEQ ID NO: 1. In some embodiments, the engineered polypeptide comprises an enhanced nuclease activity relative to a polypeptide consisting of the amino acid sequence of SEQ ID NO: 1 as measured by a cleavage assay. In some embodiments, the engineered polypeptide comprises an enhanced binding affinity and/or binding specificity for a guide nucleic acid, target nucleic acid, or combination thereof, relative to a polypeptide consisting of the amino acid sequence of SEQ ID NO: 1 as measured by a binding assay. In some embodiments, at least one of the one or more amino acid alterations is in a region of the engineered polypeptide that interacts with a target nucleic acid, guide nucleic acid, or combination thereof. In some embodiments, the one or more amino acid alterations are one or more amino acid substitutions selected from: 12R, TSR, K15R, R18R, H20R, S21R, L26R, L26K, N30R, E33R, E34R, A35R, K37R, K38R, R41R, N43R, Q54R, Q79R, K92E, K99R, S108R, E109R, H110R, G111R, D113R, T114R, P116R, K118R, E119S, A121Q, N132R, K135R, Q138R, V139R, L149R, Y180R, L182R, Q183R, K184R, S186R, K189R, K189P, S196R, S198R, K200R, 1203R, S205R, K206R, Y207R, H208R, N209R, Y220S, S223P, E258K, K281R, K348R, N355R, N406K, K435Q, I471T, V521T, N568D, S579R, Q612R, S638K, F701R, or P707R. In some embodiments, the one or more amino acid alterations are one or more amino acid substitutions selected from: TSR, L26K, A121Q, S198R, S223P, E258K, I471T, S579R, or F701R. In some embodiments, the engineered polypeptide comprises 2, 3, 4, 5, 6, 7, 8, 9, or 10 amino acid alterations. In some embodiments, the engineered polypeptide comprises a combination of amino acid alterations as recited in TABLE 1.1. In some embodiments, the engineered polypeptide comprises a combination of amino acid alterations as recited TABLE 1.2. In some embodiments, the engineered polypeptide comprises a combination of amino acid alterations as recited TABLE 1.3. In some embodiments, the engineered polypeptide comprises an amino acid substitution at a residue corresponding to position 26 relative to SEQ ID NO: 1; in some embodiments, the amino acid substitution is selected from L26R and L26K. In some embodiments, the engineered polypeptide comprises at least one amino acid alteration that is located at a position in a RuvC domain of the engineered polypeptide. In some embodiments, the one or more amino acid alteration are at residue 369, 567, or 658 relative to SEQ ID NO: 1. In some embodiments, the one or more amino acid alterations are one or more amino acid substitutions selected from: D369A, D369N, D658A, D658N, E567A, E567Q, and a combination thereof. In some embodiments, the engineered polypeptide is fused to a fusion partner. In some embodiments, the fusion partner is selected from an exonuclease, a reverse transcriptase, a deaminase, a transcriptional activator, a transcriptional repressor, or a functional domain thereof. In some embodiments, the fusion partner is an exonuclease. In some embodiments, the engineered polypeptide is fused to a nuclear localization signal (NLS). In some embodiments, the engineered polypeptide recognizes a protospacer adjacent motif (PAM) sequence adjacent to a target sequence in a target nucleic acid, and wherein the PAM sequence comprises any one of the nucleotide sequences of TABLE 1.5. In some embodiments, compositions described herein comprise an engineered guide nucleic acid or a nucleic acid encoding an engineered guide nucleic acid. In some embodiments, the engineered guide nucleic acid comprises a first region and a second region, wherein: the first region comprises a spacer sequence that is capable of hybridizing to a target sequence in a target nucleic acid; the second region comprises a repeat sequence that is at least 90% identical to any one of the nucleotide sequences set forth in TABLE 3. In some embodiments, the spacer sequence comprises at least 10 contiguous nucleotides that are complementary to a eukaryotic sequence. In some embodiments, the composition comprises a donor nucleic acid.

Also provided herein are methods of modifying a target nucleic acid, the method comprising contacting the target nucleic acid with any of the compositions described herein. In some embodiments, methods comprise contacting a cell comprising the target nucleic acid with the composition.

Also provided herein are methods of modifying a target nucleic acid in a human subject, comprising administering any of the compositions described herein to the human subject. In some embodiments, methods comprise administering the engineered polypeptide or nucleic acid encoding the engineered polypeptide and an engineered guide nucleic acid to the human subject. In some embodiments, the engineered polypeptide or nucleic acid encoding the engineered polypeptide is administered in a first formulation and the engineered guide nucleic acid is administered in a second formulation, wherein the first formulation and the second formulation are separate. In some embodiments, the engineered polypeptide or nucleic acid encoding the engineered polypeptide and the engineered guide nucleic acid are not administered to the subject at the same time. In some embodiments, the target nucleic acid is any one of the nucleic acids set forth in TABLE 6. In some embodiments, the target nucleic acid is associated with any one of the diseases set forth in TABLE 6.1.

Also provided herein are methods of integrating a donor nucleic acid into a target nucleic acid, the method comprising contacting the target nucleic acid with any of the compositions described herein comprising a donor nucleic acid. In some embodiments, methods comprise contacting a cell comprising the target nucleic acid with the composition. In some embodiments, the one or more amino acid alteration is a substitution with an L26R, relative to SEQ ID NO: 1.

Also provided herein are cells modified by any of the compositions described herein or any of the method described herein. Also provided herein are cells comprising any of the compositions described herein. In some embodiments, the cell is a eukaryotic cell. In some embodiments, the cell is a human cell. In some embodiments, the cell is selected from an induced pluripotent stem cell (iPSC), a T cell, a hepatocyte, a cardiomyocyte, a myoblast, or a pancreatic cell.

Also provided herein are pharmaceutical compositions, comprising any of the compositions described herein, and a pharmaceutically acceptable excipient.

Also provided herein are methods of treating a disease associated with a mutation of a human gene in a subject in need thereof, the method comprising administering to the subject any of the compositions described herein, any of the cells described herein, or any of the pharmaceutical compositions described herein. In some embodiments, the gene is selected from the genes recited in TABLE 6. In some embodiments, the disease is any one of the diseases recited in TABLE 6.1. In some embodiments, the human gene is KRAS. In some embodiments, the disease is pancreatic cancer.

Also provided herein are methods of modifying a cell without resulting in or fewer translocations or chromosomal rearrangements in the cell, wherein the cell is contacted with any of the compositions described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows that variant enzymes can bind two genome loci of mammalian cells and edit the genome at the locus with varying efficacy normalized to the wild-type. The x and y-axis of the plot corresponds to various targeted loci. The identifier next to each plotted data point denotes the amino acid residue alteration and position in reference to SEQ ID NO: 1.

FIG. 2A shows indel activity of variant enzymes. The identifier under to each pbar denotes the amino acid residue alteration and position in reference to SEQ ID NO: 1.

FIG. 2B shows indel activity of variant enzymes normalized to WT (SEQ ID NO: 1). The identifier under to each pbar denotes the amino acid residue alteration and position in reference to SEQ ID NO: 1.

FIG. 3A shows indel formation by CasPhi.12 L26R Variant as compared to CasPhi.12 WT and a control in a pancreatic cell line (AsPC-1) expressing mutant KRAS (G12D).

FIG. 3B shows indel formation by CasPhi.12 L26R Variant as compared to CasPhi.12 WT and a control in a pancreatic cell line (BxPC-3) expressing wild-type KRAS (WT).

FIG. 4A shows dose-dependent indel formation by CasPhi.12 WT in pancreatic cell lines (BxPC-3) expressing mutant KRAS (G12D) or wild-type KRAS (WT), respectively.

FIG. 4B shows dose-dependent indel formation by CasPhi.12 L26R Variant in pancreatic cell lines (BxPC-3) expressing mutant KRAS (G12D) or wild-type KRAS (WT), respectively.

FIG. 4C shows dose-dependent indel formation by CasPhi.12 L26R Variant compared to CasPhi.12 WT in pancreatic cell lines (BxPC-3) expressing mutant KRAS (G12D).

FIG. 5 illustrate the effects of exonuclease fusion partners on CasPhi.12 nuclease activity for two target nucleic acids (target A and target B), in accordance with an embodiment of the present disclosure. For each fusion protein, two columns are depicted that show % indel generated at 15 ng dose (left column) and 150 ng dose (right column), respectively.

FIGS. 6A-6C show results of indel precision of wildtype CasPhi.12 protein (FIG. 6A), exo5-CasPhi.12 fusion protein (FIG. 6B) and sbcB-CasPhi.12 fusion protein (FIG. 6C) on a target nucleic acid in accordance with an embodiment of the present disclosure at a dose ratio of 1:10 (15 ng of effector protein:150 ng of guide RNA). FIGS. 6A-6C disclose SEQ ID NOS: 78-80, respectively, in order of appearance.

FIGS. 7A-7C show results of indel precision of wildtype CasPhi.12 protein (FIG. 7A), exo5-CasPhi.12 fusion protein (FIG. 7B) and sbcB-CasPhi.12 fusion protein (FIG. 7C) on a target nucleic acid in accordance with an embodiment of the present disclosure at a dose ratio of 1:10 (150 ng of effector protein:150 ng of guide RNA). FIGS. 7A-7C disclose SEQ ID NOS 81-83, respectively, in order of appearance.

FIGS. 8A-8C summarize nuclease activity of CasPhi.12 effector protein, exo5-CasPhi.12 fusion protein and sbcB-CasPhi.12 fusion protein, respectively, on target nucleic acids in accordance with an embodiment of the present disclosure. FIG. 8A shows results of nuclease activity of the CasPhi.12 effector protein at a dose ratio of 1:1 (150 ng of effector protein:150 ng of guide RNA). FIG. 8B shows results of nuclease activity of the exo5-CasPhi.12 fusion protein at a dose ratio of 1:1 (150 ng of effector protein:150 ng of guide RNA). FIG. 8C shows results of nuclease activity of the sbcB-CasPhi.12 fusion protein at a dose ratio of 1:1 (150 ng of effector protein:150 ng of guide RNA).

FIGS. 9A-9B show results of indel precision of sbcB-CasPhi.12-exo5 fusion protein (FIG. 9A) and recJ-CasPhi.12-exo5 fusion protein (FIG. 9B) on a target nucleic acid in accordance with an embodiment of the present disclosure at a dose ratio of 1:10 (15 ng of effector protein:150 ng of guide RNA). FIGS. 9A-9B disclose SEQ ID NOS: 84 and 80, respectively, in order of appearance.

FIGS. 10A-10B show results of indel precision of sbcB-CasPhi.12-exo5 fusion protein (FIG. 10A) and recJ-CasPhi.12-exo5 fusion protein (FIG. 10B) on a target nucleic acid in accordance with an embodiment of the present disclosure at a dose ratio of 1:10 (150 ng of effector protein:150 ng of guide RNA). FIGS. 10A-10B disclose SEQ ID NOS: 85 and 81, respectively, in order of appearance

FIGS. 11A-11B summarizes nuclease activity of sbcB-CasPhi.12-exo5 fusion protein and recJ-CasPhi.12-exo5 fusion protein, respectively, on target nucleic acids in accordance with an embodiment of the present disclosure. FIG. 11A shows results of nuclease activity of the sbcB-CasPhi.12-exo5 fusion protein at a dose ratio of 1:1 (150 ng of effector protein:150 ng of guide RNA). FIG. 11B shows results of nuclease activity of the recJ-CasPhi.12-exo5 fusion protein at a dose ratio of 1:1 (150 ng of effector protein:150 ng of guide RNA).

FIGS. 12A-12B shows in vivo effect of CasPhi.12 system comprising AAV8 vector encoding CasPhi.12 L26R variant and a guide RNA targeting the PCSK9 gene and serum concentration of PCSK9 protein in mice following treatment. FIG. 12A shows % indel mutations generated in the PCSK9 gene in mice liver post AAV8 vector injection. FIG. 12B shows serum PCSK9 protein concentration in mice post AAV8 vector injection.

FIG. 13 shows gel electrophoresis analysis of cis cleavage activity by CasPhi.12 and variants thereof.

FIG. 14 shows schematics of fluorescence polarization assay using a duplex substrate and a non-paired DNA substrate.

FIGS. 15A-15B show binding affinity curves for the CasPhi.12 based variant effector proteins relative to corresponding wildtype effector protein, wherein the polarization (mP) observed is plotted against concentration of the effector protein using a normal duplex (FIG. 15A) or a non-paired protospacer (FIG. 15B).

FIG. 16 shows a plateau amplitude curve for the wildtype CasPhi.12 protein and variants thereof.

FIG. 17 shows both, K_{D} and plateau polarization, values for the wildtype CasPhi.12 protein and variants thereof using normal duplex DNA substrate.

FIG. 18 shows that multiple variants, including L26R, K118R, S186R, S198R, K348R, Q612R, F701R, and S579R variants, of CasPhi.12 had more indel activity than WT CasPhi.12 (SEQ ID NO: 1).

FIG. 19 demonstrates the activity of variants with double mutations relative to that of WT CasPhi.12 (SEQ ID NO: 1). Unless otherwise indicated, the dark grey dots indicate notable variants with increased potency as described in TABLE 15.

FIG. 20 demonstrates the results of a dose titration experiment of double mutants. Variants with TSR, V139R and L26R, P707R mutations outperform the L26R Variant.

FIG. 21 demonstrates the results of variants engineered using rational design. I471T, L26K, K189P, S638K, Q54R, A121Q, E258K, Q79R, Y220S, N406K, E119S, S223P, K92E, K435Q, N568D, V521T Variants outperformed WT CasPhi.12 (SEQ ID NO: 1).

FIG. 22 shows results of an NGS analysis for CasPhi.12 L26R effector protein system relative to Cas9 effector protein system, wherein the effector protein systems were tested for their ability to generate % indels in B2M gene, TRAC gene, and CIITA gene, individually or simultaneously.

FIG. 23A shows results of an FACS analysis for CasPhi.12 L26R effector protein system relative to Cas9 effector protein system, wherein the effector protein systems were tested for their ability to knockout B2M gene expression by targeting B2M gene individually or simultaneous targeting B2M gene, TRAC gene, and CIITA gene.

FIG. 23B shows results of an FACS analysis for CasPhi.12 L26R effector protein system relative to Cas9 effector protein system, wherein the effector protein systems were tested for their ability to knockout CD3 expression by targeting TRAC gene individually or simultaneous targeting B2M gene, TRAC gene, and CIITA gene.

FIG. 23C shows results of an FACS analysis for CasPhi.12 L26R effector protein system relative to Cas9 effector protein system, wherein the effector protein systems were tested for their ability to generate % indels in CIITA gene, wherein the effector protein systems were targeting CIITA gene individually, or B2M gene, TRAC gene and CIITA gene simultaneously.

FIG. 24 shows results of translocation rates based on dGH assay for CasPhi.12 L26R effector protein system relative to Cas9 effector protein system, wherein the effector protein systems were tested for their ability to generate % indels in B2M gene, TRAC gene, and CIITA gene, individually or simultaneously.

FIG. 25 shows results of reciprocal translocations rates based on dGH assay for CasPhi.12 L26R effector protein system relative to Cas9 effector protein system, wherein the effector protein systems were tested for their ability to generate % indels in B2M gene, TRAC gene, and CIITA gene, individually or simultaneously.

FIG. 26A shows results of cell counts for CasPhi.12 L26R effector protein system edited target nucleic acids relative to Cas9 effector protein system edited target nucleic acids (B2M gene, TRAC gene, and CIITA gene, individually or simultaneously).

FIG. 26B shows results of cell viability for CasPhi.12 L26R effector protein system edited target nucleic acids relative to Cas9 effector protein system edited target nucleic acids (B2M gene, TRAC gene, and CIITA gene, individually or simultaneously).

FIG. 27 shows results of an NGS analysis for CasPhi.12 L26R effector protein system relative to Cas9 effector protein system, wherein the effector protein systems were tested for their ability to generate indels within target nucleic acids (B2M gene, TRAC gene, and CIITA gene, individually or simultaneously).

FIG. 28A shows results of an NGS analysis for CasPhi.12 L26R effector protein system relative to Cas9 effector protein system, wherein the effector protein systems were tested for their ability to generate indels within target nucleic acids (B2M gene, TRAC gene, and CIITA gene, individually or simultaneously) at 6 μg. For each effector protein, up to six columns are depicted that show % indel generated when targeting B2M gene individually or simultaneously, TRAC gene individually or simultaneously, or the CIITA gene individually or simultaneously, from left to right respectively.

FIG. 28B shows results of an NGS analysis for CasPhi.12 L26R effector protein system relative to Cas9 effector protein system, wherein the effector protein systems were tested for their ability to generate indels within target nucleic acids (B2M gene, TRAC gene, and CIITA gene, individually or simultaneously) at 9 μg. For each effector protein, up to six columns are depicted that show % indel generated when targeting B2M gene individually or simultaneously, TRAC gene individually or simultaneously, or the CIITA gene individually or simultaneously, from left to right respectively.

FIG. 29A shows results of FACS analysis for CasPhi.12 L26R effector protein system relative to Cas9 effector protein system, wherein the effector protein systems were tested for their ability to knock out target nucleic acids (B2M gene, TRAC gene, and CIITA gene, individually or simultaneously) at 6 μg. For each effector protein, up to four columns are depicted that show % indel generated when targeting B2M gene individually or simultaneously, or the TRAC gene individually or simultaneously, from left to right respectively.

FIG. 29B show results of FACS analysis for CasPhi.12 L26R effector protein system relative to Cas9 effector protein system, wherein the effector protein systems were tested for their ability to knock out target nucleic acids (B2M gene, TRAC gene, and CIITA gene, individually or simultaneously) at 9 μg. For each effector protein, up to four columns are depicted that show % indel generated when targeting B2M gene individually or simultaneously, or the TRAC gene individually or simultaneously, from left to right respectively.

FIG. 30A shows results of cell counts analysis for CasPhi.12 L26R effector protein system relative to Cas9 effector protein system, wherein the effector protein systems were tested for their ability to generate indels within target nucleic acids (B2M gene, TRAC gene, and CIITA gene, individually or simultaneously) at 6 μg.

FIG. 30B shows results of cell counts analysis for CasPhi.12 L26R effector protein system relative to Cas9 effector protein system, wherein the effector protein systems were tested for their ability to generate indels within target nucleic acids (B2M gene, TRAC gene, and CIITA gene, individually or simultaneously) at 9 μg.

FIG. 31A shows results of cell viability analysis for CasPhi.12 L26R effector protein system relative to Cas9 effector protein system, wherein the effector protein systems were tested for their ability to generate indels within target nucleic acids (B2M gene, TRAC gene, and CIITA gene, individually or simultaneously) at 6 μg.

FIG. 31B shows results of cell viability analysis for CasPhi.12 L26R effector protein system relative to Cas9 effector protein system, wherein the effector protein systems were tested for their ability to generate indels within target nucleic acids (B2M gene, TRAC gene, and CIITA gene, individually or simultaneously) at 9 μg.

FIG. 32A shows indel activity of the effector protein system (e.g., CasPhi.12 L26R) within or adjacent to intron 1 of human albumin gene as related to different concentrations of RNA and MOI and as compared to positive control (e.g., SpyCas9). For each plasmid construct, up to nine columns are depicted that show % indel generated at 25 ng RNA and 2.5e3 MOI dose, 25 ng RNA and 1e4 MOI dose, 25 ng RNA and 4e4 MOI dose, 100 ng RNA and 2.5e3 MOI dose, 100 ng RNA and 1e4 MOI dose, 100 ng RNA and 4e4 MOI dose, 400 ng RNA and 2.5e3 MOI dose, 400 ng RNA and 1e4 MOI dose, 400 ng RNA and 4e4 MOI dose from left to right respectively.

FIG. 32B shows relative light units (RLU) as a measure of integration activity of the effector protein system (e.g., CasPhi.12 L26R) within or adjacent to intron 1 of human albumin gene as related to different concentrations of RNA and MOI and as compared to positive control (e.g., SpyCas9). For each plasmid construct, up to nine columns are depicted that show relative light units (RLU) as a measure of integration activity of the effector protein system at 25 ng RNA and 2.5e3 MOI dose, 25 ng RNA and 1e4 MOI dose, 25 ng RNA and 4e4 MOI dose, 100 ng RNA and 2.5e3 MOI dose, 100 ng RNA and 1e4 MOI dose, 100 ng RNA and 4e4 MOI dose, 400 ng RNA and 2.5e3 MOI dose, 400 ng RNA and 1e4 MOI dose, 400 ng RNA and 4e4 MOI dose from left to right respectively.

FIG. 33 shows % integration products as a measure of integration activity of the effector protein system (e.g., CasPhi.12 L26R) within or adjacent to intron 1 of human albumin gene as compared to positive control (e.g., SpyCas9) as measured via reverse transcription droplet digital PCR (RT-ddPCR). For each plasmid constructs, two columns are depicted that show two different human donors of the primary hepatocytes.

### DETAILED DESCRIPTION OF THE INVENTION

It is to be understood that both the foregoing general description and the following detailed description are exemplary, and explanatory only, and are not restrictive of the disclosure.

The section headings used herein are for organizational purposes only and are not to be construed as limiting the subject matter described.

### Definitions

Unless otherwise indicated, all technical terms used herein have the same meaning as commonly understood by one of ordinary skill in the art to which this invention belongs. Unless otherwise indicated or obvious from context, the following terms have the following meanings:

As used in this specification and the appended claims, the singular forms “a,” “an,” and “the” include plural references unless the context clearly dictates otherwise.

Any reference to “or” herein is intended to encompass “and/or” unless otherwise stated. As used herein, the term “and/or” includes any and all combinations of one or more of the associated listed items.

Use of the term “including” as well as other forms, such as “includes” and “included,” is not limiting.

As used herein, the term, “comprise” and its grammatical equivalents, specifies the presence of stated features, integers, steps, operations, elements, and/or components, but does not preclude the presence or addition of one or more other features, integers, steps, operations, elements, components, and/or groups thereof.

As used herein, the term, “about,” in reference to a number or range of numbers, is understood to mean the stated number and numbers +/−10% thereof, or 10% below the lower listed limit and 10% above the higher listed limit for the values listed for a range.

The terms, “percent identity,” “% identity,” and % “identical,” or grammatical equivalents thereof, as used herein, refer to the extent to which two sequences (nucleotide or amino acid) have the same residue at the same positions in an alignment. For example, “an amino acid sequence is X % identical to SEQ ID NO: Y” can refer to % identity of the amino acid sequence to SEQ ID NO: Y and is elaborated as X % of residues in the amino acid sequence are identical to the residues of sequence disclosed in SEQ ID NO: Y. Generally, computer programs can be employed for such calculations. Illustrative programs that compare and align pairs of sequences, include ALIGN (Myers and Miller, Comput Appl Biosci. 1988 Mar. 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12 (1 Pt 1):387-95).

The term, “% similarity,” as used herein, in the context of an amino acid sequence, refers to a value that is calculated by dividing a similarity score by the length of the alignment. The similarity of two amino acid sequences can be calculated by using a BLOSUM62 similarity matrix (Henikoff and Henikoff, Proc. Natl. Acad. Sci. USA., 89:10915-10919 (1992)) that is transformed so that any value ≥1 is replaced with +1 and any value ≤0 is replaced with 0. For example, an Ile (I) to Leu (L) substitution is scored at +2.0 by the BLOSUM62 similarity matrix, which in the transformed matrix is scored at +1. This transformation allows the calculation of percent similarity, rather than a similarity score. Alternately, when comparing two full protein sequences, the proteins can be aligned using pairwise MUSCLE alignment. Then, the % similarity can be scored at each residue and divided by the length of the alignment. For determining % similarity over a protein domain or motif, a multilevel consensus sequence (or PROSITE motif sequence) can be used to identify how strongly each domain or motif is conserved. In calculating the similarity of a domain or motif, the second and third levels of the multilevel sequence are treated as equivalent to the top level. Additionally, if a substitution could be treated as conservative with any of the amino acids in that position of the multilevel consensus sequence, +1 point is assigned. For example, given the multilevel consensus sequence: RLG and YCK, the test sequence QIQ would receive three points. This is because in the transformed BLOSUM62 matrix, each combination is scored as: Q-R: +1; Q-Y: +0; I-L: +1; I-C: +0; Q-G: +0; Q-K: +1. For each position, the highest score is used when calculating similarity. The % similarity can also be calculated using commercially available programs, such as the Geneious Prime software given the parameters matrix=BLOSUM62 and threshold ≥1.

The term, “amplification,” “amplifying,” or grammatical equivalents thereof, as used herein, refers to a process by which a nucleic acid molecule is enzymatically copied to generate a plurality of nucleic acid molecules containing the same sequence as the original nucleic acid molecule or a distinguishable portion thereof.

The term, “base editing enzyme,” as used herein, refers to a protein, polypeptide, or fragment thereof that is capable of catalyzing the chemical modification of a nucleobase of a deoxyribonucleotide or a ribonucleotide. Such a base editing enzyme, for example, is capable of catalyzing a reaction that modifies a nucleobase that is present in a nucleic acid molecule, such as DNA or RNA (single stranded or double stranded). Non-limiting examples of the type of modification that a base editing enzyme is capable of catalyzing includes converting an existing nucleobase to a different nucleobase, such as converting a cytosine to a guanine or thymine or converting an adenine to a guanine, hydrolytic deamination of an adenine or adenosine, or methylation of cytosine (e.g., CpG, CpA, CpT or CpC). A base editing enzyme itself may or may not bind to the nucleic acid molecule containing the nucleobase.

The term, “base editor,” as used herein, refers to a fusion protein comprising a base editing enzyme fused to an effector protein. The base editor is functional when the effector protein is coupled to a guide nucleic acid. The guide nucleic acid imparts sequence specific activity to the base editor. By way of non-limiting example, the effector protein may comprise a catalytically inactive effector protein. Also, by way of non-limiting example, the base editing enzyme may comprise deaminase activity. Additional base editors are described herein.

The term, “catalytically inactive effector protein,” as used herein, refers to an effector protein that is modified relative to a naturally-occurring effector protein to have a reduced or eliminated catalytic activity relative to that of the naturally-occurring effector protein, but retains its ability to interact with a guide nucleic acid. The catalytic activity that is reduced or eliminated is often a nuclease activity. The naturally-occurring effector protein may be a wildtype protein. In some embodiments, the catalytically inactive effector protein is referred to as a catalytically inactive variant of an effector protein, e.g., a Cas effector protein.

The term, “cis cleavage,” as used herein, refers to cleavage (hydrolysis of a phosphodiester bond) of a target nucleic acid by an effector protein complexed with a guide nucleic acid refers to cleavage of a target nucleic acid that is hybridized to a guide nucleic acid, wherein cleavage occurs within or directly adjacent to the region of the target nucleic acid that is hybridized to the guide nucleic acid.

The terms, “complementary” and complementarity,” as used herein with reference to a nucleic acid molecule or nucleotide sequence, refer to the characteristic of a polynucleotide having nucleotides that base pair with their Watson-Crick counterparts (C with G; or A with T) in a reference nucleic acid. For example, when every nucleotide in a polynucleotide forms a base pair with a reference nucleic acid, that polynucleotide is said to be 100% complementary to the reference nucleic acid. In a double stranded DNA or RNA sequence, the upper (sense) strand sequence is in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the ‘reverse complement’ sequence or the ‘reverse complementary’ sequence is understood as the sequence of the lower strand in the direction of its 5′- to its 3′-end. Each nucleotide in a double stranded DNA or RNA molecule that is paired with its Watson-Crick counterpart called its complementary nucleotide.

The term, “cleavage assay,” as used herein, refers to an assay designed to visualize, quantitate, or identify cleavage of a nucleic acid. In some cases, the cleavage activity may be cis cleavage activity. In some cases, the cleavage activity may be trans cleavage activity.

The term, “clustered regularly interspaced short palindromic repeats (CRISPR),” as used herein, refers to a segment of DNA found in the genomes of certain prokaryotic organisms, including some bacteria and archaea, that includes repeated short sequences of nucleotides interspersed at regular intervals between unique sequences of nucleotides derived from the DNA of a pathogen (e.g., virus) that had previously infected the organism and that functions to protect the organism against future infections by the same pathogen.

The term, “CRISPR RNA” or “crRNA,” as used herein, refers to a type of guide nucleic acid, wherein the nucleic acid is RNA, comprising a first sequence, often referred to herein as a “spacer sequence,” that hybridizes to a target sequence of a target nucleic acid, and a second sequence, often referred to herein as a “repeat sequence,” that is capable of connecting a crRNA to an effector protein by being non-covalently bound by an effector protein.

The term, “detectable signal,” as used herein, refers to a signal that can be detected using optical, fluorescent, chemiluminescent, electrochemical, and other detection methods known in the art.

The term “donor nucleic acid,” as used herein refers to nucleic acid that is incorporated into a target nucleic acid.

The term, “donor nucleotide,” as used herein, refers to a single nucleotide that is incorporated into a target nucleic acid. A nucleotide is typically inserted at a site of cleavage by an effector protein.

The term, “effector protein,” as used herein, refers to a protein, polypeptide, or peptide that non-covalently binds to a guide nucleic acid to form a complex that contacts a target nucleic acid, wherein at least a portion of the guide nucleic acid hybridizes to a target sequence of the target nucleic acid. In some embodiments, the complex between an effector protein and a guide nucleic acid can include multiple effector proteins or a single effector protein. In some embodiments, the effector protein modifies the target nucleic acid when the complex contacts the target nucleic acid. In some embodiments, the effector protein does not modify the target nucleic acid, but it is fused to a fusion partner protein that modifies the target nucleic acid when the complex contacts the target nucleic acid. A non-limiting example of modifying a target nucleic acid is cleaving (hydrolysis) of a phosphodiester bond of the target nucleic acid. Additional examples of modifying target nucleic acids are described herein and throughout.

The term, “functional domain,” as used herein, refers to a region of one or more amino acids in a protein that is required for an activity of the protein, or the full extent of that activity, as measured in an in vitro assay. Activities include, but are not limited to nucleic acid binding, nucleic acid modification, nucleic acid cleavage, protein binding. The absence of the functional domain, including mutations of the functional domain, would abolish or reduce activity.

The term, “functional fragment,” as used herein, refers to a fragment of a protein that retains some function relative to the entire protein. Non-limiting examples of functions are nucleic acid binding, protein binding, nuclease activity, nickase activity, deaminase activity, demethylase activity, or acetylation activity.

The terms, “fusion effector protein,” “fusion protein,” and “fusion polypeptide,” as used herein, refer to a protein comprising at least two heterologous polypeptides. Often a fusion effector protein comprises an effector protein and a fusion partner protein. In general, the fusion partner protein is not an effector protein. Examples of fusion partner proteins are provided herein.

The term, “fusion partner protein” or “fusion partner,” as used herein, refers to a protein, polypeptide or peptide that is fused to an effector protein. The fusion partner generally imparts some function to the fusion protein that is not provided by the effector protein. The fusion partner may provide a detectable signal. The fusion partner may modify a target nucleic acid, including changing a nucleobase of the target nucleic acid and making a chemical modification to one or more nucleotides of the target nucleic acid. The fusion partner may be capable of modulating the expression of a target nucleic acid. The fusion partner may inhibit, reduce, activate, or increase expression of a target nucleic acid via additional proteins or nucleic acid modifications to the target sequence.

“Gene therapy”, as used herein, comprises use of a recombinant nucleic acid (DNA or RNA), administered for the purpose to adjust, repair, replace, add, or remove a gene sequence.

A “genetic disease”, as used herein, refers to a disease, disorder, condition, or syndrome caused by one or more mutations in the DNA of an organism. Mutations can be due to several different cellular mechanisms, including, but not limited to, an error in DNA replication, recombination, or repair, or due to environmental factors. A genetic disease comprises, in some embodiments, a single gene disorder, a chromosome disorder, or a multifactorial disorder.

The term, “guide nucleic acid,” as used herein, refers to at least one nucleic acid comprising: a first nucleotide sequence that hybridizes to a target nucleic acid; and a second nucleotide sequence that is capable of connecting an effector protein to the nucleic acid by being non-covalently bound by an effector protein. The first sequence may be referred to herein as a spacer sequence. In some embodiments, the first sequence is covalently linked to the second sequence, either directly (e.g., by a phosphodiester bond) or indirectly (e.g., by one more nucleotides). In some embodiments, the first sequence is located 5′ of the second nucleotide sequence. In some embodiments, the first sequence is located 3′ of the second nucleotide sequence.

The term, “heterologous,” as used herein, means a nucleotide or polypeptide sequence that is not found in a native nucleic acid or protein, respectively. In some embodiments, fusion proteins comprise an effector protein and a fusion partner protein, wherein the fusion partner protein is heterologous to an effector protein. These fusion proteins may be referred to as a “heterologous protein.” A protein that is heterologous to the effector protein is a protein that is not covalently linked via an amide bond to the effector protein in nature. In some embodiments, a heterologous protein is not encoded by a species that encodes the effector protein. In some embodiments, the heterologous protein exhibits an activity (e.g., enzymatic activity) when it is fused to the effector protein. In some embodiments, the heterologous protein exhibits increased or reduced activity (e.g., enzymatic activity) when it is fused to the effector protein, relative to when it is not fused to the effector protein. In some embodiments, the heterologous protein exhibits an activity (e.g., enzymatic activity) that it does not exhibit when it is fused to the effector protein. A guide nucleic acid may comprise a first sequence and a second sequence, wherein the first sequence and the second sequence are not found covalently linked via a phosphodiester bond in nature. Thus, the first sequence is considered to be heterologous with the second sequence, and the guide nucleic acid may be referred to as a heterologous guide nucleic acid.

The term, “in vitro,” as used herein, is used to describe an event that takes places contained in a container for holding laboratory reagents such that it is separated from the biological source from which the material is obtained. In vitro assays can encompass cell-based assays in which living or dead cells are employed. In vitro assays can also encompass a cell-free assay in which no intact cells are employed. The term, “in vivo,” is used to describe an event that takes place in a subject's body. The term, “ex vivo,” is used to describe an event that takes place outside of a subject's body. An ex vivo assay is not performed on a subject. Rather, it is performed upon a sample separate from a subject. An example of an ex vivo assay performed on a sample is an “in vitro” assay.

The term, “linked amino acids,” as used herein, refers to at least two amino acids linked by an amide bond.

The term, “linker,” as used herein, refers to a bond or molecule that links a first polypeptide to a second polypeptide or a first nucleic acid to a second nucleic acid. A “peptide linker” comprises at least two amino acids linked by an amide bond.

The term, “modified target nucleic acid,” as used herein, refers to a target nucleic acid, wherein the target nucleic acid has undergone a modification, for example, after contact with an effector protein. In some cases, the modification is an alteration in the sequence of the target nucleic acid. In some cases, the modified target nucleic acid comprises an insertion, deletion, or replacement of one or more nucleotides compared to the unmodified target nucleic acid.

The term, “mutation associated with a disease,” as used herein, refers to the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation.

The terms, “non-naturally occurring” and “engineered,” as used herein, are used interchangeably and indicate the involvement of the hand of man. The terms, when referring to a nucleic acid, nucleotide, protein, polypeptide, peptide or amino acid, refer to a nucleic acid, nucleotide, protein, polypeptide, peptide or amino acid that is at least substantially free from at least one other feature with which it is naturally associated in nature and as found in nature, and/or contains a modification (e.g., chemical modification, nucleotide sequence, or amino acid sequence) that is not present in the naturally occurring nucleic acid, nucleotide, protein, polypeptide, peptide, or amino acid. The terms, when referring to a composition or system described herein, refer to a composition or system having at least one component that is not naturally associated with the other components of the composition or system. By way of a non-limiting example, a composition may include an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by the hand of man.

The term, “nucleic acid expression vector,” as used herein, refers to a plasmid that can be used to express a nucleic acid of interest.

The term, “nuclear localization signal,” as used herein, refers to an entity (e.g., peptide) that facilitates localization of a nucleic acid, protein, or small molecule to the nucleus, when present in a cell that contains a nuclear compartment.

The term, “nuclease activity,” as used herein, refers to the enzymatic activity of an enzyme which allows the enzyme to cleave the phosphodiester bonds between the nucleotide subunits of nucleic acids; the term, “endonuclease activity,” refers to the enzymatic activity of an enzyme which allows the enzyme to cleave the phosphodiester bond within a polynucleotide chain. An enzyme with nuclease activity may be referred to as a “nuclease.”

The terms, “nucleotide” and “nucleoside,” when used in the context of a nucleic acid molecule having multiple residues, are used interchangeably and mean the sugar and base of the residue contained in the nucleic acid molecule. The term, “nucleobase,” when used in the context of a nucleic acid molecule, can refer to the base of the residue contained in the nucleic acid molecule, for example, the base of a nucleotide or a nucleoside.

The term, “prime editing enzyme,” as used herein, refers to a protein, polypeptide, or fragment thereof that is capable of catalyzing the modification (insertion, deletion, or base-to-base conversion) of a target nucleotide or nucleotide sequence in a nucleic acid. A prime editing enzyme capable of catalyzing such a reaction includes a reverse transcriptase. A prime editing enzyme may require a prime editing guide RNA (pegRNA) to catalyze the modification. Such a pegRNA can be capable of identifying the nucleotide or nucleotide sequence in the target nucleic acid to be edited and encoding the new genetic information that replaces the targeted nucleotide or nucleotide sequence in the nucleic acid. A prime editing enzyme may require a prime editing guide RNA (pegRNA) and a single guide RNA to catalyze the modification.

The term, “protospacer adjacent motif (PAM),” as used herein, refers to a nucleotide sequence found in a target nucleic acid that directs an effector protein to modify the target nucleic acid at a specific location. A PAM sequence may be required for a complex having an effector protein and a guide nucleic acid to hybridize to and modify the target nucleic acid. However, a given effector protein may not require a PAM sequence being present in a target nucleic acid for the effector protein to modify the target nucleic acid.

The term, “recombinant,” as used herein, as applied to proteins, polypeptides, peptides, and nucleic acids, refers to proteins, polypeptides, peptides and nucleic acids that are products of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions and may act to modulate production of a desired product by various mechanisms (see “DNA regulatory sequences”, below).

The term, “recombinant” polynucleotide or “recombinant” nucleic acid, refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. The term, “recombinant polypeptide,” or “recombinant protein,” refers to a polypeptide which is not naturally occurring, e.g., is made by the artificial combination of two otherwise separated segments of amino sequences through human intervention. Thus, e.g., a polypeptide that comprises a heterologous amino acid sequence is a recombinant polypeptide.

The terms, “reporter,” “reporter nucleic acid,” and “reporter molecule,” are used interchangeably herein to refer to a non-target nucleic acid molecule that can provide a detectable signal upon cleavage by an effector protein. Examples of detectable signals and detectable moieties that generate detectable signals are provided herein.

The term, “sample,” as used herein, generally refers to something comprising a target nucleic acid. In some embodiments, the sample is a biological sample, such as a biological fluid or tissue sample. In some embodiments, the sample is an environmental sample. The sample may be a biological sample or environmental sample that is modified or manipulated. By way of non-limiting example, samples may be modified or manipulated with purification techniques, heat, nucleic acid amplification, salts, and buffers.

The term, “subject,” as used herein, can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some embodiments, the subject is not necessarily diagnosed or suspected of being at high risk for the disease.

A “syndrome”, as used herein, refers to a group of symptoms which, taken together, characterize a condition.

The term, “target nucleic acid,” as used herein, refers to a nucleic acid that is selected as the nucleic acid for modification, binding, hybridization, or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA).

The term, “target sequence,” as used herein, when used in reference to a target nucleic acid, refers to a sequence of nucleotides that hybridizes to an equal length portion of a guide nucleic acid. Hybridization of the guide nucleic acid to the target sequence may bring an effector protein into contact with the target nucleic acid.

The term, “trans cleavage,” is used herein in reference to cleavage (hydrolysis of a phosphodiester bond) of one or more nucleic acids by an effector protein that is complexed with a guide nucleic acid and a target nucleic acid. The one or more nucleic acids may include the target nucleic acid as well as non-target nucleic acids.

The term, “trans-activating RNA (tracrRNA),” as used herein, refers to a nucleic acid that comprises a first sequence that is capable of being non-covalently bound by an effector protein. TracrRNAs may comprise a second sequence that hybridizes to a portion of a crRNA, which may be referred to as a repeat sequence. In some embodiments, tracrRNAs are covalently linked to a crRNA.

The term, “transcriptional activator,” as used herein, refers to a polypeptide or a fragment thereof that can activate or increase transcription of a target nucleic acid molecule.

The term, “transcriptional repressor,” as used herein, refers to a polypeptide or a fragment thereof that is capable of arresting, preventing, or reducing transcription of a target nucleic acid.

The terms, “treatment” and “treating,” as used herein, are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term, “viral vector,” as used herein, refers to a nucleic acid to be delivered into a host cell via a recombinantly produced virus or viral particle. The nucleic acid may be single-stranded or double stranded, linear or circular, segmented or non-segmented. The nucleic acid may comprise DNA, RNA, or a combination thereof. Non-limiting examples of viruses or viral particles that can deliver a viral vector include retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. A viral vector delivered by such viruses or viral particles may be referred to by the type of virus to deliver the viral vector (e.g., an AAV viral vector is a viral vector that is to be delivered by an adeno-associated virus). A viral vector referred to by the type of virus to be delivered by the viral vector can contain viral elements (e.g., nucleotide sequences) necessary for packaging of the viral vector into the virus or viral particle, replicating the virus, or other desired viral activities. A virus containing a viral vector may be replication competent, replication deficient or replication defective.

### I. Introduction

Disclosed herein are compositions, systems and methods comprising:
a) a polypeptide or a nucleic acid encoding the polypeptide; and
b) a guide nucleic acid or a nucleic acid encoding the guide nucleic acid.

Further described herein are polypeptides that can bind and, optionally, cleave nucleic acids in a sequence-specific manner. Such a polypeptide can bind a target region of a target nucleic acid and cleave the target nucleic acid within the target region or at a position adjacent to the target region. In some embodiments, polypeptide can be activated when it binds a target region of a target nucleic acid to cleave regions of the nucleic acid that are near, but not adjacent to the target region. A polypeptide can be an effector protein, such as a CRISPR-associated (Cas) protein, which may be coupled to a guide nucleic acid that imparts activity or sequence selectivity to the polypeptide. An effector protein may also be referred to as a programmable nuclease because the nuclease activity of the protein may be directed to different target nucleic acids by way of revising the guide nucleic acid that the protein binds.

In some embodiments, compositions, systems, and methods described herein comprising a guide nucleic acid (also referred to herein as a “guide RNA”) comprising a second region or sequence that is similar to identical to a repeat sequence. In some embodiments, compositions, systems, and methods comprising guide nucleic acids comprise a first region or sequence that is partially complementary to a target nucleic acid and which may be referred to as a spacer sequence. In general, guide nucleic acids comprise a CRISPR RNA (crRNA) that is at least partially complementary to a target nucleic acid.

Polypeptides disclosed herein may bind and/or cleave nucleic acids, including double stranded RNA (dsRNA), single stranded RNA (ssRNA), double stranded DNA (dsDNA), and single stranded DNA (ssDNA). Polypeptides disclosed herein may provide binding activity, cis cleavage activity, trans cleavage activity, nickase activity, nuclease activity, or a combination thereof. Cis cleavage activity is cleavage of a target nucleic acid that is hybridized to a guide RNA (crRNA), wherein cleavage occurs within or directly adjacent to the region of the target nucleic acid that is hybridized to guide RNA. Trans cleavage activity is cleavage of ssDNA or ssRNA that is near, but not hybridized to the guide RNA. Trans cleavage activity is triggered by the hybridization of guide RNA to the target nucleic acid. Nickase activity is the selective cleavage of one strand of a dsDNA molecule. In some embodiments, when describing cleavage of a nucleic acid molecule or nuclease activity of an effector protein, reference is made to the hydrolysis of a phosphodiester bond of a nucleic acid molecule that results in breakage of that bond. The result of this breakage can be a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the effector protein.

Programmable CRISPR-associated (Cas) nucleases, through their ability to cleave DNA at a precise target location in the genome of a wide variety of cells and organisms, allow for precise and efficient editing of DNA sequences of interest. SSBs and DSBs are an effective way to disrupt a gene of interest, generate DNA or RNA modifications, and to treat genetic disease through gene correction.

Disclosed herein are non-naturally occurring compositions, systems and methods comprising at least one of an engineered polypeptide or effector protein and an engineered guide nucleic acid, which may simply be referred to herein as a polypeptide or effector protein and a guide nucleic acid, respectively. A polypeptide or effector protein described herein may be an engineered or isolated polypeptide or protein. In some embodiments, compositions, systems and methods described herein comprise an engineered protein or a use thereof. In some embodiments, composition, systems, and methods described herein comprise an isolated polypeptide or use thereof. In general, an effector protein and a guide nucleic acid refer to an effector protein and a guide nucleic acid, respectively, that are not found in nature. In some embodiments, compositions, methods and systems described herein comprise at least one non-naturally occurring component. For example, disclosed compositions, methods and systems may comprise a guide nucleic acid, wherein the sequence of the guide nucleic acid is different or modified from that of a naturally-occurring guide nucleic acid. In some embodiments, disclosed compositions, systems and methods comprise at least two components that do not naturally occur together. For example, disclosed compositions, methods and systems may comprise a guide nucleic acid comprising a repeat sequence or a second region, at least a portion of which interacts with a polypeptide, and a spacer sequence, or a first region, at least a portion of which is at least partially complementary to a target sequence in a target nucleic acid, the regions of which do not naturally occur together. Also, by way of example, disclosed compositions, methods and systems may comprise a guide nucleic acid and an effector protein that do not naturally occur together and/or are heterologous to each other. Likewise, and by way of non-limiting example, disclosed compositions, systems and methods may comprise a ribonucleotide protein complex (RNP) comprising an effector protein and a guide nucleic acid that do not occur together in nature. Conversely, and for clarity, an effector protein or guide nucleic acid that is “natural,” “naturally-occurring,” or “found in nature” includes effector proteins and guide nucleic acids from cells or organisms that have not been genetically modified by a human or machine.

In some embodiments, the guide nucleic acid comprises a non-natural nucleotide sequence. In some embodiments, the non-natural nucleotide sequence is a nucleotide sequence that is not found in nature. The non-natural nucleotide sequence may comprise a portion of a naturally-occurring sequence, wherein the portion of the naturally-occurring sequence is not present in nature absent the remainder of the naturally-occurring sequence. In some embodiments, the guide nucleic acid comprises two naturally-occurring sequences arranged in an order or proximity that is not observed in nature. In some embodiments, compositions and systems comprise a ribonucleotide complex comprising an effector protein and a guide nucleic acid that do not occur together in nature. In some embodiments, compositions, systems, and methods described herein comprise at least two components that do not occur together in nature, wherein the at least two components comprise at least one of an effector protein, an effector partner (e.g., fusion partner), and a guide nucleic acid. Engineered guide nucleic acids may comprise a first sequence and a second sequence that do not occur naturally together. For example, a guide nucleic acid may comprise a sequence of a naturally-occurring repeat sequence and a spacer sequence that is complementary to a naturally-occurring eukaryotic sequence. The guide nucleic acid may comprise a sequence of a repeat sequence that occurs naturally in an organism and a spacer sequence that does not occur naturally in that organism. A guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. In some embodiments, the guide nucleic acid comprises two heterologous sequences arranged in an order or proximity that is not observed in nature. Therefore, compositions and systems described herein are not naturally occurring.

In some embodiments, compositions, methods and systems described herein comprise a polypeptide (e.g., an effector protein, an effector partner such as a fusion partner, a fusion protein, or a combination thereof) that is similar to a naturally occurring polypeptide. The polypeptide (e.g., the effector protein) may lack a portion of the naturally occurring polypeptide. The polypeptide (e.g., the effector protein) may comprise an alteration relative to the naturally-occurring polypeptide, wherein the alteration is not found in nature. The polypeptide (e.g., the effector protein) may also comprise at least one additional amino acid relative to the naturally-occurring polypeptide. For example, the polypeptide (e.g., the effector protein) may comprise an addition of a nuclear localization signal relative to the natural occurring polypeptide. In certain embodiments, the nucleotide sequence encoding the polypeptide (e.g., the effector protein) is codon optimized (e.g., for expression in a eukaryotic cell) relative to the naturally occurring sequence.

### II. Polypeptide Systems

Provided herein are compositions, systems and methods comprising a polypeptide or polypeptide system, wherein the polypeptide or polypeptide system described herein comprises one or more effector proteins or variants thereof, one or more effector partners such as fusion partners or variants thereof, one or more linkers for peptides, or combinations thereof. Unless otherwise indicated, reference to effector proteins throughout the disclosure includes variant effector proteins, and vice versa.

In some embodiments, when describing a polypeptide or peptide, reference is made to a polymeric form of amino acids. A polypeptide may include coded and non-coded amino acids, chemically or biochemically modified or derivatized amino acids, and polypeptides having modified peptide backbones. Accordingly, polypeptides as described herein may comprise one or more mutations, one or more engineered modifications, or both. It is understood that when describing coding sequences of polypeptides described herein, said coding sequences do not necessarily require a codon encoding an N-terminal Methionine (M) or a Valine (V) as described for the effector proteins described herein. One skilled in the art would understand that a start codon could be replaced or substituted with a start codon that encodes for an amino acid residue sufficient for initiating translation in a host cell. In some instances, when a heterologous peptide, such as an effector partner (e.g., fusion partner), protein tag or NLS, is located at the N terminus of the effector protein, a start codon for the heterologous peptide serves as a start codon for the effector protein as well. Thus, the natural start codon encoding an amino acid residue sufficient for initiating translation (e.g., Methionine (M) or a Valine (V)) of the effector protein may be removed or absent.

### Effector Proteins

Provided herein, are effector proteins and in certain embodiments, are compositions, systems and methods that comprise one or more effector proteins or a use thereof.

An effector protein provided herein interacts with a guide nucleic acid to form a complex (i.e., an RNP). In some embodiments, when describing an RNP (i.e., a ribonucleotide protein complex), reference is made to a complex of one or more nucleic acids and one or more polypeptides described herein. While the term utilizes “ribonucleotides” it is understood that the one or more nucleic acid may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

In some embodiments, the complex interacts with a target nucleic acid. In some embodiments, when describing binding or interacting, and grammatical equivalents thereof, reference is made to a non-covalent interaction between macromolecules (e.g., between two polypeptides, between a polypeptide and a nucleic acid; between a polypeptide/guide nucleic acid complex and a target nucleic acid; and the like). While in a state of noncovalent interaction, the macromolecules are said to be associated or interacting or binding (e.g., when a molecule X is said to interact with a molecule Y, it is meant the molecule X binds to molecule Y in a non-covalent manner). Non-limiting examples of non-covalent interactions are ionic bonds, hydrogen bonds, van der Waals and hydrophobic interactions. Not all components of a binding interaction need be sequence-specific (e.g., contacts with phosphate residues in a DNA backbone), but some portions of a binding interaction may be sequence-specific.

In some embodiments, an interaction between the complex and a target nucleic acid comprises one or more of: recognition of a protospacer adjacent motif (PAM) sequence within the target nucleic acid by the effector protein, hybridization of the guide nucleic acid to the target nucleic acid, modification of the target nucleic acid by the effector protein, and any combinations thereof. In some embodiments, recognition of a PAM sequence within a target nucleic acid may direct the modification activity of an effector protein. In some embodiments, recognition of a PAM sequence adjacent to a target sequence of a target nucleic acid may direct the modification activity of an effector protein.

Modification activity of an effector protein or an engineered protein described herein may be cleavage activity, binding activity, insertion activity, substitution activity, and the like. Modification activity of an effector protein may result in: cleavage of at least one strand of a target nucleic acid, deletion of one or more nucleotides of a target nucleic acid, insertion of one or more nucleotides into a target nucleic acid, substitution of one or more nucleotides of a target nucleic acid with an alternative nucleotide, more than one of the foregoing, and any combinations thereof. In some embodiments, modification of a target nucleic acid comprises introducing or removing epigenetic modification(s). In some embodiments, an ability of an effector protein to edit a target nucleic acid may depend upon the effector protein being complexed with a guide nucleic acid, the guide nucleic acid being hybridized to a target sequence of the target nucleic acid, the distance between the target sequence and a PAM sequence, or combinations thereof. A target nucleic acid comprises a target strand and a non-target strand. Accordingly, in some embodiments, the effector protein may edit a target strand and/or a non-target strand of a target nucleic acid.

An effector protein may be brought into proximity of a target nucleic acid in the presence of a guide nucleic acid when the guide nucleic acid includes a nucleotide sequence that is complementary with a target sequence in the target nucleic acid. The ability of an effector protein to modify a target nucleic acid may be dependent upon the effector protein being bound to a guide nucleic acid and the guide nucleic acid being hybridized to a target nucleic acid. An effector protein may also recognize a protospacer adjacent motif (PAM) sequence present in the target nucleic acid, which may direct the modification activity of the effector protein. An effector protein may modify a nucleic acid by cis cleavage or trans cleavage.

In some embodiments, effector proteins disclosed herein may provide cleavage activity, such as cis cleavage activity, trans cleavage activity, nickase activity, nuclease activity, or a combination thereof. In general, effector proteins described herein edit a target nucleic acid by cis cleavage activity on the target nucleic acid. Effector proteins disclosed herein may cleave nucleic acids, including single stranded RNA (ssRNA), double stranded DNA (dsDNA), and single-stranded DNA (ssDNA).

The modification of the target nucleic acid generated by an effector protein may, as a non-limiting example, result in modulation of the expression of the nucleic acid (e.g., increasing or decreasing expression of the nucleic acid) or modulation of the activity of a translation product of the target nucleic acid (e.g., inactivation of a protein binding to an RNA molecule or hybridization). Accordingly, in some embodiments, provided herein are methods of editing a target nucleic acid using an effector protein of the present disclosure, or compositions or systems thereof. Also provided herein are methods of modulating expression of a target nucleic acid using an effector protein of the present disclosure, or compositions or systems thereof. Further provided herein are methods of modulating the activity of a translation product of a target nucleic acid using an effector protein of the present disclosure, or compositions or systems thereof.

In some embodiments, effector proteins disclosed herein may provide catalytic activity (e.g., cleavage activity, nickase activity, nuclease activity, other activity, or combinations thereof) similar to that of a naturally-occurring effector protein, such as, for example, a naturally-occurring effector protein with reduced cleavage activity (e.g., Cas 14) including cis cleavage activity, trans cleavage activity, or combinations thereof. In some embodiments, effector proteins disclosed herein may be fused to effector partners (e.g., fusion partners) or fusion proteins wherein the effector partners (e.g., fusion partners) or fusion proteins are capable of some function or activity not provided by an effector protein.

An effector protein may be a CRISPR-associated (“Cas”) protein. An effector protein may function as a single protein, including a single protein that is capable of binding to a guide nucleic acid and modifying a target nucleic acid. Alternatively, an effector protein may function as part of a multiprotein complex, including, for example, a complex having two or more effector proteins, including two or more of the same effector proteins (e.g., dimer or multimer). An effector protein, when functioning in a multiprotein complex, may have only one functional activity (e.g., binding to a guide nucleic acid), while other effector proteins present in the multiprotein complex are capable of the other functional activity (e.g., modifying a target nucleic acid). In some embodiments, effector proteins disclosed herein may provide catalytic activity (e.g., cleavage activity, nickase activity, nuclease activity, other activity, or combinations thereof) similar to that of a naturally-occurring effector protein, such as, for example, a naturally-occurring effector protein with reduced cleavage activity (e.g., Cas 14) including cis cleavage activity, trans cleavage activity, or combinations thereof. In some embodiments, effector proteins disclosed herein may be fused to effector partners (e.g., fusion partners) or fusion proteins wherein the effector partners (e.g., fusion partners) or fusion proteins are capable of some function or activity not provided by an effector protein.

An effector protein may be a modified effector protein having increased modification activity and/or increased substrate binding activity (e.g., substrate selectivity, specificity, and/or affinity). Alternatively, or in addition, an effector protein may be a modified effector protein having reduced modification activity (e.g., a catalytically defective effector protein) or no modification activity (e.g., a catalytically inactive effector protein). Accordingly, an effector protein as used herein encompasses a modified polypeptide that does not have nuclease activity.

In certain embodiments, effector proteins described herein can comprise one or more functional domains. In certain embodiments, effector proteins described herein can comprise one or more functional domains comprising a protospacer adjacent motif (PAM)-interacting domain, an oligonucleotide-interacting domain, one or more recognition domains, a non-target strand interacting domain, and a RuvC, domain.

A PAM interacting domain can be a target strand PAM interacting domain (TPID) or a non-target strand PAM interacting domain (NTPID). In some embodiments, a PAM interacting domain, such as a TPID or a NTPID, on an effector protein describes a region of an effector protein that interacts with target nucleic acid.

In some embodiments, the effector proteins comprise a RuvC domain. In some embodiments, when describing a RuvC domain, reference is made to a region of an effector protein that is capable of cleaving a target nucleic acid, and in certain instances, of processing a pre-crRNA. In some instances, the RuvC domain is located near the C-terminus of the effector protein. In some embodiments, a RuvC domain comprises substrate binding activity, catalytic activity, or both. In some embodiments, the RuvC domain may be defined by a single, contiguous sequence, or a set of RuvC subdomains that are not contiguous with respect to the primary amino acid sequence of the protein. An effector protein of the present disclosure may include multiple RuvC subdomains, which may combine to generate a RuvC domain with substrate binding or catalytic activity. For example, an effector protein may include three RuvC subdomains (RuvC-I, RuvC-II, and RuvC-III) that are not contiguous with respect to the primary amino acid sequence of the effector protein, but form a RuvC domain once the protein is produced and folds.

In some embodiments, effector proteins comprise one or more recognition domains (REC domain) with a binding affinity for a guide nucleic acid or for a guide nucleic acid-target nucleic acid heteroduplex. In some embodiments, when describing a REC domain reference is made to a domain in an α-helical recognition region or lobe. An effector protein may contain at least one REC domain (e.g., REC1, REC2) which can help to accommodate and stabilize the guide nucleic acid and target nucleic acid hybrid. An effector protein may comprise a zinc finger domain.

An effector protein may be small, which may be beneficial for nucleic acid detection or editing (for example, the effector protein may be less likely to adsorb to a surface or another biological species due to its small size). The smaller nature of these effector proteins may allow for them to be more easily packaged and delivered with higher efficiency in the context of genome editing and more readily incorporated as a reagent in an assay. In some embodiments, the length of the effector protein is at least 400 linked amino acid residues. In some embodiments, the length of the effector protein is less than 500 linked amino acid residues. In some embodiments, the length of the effector protein is about 400 to about 500 linked amino acid residues. In some embodiments, the length of the effector protein is about 450 to about 550, about 400 to about 420, about 420 to about 440, about 440 to about 460, about 460 to about 480, about 480 to about 500, about 500 to about 520, about 520 to about 540, about 540 to about 560, about 560 to about 580, about 580 to about 600, about 600 to about 620, about 620 to about 640, about 640 to about 660, about 660 to about 680, about 680 to about 700, or about 700 to 720 linked amino acids.

In some embodiments, an effector protein described herein is at least about 65%, about 70%, about 75%, about 80%, about 85%, about 90%, about 95%, about 97%, about 98%, or about 99% identical to the sequence of SEQ ID NO: 1, but is not 100% identical to a WT effector protein described herein. In some embodiments, an effector protein described herein is at least about 65%, about 70%, about 75%, about 80%, about 85%, about 90%, about 95%, about 97%, about 98%, or about 99% similar to the sequence of SEQ ID NO: 1, but is not 100% similar to a WT effector protein described herein.

In some embodiments, a WT effector protein is a CasPhi.12 effector protein and comprises a sequence of:

(SEQ ID NO: 1)
MIKPTVSQFLTPGFKLIRNHSRTAGLKLKNEGEEACKKFVRENEIPKDEC
PNFQGGPAIANIIAKSREFTEWEIYQSSLAIQEVIFTLPKDKLPEPILKE
EWRAQWLSEHGLDTVPYKEAAGLNLIIKNAVNTYKGVQVKVDNKNKNNLA
KINRKNEIAKLNGEQEISFEEIKAFDDKGYLLQKPSPNKSIYCYQSVSPK
PFITSKYHNVNLPEEYIGYYRKSNEPIVSPYQFDRLRIPIGEPGYVPKWQ
YTFLSKKENKRRKLSKRIKNVSPILGIICIKKDWCVFDMRGLLRTNHWKK
YHKPTDSINDLFDYFTGDPVIDTKANVVRFRYKMENGIVNYKPVREKKGK
ELLENICDQNGSCKLATVDVGQNNPVAIGLFELKKVNGELTKTLISRHPT
PIDFCNKITAYRERYDKLESSIKLDAIKQLTSEQKIEVDNYNNNFTPQNT
KQIVCSKLNINPNDLPWDKMISGTHFISEKAQVSNKSEIYFTSTDKGKTK
DVMKSDYKWFQDYKPKLSKEVRDALSDIEWRLRRESLEFNKLSKSREQDA
RQLANWISSMCDVIGIENLVKKNNFFGGSGKREPGWDNFYKPKKENRWWI
NAIHKALTELSQNKGKRVILLPAMRTSITCPKCKYCDSKNRNGEKFNCLK
CGIELNADIDVATENLATVAITAQSMPKPTCERSGDAKKPVRARKAKAPE
FHDKLAPSYTVVLREAV.

Provided herein is an effector protein that is a variant of a wild-type effector protein (e.g., SEQ ID NO: 1). In some embodiments, when describing a variant polypeptide reference is made to a form or version of a protein that differs from the wild-type protein. A variant may have a different function or activity relative to the wild-type protein. In some embodiments, effector proteins described herein are variants of a wild-type effector protein (e.g., SEQ ID NO: 1), wherein the effector protein comprises one or more amino acid alterations relative to the sequence of the wild-type protein (e.g., SEQ ID NO: 1). Unless specified otherwise, it is understood that references to an effector protein herein also includes effector protein variants as described herein.

In some embodiments, the one or more amino acid alterations comprise substitutions, deletions, insertions, or any combination thereof. In some embodiments, the one or more amino acid alterations comprises one or more deletions of one or more amino acids. In some embodiments, the one or more amino acid alterations comprises one or more insertions of one or more amino acid. In some embodiments, the one or more amino acid alterations comprises one or more conservative substitutions, one or more non-conservative substitutions, and combinations thereof, of one or more amino acids. Such an effector protein includes one or more alterations wherein at least one alteration is a conservative or non-conservative substitution. In some aspects, such a conservative amino acid substitution is a chemically conservative or an evolutionary conservative amino acid substitution. Methods of identifying conservative amino acids are well known to one of skill in the art, any one of which can be used to generate the effector proteins described herein.

When describing a conservative substitution herein, reference is made to the replacement of one amino acid for another such that the replacement takes place within a family of amino acids that are related in their side chains. Conversely, when describing a non-conservative substitution, reference is made to the replacement of one amino acid residue for another that does not have a related side chain. It is understood that genetically encoded amino acids can be divided into four families having related side chains: (1) acidic (negatively charged): Asp (D), Glu (E); (2) basic (positively charged): Lys (K), Arg (R), His (H); (3) non-polar (hydrophobic): Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic: Ala (A), Val (V), Leu (L), Ile (I), Met (M), Phe (F); and (ii) moderately hydrophobic: Gly (G), Pro (P), Cys (C), Tyr (Y), Trp (W); and (4) uncharged polar: Asn (N), Gln (Q), Ser (S), Thr (T). Amino acids may be related by aliphatic side chains: Gly (G), Ala (A), Val (V), Leu (L), Ile (I), Ser (S), Thr (T), with Ser (S) and Thr (T) optionally being grouped separately as aliphatic-hydroxyl. Amino acids may be related by aromatic side chains: Phe (F), Tyr (Y), Trp (W). Amino acids may be related by amide side chains: Asn (N), Gln (Q). Amino acids may be related by sulfur-containing side chains: Cys (C) and Met (M). As a non-limiting example, a conservative substitution of a basic amino acid of the amino acid sequence recited in SEQ ID NO: 1 is a substitution for another basic (positively charged) amino acid (e.g., Arg (R), or His (H)). As a non-limiting example, a non-conservative substitution of acidic (negatively charged) amino acid of the amino acid sequence recited in SEQ ID NO: 1 is a substitution for a basic (positively charged) amino acid (e.g., Lys (K), Arg (R), or His (H)).

It is understood that effector proteins as described herein can carry out a similar enzymatic reaction as the WT effector protein (SEQ ID NO: 1) as discussed above. In some embodiments, an effector protein described herein may be engineered to show an improved activity (e.g., nucleic acid binding activity, enhanced nuclease activity, enhanced potency of nuclease activity, or enhanced precision of nuclease activity) relative to the wildtype counterpart. For example, such an effector protein includes one or more alterations at a position described in TABLE 1, TABLE 1.1, TABLE 1.2, TABLE 1.3, TABLE 7, TABLE 9, TABLE 14, TABLE 15, TABLE 16, TABLE 17, or a combination thereof, and, in some embodiments, a combination of alterations, e.g., a combination of alterations as described in TABLE 1, TABLE 1.1, TABLE 1.2, TABLE 1.3, TABLE 7, TABLE 9, TABLE 14, TABLE 15, TABLE 16, TABLE 17, or a combination thereof, and has higher activity relative to the WT effector protein (SEQ ID NO: 1) as described herein.

In some embodiments, enzymatic activity of an effector protein described herein refers to substrate specificity, substrate affinity, or both. In some embodiments, enzymatic activity includes cleavage activity, such as nickase or nuclease activity, precision of nuclease activity, and potency of nuclease activity, or combinations thereof. Precision of nuclease activity may be assessed by measuring the number of nucleotides that are deleted in a target nucleic acid, e.g., by sequencing. Nuclease activity is more precise if it deletes fewer nucleotides around a target site as compared to nuclease activity that is less precise and deletes more nucleotides around a target site. In some embodiments, introduction of a positive charge within a DNA binding region of the effector protein may strengthen the interaction between the effector protein and the negatively charged DNA backbone. In some embodiments, an engineered effector protein comprises addition of one or more positively charged amino acids, substitution of one or more amino acids with positively charged amino acids, deletion of one or more negatively charged amino acids, and combinations thereof. In some embodiments, the positively charged amino acid residues are independently selected from arginine, lysine and histidine. In some embodiments, the positively charged amino acid residue is arginine. In some embodiments, the introduction of the positive charge enhances nuclease activity relative to the counterpart wildtype protein. In some embodiments, the introduction of the positive charge enhances nuclease activity of the effector protein.

It is therefore understood that the variants of the WT effector protein described herein can include alterations that provide a beneficial characteristic to effector proteins described herein, including but not limited to, increased activity (e.g., indel activity, catalytic activity, specificity or selectivity and/or affinity for a substrate, such as a target nucleic acid and/or a guide nucleic acid) as described herein (see, e.g., Example 1 and 2). In some embodiments, effector proteins described herein can exhibit an activity that is at least the same or higher than the wild-type effector protein (SEQ ID NO: 1), that is, it has activity that is the same or higher than the WT effector protein without the variant at the same amino acid position(s). In some embodiments, effector proteins described herein can exhibit two or more activities (e.g., indel activity, catalytic activity of a substrate, specificity or selectivity for a substrate and binding affinity of a substrate) that are at least the same or higher than the wild-type effector protein (SEQ ID NO: 1), that is, it has two or more activities that are the same or higher than the effector protein (SEQ ID NO: 1) without the variant at the same amino acid position(s). For example, effector proteins described herein can have one or more activities that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher over a wild-type effector protein (SEQ ID NO: 1) (see, e.g., Example 1 and 2). In some embodiments, the one or more amino acid alterations provide a beneficial characteristic to effector proteins described herein, wherein the beneficial characteristic is a decrease of activity. Effector proteins comprising decreased activity are described herein, for example in the Engineered Proteins section.

In some embodiments, activity of effector proteins described herein can be measured relative to a WT effector protein (SEQ ID NO: 1) in a cleavage assay. It is understood that activity refers to activity relative to a WT effector protein (SEQ ID NO: 1) under the same assay conditions, such as those described herein (see, e.g., Example 1 and 2).

The one or more alterations may be located at one or more positions located in a region of the polypeptide that comprises substrate binding activity, catalytic activity, and/or binding affinity for a substrate such as a target nucleic acid, an engineered guide nucleic acid or a guide nucleic acid-target nucleic acid heteroduplex.

In some embodiments, effector proteins provided herein are a variant of a reference polypeptide, wherein the reference polypeptide has an amino acid sequence of SEQ ID NO: 1, and the effector protein comprises one or more amino acid alterations in one or more regions that interact with a substrate such as a target nucleic acid, an engineered guide nucleic acid or a guide nucleic acid-target nucleic acid heteroduplex. In some embodiments, effector proteins provided herein are a variant of a reference polypeptide, wherein the reference polypeptide has an amino acid sequence of SEQ ID NO: 1, and the effector protein comprises one or more amino acid alterations in a region of the polypeptide that comprises substrate binding activity, catalytic activity, and/or binding affinity for a substrate such as a target nucleic acid, an engineered guide nucleic acid or a guide nucleic acid-target nucleic acid heteroduplex. In some embodiments, effector proteins provided herein are a variant of a reference polypeptide, wherein the reference polypeptide has an amino acid sequence of SEQ ID NO: 1, and the effector protein comprises one or more amino acid alterations in a RuvC domain, a REC domain, TPID, NTPID, or any combination thereof.

The one or more alterations may be located at one or more positions corresponding to the one or more positions described in TABLE 1, TABLE 1.1, TABLE 1.2, TABLE 1.3, TABLE 7, TABLE 9, TABLE 14, TABLE 15, TABLE 16, or TABLE 17. The one or more alterations may be located at one or more positions corresponding to one or more positions in SEQ ID NO: 1. As used herein, the phrase “a residue corresponding to position X in SEQ ID NO: Y” refers to a residue at a corresponding position following an alignment of two sequences. For example, the residue in SEQ ID NO: 2 corresponding to position 26 in SEQ ID NO: 1 is the residue at position 26 in SEQ ID NO: 1. In some embodiments, a reference sequence is an effector protein that is not SEQ ID NO: 2, 3, or 4.

In some embodiments, effector proteins provided herein are a variant of a reference polypeptide, wherein the reference polypeptide has an amino acid sequence of SEQ ID NO: 1, and the effector protein comprises one or more amino acid alterations. In some embodiments, effector proteins provided herein are a variant of a reference polypeptide, wherein the reference polypeptide has an amino acid sequence of SEQ ID NO: 1, and the effector protein comprises one or more amino acid alterations comprising substitutions, deletions, insertions, or any combination thereof. In some embodiments, effector proteins provided herein are a variant of a reference polypeptide, wherein the reference polypeptide has an amino acid sequence of SEQ ID NO: 1, and the effector protein comprises one or more conservative or non-conservative amino acid substitutions. As a non-limiting example, a conservative substitution of K184 of SEQ ID NO: 1 is for another basic (positively charged) amino acid (e.g., Arg (R), or His (H)). In some embodiments, effector proteins provided herein are a variant of a reference polypeptide, wherein the reference polypeptide has an amino acid sequence of SEQ ID NO: 1, and the effector protein comprises one or more amino acid alterations that are non-conservative amino acid alterations. As a non-limiting example, a non-conservative substitution of L26 of SEQ ID NO: 1 for a basic (positively charged) amino acid (e.g., Lys (K), Arg (R), or His (H)).

In some instances, an effector protein disclosed herein comprises an amino acid sequence that is at least about 80%, at least about 85%, at least about 90%, at least about 95%, at least about 98%, or at least about 99% identical to SEQ ID NO: 1 and comprises at least one amino acid alteration relative to SEQ ID NO: 1. In some instances, an effector protein disclosed herein comprises an amino acid sequence that is at least about 80%, at least about 85%, at least about 90%, at least about 95%, at least about 98%, or at least about 99% identical to SEQ ID NO: 1 and comprises at least one conservative or non-conservative amino acid substitution relative to SEQ ID NO: 1. In some instances, an effector protein disclosed herein comprises an amino acid sequence that is at least about 80%, at least about 85%, at least about 90%, at least about 95%, at least about 98%, or at least about 99% identical to SEQ ID NO: 1, wherein all but 1, 2, 3, 4, 5, 6, 7, 8, 9, 10, or more amino acids alterations relative to SEQ ID NO: 1 are conservative or non-conservative amino acid substitutions, or combinations thereof. In some instances, an effector protein disclosed herein comprises an amino acid sequence that is identical to SEQ ID NO: 1 with the exception of 1, 2, 3, 4, 5, 6, 7, 8, 910, or more conservative or non-conservative amino acid substitutions, or combinations thereof.

In some embodiments, an effector protein provided herein is a variant of a reference polypeptide, wherein the reference polypeptide has an amino acid sequence of SEQ ID NO: 1, and the effector protein has one or more alterations at one or more positions relative to SEQ ID NO: 1. In some embodiments, an effector protein provided herein is a variant of a reference polypeptide, wherein the reference polypeptide has an amino acid sequence of SEQ ID NO: 1, and the effector protein has one or more alterations at a position described in TABLE 1, TABLE 1.1, TABLE 3, TABLE 7, TABLE 9, TABLE 14, TABLE 15, TABLE 16, TABLE 17, or any combination thereof relative to SEQ ID NO: 1. When describing the amino acid sequences of effector proteins described herein, a person of ordinary skill in the art understands that reference of the one or more amino acid alterations at the positions described herein (e.g., in TABLE 1, TABLE 1.1, TABLE 1.2, TABLE 1.3, TABLE 7, TABLE 9, TABLE 14, TABLE 15, TABLE 16, and TABLE 17), and the percent identity to a reference sequence (e.g., SEQ ID NO: 1) describes the amino acid sequence of the effector protein itself, such that the amino acid sequence of the effector protein has the amino acid sequence of the reference sequence, but with a certain percent identity or similarity to the reference sequence while retaining the one or more amino acid alterations that the effector protein is described as having.

TABLE 1 provides illustrative alterations relative to SEQ ID NO: 1 of effector proteins described herein. Accordingly, in some embodiments, an effector protein provided herein includes one or more amino acid alterations at one or more residues corresponding to position 2, 5, 11, 13, 14, 15, 16, 17, 18, 19, 20, 21, 22, 23, 24, 25, 26, 28, 29, 30, 31, 32, 33, 34, 35, 36, 37, 38, 39, 40, 41, 42, 43, 44, 46, 48, 50, 51, 52, 53, 54, 55, 56, 57, 58, 59, 62, 65, 68, 70, 71, 73, 74, 75, 77, 78, 79, 80, 83, 84, 87, 89, 90, 92, 94, 95, 96, 97, 99, 100, 101, 102, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 146, 147, 148, 149, 150, 151, 153, 157, 158, 159, 160, 163, 168, 169, 171, 175, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 218, 220, 223, 225, 227, 228, 229, 230, 231, 232, 233, 234, 236, 238, 239, 240, 241, 242, 243, 244, 245, 246, 247, 248, 250, 252, 253, 254, 255, 256, 257, 258, 259, 260, 262, 264, 265, 266, 268, 272, 273, 276, 279, 280, 281, 285, 286, 295, 297, 298, 301, 302, 304, 306, 311, 312, 315, 316, 328, 329, 334, 338, 340, 348, 355, 356, 357, 360, 361, 363, 366, 368, 369, 370, 384, 390, 391, 392, 393, 395, 397, 399, 400, 405, 406, 407, 435, 445, 471, 472, 480, 483, 497, 501, 503, 509, 511, 512, 513, 514, 515, 516, 517, 521, 523, 526, 529, 531, 536, 540, 541, 542, 543, 544, 545, 546, 549, 567, 568, 577, 579, 585, 590, 591, 592, 593, 594, 595, 596, 599, 602, 603, 604, 605, 606, 607, 608, 612, 617, 620, 624, 634, 638, 639, 653, 658, 673, 674, 678, 679, 682, 684, 685, 696, 699, 701, 703, 707, 709, 715, 716, more than one of the foregoing, or a combination thereof, relative to SEQ ID NO: 1.

In some embodiments, an effector protein provided herein includes one or more amino acid alterations relative to SEQ ID NO: 1 that includes an alteration at a residue corresponding to position 2, 5, 15, 18, 20, 21, 26, 30, 33, 34, 35, 37, 38, 41, 43, 54, 79, 92, 99, 108, 109, 110, 111, 113, 114, 116, 118, 119, 121, 132, 135, 138, 139, 149, 180, 182, 183, 184, 186, 189, 196, 198, 200, 203, 205, 206, 207, 208, 209, 220, 223, 258, 281, 348, 355, 369, 406, 435, 471, 521, 567, 568, 579, 612, 638, 658, 701, 707 relative to SEQ ID NO: 1.

In some embodiments, an effector protein provided herein includes one or more amino acid alterations relative to SEQ ID NO: 1 that includes an alteration at a residue corresponding to position 5, 26, 121, 198, 223, 258, 471, 579, 701, or a combination thereof relative to SEQ ID NO: 1. In some embodiments, an effector protein provided herein includes one or more amino acid alterations relative to SEQ ID NO: 1 that includes an alteration at a residue corresponding to position 369, 567, 658, or a combination thereof relative to SEQ ID NO: 1.

In some embodiments, an effector protein comprises one or more amino acid alterations described in TABLE 1 and the amino acid sequence of the effector protein is at least about 65%, at least about 70%, at least about 75%, at least about 80%, at least about 85%, at least about 90%, or at least about 95% identical, or more to SEQ ID NO: 1. In some embodiments, compositions, methods and systems provided herein comprise an effector protein, wherein the amino acid sequence of the effector protein, other than the one or more amino acid residue alteration at any one or more of the positions described in TABLE 1, or a combination thereof, comprises at least about 65%, at least about 70%, at least about 75%, at least about 80%, at least about 85%, at least about 90%, or at least about 95%, or more, sequence identity to SEQ ID NO: 1.

In some embodiments, an effector protein comprises one or more amino acid alterations described in TABLE 1 and the amino acid sequence of the effector protein is at least about 65%, at least about 70%, at least about 75%, at least about 80%, at least about 85%, at least about 90%, or at least about 95% similar, or more to SEQ ID NO: 1. In some embodiments, compositions, methods and systems provided herein comprise an effector protein, wherein the amino acid sequence of the effector protein, other than the one or more amino acid residue alteration at any one or more of the positions described in TABLE 1, or a combination thereof, comprises at least about 65%, at least about 70%, at least about 75%, at least about 80%, at least about 85%, at least about 90%, or at least about 95%, or more, sequence similarity to SEQ ID NO: 1.

In certain embodiments, the amino acid sequence of an effector protein provided herein, other than the one or more amino acid residue alteration at any one of more of the positions described in TABLE 1, or a combination thereof, comprises at least about 200, at least about 220, at least about 240, at least about 260, at least about 280, at least about 300, at least about 320, at least about 340, at least about 360, at least about 380, or at least about 400 contiguous amino acids of SEQ ID NO: 1. In certain embodiments, compositions, methods and systems provided herein comprise an effector protein, wherein the amino acid sequence of the effector protein, other than the one or more amino acid residue alteration at any one of more of the positions described in TABLE 1, or a combination thereof, comprises at least about 200, at least about 220, at least about 240, at least about 260, at least about 280, at least about 300, at least about 320, at least about 340, at least about 360, at least about 380, or at least about 400 contiguous amino acids of the sequence of SEQ ID NO: 1.

In some embodiments, an effector protein provided herein, other than the one or more amino acid residue alteration at position 2, 5, 15, 18, 20, 21, 26, 30, 33, 34, 35, 37, 38, 41, 43, 54, 79, 92, 99, 108, 109, 110, 111, 113, 114, 116, 118, 119, 121, 132, 135, 138, 139, 149, 180, 182, 183, 184, 186, 189, 196, 198, 200, 203, 205, 206, 207, 208, 209, 220, 223, 258, 281, 348, 355, 369, 406, 435, 471, 521, 567, 568, 579, 612, 638, 658, 701, 707, or a combination thereof, as described in TABLE 1, comprises an amino acid sequence that is at least about 65%, about 70%, about 75%, about 80%, about 85%, about 90%, about 95%, about 97%, or about 98%, about 99%, or is identical to the sequence of SEQ ID NO: 1. In certain embodiments, compositions, methods and systems provided herein comprise an effector protein, wherein the amino acid sequence of the effector protein, other than the one or more amino acid residue alteration at position 2, 5, 15, 18, 20, 21, 26, 30, 33, 34, 35, 37, 38, 41, 43, 54, 79, 92, 99, 108, 109, 110, 111, 113, 114, 116, 118, 119, 121, 132, 135, 138, 139, 149, 180, 182, 183, 184, 186, 189, 196, 198, 200, 203, 205, 206, 207, 208, 209, 220, 223, 258, 281, 348, 355, 369, 406, 435, 471, 521, 567, 568, 579, 612, 638, 658, 701, 707, or a combination thereof, is at least about 65%, about 70%, about 75%, about 80%, about 85%, about 90%, about 95%, about 98%, about 99%, or is identical to the sequence of SEQ ID NO: 1.

In some embodiments, an effector protein provided herein, other than the one or more amino acid residue alteration at position 2, 5, 15, 18, 20, 21, 26, 30, 33, 34, 35, 37, 38, 41, 43, 54, 79, 92, 99, 108, 109, 110, 111, 113, 114, 116, 118, 119, 121, 132, 135, 138, 139, 149, 180, 182, 183, 184, 186, 189, 196, 198, 200, 203, 205, 206, 207, 208, 209, 220, 223, 258, 281, 348, 355, 369, 406, 435, 471, 521, 567, 568, 579, 612, 638, 658, 701, 707, or a combination thereof, as described in TABLE 1, comprises an amino acid sequence that is at least about 65%, about 70%, about 75%, about 80%, about 85%, about 90%, about 95%, about 97%, or about 98%, about 99%, or 100% similar to the sequence of SEQ ID NO: 1. In certain embodiments, compositions, methods and systems provided herein comprise an effector protein, wherein the amino acid sequence of the effector protein, other than the one or more amino acid residue alteration at position 2, 5, 15, 18, 20, 21, 26, 30, 33, 34, 35, 37, 38, 41, 43, 54, 79, 92, 99, 108, 109, 110, 111, 113, 114, 116, 118, 119, 121, 132, 135, 138, 139, 149, 180, 182, 183, 184, 186, 189, 196, 198, 200, 203, 205, 206, 207, 208, 209, 220, 223, 258, 281, 348, 355, 369, 406, 435, 471, 521, 567, 568, 579, 612, 638, 658, 701, 707, or a combination thereof, is at least about 65%, about 70%, about 75%, about 80%, about 85%, about 90%, about 95%, about 98%, about 99%, or 100% similar to the sequence of SEQ ID NO: 1.

In some embodiments, each one or more amino acid residue alteration is independently a substitution with a basic (positively charged) amino acid, an acidic (negatively-charged) amino acid, a non-polar (hydrophobic) amino acid, or an uncharged polar amino acid, or any combination thereof. In some embodiments, a substitution with a basic (positively charged) amino acid is a substitution of an amino acid residue with a Lys (K), Arg (R), or His (H). In some embodiments, a substitution with an acidic (negatively charged) amino acid is a substitution of an amino acid residue with an Asp (D) or Glu (E). In some embodiments, a substitution with a non-polar (hydrophobic) amino acid is a substitution of an amino acid residue with a Cys (C), Ala (A), Val (V), Leu (L), Ile (I), Pro (P), Phe (F), Met (M), Trp (W), Gly (G), or Tyr (Y). In some embodiments, a substitution with an uncharged polar amino acid is a substitution of an amino acid residue with an Asn (N), Gln (Q), Ser (S), or Thr (T). In some embodiments, the one or more amino acid alterations are each a substitution of an amino acid residue with an A, N, R, K, E, S, Q, P, T, G, F or D. In some embodiments, the one or more amino acid alterations are each a substitution of an amino acid residue with an A, Q or N. In some embodiments, the one or more amino acid alterations are each a substitution of an amino acid residue with a R, K, E, S, Q, P, T, G, F or D. In some embodiments, the one or more amino acid alterations are each an alteration as described in any of TABLE 1, TABLE 1.1, TABLE 1.2, TABLE 1.3, TABLE 7, TABLE 9, TABLE 14, TABLE 15, TABLE 16, TABLE 17, TABLE 18, or any combination thereof.

In some embodiments, the one or more amino acid alteration comprises 12R, TSR, K15R, R18R, H20R, S21R, L26R, L26K, N30R, E33R, E34R, A35R, K37R, K38R, R41R, N43R, Q54R, Q79R, K92E, K99R, S108R, E109R, H110R, G111R, D113R, T114R, P116R, K118R, E119S, A121Q, N132R, K135R, Q138R, V139R, L149R, Y180R, L182R, Q183R, K184R, S186R, K189R, K189P, S196R, S198R, K200R, 1203R, S205R, K206R, Y207R, H208R, N209R, Y220S, S223P, E258K, K281R, K348R, N355R, N406K, K435Q, I471T, V521T, N568D, S579R, Q612R, S638K, F701R, P707R, or any combinations thereof. In some embodiments, the one or more amino acid alteration comprises TSR, L26K, A121Q, S198R, S223P, E258K, I471T, S579R, or F701R, or any combinations thereof. In some embodiments, the one or more amino acid alteration comprises D369A, D369N, D658A, D658N, E567A, E567Q, or any combinations thereof. In some embodiments, the one or more amino acid alteration comprises E567A or E567Q.

A variant effector protein provided herein may comprise a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 or more amino acid alterations relative to a wild-type effector protein (e.g., SEQ ID NO: 1). For example, an effector protein provided herein may comprise a combination of 2 to 20, or more amino acid alterations relative to a wild-type effector protein (SEQ ID NO: 1).

Combinations of exemplary amino acid alteration may each be independently a conservative substitution or a non-conservative substitution. For example, a variant effector protein provided herein may comprise a combination of 2, 3, 4, 5, 6, 7, 8, 9, 10, or more conservative amino acid substitutions relative to a wild-type effector protein (e.g., SEQ ID NO: 1). In another example, a variant effector protein provided herein may comprise a combination of 2, 3, 4, 5, 6, 7, 8, 9, 10, or more non-conservative amino acid substitutions relative to a wild-type effector protein (e.g., SEQ ID NO: 1). In another example, a variant effector protein provided herein may comprise a combination of 2, 3, 4, 5, 6, 7, 8, 9, 10, or more amino acid alterations relative to a wild-type effector protein (e.g., SEQ ID NO: 1), wherein the amino acid alterations are a combination of conservative and non-conservative substitution.

Exemplary variant effector proteins that comprise 1 amino acid alterations relative to a wild-type effector protein (SEQ ID NO: 1) are set forth in TABLE 1 above. Exemplary variant effector proteins that comprise more than 1 amino acid alteration relative to a wild-type effector protein (SEQ ID NO: 1), comprises at least 1 amino acid alteration set forth in TABLE 1 above in combination with another amino acid alteration or a combination of two, three, four, or more amino acid alterations as set forth in TABLE 1. For example, exemplary variant effector proteins can comprise a combination of 2 amino acid alterations relative to a wild-type effector protein (SEQ ID NO: 1) as set forth in TABLE 1.1 or TABLE 15 below. In another example, exemplary variant effector proteins can comprise a combination of 3 amino acid alterations relative to a wild-type effector protein (SEQ ID NO: 1) as set forth in TABLE 1.2 below. In an additional example, exemplary variant effector proteins can comprise a combination of 4 amino acid alterations relative to a wild-type effector protein (SEQ ID NO: 1) as set forth in TABLE 1.3 below.

A person of ordinary skill in the art would understand from the present disclosure that one or more amino acid alteration resulting in a substitution of the amino acid for an amino acid from a specific family having a certain side chain (i.e., a basic (positively charged) amino acid, an acidic (negatively-charged) amino acid, a non-polar (hydrophobic) amino acid, or an uncharged polar amino acid)), that the amino acid alteration may also be a substitution with any other amino acid in the described family. For example, and as described herein, any exemplary amino acid alteration resulting in an arginine (R) substitution described in TABLES 1, 1.1, 1.2, 1.3, 14, 15, 16 and 17 may be a substitution with any basic (positively charged) amino acid instead of just an arginine (R). By describing the amino acid alteration as substituting an amino acid at a position described in TABLES 1, 1.1, 1.2, 1.3, 14, 15, 16, or 17 with a R, such disclosure is also describing substituting the amino acid at that same position with an H or a K instead. Such amino acid alterations are independent of each other. For example, by describing the amino acid alterations of 26R and 109R combined with the disclosure found herein, such disclosure also describes the amino acid alterations of: 26H and 109R; 26R and 109H; 26H and 109H; 26K and 109R; 26R and 109K; 26K and 109K; 26H and 109K; and 26K and 109H.

In some embodiments, an effector protein provided herein, other than the one or more amino acid residue alteration at a position described in TABLE 1, such as position 2, 5, 15, 18, 20, 21, 26, 30, 33, 34, 35, 37, 38, 41, 43, 54, 79, 92, 99, 108, 109, 110, 111, 113, 114, 116, 118, 119, 121, 132, 135, 138, 139, 149, 180, 182, 183, 184, 186, 189, 196, 198, 200, 203, 205, 206, 207, 208, 209, 220, 223, 258, 281, 348, 355, 369, 406, 435, 471, 521, 567, 568, 579, 612, 638, 658, 701, 707, or a combination thereof, comprises an amino acid sequence that is at least about 65%, about 70%, about 75%, about 80%, about 85%, about 90%, about 95%, about 97%, or about 98%, about 99% identical to the sequence of SEQ ID NO: 1, and comprises at least one amino acid residue alteration relative to the sequence of SEQ ID NO: 1, for example any amino acid alteration set forth in TABLE 1. In certain embodiments, compositions, methods and systems provided herein comprise an effector protein, wherein the amino acid sequence of the effector protein, other than the one or more amino acid residue alteration at position 2, 5, 15, 18, 20, 21, 26, 30, 33, 34, 35, 37, 38, 41, 43, 54, 79, 92, 99, 108, 109, 110, 111, 113, 114, 116, 118, 119, 121, 132, 135, 138, 139, 149, 180, 182, 183, 184, 186, 189, 196, 198, 200, 203, 205, 206, 207, 208, 209, 220, 223, 258, 281, 348, 355, 369, 406, 435, 471, 521, 567, 568, 579, 612, 638, 658, 701, 707, or a combination thereof, is at least about 65%, about 70%, about 75%, about 80%, about 85%, about 90%, about 95%, about 98%, about 99% identical to the sequence of SEQ ID NO: 1, and comprises at least one amino acid residue alteration relative to the sequence of SEQ ID NO: 1.

In some embodiments, an effector protein provided herein, other than the one or more amino acid residue alterations at a position described in TABLE 1, such as position 2, 5, 15, 18, 20, 21, 26, 30, 33, 34, 35, 37, 38, 41, 43, 54, 79, 92, 99, 108, 109, 110, 111, 113, 114, 116, 118, 119, 121, 132, 135, 138, 139, 149, 180, 182, 183, 184, 186, 189, 196, 198, 200, 203, 205, 206, 207, 208, 209, 220, 223, 258, 281, 348, 355, 369, 406, 435, 471, 521, 567, 568, 579, 612, 638, 658, 701, 707, or a combination thereof, comprises an amino acid sequence that is at least about 65%, about 70%, about 75%, about 80%, about 85%, about 90%, about 95%, about 97%, or about 98%, about 99%, or 100% similar to the sequence of SEQ ID NO: 1, and comprises at least one amino acid residue alteration relative to the sequence of SEQ ID NO: 1, for example any amino acid alteration set forth in TABLE 1. In certain embodiments, compositions, methods and systems provided herein comprise an effector protein, wherein the amino acid sequence of the effector protein, other than the amino acid residue at position 2, 5, 15, 18, 20, 21, 26, 30, 33, 34, 35, 37, 38, 41, 43, 54, 79, 92, 99, 108, 109, 110, 111, 113, 114, 116, 118, 119, 121, 132, 135, 138, 139, 149, 180, 182, 183, 184, 186, 189, 196, 198, 200, 203, 205, 206, 207, 208, 209, 220, 223, 258, 281, 348, 355, 369, 406, 435, 471, 521, 567, 568, 579, 612, 638, 658, 701, 707, or a combination thereof, is at least about 65%, about 70%, about 75%, about 80%, about 85%, about 90%, about 95%, about 98%, about 99%, or 100% similar to the sequence of SEQ ID NO: 1, and comprises at least one amino acid residue alteration relative to the sequence of SEQ ID NO: 1. In some embodiments, the one or more amino acid alteration is at one or more residues corresponding to one or more positions comprising 5, 26, 121, 198, 223, 258, 471, 579, 701, or any combination thereof, relative to SEQ ID NO: 1.

In some embodiments, the at least one amino acid alteration is each a deletion, insertion, or a substitution. In some embodiments, the at least one amino acid residue alteration is a conservative or non-conservative amino acid substitution. In some embodiments, the at least one amino acid residue alteration is each independently a substitution with a basic (positively charged) amino acid, an acidic (negatively-charged) amino acid, a non-polar (hydrophobic) amino acid, or an uncharged polar amino acid, or any combination thereof.

In some embodiments, a substitution with a basic (positively charged) amino acid is a substitution of an amino acid residue with a Lys (K), Arg (R), or His (H). In some embodiments, a substitution with a basic (positively charged) amino acid is a substitution of an amino acid residue with a Lys (K) or Arg (R). In some embodiments, a substitution with an acidic (negatively charged) amino acid is a substitution of an amino acid residue with an Asp (D) or Glu (E). In some embodiments, a substitution with a non-polar (hydrophobic) amino acid is a substitution of an amino acid residue with a Cys (C), Ala (A), Val (V), Leu (L), Ile (I), Pro (P), Phe (F), Met (M), Trp (W), Gly (G), or Tyr (Y). In some embodiments, a substitution with a non-polar (hydrophobic) amino acid is a substitution with a non-polar (hydrophobic) amino acid is a substitution of an amino acid residue with a Pro (P). In some embodiments, a substitution with an uncharged polar amino acid is a substitution of an amino acid residue with a Asn (N), Gln (Q), Ser (S), or Thr (T). In some embodiments, a substitution with an uncharged polar amino acid is a substitution with an uncharged polar amino acid is a substitution of an amino acid residue with a Gln (Q), Ser (S), or Thr (T). In some embodiments, the one or more amino acid alterations are each a substitution of an amino acid residue with a G, R, K, E, S, Q, P, T, or D. In some embodiments, the one or more amino acid alterations are each an alteration as described in any of TABLE 1, TABLE 1.1, TABLE 1.2, TABLE 1.3, TABLE 7, TABLE 9, TABLE 14, TABLE 15, TABLE 16, TABLE 17, TABLE 18, or any combination thereof.

An effector protein provided herein can include any combination of the alterations set forth in TABLE 1, TABLE 1.1, TABLE 1.2, TABLE 1.3, TABLE 7, TABLE 9, TABLE 14, TABLE 15, TABLE 16, TABLE 17, or any combination thereof. One alteration alone, or in combination, can produce an effector protein that retains or improves the activity as described herein relative to a reference polypeptide, for example, the wild-type effector protein (SEQ ID NO: 1). In some embodiments, an effector protein provided herein includes at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 alterations as set forth in TABLE 1, TABLE 1.1, TABLE 1.2, TABLE 1.3, TABLE 7, TABLE 9, TABLE 14, TABLE 15, TABLE 16, TABLE 17, or any combination thereof, including up to an alteration at all of the positions identified in TABLE 1, TABLE 1.1, TABLE 1.2, TABLE 1.3, TABLE 7, TABLE 9, TABLE 14, TABLE 15, TABLE 16, TABLE 17, or any combination thereof.

In some embodiments, an effector protein described herein has a combination of alterations comprising a first alteration at a residue corresponding to any position set forth in TABLE 1 relative to SEQ ID NO: 1, and one or more alterations at one or more residues corresponding to any position set forth in TABLE 1 relative to SEQ ID NO: 1 that is not at the position of the first alteration. For example, an effector protein described herein has a combination of alterations comprising a first alteration at a residue corresponding to position 26 relative to SEQ ID NO: 1, and one or more alterations at one or more residues that is not at position 26.

In some embodiments, an effector protein described herein has a combination of alterations comprising a first alteration at a residue corresponding to position 2, 5, 15, 18, 20, 21, 26, 30, 33, 34, 35, 37, 38, 41, 43, 54, 79, 92, 99, 108, 109, 110, 111, 113, 114, 116, 118, 119, 121, 132, 135, 138, 139, 149, 180, 182, 183, 184, 186, 189, 196, 198, 200, 203, 205, 206, 207, 208, 209, 220, 223, 258, 281, 348, 355, 406, 435, 471, 521, 568, 579, 612, 638, 701, or 707, and one or more alterations at one or more residues corresponding to any position set forth in TABLE 1 relative to SEQ ID NO: 1 that is not at the position of the first alteration. For example: in some embodiments, an effector protein described herein has a combination of alterations comprising a first alteration at a residue corresponding to position 26 relative to SEQ ID NO: 1, and one or more alterations at one or more residues corresponding to position 2, 5, 11, 13, 14, 15, 16, 17, 18, 19, 20, 21, 22, 23, 24, 25, 28, 29, 30, 31, 32, 33, 34, 35, 36, 37, 38, 39, 40, 41, 42, 43, 44, 46, 48, 50, 51, 52, 53, 54, 55, 56, 57, 58, 59, 62, 65, 68, 70, 71, 73, 74, 75, 77, 78, 79, 80, 83, 84, 87, 89, 90, 92, 94, 95, 96, 97, 99, 100, 101, 102, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 146, 147, 148, 149, 150, 151, 153, 157, 158, 159, 160, 163, 168, 169, 171, 175, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 218, 220, 223, 225, 227, 228, 229, 230, 231, 232, 233, 234, 236, 238, 239, 240, 241, 242, 243, 244, 245, 246, 247, 248, 250, 252, 253, 254, 255, 256, 257, 258, 259, 260, 262, 264, 265, 266, 268, 272, 273, 276, 279, 280, 281, 285, 286, 295, 297, 298, 301, 302, 304, 306, 311, 312, 315, 316, 328, 329, 334, 338, 340, 348, 355, 356, 357, 360, 361, 363, 366, 368, 369, 370, 384, 390, 391, 392, 393, 395, 397, 399, 400, 405, 406, 407, 435, 445, 471, 472, 480, 483, 497, 501, 503, 509, 511, 512, 513, 514, 515, 516, 517, 521, 523, 526, 529, 531, 536, 540, 541, 542, 543, 544, 545, 546, 549, 567, 568, 577, 579, 585, 590, 591, 592, 593, 594, 595, 596, 599, 602, 603, 604, 605, 606, 607, 608, 612, 617, 620, 624, 634, 638, 639, 653, 658, 673, 674, 678, 679, 682, 684, 685, 696, 699, 701, 703, 707, 709, 715, 716, or any combination thereof, relative to SEQ ID NO: 1; in some embodiments, an effector protein described herein has a combination of alterations comprising a first alteration at a residue corresponding to position 5 relative to SEQ ID NO: 1, and one or more alterations at one or more residues corresponding to position 2, 11, 13, 14, 15, 16, 17, 18, 19, 20, 21, 22, 23, 24, 25, 26, 28, 29, 30, 31, 32, 33, 34, 35, 36, 37, 38, 39, 40, 41, 42, 43, 44, 46, 48, 50, 51, 52, 53, 54, 55, 56, 57, 58, 59, 62, 65, 68, 70, 71, 73, 74, 75, 77, 78, 79, 80, 83, 84, 87, 89, 90, 92, 94, 95, 96, 97, 99, 100, 101, 102, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 146, 147, 148, 149, 150, 151, 153, 157, 158, 159, 160, 163, 168, 169, 171, 175, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 218, 220, 223, 225, 227, 228, 229, 230, 231, 232, 233, 234, 236, 238, 239, 240, 241, 242, 243, 244, 245, 246, 247, 248, 250, 252, 253, 254, 255, 256, 257, 258, 259, 260, 262, 264, 265, 266, 268, 272, 273, 276, 279, 280, 281, 285, 286, 295, 297, 298, 301, 302, 304, 306, 311, 312, 315, 316, 328, 329, 334, 338, 340, 348, 355, 356, 357, 360, 361, 363, 366, 368, 369, 370, 384, 390, 391, 392, 393, 395, 397, 399, 400, 405, 406, 407, 435, 445, 471, 472, 480, 483, 497, 501, 503, 509, 511, 512, 513, 514, 515, 516, 517, 521, 523, 526, 529, 531, 536, 540, 541, 542, 543, 544, 545, 546, 549, 567, 568, 577, 579, 585, 590, 591, 592, 593, 594, 595, 596, 599, 602, 603, 604, 605, 606, 607, 608, 612, 617, 620, 624, 634, 638, 639, 653, 658, 673, 674, 678, 679, 682, 684, 685, 696, 699, 701, 703, 707, 709, 715, 716, or any combination thereof relative to SEQ ID NO: 1; in some embodiments, an effector protein described herein has a combination of alterations comprising a first alteration at a residue corresponding to position 2 relative to SEQ ID NO: 1, and one or more alterations at one or more residues corresponding to position 5, 11, 13, 14, 15, 16, 17, 18, 19, 20, 21, 22, 23, 24, 25, 26, 28, 29, 30, 31, 32, 33, 34, 35, 36, 37, 38, 39, 40, 41, 42, 43, 44, 46, 48, 50, 51, 52, 53, 54, 55, 56, 57, 58, 59, 62, 65, 68, 70, 71, 73, 74, 75, 77, 78, 79, 80, 83, 84, 87, 89, 90, 92, 94, 95, 96, 97, 99, 100, 101, 102, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 146, 147, 148, 149, 150, 151, 153, 157, 158, 159, 160, 163, 168, 169, 171, 175, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 218, 220, 223, 225, 227, 228, 229, 230, 231, 232, 233, 234, 236, 238, 239, 240, 241, 242, 243, 244, 245, 246, 247, 248, 250, 252, 253, 254, 255, 256, 257, 258, 259, 260, 262, 264, 265, 266, 268, 272, 273, 276, 279, 280, 281, 285, 286, 295, 297, 298, 301, 302, 304, 306, 311, 312, 315, 316, 328, 329, 334, 338, 340, 348, 355, 356, 357, 360, 361, 363, 366, 368, 369, 370, 384, 390, 391, 392, 393, 395, 397, 399, 400, 405, 406, 407, 435, 445, 471, 472, 480, 483, 497, 501, 503, 509, 511, 512, 513, 514, 515, 516, 517, 521, 523, 526, 529, 531, 536, 540, 541, 542, 543, 544, 545, 546, 549, 567, 568, 577, 579, 585, 590, 591, 592, 593, 594, 595, 596, 599, 602, 603, 604, 605, 606, 607, 608, 612, 617, 620, 624, 634, 638, 639, 653, 658, 673, 674, 678, 679, 682, 684, 685, 696, 699, 701, 703, 707, 709, 715, 716, or any combination thereof, relative to SEQ ID NO: 1; in some embodiments, an effector protein described herein has a combination of alterations comprising a first alteration at a residue corresponding to position 99 relative to SEQ ID NO: 1, and one or more alteration corresponding to one or more residues at position 2, 5, 11, 13, 14, 15, 16, 17, 18, 19, 20, 21, 22, 23, 24, 25, 26, 28, 29, 30, 31, 32, 33, 34, 35, 36, 37, 38, 39, 40, 41, 42, 43, 44, 46, 48, 50, 51, 52, 53, 54, 55, 56, 57, 58, 59, 62, 65, 68, 70, 71, 73, 74, 75, 77, 78, 79, 80, 83, 84, 87, 89, 90, 92, 94, 95, 96, 97, 100, 101, 102, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 146, 147, 148, 149, 150, 151, 153, 157, 158, 159, 160, 163, 168, 169, 171, 175, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 218, 220, 223, 225, 227, 228, 229, 230, 231, 232, 233, 234, 236, 238, 239, 240, 241, 242, 243, 244, 245, 246, 247, 248, 250, 252, 253, 254, 255, 256, 257, 258, 259, 260, 262, 264, 265, 266, 268, 272, 273, 276, 279, 280, 281, 285, 286, 295, 297, 298, 301, 302, 304, 306, 311, 312, 315, 316, 328, 329, 334, 338, 340, 348, 355, 356, 357, 360, 361, 363, 366, 368, 369, 370, 384, 390, 391, 392, 393, 395, 397, 399, 400, 405, 406, 407, 435, 445, 471, 472, 480, 483, 497, 501, 503, 509, 511, 512, 513, 514, 515, 516, 517, 521, 523, 526, 529, 531, 536, 540, 541, 542, 543, 544, 545, 546, 549, 567, 568, 577, 579, 585, 590, 591, 592, 593, 594, 595, 596, 599, 602, 603, 604, 605, 606, 607, 608, 612, 617, 620, 624, 634, 638, 639, 653, 658, 673, 674, 678, 679, 682, 684, 685, 696, 699, 701, 703, 707, 709, 715, 716, or any combination thereof, relative to SEQ ID NO: 1.

In some embodiments, an effector protein described herein has a combination of alterations comprising a first alteration at a residue corresponding to any position set forth in TABLE 1 relative to SEQ ID NO: 1, and a second alteration at a residue corresponding to any position set forth in TABLE 1 relative to SEQ ID NO: 1 that is not at the position of the first alteration. In some embodiments, an effector protein described herein has a combination of alterations comprising a first alteration at a residue corresponding to any position set forth in TABLE 1 relative to SEQ ID NO: 1, a second alteration at a residue corresponding to any position set forth in TABLE 1 relative to SEQ ID NO: 1 that is not at the position of the first alteration, and a third alteration at a residue corresponding to any position set forth in TABLE 1 relative to SEQ ID NO: 1 that is not at the position of the first alteration or the second alteration. Likewise, in some embodiments, an effector protein described herein has a combination of alterations comprising a first alteration at a residue corresponding to any position set forth in TABLE 1 relative to SEQ ID NO: 1, a second alteration at a residue corresponding to any position set forth in TABLE 1 relative to SEQ ID NO: 1 that is not at the position of the first alteration, a third alteration at a residue corresponding to any position set forth in TABLE 1 relative to SEQ ID NO: 1 that is not at the position of the first alteration or the second alteration, and a fourth alteration at a residue corresponding to any position set forth in TABLE 1 relative to SEQ ID NO: 1 that is not at the position of the first alteration, the second alteration, or the third alteration. A person of ordinary skill in the art would readily understand when combinations of alterations are described herein, each alteration is at a different amino acid position.

In some embodiments, an effector protein described herein has a combination of alterations comprising a first alteration at a residue corresponding to position 2, 5, 26, 99, 118, 184, 198, 348, 579, 612, or 701 relative to SEQ ID NO: 1. In some embodiments, the first amino acid alteration is a substitution with a basic (positively charged) amino acid, an acidic (negatively-charged) amino acid, a non-polar (hydrophobic) amino acid, or an uncharged polar amino acid. In some embodiments, the first amino acid alteration is a substitution with an Arg (R).

In some embodiments, an effector protein described herein has a combination of alterations comprising a second alteration at a residue corresponding to position 16, 26, 50, 57, 59, 70, 73, 83, 92, 94, 96, 97, 100, 109, 119, 121, 139, 150, 153, 157, 158, 186, 189, 199, 220, 223, 227, 228, 229, 230, 231, 232, 233, 234, 236, 238, 239, 241, 242, 243, 244, 245, 246, 247, 248, 250, 252, 253, 254, 255, 256, 257, 258, 259, 260, 264, 265, 266, 268, 279, 297, 361, 405, 406, 435, 471, 472, 497, 521, 568, 585, 638, 673, 674, 678, 679, 682, 684, 685, 696, 699, 703, 709, 715, or 716 relative to SEQ ID NO: 1. In some embodiments, the second amino acid alteration is a substitution with a basic (positively charged) amino acid, an acidic (negatively-charged) amino acid, a non-polar (hydrophobic) amino acid, or an uncharged polar amino acid. In some embodiments, the second amino acid alteration is a substitution with a G, R, K, E, S, Q, P, T, D, or F.

In some embodiments, an effector protein described herein has a combination of alterations comprising a third alteration at a residue corresponding to position 208 or 184 relative to SEQ ID NO: 1. In some embodiments, the third amino acid alteration is a substitution with a basic (positively charged) amino acid, an acidic (negatively-charged) amino acid, a non-polar (hydrophobic) amino acid, or an uncharged polar amino acid. In some embodiments, the second amino acid alteration is a substitution with a R.

In some embodiments, an effector protein described herein has a combination of alterations comprising a fourth alteration at a residue corresponding to position 114 relative to SEQ ID NO: 1. In some embodiments, the third amino acid alteration is a substitution with a basic (positively charged) amino acid, an acidic (negatively-charged) amino acid, a non-polar (hydrophobic) amino acid, or an uncharged polar amino acid. In some embodiments, the second amino acid alteration is a substitution with a R.

In some embodiments, the first amino acid alteration is at a residue corresponding to position 26 relative to SEQ ID NO: 1; in some embodiments, the first amino acid alteration is a substitution with an R; the second amino acid alteration is at a residue corresponding to position 16, 30, 38, 50, 57, 59, 70, 73, 83, 94, 96, 97, 99, 100, 108, 109, 114, 119, 149, 150, 153, 157, 158, 182, 183, 184, 198, 199, 208, 220, 223, 227, 228, 229, 230, 231, 232, 233, 234, 236, 238, 239, 241, 242, 243, 244, 245, 246, 247, 248, 250, 252, 253, 254, 255, 256, 257, 258, 259, 260, 264, 265, 266, 268, 279, 281, 297, 348, 355, 361, 405, 435, 471, 472, 497, 521, 568, 585, 638, 673, 674, 678, 679, 682, 684, 685, 696, 699, 703, 707, 709, 715, or 716 relative to SEQ ID NO: 1; the second amino acid alteration is a substitution with a G, R, Q, K, E, P, T, S, D, or F; in some embodiments, the third amino acid alteration is at a residue corresponding to position 208 or 184 relative to SEQ ID NO: 1; the third amino acid alteration is a substitution with an R; the fourth amino acid alteration at a residue corresponding to position 114 relative to SEQ ID NO: 1; in some embodiments, the fourth amino acid alteration is a substitution with an R; or any combination thereof.

In some embodiments, the first amino acid alteration is at a residue corresponding to position 184 relative to SEQ ID NO: 1; in some embodiments, the first amino acid alteration is a substitution with an R; the second amino acid alteration at a residue corresponding to position 183, 114, 109, 198, 208, 182, 108, or 38 relative to SEQ ID NO: 1; in some embodiments, the second amino acid alteration is a substitution with an R; or any combinations thereof.

In some embodiments, the first amino acid alteration is at residue corresponding to position 5 relative to SEQ ID NO: 1; in some embodiments, the first amino acid alteration is a substitution with an R; the second amino acid alteration at a residue corresponding to position 92, 121, 139, 189, 220, 223, 258, 406, 435, 471, 521, 568, or 638 relative to SEQ ID NO: 1; in some embodiments, the second amino acid alteration is a substitution with an R, Q, P, S, K, T, D, or E; or any combinations thereof.

In some embodiments, the first amino acid alteration is at residue corresponding to position 2 relative to SEQ ID NO: 1; in some embodiments, the first amino acid alteration is a substitution with an R; and the second amino acid alteration at a residue corresponding to position 139 relative to SEQ ID NO: 1; in some embodiments, the second amino acid alteration is a substitution with an R.

In some embodiments, the first amino acid alteration is at residue corresponding to position 99 relative to SEQ ID NO: 1; in some embodiments, the first amino acid alteration is a substitution with an R; and the second amino acid alteration at a residue corresponding to position 186 relative to SEQ ID NO: 1; in some embodiments, the second amino acid alteration is a substitution with an R.

In some embodiments, the first amino acid alteration is at residue corresponding to position 118 relative to SEQ ID NO: 1; in some embodiments, the first amino acid alteration is a substitution with an R; the second amino acid alteration is at residue corresponding to position 92, 189, or 568 relative to SEQ ID NO: 1; in some embodiments, the second amino acid alteration is a substitution with a P, E or D; or any combination thereof.

In some embodiments, the first amino acid alteration is at residue corresponding to position 186 relative to SEQ ID NO: 1; in some embodiments, the first amino acid alteration is a substitution with an R; the second amino acid alteration is at residue corresponding to position 258, 521, 568 relative to SEQ ID NO: 1; in some embodiments, the second amino acid alteration is a substitution with a K, T or D; or any combination thereof.

In some embodiments, the first amino acid alteration is at residue corresponding to position 198 relative to SEQ ID NO: 1; in some embodiments, the first amino acid alteration is a substitution with an R; the second amino acid alteration is at residue corresponding to position 92, 119, 189, 220, 223, 258, 406, 471, 521, 568, or 638 relative to SEQ ID NO: 1; in some embodiments, the second amino acid alteration is a substitution with a E, S, P, K, T or D; or any combination thereof.

In some embodiments, the first amino acid alteration is at residue corresponding to position 348 relative to SEQ ID NO: 1; in some embodiments, the first amino acid alteration is a substitution with an R; the second amino acid alteration is at residue corresponding to position 26, 92, 119, 121, 189, 220, 223, 258, 406, 435, 471, 521, or 568 relative to SEQ ID NO: 1; in some embodiments, the second amino acid alteration is a substitution with a S, Q, P, K, T, D, or E; or any combination thereof.

In some embodiments, the first amino acid alteration is at residue corresponding to position 579 relative to SEQ ID NO: 1; in some embodiments, the first amino acid alteration is a substitution with an R; the second amino acid alteration is at residue corresponding to position 26, 92, 119, 121, 189, 220, 223, 258, 406, 435, 471, 521, 568, or 638 relative to SEQ ID NO: 1; in some embodiments, the second amino acid alteration is a substitution with a S, Q, P, K, T, D, or E; or any combination thereof.

In some embodiments, the first amino acid alteration is at residue corresponding to position 612 relative to SEQ ID NO: 1; in some embodiments, the first amino acid alteration is a substitution with an R.; the second amino acid alteration is at residue corresponding to position 26, 92, 119, 121, 189, 220, 223, 258, 406, 435, 471, 521, 568, or 638 relative to SEQ ID NO: 1; in some embodiments, the second amino acid alteration is a substitution with a S, Q, P, K, T, D, or E; or any combination thereof.

In some embodiments, the first amino acid alteration is at residue corresponding to position 701 relative to SEQ ID NO: 1; in some embodiments, the first amino acid alteration is a substitution with an R; the second amino acid alteration is at residue corresponding to position 26, 92, 119, 121, 189, 220, 223, 258, 406, 435, 471, 521, 568, or 638 relative to SEQ ID NO: 1; in some embodiments, the second amino acid alteration is a substitution with a S, Q, P, K, T, D, or E; or any combination thereof.

In some embodiments, an effector protein described herein comprises an L26K alteration relative to relative to SEQ ID NO: 1. In some embodiments, an effector protein described herein comprises an T5R alteration relative to relative to SEQ ID NO: 1. In some embodiments, an effector protein described herein comprises an I471T alteration relative to relative to SEQ ID NO: 1. In some embodiments, an effector protein described herein comprises an S579R alteration relative to relative to SEQ ID NO: 1. In some embodiments, an effector protein described herein comprises an F701R alteration relative to relative to SEQ ID NO: 1. In some embodiments, an effector protein described herein comprises an S223P alteration relative to relative to SEQ ID NO: 1. In some embodiments, an effector protein described herein comprises an A121Q alteration relative to relative to SEQ ID NO: 1. In some embodiments, an effector protein described herein comprises an E258K alteration relative to relative to SEQ ID NO: 1. In some embodiments, an effector protein described herein comprises an S198R alteration relative to relative to SEQ ID NO: 1.

In some embodiments, an effector protein described herein has a combination of alterations comprising L26R and K184R relative to SEQ ID NO: 1. In some embodiments, an effector protein described herein has a combination of alterations comprising L26R and Q183R relative to SEQ ID NO: 1. In some embodiments, an effector protein described herein has a combination of alterations comprising L26R and Ti14R relative to SEQ ID NO: 1. In some embodiments, an effector protein described herein has a combination of alterations comprising L26R and E109R relative to SEQ ID NO: 1. In some embodiments, an effector protein described herein has a combination of alterations comprising L26R and S198R relative to SEQ ID NO: 1. In some embodiments, an effector protein described herein has a combination of alterations comprising L26R and H208R relative to SEQ ID NO: 1. In some embodiments, an effector protein described herein has a combination of alterations comprising L26R and L182R relative to SEQ ID NO: 1. In some embodiments, an effector protein described herein has a combination of alterations comprising L26R and S108R relative to SEQ ID NO: 1. In some embodiments, an effector protein described herein has a combination of alterations comprising L26R and K38R relative to SEQ ID NO: 1. In some embodiments, an effector protein described herein has a combination of alterations comprising K184R and Q183R relative to SEQ ID NO: 1. In some embodiments, an effector protein described herein has a combination of alterations comprising K184R and T114R relative to SEQ ID NO: 1. In some embodiments, an effector protein described herein has a combination of alterations comprising K184R and E109R relative to SEQ ID NO: 1. In some embodiments, an effector protein described herein has a combination of alterations comprising K184R and S198R relative to SEQ ID NO: 1. In some embodiments, an effector protein described herein has a combination of alterations comprising K184R and H208R relative to SEQ ID NO: 1. In some embodiments, an effector protein described herein has a combination of alterations comprising K184R and L182R relative to SEQ ID NO: 1. In some embodiments, an effector protein described herein has a combination of alterations comprising K184R and S108R relative to SEQ ID NO: 1. In some embodiments, an effector protein described herein has a combination of alterations comprising K184R and K38R relative to SEQ ID NO: 1. In some embodiments, an effector protein described herein has a combination of alterations comprising L26R, K184R, and H208R relative to SEQ ID NO: 1. In some embodiments, an effector protein described herein has a combination of alterations comprising L26R, Q183R, and K184R relative to SEQ ID NO: 1. In some embodiments, an effector protein described herein has a combination of alterations comprising L26R, Q183R, and H208R relative to SEQ ID NO: 1. In some embodiments, an effector protein described herein has a combination of alterations comprising L26R, Q183R, K184R, and Ti14R relative to SEQ ID NO: 1. In some embodiments, an effector protein described herein has a combination of alterations comprising L26R and K99R relative to SEQ ID NO: 1. In some embodiments, an effector protein described herein has a combination of alterations comprising L26R and P707R relative to SEQ ID NO: 1. In some embodiments, an effector protein described herein has a combination of alterations comprising L26R and L149R relative to SEQ ID NO: 1. In some embodiments, an effector protein described herein has a combination of alterations comprising L26R and N30R relative to SEQ ID NO: 1. In some embodiments, an effector protein described herein has a combination of alterations comprising L26R and N355R relative to SEQ ID NO: 1. In some embodiments, an effector protein described herein has a combination of alterations comprising L26R and K281R relative to SEQ ID NO: 1. In some embodiments, an effector protein described herein has a combination of alterations comprising L26R and S108R relative to SEQ ID NO: 1. In some embodiments, an effector protein described herein has a combination of alterations comprising L26R and K348R relative to SEQ ID NO: 1. In some embodiments, an effector protein described herein has a combination of alterations comprising T5R and V139R relative to SEQ ID NO: 1. In some embodiments, an effector protein described herein has a combination of alterations comprising I2R and V139R relative to SEQ ID NO: 1. In some embodiments, an effector protein described herein has a combination of alterations comprising K99R and S186R relative to SEQ ID NO: 1. In some embodiments, the effector protein comprises D369A or D369N alteration relative to SEQ ID NO: 1. In some embodiments, the effector protein comprises E567A or E567Q alteration relative to SEQ ID NO: 1. In some embodiments, the effector protein comprises D658A or D658N alteration relative to SEQ ID NO: 1. In some embodiments, an effector protein described herein has a combination of alterations comprising L26R and A673G relative to SEQ ID NO: 1. In some embodiments, an effector protein described herein has a combination of alterations comprising L26R and Q674R relative to SEQ ID NO: 1. In some embodiments, an effector protein described herein has a combination of alterations comprising S579R and L26K relative to SEQ ID NO: 1. In some embodiments, an effector protein described herein has a combination of alterations comprising F701R and E258K relative to SEQ ID NO: 1. In some embodiments, an effector protein described herein has a combination of alterations comprising T5R and L26K relative to SEQ ID NO: 1. In some embodiments, an effector protein described herein has a combination of alterations comprising L26R and K435Q relative to SEQ ID NO: 1. In some embodiments, an effector protein described herein has a combination of alterations comprising L26R and G685R relative to SEQ ID NO: 1. In some embodiments, an effector protein described herein has a combination of alterations comprising L26R and Q674K relative to SEQ ID NO: 1. In some embodiments, an effector protein described herein has a combination of alterations comprising L26R and P699R relative to SEQ ID NO: 1. In some embodiments, an effector protein described herein has a combination of alterations comprising L26R and T252R relative to SEQ ID NO: 1. In some embodiments, an effector protein described herein has a combination of alterations comprising L26R and P679R relative to SEQ ID NO: 1. In some embodiments, an effector protein described herein has a combination of alterations comprising L26R and S223P relative to SEQ ID NO: 1. In some embodiments, an effector protein described herein has a combination of alterations comprising S198R and 147IT relative to SEQ ID NO: 1. In some embodiments, an effector protein described herein has a combination of alterations comprising L26R and E682R relative to SEQ ID NO: 1. In some embodiments, an effector protein described herein has a combination of alterations comprising L26R and I471T relative to SEQ ID NO: 1. In some embodiments, an effector protein described herein has a combination of alterations comprising L26R and S638K relative to SEQ ID NO: 1. In some embodiments, an effector protein described herein has a combination of alterations comprising L26R and A150K relative to SEQ ID NO: 1.

An effector protein provided herein may comprise at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or at most 250 alterations relative to a wild-type effector protein (SEQ ID NO: 1).

Provided herein is a composition comprising a polypeptide or a nucleic acid encoding the polypeptide, wherein the polypeptide comprises an amino acid sequence that, other than the amino acid residue at position 26, is at least 90% identical to any one of the amino acid sequences recited in TABLE 1. Also provided herein are compositions comprising a polypeptide or a nucleic acid encoding a polypeptide, wherein the polypeptide comprises an amino acid sequence that, other than the amino acid residue at position 26, is at least 95% identical to any one of the amino acid sequences recited in TABLE 1. Also provided herein are compositions comprising a polypeptide or a nucleic acid encoding a polypeptide, wherein the polypeptide comprises an amino acid sequence that, other than the amino acid residue at position 26, is at least 98% identical to any one of the amino acid sequences recited in TABLE 1. Also provided herein are compositions comprising a polypeptide or a nucleic acid encoding a polypeptide, wherein the polypeptide comprises an amino acid sequence that, other than the amino acid residue at position 26, is at least 99% identical to any one of the amino acid sequences recited in TABLE 1. Also provided herein are compositions comprising a polypeptide or a nucleic acid encoding a polypeptide, wherein the polypeptide comprises an amino acid sequence recited in TABLE 1. Also provided herein are compositions comprising a polypeptide or a nucleic acid encoding a polypeptide, wherein the amino acid sequence of the polypeptide is SEQ ID NO: 2.

Also provided herein are compositions comprising a polypeptide or a nucleic acid encoding a polypeptide, wherein the amino acid sequence comprises at least one conservative amino acid substitution at a position other than position 26. Also provided herein are compositions comprising a polypeptide or a nucleic acid encoding a polypeptide, wherein the amino acid sequence comprises at least one non-conservative amino acid substitution at a position other than position 26. Also provided herein are compositions comprising a polypeptide or a nucleic acid encoding a polypeptide, wherein the polypeptide comprises no more than 1, 2, 3, 4, 5, 6, 7, 8, 9 or 10 non-conservative amino acid substitutions relative to the amino acid sequence of any one of the sequences recited in TABLE 1. Also provided herein are compositions comprising a polypeptide or a nucleic acid encoding a polypeptide comprising at least one non-conservative amino acid substitution, wherein the non-conservative amino acid substitution is to substitute an amino acid residue with a basic (positively charged) amino acid substitution. Also provided herein are compositions comprising a polypeptide or a nucleic acid encoding a polypeptide comprising at least one non-conservative amino acid substitution, wherein the non-conservative amino acid substitution is to substitute an amino acid residue with a Lys (K), Arg (R), or His (H). Also provided herein are compositions comprising a polypeptide or a nucleic acid encoding a polypeptide comprising at least one conservative or non-conservative amino acid substitution, wherein the conservative or non-conservative amino acid substitution is in a region of the polypeptide that interacts with a target nucleic acid.

Also provided herein are compositions comprising a polypeptide or a nucleic acid encoding a polypeptide, wherein the polypeptide comprises at least one amino acid alteration in a domain of the polypeptide that interacts with a target nucleic acid. Also provided herein are compositions comprising a polypeptide or a nucleic acid encoding a polypeptide, wherein the polypeptide generates increased indels in a target nucleic acid relative to a polypeptide consisting of the amino acid sequence of SEQ ID NO: 1. Also provided herein are compositions comprising a polypeptide or a nucleic acid encoding a polypeptide, wherein the polypeptide generates at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% more indels in a population of cells relative to the number of indels generated by an effector protein consisting of the amino acid sequence of SEQ ID NO: 1, as measured in a cleavage assay.

Also provided herein are compositions comprising a polypeptide or a nucleic acid encoding a polypeptide, wherein the polypeptide recognizes a target nucleic acid, and wherein the target nucleic acid comprises a protospacer adjacent motif (PAM) sequence of 5′-NTTN-3′ that is located adjacent to a target sequence.

Also provided herein are compositions comprising a polypeptide or a nucleic acid encoding a polypeptide, wherein the polypeptide comprises at least one nuclear localization signal. Also provided herein are compositions comprising a polypeptide or a nucleic acid encoding a polypeptide, wherein the polypeptide comprises a sequence selected from of any one of SEQ ID NOS: 5-13.

Also provided herein are compositions comprising a polypeptide or a nucleic acid encoding a polypeptide, comprising a fusion partner protein linked to the polypeptide. Also provided herein are compositions comprising a polypeptide or a nucleic acid encoding a polypeptide and a fusion partner, wherein the fusion partner protein is fused to the N terminus or C terminus of the polypeptide via an amide bond or at least one linker. Also provided herein are compositions comprising a polypeptide or a nucleic acid encoding a polypeptide, wherein the polypeptide comprises at least one mutation that reduces its nuclease activity relative to a polypeptide consisting of the amino acid sequence of SEQ ID NO: 1, as measured in a cleavage assay.

Also provided herein are compositions comprising a polypeptide or a nucleic acid encoding a polypeptide comprising at least one mutation, wherein the at least one mutation that reduces its nuclease activity is located at a position in a RuvC domain.

Exemplary sequences of variant effector proteins described herein are set forth in TABLE 1.4.

In some embodiments, the effector proteins function as an endonuclease that catalyzes cleavage within a target nucleic acid. In some embodiments, the effector proteins are capable of catalyzing non-sequence-specific cleavage of a single stranded nucleic acid. In some embodiments, the effector proteins (e.g., the effector proteins comprising the one or more alterations set forth in TABLE 1, TABLE 1.1, TABLE 1.2, TABLE 1.3,) are activated to perform trans cleavage activity after binding of a guide nucleic acid with a target nucleic acid. Trans cleavage activity may be non-specific cleavage of nearby single-stranded nucleic acid by the activated effector protein, such as trans cleavage of detector nucleic acids with a detection moiety.

Effector proteins disclosed herein may function as an endonuclease that catalyzes cleavage at a specific position (e.g., at a specific nucleotide within a nucleic acid sequence) in a target nucleic acid. The target nucleic acid may be single stranded RNA (ssRNA), double stranded DNA (dsDNA) or single-stranded DNA (ssDNA). In some embodiments, the target nucleic acid is single-stranded DNA. In some embodiments, the target nucleic acid is single-stranded RNA. The effector proteins may provide cis cleavage activity, trans cleavage activity, nickase activity, or a combination thereof. Cis cleavage activity is cleavage of a target nucleic acid that is hybridized to a guide RNA (e.g., a crRNA), wherein cleavage occurs within or directly adjacent to the region of the target nucleic acid that is hybridized to guide RNA. Trans cleavage activity (also referred to as transcollateral cleavage) is cleavage of ssDNA or ssRNA that is near, but not hybridized to the guide RNA. Trans cleavage may occur near, but not within or directly adjacent to, the region of the target nucleic acid that is hybridized to the guide nucleic acid. Trans cleavage activity may be triggered by the hybridization of the guide nucleic acid to the target nucleic acid. Nickase activity is a selective cleavage of one strand of a dsDNA.

### Effector Partners

Provided herein are compositions, systems, and methods comprising one or more effector partners or uses thereof. In some embodiments, the effector partner is a heterologous protein or an effector protein described herein. In some embodiments, the effector partner is not an effector protein as described herein. In some embodiments, the effector partner is capable of imparting a function or activity that is not provided by an effector protein as described herein. In some embodiments, the effector partner comprises a second effector protein or a multimeric form thereof. In some embodiments, an effector partner includes or is a fusion partner. In some embodiments, an effector partner is referred to interchangeably herein as a fusion partner, and vice versa.

In some embodiments, a fusion effector protein, a fusion protein, or a fusion polypeptide, as referred to interchangeably herein, comprise a protein comprising at least two heterologous polypeptides. Often a fusion effector protein comprises an effector protein and a fusion partner protein. A fusion partner protein is also simply referred to herein as a fusion partner. In general, the fusion partner is not an effector protein. In some cases, an effector partner or a fusion partner comprises a polypeptide or peptide that is fused to an effector protein. In some embodiments, when describing components that are fused, reference is made to at least two sequences that are connected together, such as by a covalent bond (e.g., an amide bond or a phosphodiester bond) or by a linker. The covalent bond can be formed by a conjugation (e.g., chemical conjugation or enzymatic conjugation) reaction.

The fusion partner generally imparts some function to the fusion protein that is not provided by the effector protein. Unless otherwise indicated, reference to effector proteins (e.g., a CasPhi.12 variant) throughout the present disclosure include fusion proteins thereof.

In some embodiments, the effector partner (e.g., the fusion partner) is fused or linked to an effector protein described herein. In some embodiments, the amino terminus of the effector partner (e.g., the fusion partner) is linked to the carboxy terminus of the effector protein directly or by a linker. In some embodiments, the carboxy terminus of the effector partner (e.g., the fusion partner) is linked to the amino terminus of the effector protein directly or by a linker. In some embodiments, the effector partner (e.g., the fusion partner) may be functional when the effector protein is coupled to a guide nucleic acid. In some embodiments, the effector partner (e.g., the fusion partner) may be functional when the effector protein is coupled to a target nucleic acid. In some embodiments, the guide nucleic acid imparts sequence specific activity to the effector partner (e.g., the fusion partner). By way of non-limiting example, the effector protein may comprise a catalytically inactive effector protein (e.g., a catalytically inactive variant of an effector protein described herein) when fused or linked to an effector partner (e.g., the fusion partner).

In some embodiments, an effector protein described herein, or a fusion protein thereof functions as a multimeric complex. In some embodiments, effector proteins form a homodimer. In some embodiments, fusion proteins described herein form a homodimer. In some embodiments, fusion proteins described herein form a heterodimer. In some embodiments, the effector proteins of the multimeric complex dimerize, thereby bringing multiple fusion partners into proximity of one another.

In some embodiments, an effector partner (e.g., a fusion partner), imparts a function or activity to a fusion protein comprising an effector protein that is not provided by the effector protein, including but not limited to nuclease activity, methyltransferase activity, demethylase activity, DNA repair activity, DNA damage activity, deamination activity, dismutase activity, alkylation activity, depurination activity, oxidation activity, dimer forming activity (e.g., pyrimidine dimer forming activity), integrase activity, transposase activity, recombinase activity, polymerase activity, ligase activity, helicase activity, photolyase activity, glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity, modification of a polypeptide associated with target nucleic acid (e.g., a histone), and/or signaling activity.

In some embodiments, the effector partner (e.g., fusion partner) may directly or indirectly edit a target nucleic acid. Edits can be of a nucleobase, nucleotide, or nucleotide sequence of a target nucleic acid. For example, the fusion partner may modify a target nucleic acid, including changing a nucleobase of the target nucleic acid and making a chemical modification to one or more nucleotides of the target nucleic acid. The fusion partner may be capable of modulating the expression of a target nucleic acid. In some embodiments, the effector partner (e.g., fusion partner) may interact with additional proteins, or functional fragments thereof, to make modifications to a target nucleic acid. In other embodiments, the effector partner (e.g., fusion partner) may modify proteins associated with a target nucleic acid. In some embodiments, an effector partner (e.g., fusion partner) may modulate transcription (e.g., inhibits transcription, increases transcription) of a target nucleic acid. In yet another example, an effector partner (e.g., fusion partner) may directly or indirectly inhibit, reduce, activate or increase expression of a target nucleic acid. For example, the fusion partner may inhibit, reduce, activate or increase expression of a target nucleic acid via additional proteins or nucleic acid modifications to the target sequence. In some embodiments, an effector partner (e.g., fusion partner) may provide detectable activity. For example, the fusion partner may provide a detectable signal.

### Multimeric Complex Formation Modification Activity

In some cases, the fusion partner promotes the formation of a multimeric complex of the effector protein. In some instances, the fusion partner inhibits the formation of a multimeric complex of the effector protein. By way of non-limiting example, the fusion protein may comprise a CasPhi.12 variant, and a fusion partner comprising a Calcineurin A tag, wherein the fusion protein dimerizes in the presence of Tacrolimus (FK506). Also by way of non-limiting example, the fusion protein may comprise a CasPhi.12 variant and a SpyTag configured to dimerize or associate with another effector protein in a multimeric complex.

### Reverse Transcriptase (RT) Editing System

In some embodiments, systems and methods comprise components or uses of an RT editing system to modify a target nucleic acid. RT editing may also be referred to as prime editing or precise nucleobase editing. In some embodiments, an RT editing system comprises an effector protein and an effector partner (e.g., fusion partner) comprising an RT editing enzyme. In some embodiments, the effector protein that is linked to the RT editing enzyme. In some embodiments, an RT editing enzyme comprises a polymerase. In some embodiments, an RT editing enzyme comprises a reverse transcriptase. A non-limiting example of a reverse transcriptase is an M-MLV RT enzyme and variants thereof having polymerase activity. In some embodiments, the M-MLV RT enzyme comprises at least one mutation selected from D200N, L603W, T330P, T306K, and W313F relative to wildtype M-MLV RT enzyme. In some embodiments, systems and methods comprise an RT editing enzyme, wherein the RT editing enzyme is not fused or linked to the effector protein. In some embodiments, the RT editing enzyme comprises a recruiting moiety that recruits the RT editing enzyme to the target nucleic acid. By way of non-limiting example, the RT editing enzyme may comprise a peptide that binds an aptamer, wherein the aptamer is located on a guide RNA, template RNA, or combination thereof. Also, by way of non-limiting example, the RT editing enzyme may be linked to a protein that binds to (or is bound by) the effector protein or a protein linked/fused to the effector protein.

In some embodiments, an RT editing enzyme may require an RT editing guide RNA (pegRNA) to catalyze editing. Such a pegRNA may be capable of identifying a target nucleotide or target sequence in a target nucleic acid to be edited and encoding a new genetic information that replaces the target nucleotide or target sequence in the target nucleic acid. An RT editing enzyme may require a pegRNA and a guide RNA, such as a single guide RNA, to catalyze the editing. In some embodiments, the RT editing system comprises a template RNA comprising a primer binding sequence that hybridizes to a primer sequence of the dsDNA molecule that is formed when target nucleic acid is cleaved, and a template sequence that is complementary to at least a portion of the target sequence of the dsDNA molecule except for at least one nucleotide. In some embodiments, the template RNA is covalently linked to a guide RNA. In some embodiments, the template RNA is not covalently linked to a guide RNA. In some embodiments, at least a portion of the template RNA hybridizes to the target nucleic acid. In some embodiments, the target nucleic acid is a dsDNA molecule. In some embodiments, at least a portion of the template RNA hybridizes to a first strand of the target nucleic acid and at least a portion of the guide RNA hybridizes to a second strand of the target nucleic acid. In some embodiments, the pegRNA comprises: a guide RNA comprising a second region that is bound by the effector protein, and a first region comprising a spacer sequence that is complementary to a target sequence of the dsDNA molecule; and a template RNA comprising a primer binding sequence that hybridizes to a primer sequence of the dsDNA molecule that is formed when target nucleic acid is cleaved, and a template sequence that is complementary to at least a portion of the target sequence of the dsDNA molecule with the exception of at least one nucleotide. In some embodiments, the at least one nucleotide is incorporated into the target nucleic acid by activity of the RT editing enzyme, thereby modifying the target nucleic acid. In some embodiments, the spacer sequence is complementary to the target sequence on a target strand of the dsDNA molecule. In some embodiments, the spacer sequence is complementary to the target sequence on a non-target strand of the dsDNA molecule. In some embodiments, the primer binding sequence hybridizes to a primer sequence on the non-target strand of the dsDNA molecule. In some embodiments, the primer binding sequence hybridizes to a primer sequence on the target strand of the dsDNA molecule. In some embodiments, the target strand is cleaved. In some embodiments, the non-target strand is cleaved.

In some instances, fusion partners include, but are not limited to, a protein that directly and/or indirectly provides for increased or decreased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). In some instances, fusion partners that increase or decrease transcription include a transcription activator domain or a transcription repressor domain, respectively.

In some cases, the fusion partner modulates transcription (e.g., inhibits transcription, increases transcription) of a target nucleic acid. In some cases, the fusion partner is a protein (or a domain from a protein) that inhibits transcription, also referred to as a transcriptional repressor. Transcriptional repressors may inhibit transcription via recruitment of transcription inhibitor proteins, modification of target DNA such as methylation, recruitment of a DNA modifier, modulation of histones associated with target DNA, recruitment of a histone modifier such as those that modify acetylation and/or methylation of histones, or a combination thereof. In some cases, the fusion partner is a protein (or a domain from a protein) that increases transcription, also referred to as a transcription activator. Transcriptional activators may promote transcription via recruitment of transcription activator proteins, modification of target DNA such as demethylation, recruitment of a DNA modifier, modulation of histones associated with target DNA, recruitment of a histone modifier such as those that modify acetylation and/or methylation of histones, or a combination thereof. In some cases, the fusion partner is a reverse transcriptase. In some cases, the fusion partner is a base editor. In general, a base editor comprises a deaminase that when fused with a Cas protein changes a nucleobase to a different nucleobase, e.g., cytosine to thymine or guanine to adenine. In some instances, the base editor comprises a deaminase.

In some cases, fusion proteins are targeted by a guide nucleic acid (e.g., guide RNA) to a specific location in the target nucleic acid and exert locus-specific regulation such as blocking RNA polymerase binding to a promoter (which selectively inhibits transcription activator function), and/or modifying the local chromatin status (e.g., when a fusion sequence is used that modifies the target nucleic acid or modifies a protein associated with the target nucleic acid). In some cases, the modifications are transient (e.g., transcription repression or activation). In some cases, the modifications are inheritable. For instance, epigenetic modifications made to a target nucleic acid, or to proteins associated with the target nucleic acid, e.g., nucleosomal histones, in a cell, are observed in cells produced by proliferation of the cell.

### Nucleic Acid Modification Activity

In some cases, fusion partners provide enzymatic activity that modifies a nucleic acid, such as a target nucleic acid. Such enzymatic activities include, but are not limited to, nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, and glycosylase activity. In some cases, nuclease activity which comprises the enzymatic activity of an enzyme which allows the enzyme to cleave the phosphodiester bonds between the nucleotide subunits of nucleic acids. In some case, an enzyme with nuclease activity can comprise a nuclease.

In some instances, compositions and methods use effector proteins that are fused to a heterologous protein. Heterologous proteins include, but are not limited to, transcriptional activators, transcriptional repressors, deaminases, methyltransferases, acetyltransferases, and other nucleic acid modifying proteins. In some cases, effector proteins need not be fused to a partner protein to accomplish the required protein (expression) modification.

In some cases, fusion partners have enzymatic activity that modifies the target nucleic acid. The target nucleic acid may comprise or consist of a ssRNA, dsRNA, ssDNA, or a dsDNA. Examples of enzymatic activity that modifies the target nucleic acid include, but are not limited to: nuclease activity such as that provided by a restriction enzyme (e.g., FokI nuclease); methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants)); demethylase activity such as that provided by a demethylase (e.g., Ten-Eleven Translocation (TET) dioxygenase 1 (TET1CD), TET1, DME, DML1, DML2, ROS1); DNA repair activity; DNA damage (e.g., oxygenation) activity; deamination activity such as that provided by a deaminase (e.g., a cytosine deaminase enzyme such as rat APOBEC1); dismutase activity; alkylation activity; depurination activity; oxidation activity; pyrimidine dimer forming activity; integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase); transposase activity, recombinase activity such as that provided by a recombinase (e.g., catalytic domain of Gin recombinase); as well as polymerase activity, ligase activity, helicase activity, photolyase activity, and glycosylase activity.

In some embodiments, effector partners (e.g., fusion partners) target a ssRNA, dsRNA, ssDNA, or a dsDNA. In some embodiments, effector partners (e.g., fusion partners) target ssRNA. Non-limiting examples of fusion partners for targeting ssRNA include, but are not limited to, splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; and RNA-binding proteins. It is understood that a fusion protein may include the entire protein or in some cases may include a fragment of the protein (e.g., a functional domain). In some instances, the functional domain interacts with or binds ssRNA, including intramolecular and/or intermolecular secondary structures thereof, e.g., hairpins, stem-loops, etc.). The functional domain may interact transiently or irreversibly, directly or indirectly. In some cases, a functional domain comprises a region of one or more amino acids in a protein that is required for an activity of the protein, or the full extent of that activity, as measured in an in vitro assay. Activities include, but are not limited to nucleic acid binding, nucleic acid modification, nucleic acid cleavage, protein binding. The absence of the functional domain, including mutations of the functional domain, would abolish or reduce activity. Fusion proteins may comprise a protein or domain thereof selected from: endonucleases (e.g., RNase III, the CRR22 DYW domain, Dicer, and PIN (PilT N-terminus); SMG5 and SMG6; domains responsible for stimulating RNA cleavage (e.g., CPSF, CstF, CFIm and CFIIm); exonucleases such as XRN-1 or Exonuclease T; deadenylases such as HNT3; protein domains responsible for nonsense mediated RNA decay (e.g., UPF1, UPF2, UPF3, UPF3b, RNP Si, Y14, DEK, REF2, and SRm160); protein domains responsible for stabilizing RNA (e.g., PABP); proteins and protein domains responsible for repressing translation (e.g., Ago2 and Ago4); proteins and protein domains responsible for stimulating translation (e.g., Staufen); proteins and protein domains responsible for (e.g., capable of) modulating translation (e.g., translation factors such as initiation factors, elongation factors, release factors, etc., e.g., eIF4G); proteins and protein domains responsible for polyadenylation of RNA (e.g., PAP1, GLD-2, and Star-PAP); proteins and protein domains responsible for polyuridinylation of RNA (e.g., CI D1 and terminal uridylate transferase); proteins and protein domains responsible for RNA localization (e.g., from IMP1, ZBP1, She2p, She3p, and Bicaudal-D); proteins and protein domains responsible for nuclear retention of RNA (e.g., Rrp6); proteins and protein domains responsible for nuclear export of RNA (e.g., TAP, NXF1, THO, TREX, REF, and Aly); proteins and protein domains responsible for repression of RNA splicing (e.g., PTB, Sam68, and hnRNP A1); proteins and protein domains responsible for stimulation of RNA splicing (e.g., Serine/Arginine-rich (SR) domains); proteins and protein domains responsible for reducing the efficiency of transcription (e.g., FUS (TLS)); and proteins and protein domains responsible for stimulating transcription (e.g., CDK7 and HIV Tat). Alternatively, the effector domain may be a domain of a protein selected from the group comprising endonucleases; proteins and protein domains capable of stimulating RNA cleavage; exonucleases; deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc., e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription; and proteins and protein domains capable of stimulating transcription. Another suitable fusion partner is a PUF RNA-binding domain, which is described in more detail in WO2012068627, which is hereby incorporated by reference in its entirety.

In some instances, the fusion partner comprises an RNA splicing factor. The RNA splicing factor may be used (in whole or as fragments thereof) for modular organization, with separate sequence-specific RNA binding modules and splicing effector domains. Non-limiting examples of RNA splicing factors include members of the Serine/Arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP Al binds to exonic splicing silencers (ESSs) through its RRM domains and inhibits exon inclusion through a C-terminal Glycine-rich domain. Some splicing factors may regulate alternative use of splice site (ss) by binding to regulatory sequences between the two alternative sites. For example, ASF/SF2 may recognize ESEs and promote the use of intron proximal sites, whereas hnRNP Al may bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. The long splicing isoform Bel-xL is a potent apoptosis inhibitor expressed in long-lived postmitotic cells and is up-regulated in many cancer cells, protecting cells against apoptotic signals. The short isoform Bel-xS is a pro-apoptotic isoform and expressed at high levels in cells with a high turnover rate (e.g., developing lymphocytes). The ratio of the two Bcl-x splicing isoforms is regulated by multiple c\acute{ω}-elements that are located in either the core exon region or the exon extension region (i.e., between the two alternative 5′ splice sites). For more examples, see WO2010075303, which is hereby incorporated by reference in its entirety.

In some embodiments, a fusion partner is an exonuclease fusion partner. In some embodiments, an exonuclease fusion partner comprises an amino acid sequence that is at least 70%, at least 75%, at least 80%, at least 85%, at least 90%, at least 95%, or 100% identical to any one of the amino acid sequences recited in TABLE 2.2. In some embodiments, a fusion partner is an exonuclease fusion partner. In some embodiments, an exonuclease fusion partner comprises an amino acid sequence that is at least 70%, at least 75%, at least 80%, at least 85%, at least 90%, at least 95%, or 100% similar to any one of the amino acid sequences recited in TABLE 2.2.

Disclosed herein are fusion proteins that show an improved activity (e.g., enhanced nuclease activity, enhanced potency of nuclease activity, enhanced precision of nuclease activity) relative to the wildtype effector protein counterpart. In some embodiments, a fusion partner of the fusion protein improves the activity of the wildtype effector protein counterpart to which it is has been fused to. In some embodiments, the fusion partner can be at least one of the fusion partners having nucleic acid modification activity as described herein, including, for example, an exonuclease fusion partner. In some embodiments, the fusion partner can be any two, three, four, five, six, seven, eight, nine, or ten of the fusion partners having nucleic acid modification activity as described herein. In some embodiments, the fusion partner enhances precision of nuclease activity of the effector protein. In some embodiments, the fusion partner enhancing precision of nuclease activity of the effector protein comprises one or more exonucleases as described herein. In some embodiments, the fusion partner protein improves precision of the effector protein by at least 10%, at least 20%, at least 40%, at least 60%, at least 80%, at least 100%, at least 120%, at least 140%, at least 160%, at least 180% or at least 200% relative to the effector protein alone. Precision may be evaluated by the size of an indel activity window, also referred to in some embodiments as the cut site. The indel activity window represents where indels start and end. In some embodiments, the fusion partner protein reduces an indel activity window (cut site) of the effector protein by at least 30%, at least 40%, at least 50%, at least 60%, at least 70%, at least 80% or at least 90% relative to the indel activity window (cut site) of the effector protein alone. In some embodiments, the fusion partner protein reduces an indel activity window (cut site) of the effector protein by at least about 50% relative to the indel activity window (cut site) of the effector protein alone. In some embodiments, the fusion partner enhances nuclease activity of the effector protein. In some embodiments, the fusion partner enhancing nuclease activity of the effector protein comprises one or more exonucleases as described herein. In some embodiments, fusion partner protein improves nuclease activity of the effector protein by at least 10%, at least 20%, at least 40%, at least 60%, at least 80%, at least 100%, at least 120%, at least 140%, at least 160%, at least 180% or at least 200%.

Accordingly, disclosed herein are compositions and methods for modifying a target nucleic acid. The target nucleic acid may be a gene or a portion thereof. Methods and compositions may modify a coding portion of a gene, a non-coding portion of a gene, or a combination thereof. Modifying at least one gene using the compositions and methods described herein may reduce or increase expression of one or more genes. In some embodiments, compositions and methods reduce expression of one or more genes by at least 10%, at least 20%, at least 30%, at least 40%, at least 50%, at least 60%, at least 70%, at least 80%, at least 90%, or at least 95%. In some embodiments, compositions and methods remove all expression of a gene, also referred to as genetic knock out. In some embodiments, compositions and methods increase expression of one or more genes by at least 10%, at least 20%, at least 30%, at least 40%, at least 50%, at least 60%, at least 70%, at least 80%, at least 90%, or at least 100%.

### Base Editing Enzymes

In some embodiments, effector partners (e.g., fusion partners) modify a nucleobase of a target nucleic acid. Fusion proteins comprising such fusion partners and an effector protein may be referred to as base editors. A base editing is a system comprising an effector protein and a base editing enzyme. When a base editor is described herein, it can refer to a fusion protein comprising a base editing enzyme fused or linked to an effector protein. In some embodiments, the base editor comprises a base editing enzyme and an effector protein as independent components. Such a base editing enzyme may be referred to as an effector partner (e.g., a fusion partner) herein. In some embodiments, a base editing enzyme variant that differs from a naturally occurring base editing enzyme, but it is understood that any reference to a base editing enzyme herein also refers to a base editing enzyme variant. The base editor is functional when the effector protein is coupled to a guide nucleic acid. In some embodiments, the base editor is function when the effector protein is coupled to a target nucleic acid. The guide nucleic acid imparts sequence specific activity to the base editor. By way of non-limiting example, the effector protein may comprise a catalytically inactive effector protein (e.g., a catalytically inactive variant of an effector protein described herein). Also, by way of non-limiting example, the base editing enzyme may comprise deaminase activity. Additional base editors are described herein. Also, by way of non-limiting example, the base editing enzyme may comprise deaminase activity. Additional base editors are described herein.

In some embodiments, base editors modify a sequence of a target nucleic acid. In some embodiments, base editing enzymes are capable of catalyzing editing (e.g., a chemical modification) of a nucleobase of a nucleic acid molecule, such as DNA or RNA (single stranded or double stranded). In some embodiments, base editors provide a nucleobase change in a DNA molecule. In some embodiments, a base editing enzyme, and therefore a base editor, is capable of converting an existing nucleobase to a different nucleobase. In some embodiments, the nucleobase change in the DNA molecule is selected from: an adenine (A) to guanine (G); cytosine (C) to thymine (T); and cytosine (C) to guanine (G). In some embodiments, base editors provide a nucleobase change in an RNA molecule. In some embodiments, the nucleobase change in the RNA molecule is selected from: adenine (A) to guanine (G); uracil (U) to cytosine (C); cytosine (C) to guanine (G); guanine (G) to adenine (A); and hydrolytic deamination of an adenine or adenosine, or methylation of cytosine (e.g., CpG, CpA, CpT or CpC).

Some base editors modify a nucleobase of on a single strand of DNA. In some embodiments, base editors modify a nucleobase on both strands of dsDNA. In some embodiments, base editing enzymes edit a nucleobase of an RNA.

In some embodiments, a base editing enzyme itself may or may not bind to the nucleic acid molecule containing the nucleobase. In some embodiments, upon binding to its target locus in a target nucleic acid (e.g., a DNA molecule), base pairing between the guide nucleic acid and target strand leads to displacement of a small segment of single-stranded DNA in an “R-loop”. In some embodiments, one or more DNA bases within the R-loop are modified by the base editing enzyme having the deaminase enzyme. In some embodiments, base editing systems for improved efficiency in eukaryotic cells comprise a catalytically inactive effector protein that may generate a nick in the non-edited strand, inducing repair of the non-edited strand using the edited strand as a template.

Some base editors modify a nucleobase of an RNA. In some embodiments, RNA base editors comprise an adenosine deaminase. In some embodiments, ADAR proteins bind to RNAs and alter their sequence by changing an adenosine into an inosine. In some embodiments, RNA base editors comprise an effector protein that is activated by or binds RNA.

In some embodiments, fusion partners comprise a base editing enzyme. When a base editing enzyme is described herein, it can refer to a protein, polypeptide, or fragment thereof that is capable of catalyzing the chemical modification of a nucleobase of a deoxyribonucleotide or a ribonucleotide. Such a base editing enzyme, for example, is capable of catalyzing a reaction that modifies a nucleobase that is present in a nucleic acid molecule, such as DNA or RNA (single stranded or double stranded). Non-limiting examples of the type of modification that a base editing enzyme is capable of catalyzing includes converting an existing nucleobase to a different nucleobase, such as converting a cytosine to a guanine or thymine or converting an adenine to a guanine, hydrolytic deamination of an adenine or adenosine, or methylation of cytosine (e.g., CpG, CpA, CpT or CpC). A base editing enzyme itself may or may not bind to the nucleic acid molecule containing the nucleobase.

In some embodiments, the base editing enzyme modifies the nucleobase of a deoxyribonucleotide. In some embodiments, the base editing enzyme modifies the nucleobase of a ribonucleotide. A base editing enzyme that converts a cytosine to a guanine or thymine may be referred to as a cytosine base editing enzyme. A base editing enzyme that converts an adenine to a to a guanine may be referred to as an adenine base editing enzyme. In some embodiments, the base editing enzyme comprises a deaminase enzyme. In some embodiments, the deaminase functions as a monomer. In some embodiments, the deaminase functions as heterodimer with an additional protein. In some embodiments, base editors comprise a DNA glycosylase inhibitor. In some embodiments, base editors comprise a uracil glycosylase inhibitor (UGI) or uracil N-glycosylase (UNG). In some embodiments, base editors do not comprise a UGI. In some embodiments, base editors do not comprise a UNG. In some embodiments, base editors do not comprise a functional fragment of a UGI. A functional fragment of a UGI is a fragment of a UGI that is capable of excising a uracil residue from DNA by cleaving an N-glycosydic bond. In some cases, a functional fragment comprises a fragment of a protein that retains some function relative to the entire protein. Non-limiting examples of functions are nucleic acid binding, protein binding, nuclease activity, nickase activity, deaminase activity, demethylase activity, or acetylation activity.

In some embodiments, a base editing enzyme can comprise a protein, polypeptide or fragment thereof that is capable of catalyzing the chemical modification of a nucleobase of a deoxyribonucleotide or a ribonucleotide. Such a base editing enzyme, for example, is capable of catalyzing a reaction that modifies a nucleobase that is present in a nucleic acid molecule, such as DNA or RNA (single stranded or double stranded). Non-limiting examples of the type of modification that a base editing enzyme is capable of catalyzing includes converting an existing nucleobase to a different nucleobase, such as converting a cytosine to a guanine or thymine or converting an adenine to a guanine, hydrolytic deamination of an adenine or adenosine, or methylation of cytosine (e.g., CpG, CpA, CpT or CpC). A base editing enzyme itself may or may not bind to the nucleic acid molecule containing the nucleobase. In some cases, a base editor can be a fusion protein comprising a base editing enzyme fused to an effector protein. The base editor is functional when the effector protein is coupled to a guide nucleic acid. The guide nucleic acid imparts sequence specific activity to the base editor. By way of non-limiting example, the effector protein may comprise a catalytically inactive effector protein. Also, by way of non-limiting example, the base editing enzyme may comprise deaminase activity. Additional base editors are described herein.

In some embodiments, the base editor is a cytidine deaminase base editor generated by ancestral sequence reconstruction as described in WO2019226953, which is hereby incorporated by reference in its entirety.

In some embodiments, a base editing enzyme comprises a deaminase enzyme. Exemplary deaminases are described in US20210198330, WO2021041945, WO2021050571A1, and WO2020123887, all of which are incorporated herein by reference in their entirety. Exemplary deaminase domains are described WO 2018027078 and WO2017070632, and each are hereby incorporated in its entirety by reference. Also, additional exemplary deaminase domains are described in Komor et al., Nature, 533, 420-424 (2016); Gaudelli et al., Nature, 551, 464-471 (2017); Komor et al., Science Advances, 3:eaao4774 (2017), and Rees et al., Nat Rev Genet. 2018 Dec. 19(12):770-788. doi: 10.1038/s41576-018-0059-1, which are hereby incorporated by reference in their entirety.

In some embodiments, the base editor is a cytosine base editor (CBE). In general, a CBE comprises a cytosine base editing enzyme and a catalytically inactive effector protein. In some embodiments, the catalytically inactive effector protein is a catalytically inactive variant of an effector protein described herein. The CBE may convert a cytosine to a thymine. In some embodiments, the base editor is an adenine base editor (ABE). In general, an ABE comprises an adenine base editing enzyme and a catalytically inactive effector protein. In some embodiments, the catalytically inactive effector protein is a catalytically inactive variant of an effector protein described herein. The ABE generally converts an adenine to a guanine. In some embodiments, the base editor is a cytosine to guanine base editor (CGBE). In general, a CGBE converts a cytosine to a guanine.

In some embodiments, the base editor is a CBE. In some embodiments, the cytosine base editing enzyme is a cytidine deaminase. In some embodiments, the cytosine deaminase is an APOBEC1 cytosine deaminase, which accept ssDNA as a substrate but is incapable of cleaving dsDNA, fused to a catalytically inactive effector protein. In some embodiments, when bound to its cognate DNA, the catalytically inactive effector protein performs local denaturation of the DNA duplex to generate an R-loop in which the DNA strand not paired with the guide RNA exists as a disordered single-stranded bubble. In some embodiments, the catalytically inactive effector protein generated ssDNA R-loop enables the CBE to perform efficient and localized cytosine deamination in vitro. In some examples, deamination activity is exhibited in a window of about 4 to about 10 base pairs. In some embodiments, fusion to the catalytically inactive effector protein presents the target site to APOBEC1 in high effective molarity, enabling the CBE to deaminate cytosines located in a variety of different sequence motifs, with differing efficacies. In some embodiments, the CBE is capable of mediating RNA-programmed deamination of target cytosines in vitro. In some embodiments, the CBE is capable of mediating RNA-programmed deamination of target cytosines in vivo. In some embodiments, the cytosine base editing enzyme is a cytosine base editing enzyme described by Koblan et al. (2018) Nature Biotechnology 36:848-846; Komor et al. (2016) Nature 533:420-424; Koblan et al. (2021) “Efficient C•G-to-G•C base editors developed using CRISPRi screens, target-library analysis, and machine learning,” Nature Biotechnology; Kurt et al. (2021) Nature Biotechnology 39:41-46; Zhao et al. (2021) Nature Biotechnology 39:35-40; and Chen et al. (2021) Nature Communications 12:1384, all incorporated herein by reference.

In some embodiments, CBEs comprise a uracil glycosylase inhibitor (UGI) or uracil N-glycosylase (UNG). In some embodiments, base excision repair (BER) of U•G in DNA is initiated by a UNG, which recognizes the U•G mismatch and cleaves the glyosidic bond between uracil and the deoxyribose backbone of DNA. In some embodiments, BER results in the reversion of the U•G intermediate created by the first CBE back to a C•G base pair. In some embodiments, UNG may be inhibited by fusion of uracil DNA glycosylase inhibitor (UGI), in some embodiments, a small protein from bacteriophage PBS, to the C-terminus of the CBE. In some embodiments, UGI is a DNA mimic that potently inhibits both human and bacterial UNG. In some embodiments, a UGI inhibitor is any protein or polypeptide that inhibits UNG. In some embodiments, the CBE mediates efficient base editing in bacterial cells and moderately efficient editing in mammalian cells, enabling conversion of a C•G base pair to a T•A base pair through a U•G intermediate. In some embodiments, the CBE is modified to increase base editing efficiency while editing more than one strand of DNA.

In some embodiments, the CBE nicks the non-edited DNA strand. In some embodiments, the non-edited DNA strand nicked by the CBE biases cellular repair of the U•G mismatch to favor a U•A outcome, elevating base editing efficiency. In some embodiments, the APOBEC1-nickase-UGI fusion efficiently edits in mammalian cells, while minimizing frequency of non-target indels.

In some embodiments, the cytidine deaminase is selected from APOBEC1, APOBEC2, APOBEC3C, APOBEC3D, APOBEC3F, APOBEC3G, APOBEC3H, APOBEC4, APOBEC3A, BE1 (APOBEC1-XTEN-dCas9), BE2 (APOBEC1-XTEN-dCas9-UGI), BE3 (APOBEC1-XTEN-dCas9(A840H)-UGI), BE3-Gam, saBE3, saBE4-Gam, BE4, BE4-Gam, saBE4, or saBE4-Gam as described in WO2021163587, WO202108746, WO2021062227, and WO2020123887, which are incorporated herein by reference in their entirety.

In some embodiments, base editors do not comprise a functional fragment of the base editing enzyme. In some embodiments, base editors do not comprise a function fragment of a UGI, where such a fragment may be capable of excising a uracil residue from DNA by cleaving an N-glycosidic bond. In some embodiments, the fusion protein further comprises a non-protein uracil-DNA glycosylase inhibitor (npUGI). In some embodiments, the npUGI is selected from a group of small molecule inhibitors of uracil-DNA glycosylase (UDG), or a nucleic acid inhibitor of UDG. In some embodiments, the non-protein uracil-DNA glcosylase inhibitor (npUGI) is a small molecule derived from uracil. Examples of small molecule non-protein uracil-DNA glcosylase inhibitors, fusion proteins, and Cas-CRISPR systems comprising base editing activity are described in WO2021087246, which is incorporated by reference in its entirety.

In some embodiments, the fusion partner is a deaminase, e.g., ADAR1/2, ADAR-2, or AID.

In some embodiments, a base editor is an adenine base editor (ABE), wherein the base editing enzyme is an adenine base editing enzyme. In some embodiments, the adenine base editing enzyme, and therefore the ABE, may convert an adenine to a guanine. In some embodiments, an ABE converts an A•T base pair to a G•C base pair. In some embodiments, the ABE converts a target A•T base pair to G•C in vivo. In some embodiments, the ABE converts a target A•T base pair to G•C in vitro. In some embodiments, ABEs provided herein reverse spontaneous cytosine deamination, which has been linked to pathogenic point mutations. In some embodiments, ABEs provided herein enable correction of pathogenic SNPs (˜47% of disease-associated point mutations). In some embodiments, the adenine comprises exocyclic amine that has been deaminated (e.g., resulting in altering its base pairing preferences). In some embodiments, deamination of adenosine yields inosine. In some embodiments, inosine exhibits the base-pairing preference of guanine in the context of a polymerase active site, although inosine in the third position of a tRNA anticodon is capable of pairing with A, U, or C in mRNA during translation. In some embodiments, the adenine base editing enzyme is selected from ABE8e, ABE8.20m, APOBEC3A, Anc APOBEC (a.k.a. AncBE4Max), and BtAPOBEC2. In some embodiments, the deaminase or enzyme with deaminase activity is selected from ABE7, ABE8.1m, ABE8.2m, ABE8.3m, ABE8.4m, ABE8.5m, ABE8.6m, ABE8.7m, ABE8.8m, ABE8.9m, ABE8.10m, ABE8.11m, ABE8.12m, ABE8.13m, ABE8.14m, ABE8.15m, ABE8.16m, ABE8.17m, ABE8.18m, ABE8.19m, ABE8.20m, ABE8.21m, ABE8.22m, ABE8.23m, ABE8.24m, ABE8.1d, ABE8.2d, ABE8.3d, ABE8.4d, ABE8.5d, ABE8.6d, ABE8.7d, ABE8.8d, ABE8.9d, ABE8.10d, ABE8.11d, ABE8.12d, ABE8.13d, ABE8.14d, ABE8.15d, ABE8.16d, ABE8.17d, ABE8.18d, ABE8.19d, ABE8.20d, ABE8.21d, ABE8.22d, ABE8.23d, or ABE8.24d. In some embodiments, the adenine base editing enzyme is ABE8.1d. In some embodiments, the adenosine base editor is ABE9. Exemplary deaminases are described in US20210198330, WO2021041945, WO2021050571A1, and WO2020123887, all of which are incorporated herein by reference in their entirety. In some embodiments, the adenine base editing enzyme is an adenine base editing enzyme described in Chu et al., (2021) The CRISPR Journal 4:2:169-177, incorporated herein by reference. In some embodiments, the adenine deaminase is an adenine deaminase described by Koblan et al. (2018) Nature Biotechnology 36:848-846, incorporated herein by reference. In some embodiments, the adenine base editing enzyme is an adenine base editing enzyme described by Tran et al. (2020) Nature Communications 11:4871. Additional examples of deaminase domains are also described in WO2018027078 and WO2017070632, which are hereby incorporated by reference in their entirety. In some embodiments, an ABE comprises an engineered adenosine deaminase enzyme capable of acting on ssDNA. In some embodiments, the ABE described herein is capable of targeting polyA signals, splice site acceptors, and start codons. In some embodiments, the ABE cannot create stop codons for knock-down.

In some embodiments, a base editor comprises an adenosine deaminase variant that differs from a naturally occurring deaminase. Relative to the naturally occurring deaminase, the adenosine deaminase variant may comprise a V82S alteration, a T166R alteration, or a combination thereof. In some embodiments, the adenosine deaminase variant comprises at least one of the following alterations relative to a naturally occurring adenosine deaminase: V82S, T166R, Y147T, Y147R, Q154S, Y123H, and Q154R, or any combination thereof which are incorporated herein by reference in their entirety.

In some embodiments, the deaminase functions as a monomer. In some embodiments, the deaminase functions as heterodimer with an additional protein. In some embodiments, a base editor comprises a deaminase dimer. In some embodiments, a base editor is a deaminase dimer further comprising a base editing enzyme and an adenine deaminase (e.g., TadA).

In some embodiments, the adenosine deaminase is a TadA monomer (e.g., Tad*7.10, TadA*8 or TadA*9). In some embodiments, the adenosine deaminase is a TadA*8 variant. Such a TadA*8 variant includes TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24 as described in WO2021163587 and WO2021050571, which are each hereby incorporated by reference in its entirety. In some embodiments, a base editor is a deaminase dimer comprising a base editing enzyme fused to TadA via a linker.

In some embodiments, the base editing enzyme is fused to TadA at the N-terminus. In some embodiments, the base editing enzyme is fused to TadA at the C-terminus. In some embodiments, the base editing enzyme is a deaminase dimer comprising an ABE. In some embodiments, the deaminase dimer comprises an adenosine deaminase. In some embodiments, the deaminase dimer comprises TadA fused to an adenine base editing enzyme selected from ABE8e, ABE8.20m, APOBEC3A, Anc APOBEC (a.k.a. AncBE4Max), and BtAPOBEC2. In some embodiments TadA is fused to ABE8e or a variant thereof. In some embodiments TadA is fused to ABE8e or a variant thereof at the amino-terminus (ABE8e-TadA). In some embodiments, TadA is fused to ABE8e or a variant thereof at the carboxy terminus (ABE8e-TadA).

In some embodiments, a base editor is an RNA base editor, wherein the base editing enzyme is an RNA base editing enzyme. In some embodiments, the RNA base editing enzyme comprises an adenosine deaminase. In some embodiments, ADAR proteins bind to RNAs and alter their sequence by changing an adenosine into an inosine. In some embodiments, RNA base editors comprise an effector protein that is activated by or binds RNA.

In some embodiments, base editing enzymes, and therefore base editors, are used for treating a subject having or a subject suspected of having a disease related to a gene of interest. In some embodiments, base editing enzymes, and therefore base editors, are useful for treating a disease or a disorder caused by a point mutation in a gene of interest. In some embodiments, compositions, systems, and methods described herein comprise a base editor and a guide nucleic acid, wherein the base editor comprises an effector protein and a base editing enzyme, and wherein the guide nucleic acid directs the base editor to a sequence in a target gene.

### Prime Editing

In some embodiments, a fusion protein and/or a fusion partner can comprise a prime editing enzyme. When used herein, a prime editing enzyme can describe a protein, polypeptide, or fragment thereof that is capable of catalyzing the modification (insertion, deletion, or base-to-base conversion) of a target nucleotide or nucleotide sequence in a nucleic acid. A prime editing enzyme capable of catalyzing such a reaction includes a reverse transcriptase. A prime editing enzyme may require a prime editing guide RNA (pegRNA) to catalyze the modification. Such a pegRNA can be capable of identifying the nucleotide or nucleotide sequence in the target nucleic acid to be edited and encoding the new genetic information that replaces the targeted nucleotide or nucleotide sequence in the nucleic acid. A prime editing enzyme may require a prime editing guide RNA (pegRNA) and a single guide RNA to catalyze the modification.

In some embodiments, a prime editing enzyme is a protein, a polypeptide or a fragment thereof that is capable of catalyzing the modification (insertion, deletion, or base-to-base conversion) of a target nucleotide or nucleotide sequence in a nucleic acid. A prime editing enzyme capable of catalyzing such a reaction includes a reverse transcriptase. A prime editing enzyme may require a prime editing guide RNA (pegRNA) to catalyze the modification. Such a pegRNA can be capable of identifying the nucleotide or nucleotide sequence in the target nucleic acid to be edited and encoding the new genetic information that replaces the targeted nucleotide or nucleotide sequence in the nucleic acid. A prime editing enzyme may require a prime editing guide RNA (pegRNA) and a single guide RNA to catalyze the modification. In some embodiments, the target nucleic acid is a dsDNA molecule. In some embodiments, the pegRNA comprises a guide RNA comprising a first region that is bound by the effector protein, and a second region comprising a spacer sequence that is complementary to a target sequence of the dsDNA molecule; a template RNA comprising a primer binding sequence that hybridizes to a primer sequence of the dsDNA molecule that is formed when target nucleic acid is cleaved, and a template sequence that is complementary to at least a portion of the target sequence of the dsDNA molecule with the exception of at least one nucleotide. In some embodiments, the spacer sequence is complementary to the target sequence on a target strand of the dsDNA molecule. In some embodiments, the spacer sequence is complementary to the target sequence on a non-target strand of the dsDNA molecule. In some embodiments, the primer binding sequence hybridizes to a primer sequence on the non-target strand of the dsDNA molecule. In some embodiments, the primer binding sequence hybridizes to a primer sequence on the target strand of the dsDNA molecule. In some embodiments, the target strand is cleaved. In some embodiments, the non-target strand is cleaved.

In some embodiments, such a prime editing enzyme is an M-MLV RT enzyme or a mutant thereof. In some embodiments, the M-MLV RT enzyme comprises at least one mutation selected from D200N, L603W, T330P, T306K, and W313F relative to wildtype M-MLV RT enzyme.

### Protein Modification Activity

In some cases, a fusion partner provides enzymatic activity that modifies a protein (e.g., a histone) associated with a target nucleic acid. Such enzymatic activities include, but are not limited to, methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

In some cases, the fusion partner has enzymatic activity that modifies a protein associated with a target nucleic acid. The protein may be a histone, an RNA binding protein, or a DNA binding protein. Examples of such protein modification activities include methyltransferase activity such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, SET1A, SET1B, MLL1 to 5, ASH1, SYMD2, NSD1, DOT1L, Pr-SET7/8, SUV4-20H1, EZH2, RIZ1); demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3); acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK); deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11); kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

### CRISPRa Fusions and CRISPRi Fusions

In some instances, fusion partners include, but are not limited to, a protein that directly and/or indirectly provides for increased or decreased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). In some instances, fusion partners that increase or decrease transcription include a transcription activator domain or a transcription repressor domain, respectively.

In some embodiments, fusion partners activate or increase expression of a target nucleic acid. Fusion proteins comprising such fusion partners and an effector protein may be referred to as CRISPRa fusions. In some embodiments, fusion partners increase expression of the target nucleic acid relative to its expression in the absence of the fusion effector protein. Relative expression, including transcription and RNA levels, may be assessed, quantified, and compared, e.g., by RT-qPCR. In some embodiments, fusion partners comprise a transcriptional activator. In some embodiments, a transcriptional activator can describe a polypeptide or a fragment thereof that can activate or increase transcription of a target nucleic acid molecule. Transcriptional activators may promote transcription via: recruitment of other transcription factor proteins; modification of target DNA such as demethylation; recruitment of a DNA modifier; modulation of histones associated with target DNA; recruitment of a histone modifier such as those that modify acetylation and/or methylation of histones; or a combination thereof.

Non-limiting examples of fusion partners that promote or increase transcription include, but are not limited to: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1; histone lysine demethylases such as JHDM2a/b, UTX, JMJD3; histone acetyltransferases such as GCN5, PCAF, CBP, p300, TAF1, TIP60/PLIP, MOZ/MYST3, MORF/MYST4, SRC1, ACTR, P160, CLOCK; and DNA demethylases such as Ten-Eleven Translocation (TET) dioxygenase 1 (TET1CD), TET1, DME, DML1, DML2, and ROS1; and functional domains thereof. Other non-limiting examples of suitable fusion partners include: proteins and protein domains responsible for stimulating translation (e.g., Staufen); proteins and protein domains responsible for (e.g., capable of) modulating translation (e.g., translation factors such as initiation factors, elongation factors, release factors, etc., e.g., eIF4G); proteins and protein domains responsible for stimulation of RNA splicing (e.g., Serine/Arginine-rich (SR) domains); and proteins and protein domains responsible for stimulating transcription (e.g., CDK7 and HIV Tat).

In some embodiments, fusions partners inhibit or reduce expression of a target nucleic acid. Fusion proteins comprising such fusion partners and an effector protein may be referred to as CRISPRi fusions. In some embodiments, fusion partners reduce expression of the target nucleic acid relative to its expression in the absence of the fusion effector protein. Relative expression, including transcription and RNA levels, may be assessed, quantified, and compared, e.g., by RT-qPCR. In some embodiments, fusion partners may comprise a transcriptional repressor. In some embodiments, a transcriptional repressor can describe a polypeptide or a fragment thereof that is capable of arresting, preventing, or reducing transcription of a target nucleic acid. Transcriptional repressors may inhibit transcription via: recruitment of other transcription factor proteins; modification of target DNA such as methylation; recruitment of a DNA modifier; modulation of histones associated with target DNA; recruitment of a histone modifier such as those that modify acetylation and/or methylation of histones; or a combination thereof.

Non-limiting examples of fusion partners that decrease or inhibit transcription include, but are not limited to: transcriptional repressors such as the Kruppel associated box (KRAB or SKD); KOX1 repression domain; the Mad mSIN3 interaction domain (SID); the ERF repressor domain (ERD), the SRDX repression domain (e.g., for repression in plants); histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY; histone lysine deacetylases such as HDAC1, HDAC2, HDAC3, HDAC8, HDAC4, HDAC5, HDAC7, HDAC9, SIRT1, SIRT2, HDAC11; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants); and periphery recruitment elements such as Lamin A, and Lamin B; and functional domains thereof. Other non-limiting examples of suitable effector partners (e.g., fusion partners) include: proteins and protein domains responsible for repressing translation (e.g., Ago2 and Ago4); proteins and protein domains responsible for repression of RNA splicing (e.g., PTB, Sam68, and hnRNP Al); proteins and protein domains responsible for reducing the efficiency of transcription (e.g., FUS (TLS)).

In some embodiments, fusion proteins comprising the described effector partners (e.g., fusion partners) and an effector protein may be referred to as CRISPRa fusions, wherein the effector partners (e.g., fusion partners) activate or increase expression of a target nucleic acid. In some embodiments, fusion proteins comprising the described effector partners (e.g., fusion partners) and an effector protein may be referred to as CRISPRi fusions, wherein the effector partners (e.g., fusion partners) inhibit or reduce expression of a target nucleic acid. In some embodiments, fusion proteins are targeted by a guide nucleic acid (e.g., guide RNA) to a specific location in a target nucleic acid and exert locus-specific regulation such as blocking RNA polymerase binding to a promoter (which selectively inhibits transcription activator function), and/or changes a local chromatin status (e.g., when a fusion sequence is used that edits the target nucleic acid or modifies a protein associated with the target nucleic acid). In some embodiments, the modifications are transient (e.g., transcription repression or activation). In some embodiments, the modifications are inheritable. For example, epigenetic modifications made to a target nucleic acid, or to proteins associated with the target nucleic acid, e.g., nucleosomal histones, in a cell, can be observed in a successive generation.

In some embodiments, the effector partner (e.g., fusion partner) comprises an RNA splicing factor. The RNA splicing factor may be used (in whole or as fragments thereof) for modular organization, with separate sequence-specific RNA binding modules and splicing effector domains. In some embodiments, the RNA splicing factors comprise members of the Serine/Arginine-rich (SR) protein family containing N-terminal RNA recognition motifs (RRMs) that bind to exonic splicing enhancers (ESEs) in pre-mRNAs and C-terminal RS domains that promote exon inclusion. In some embodiments, a hnRNP protein hnRNP Al binds to exonic splicing silencers (ESSs) through its RRM domains and inhibits exon inclusion through a C-terminal Glycine-rich domain. In some embodiments, the RNA splicing factors may regulate alternative use of splice site (ss) by binding to regulatory sequences between two alternative sites. For example, in some embodiments, ASF/SF2 may recognize ESEs and promote the use of intron proximal sites, whereas hnRNP Al may bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. Long splicing isoform Bel-xL is a potent apoptosis inhibitor expressed in long-lived postmitotic cells and is up-regulated in many cancer cells, protecting cells against apoptotic signals. Short isoform Bel-xS is a pro-apoptotic isoform and expressed at high levels in cells with a high turnover rate (e.g., developing lymphocytes). A ratio of the two Bcl-x splicing isoforms is regulated by multiple c\acute{ω}-elements that are located in either core exon region or exon extension region (i.e., between the two alternative 5′ splice sites). For more examples, see WO2010075303, which is hereby incorporated by reference in its entirety.

### Recombinases

In some embodiments, provided herein is a combinase system comprising effector proteins described herein and a fusion partner. In some embodiments, the fusion partners comprise a recombinase domain or a recombinase. In some embodiments, the effector proteins have reduced nuclease activity or no nuclease activity. In some embodiments, the enzymatically inactive protein is fused with a recombinase. In some embodiments, the recombinase is a site-specific recombinase. In some embodiments, the fusion partners comprise a recombinase domain wherein the recombinase is a site-specific recombinase. In some embodiments, described herein is a programmed nuclease comprising reduced nuclease activity or no nuclease activity and fused with a recombinase, wherein the recombinase can be a site-specific recombinase. Such polypeptides can be used for site-directed transgene insertion. In some embodiments, when describing a transgene, reference is made to a nucleotide sequence that is inserted into a cell for expression of said nucleotide sequence in the cell. A transgene is meant to include (1) a nucleotide sequence that is not naturally found in the cell (e.g., a heterologous nucleotide sequence); (2) a nucleotide sequence that is a mutant form of a nucleotide sequence naturally found in the cell into which it has been introduced; (3) a nucleotide sequence that serves to add additional copies of the same (e.g., exogenous or homologous) or a similar nucleotide sequence naturally occurring in the cell into which it has been introduced; or (4) a silent naturally occurring or homologous nucleotide sequence whose expression is induced in the cell into which it has been introduced. The cell in which transgene expression occurs can be a target cell, such as a host cell.

Non-limiting examples of site-specific recombinases include a tyrosine recombinase (e.g., Cre, Flp or lambda integrase), a serine recombinase (e.g., gamma-delta resolvase, Tn3 resolvase, Sin resolvase, Gin invertase, Hin invertase, Tn5044 resolvase, IS607 transposase and integrase), or mutants or variants thereof. In some embodiments, the recombinase is a serine recombinase. Non-limiting examples of serine recombinases include, but are not limited to, gamma-delta resolvase, Tn3 resolvase, Sin resolvase, Gin invertase, Hin invertase, Tn5044 resolvase, IS607 transposase, and IS607 integrase. In some embodiments, the site-specific recombinase is an integrase. Non-limiting examples of integrases include, but are not limited to: Bxb1, wBeta, BL3, phiR4, A118, TG1, MR11, phi370, SPBc, TP901-1, phiRV, FC1, K38, phiBT1, and phiC31. Further discussion and examples of suitable recombinase fusion partners are described in U.S. Pat. No. 10,975,392, which is incorporated herein by reference in its entirety. In some embodiments, the fusion protein comprises a linker that links the recombinase domain to the Cas-CRISPR domain of the effector protein. In some embodiments, the linker is The-Ser.

### Linkers for Peptides

In some embodiments, a linker comprises a bond or molecule that links a first polypeptide to a second polypeptide. Accordingly, in some embodiments, effector proteins, effector partners (e.g., fusion partners), or combinations thereof are connected by linkers. In some embodiments, effector proteins and fusion partners of a fusion effector protein are connected via a linker. The linker may comprise or consist of a covalent bond. The linker may comprise or consist of a chemical group. In some embodiments, the linker comprises an amino acid. In some cases, a linker comprises a bond or molecule that links a first polypeptide to a second polypeptide. In some instances, a peptide linker comprises at least two amino acids linked by an amide bond. In general, the linker connects a terminus of the effector protein to a terminus of the fusion partner. In some embodiments, the carboxy terminus of the effector protein is linked to the amino terminus of the fusion partner. In some embodiments, the carboxy terminus of the fusion partner is linked to the amino terminus of the effector protein. In some embodiments, the effector protein and the effector partner (e.g., fusion partner) are directly linked by a covalent bond.

In some embodiments, linkers comprise one or more amino acids. In some embodiments, linker is a protein. In some cases, a terminus of the effector protein is linked to a terminus of the fusion partner through an amide bond. In some cases, a terminus of the effector protein is linked to a terminus of the fusion partner through a peptide bond. In some cases, an effector protein is coupled to a fusion partner by a linker protein. The linker protein may have any of a variety of amino acid sequences. A linker protein may comprise a region of rigidity (e.g., beta sheet, alpha helix), a region of flexibility, or any combination thereof. In some instances, the linker comprises small amino acids, such as glycine and alanine, that impart high degrees of flexibility. The ordinarily skilled artisan will recognize that design of a peptide conjugated to any desired element may include linkers that are all or partially flexible, such that the linker may include a flexible linker as well as one or more portions that confer less flexible structure. In some embodiments, the linker is from 1 to 300, from 1 to 250, from 1 to 200, from 1 to 150, from 1 to 100, from 1 to 50, from 1 to 25, from 1 to 10, from 10 to 300, from 10 to 250, from 10 to 200, from 10 to 150, from 10 to 100, from 10 to 50, from 10 to 25, from 25 to 300, from 25 to 250, from 25 to 200, from 25 to 150, from 25 to 100, from 25 to 50, from 50 to 300, from 50 to 250, from 50 to 200, from 50 to 150, from 50 to 100, from 100 to 300, from 100 to 250, from 100 to 200, from 100 to 150, from 150 to 300, from 150 to 250, from 150 to 200, from 200 to 300, from 200 to 250, or from 250 to 300 amino acids in length. In some embodiments, the linker is from 1 to 100 amino acids in length. In some embodiments, the linker is more 100 amino acids in length. Suitable linkers include proteins of 4 linked amino acids to 40 linked amino acids in length, or between 4 linked amino acids and 25 linked amino acids in length. In some embodiments, the linker is from 10 to 27 amino acids in length. In some embodiments, when a linked amino acids is described herein, it can refer to at least two amino acids linked by an amide bond.

These linkers may be produced by using synthetic, linker-encoding oligonucleotides to couple the proteins, or may be encoded by a nucleic acid sequence encoding a fusion protein (e.g., an effector protein coupled to a fusion partner). Examples of linker proteins include glycine polymers (G)n, glycine-serine polymers (including, for example, (GS)n, GSGGSn (SEQ ID NO: 86), GGSGGSn (SEQ ID NO: 87), and GGGSn (SEQ ID NO: 88), where n is an integer of at least one), glycine-alanine polymers, and alanine-serine polymers. Exemplary linkers may comprise amino acid sequences including, but not limited to, GGSG (SEQ ID NO: 89), GGSGG (SEQ ID NO: 90), GSGSG (SEQ ID NO: 91), GSGGG (SEQ ID NO: 92), GGGSG (SEQ ID NO: 93), and GSSSG (SEQ ID NO: 94). In some embodiments, the linker comprises one or more repeats a tri-peptide GGS. In some embodiments, the linker is an XTEN linker. In some embodiments, the XTEN linker is an XTEN80 linker. In some embodiments, the XTEN linker is an XTEN20 linker. In some embodiments, the XTEN20 linker has an amino acid sequence of GSGGSPAGSPTSTEEGTSESATPGSG (SEQ ID NO: 54).

In some embodiments, linkers do not comprise an amino acid. In some embodiments, linkers do not comprise a peptide. In some embodiments, linkers comprise a nucleotide, a polynucleotide, a polymer, or a lipid. A non-peptide linker may be a polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacrylamide, polyacrylate, polycyanoacrylates, lipid polymers, chitins, hyaluronic acid, heparin, or an alkyl linker.

In some embodiments, a linker is recognized and cleaved by a protein described herein. In some embodiments, a linker comprises a recognition sequence that may be recognized and cleaved by the protein. In some embodiments, a guide nucleic acid comprises an aptamer, which may serve a similar function as a linker, bringing an effector protein and an effector partner (e.g., fusion partner) into proximity. The aptamer can functionally connect two proteins (e.g., effector protein, effector partner, such as a fusion partner) by interacting non-covalently with both, thereby bringing both proteins into proximity of the guide nucleic acid. In some embodiments, the first protein and/or the second protein comprise or is covalently linked to an aptamer binding moiety. In some embodiments, the aptamer is a short single stranded DNA (ssDNA) or RNA (ssRNA) molecule capable of being bound be the aptamer binding moiety. In some embodiments, the aptamer is a molecule that is capable of mimicking antibody binding activity and may be classified as a chemical antibody. In some instances, the aptamer described herein refers to artificial oligonucleotides that bind one or more specific molecules. In some embodiments, aptamers exhibit a range of affinities (KD in the pM to μM range) with little or no off-target binding.

### Engineered Proteins

In some instances, effector proteins described herein have been modified (also referred to as an engineered protein). In some embodiments, effector proteins disclosed herein are engineered proteins. Engineered proteins are not identical to a naturally-occurring protein. Unless otherwise indicated, reference to effector proteins throughout the present disclosure include engineered proteins thereof.

In some embodiments, a modification of the proteins may include addition of one or more amino acids, deletion of one or more amino acids, substitution of one or more amino acids, or combinations thereof. For example, effector proteins described herein can be modified with one or more additional modifying heterologous polypeptides. In some embodiments, the protein modified with the addition of one or more heterologous peptides may be referred to herein as a fusion protein. Such fusion proteins are described herein and throughout.

In some embodiments, a heterologous peptide comprises a subcellular localization signal (e.g., a sequence). In some cases, an effector protein is modified with a subcellular localization sequence. In certain embodiments, a subcellular localization sequence can be a nuclear localization signal (NLS) for targeting or localizing a nucleic acid, protein or small molecule to the nucleus when present in a cell that contains a nuclear compartment, a sequence to keep a protein out of the nucleus, e.g., a nuclear export sequence (NES), a sequence to keep a protein retained in the cytoplasm, a mitochondrial localization signal for targeting to the mitochondria, a chloroplast localization signal for targeting to a chloroplast, an ER retention signal, and the like.

Accordingly, an effector protein, composition, system and methods described herein may comprise a nuclear localization signal (NLS). In some cases, an NLS comprises an entity (e.g., peptide) that facilitates localization of a nucleic acid, protein, or small molecule to the nucleus, when present in a cell that contains a nuclear compartment. An NLS can be located at or near the amino terminus (N-terminus) of the effector protein disclosed herein. An NLS can be located at or near the carboxy terminus (C-terminus) of the effector protein s disclosed herein. In some embodiments, a vector encodes the effector proteins described herein, wherein the vector or vector systems disclosed herein comprises one or more NLSs, such as about or more than about 1, 2, 3, 4, 5, 6, 7, 8, 9, 10, or more NLSs. In some embodiments, an effector protein described herein comprises 1, 2, 3, 4, 5, 6, 7, 8, 9, 10, or more NLSs at or near the N-terminus, 1, 2, 3, 4, 5, 6, 7, 8, 9, 10, or more NLSs at or near the C-terminus, or a combination of these (e.g., one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In certain embodiments, an NLS described herein comprises an NLS sequence recited in TABLE 2. In some cases, an effector protein described herein is not modified with an NLS so that the polypeptide is not targeted to the nucleus, which can be advantageous depending on the circumstance (e.g., when the target nucleic acid is an RNA that is present in the cytosol).

In some embodiments, a heterologous peptide comprises a chloroplast transit peptide (CTP), also referred to as a chloroplast localization signal or a plastid transit peptide, which targets the protein to a chloroplast. Chromosomal transgenes from bacterial sources may require a sequence encoding a CTP sequence fused to a sequence encoding an expressed protein (e.g., an effector protein, and/or an effector partner, such as a fusion partner) if the expressed protein is to be compartmentalized in the plant plastid (e.g., chloroplast). The CTP may be removed in a processing step during translocation into the plastid. Accordingly, localization of the protein to a chloroplast is often accomplished by means of operably linking a polynucleotide sequence encoding a CTP sequence to the 5′ region of a polynucleotide encoding the exogenous protein.

In some embodiments, the heterologous peptide is an endosomal escape peptide (EEP). An EEP is an agent that quickly disrupts the endosome in order to minimize the amount of time that a delivered molecule, such protein, spends in the endosome-like environment, and to avoid getting trapped in the endosomal vesicles and degraded in the lysosomal compartment. An exemplary EEP is set forth in TABLE 2.

In some embodiments, the heterologous peptide is a cell penetrating peptide (CPP), also known as a Protein Transduction Domain (PTD). A CPP or PTD is a polypeptide, polynucleotide, carbohydrate, or organic or inorganic compound that facilitates traversing a lipid bilayer, micelle, cell membrane, organelle membrane, or vesicle membrane.

Further suitable heterologous peptides include, but are not limited to, proteins (or fragments/domains thereof) that are boundary elements (e.g., CTCF), proteins and fragments thereof that provide periphery recruitment (e.g., Lamin A, Lamin B, etc.), and protein docking elements (e.g., FKBP/FRB, Pil1/Aby1, etc.).

In some cases, effector proteins described herein can be modified with a tag. A tag can be a heterologous polypeptide that is detectable for use in tracking and/or purification. Accordingly, in some embodiments, an effector protein, composition, system and methods described herein may comprise a purification tag and/or a fluorescent protein. Non-limiting examples of purification tags include a histidine tag, e.g., a 6×His tag (SEQ ID NO: 95); a hemagglutinin (HA) tag; a FLAG tag; a Myc tag; and maltose binding protein (MBP). In some embodiments, the protein tag is a portion of MBP that can be detected and/or purified. Non-limiting examples of fluorescent protein tags include green fluorescent protein (GFP), yellow fluorescent protein (YFP), red fluorescent protein (RFP), cyan fluorescent protein (CFP), mCherry, and tdTomato. In certain embodiments, a tag described herein comprises a tag sequence recited in TABLE 2.

A heterologous peptide may be located at or near the amino terminus (N-terminus) of the protein (e.g., effector protein, and/or effector partner, such as a fusion partner,) disclosed herein. A heterologous peptide may be located at or near the carboxy terminus (C-terminus) of the proteins disclosed herein. In some embodiments, a heterologous peptide is located internally in the protein described herein (i.e., is not at the N- or C-terminus of the protein described herein) at a suitable insertion site.

In some embodiments, a protein (e.g., effector protein or effector partner, such as a fusion partner) described herein comprises 1, 2, 3, 4, 5, 6, 7, 8, 9, 10, or more heterologous peptides at or near the N-terminus, 1, 2, 3, 4, 5, 6, 7, 8, 9, 10, or more heterologous peptides at or near the C-terminus, or a combination of these (e.g., one or more heterologous peptides at the amino-terminus and one or more heterologous peptides at the carboxy terminus). When more than one heterologous peptide is present, each may be selected independently of the others, such that a single heterologous peptide may be present in more than one copy and/or in combination with one or more other heterologous peptides present in one or more copies. In some embodiments, a heterologous peptide is considered near the N- or C-terminus when the nearest amino acid of the heterologous peptide is within about 1, 2, 3, 4, 5, 10, 15, 20, 25, 30, 40, 50, or more amino acids along the polypeptide chain from the N- or C-terminus.

In some embodiments, a heterologous peptide described herein comprises a heterologous peptide sequence recited in TABLE 2. Accordingly in some embodiments, effector proteins described herein comprise an amino acid sequence that at least about 65%, at least about 70%, at least about 75%, at least about 80%, at least about 85%, at least about 90%, at least about 95%, at least about 97%, or at least 98%, or at least about 99% identical to the sequence of SEQ ID NO: 1 and further comprises one or more sequence set forth in TABLE 2. In some embodiments, effector proteins described herein comprise an amino acid sequence that at least about 65%, at least about 70%, at least about 75%, at least about 80%, at least about 85%, at least about 90%, at least about 95%, at least about 97%, or at least 98%, or at least about 99% similar to the sequence of SEQ ID NO: 1 and further comprises one or more sequence set forth in TABLE 2. In some embodiments, a heterologous peptide described herein may be an effector partner (e.g., a fusion partner) as described en supra.

Exemplary amino acid sequences of effector proteins and modifications as described herein can be seen in TABLE 2.1. In certain embodiments, effector proteins described herein comprise an amino acid sequence that at least about 65%, at least about 70%, at least about 75%, at least about 80%, at least about 85%, at least about 90%, at least about 95%, at least about 97%, or at least 98%, at least about 99%, or about 100% identical to any one of the sequences as set forth in TABLE 2.1. In some embodiments, effector proteins described herein comprise an amino acid sequence that at least about 65%, at least about 70%, at least about 75%, at least about 80%, at least about 85%, at least about 90%, at least about 95%, at least about 97%, or at least 98%, at least about 99%, or about 100% similar to any one of the sequences as set forth in TABLE 2.1.

In another example, effector proteins may be codon optimized. In some embodiments, effector protein described herein are encoded by a codon optimized nucleic acid. In some embodiments, a nucleic acid sequence encoding an effector protein described herein, is codon optimized. This type of optimization can entail a mutation of an effector protein encoding nucleotide sequence to mimic the codon preferences of the intended host organism or cell while encoding the same polypeptide. Thus, the codons can be changed, but the encoded protein remains unchanged. For example, if the intended target cell was a human cell, a human codon-optimized effector protein-encoding nucleotide sequence could be used. As another non-limiting example, if the intended host cell were a mouse cell, then a mouse codon-optimized effector protein-encoding nucleotide sequence could be generated. As another non-limiting example, if the intended host cell were a eukaryotic cell, then a eukaryote codon-optimized Effector protein nucleotide sequence could be generated. As another non-limiting example, if the intended host cell were a prokaryotic cell, then a prokaryote codon-optimized effector protein-encoding nucleotide sequence could be generated. Codon usage tables are readily available, for example, at the “Codon Usage Database” available at www.kazusa.or.jp/codon. Accordingly, in some embodiments, effector proteins described herein may be codon optimized for expression in a specific cell, for example, a bacterial cell, a plant cell, a eukaryotic cell, an animal cell, a mammalian cell, or a human cell. In some embodiments, the effector protein is codon optimized for a human cell.

It is understood that when describing coding sequences of polypeptides described herein, said coding sequences do not necessarily require a codon encoding a N-terminal Methionine (M) or a Valine (V) as described for the effector proteins described herein. One skilled in the art would understand that a start codon could be replaced or substituted with a start codon that encodes for an amino acid residue sufficient for initiating translation in a host cell. In some instances, when a modifying heterologous peptide, such as a fusion protein partner, is located at the N terminus of the effector protein, a start codon for the fusion protein partner serves as a start codon for the effector protein as well. Thus, the natural start codon encoding an amino acid residue sufficient for initiating translation (e.g., Methionine (M) or a Valine (V)) of the effector protein may be removed or absent.

Nuclease-Dead Effector Proteins (dCas Proteins)

An engineered protein may comprise a modified form of a wild type counterpart protein (e.g., an effector protein). For example, proteins (e.g., effector protein, effector partner, such as a fusion partner) may comprise one or more modifications, such as amino acid alterations, that may provide increased activity as compared to a naturally-occurring counterpart. As another example, proteins may provide increased catalytic activity (e.g., nickase, nuclease, binding activity) as compared to a naturally-occurring counterpart. Proteins may provide enhanced nucleic acid binding activity (e.g., enhanced binding of a guide nucleic acid, and/or target nucleic acid) as compared to a naturally-occurring counterpart. Protein may have a 1%, 5%, 10%, 20%, 30%, 40%, 50%, 60%, 70%, 80%, 90%, 100%, 120%, 140%, 160%, 180%, 200%, or more, increase of the activity of a naturally-occurring counterpart (e.g., a polypeptide consisting of the amino acid sequence of SEQ ID NO: 1). Engineered effector proteins comprising variant amino acid sequences and having enhanced activity are described in further detail en supra.

Alternatively, the modified form of the wild type counterpart may comprise an amino acid change or alteration (e.g., deletion, insertion, or substitution) that reduces the activity, such as nucleic acid-cleaving activity, of the effector protein relative to the wild type counterpart. For example, a nuclease domain (e.g., RuvC domain) of an effector protein may be deleted or mutated relative to a wild type counterpart effector protein so that it is no longer functional or comprises reduced nuclease activity. An effector protein may have a 100%, 90%, 80%, 70%, 60%, 50%, 40%, 30%, 20%, 10%, 5%, 1%, or less, decrease of the activity of a naturally occurring counterpart (e.g., a polypeptide consisting of the amino acid sequence of SEQ ID NO: 1). Decreased activity may be decreased catalytic activity (e.g., nickase, nuclease, binding, specificity activity) as compared to a naturally-occurring counterpart.

The modified form of the effector protein may have less than 100%, less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart (e.g., a polypeptide consisting of the amino acid sequence of SEQ ID NO: 1). In some embodiments, an effector protein may generate about 100%, about 90%, about 80%, about 70%, about 60%, about 50%, about 40%, about 30%, about 20%, about 10%, about 5%, or about 1% less indels in a population of cells relative to the number of indels generated by a polypeptide consisting of the amino acid sequence of SEQ ID NO: 1, as measured in a cleavage assay.

Engineered proteins may have no substantial nucleic acid-cleaving activity. Engineered proteins may be enzymatically inactive or “dead,” that is it may bind to a nucleic acid but not cleave it. An enzymatically inactive protein may comprise an enzymatically inactive domain (e.g., inactive nuclease domain). Enzymatically inactive may refer to an activity less than 1%, less than 2%, less than 3%, less than 4%, less than 5%, less than 6%, less than 7%, less than 8%, less than 9%, or less than 10% activity compared to the wild-type counterpart. A dead protein may associate with a guide nucleic acid to activate or repress transcription of a target nucleic acid. In some embodiments, the enzymatically inactive protein is fused with a protein comprising recombinase activity. In some embodiments, activity (e.g., nuclease activity) of effector proteins and/or compositions described herein can be measured relative to a WT effector protein (SEQ ID NO: 1) or compositions containing the same in a cleavage assay.

In some embodiments, the effector protein can comprise an enzymatically inactive and/or “dead” (abbreviated by “d”) effector protein in combination (e.g., fusion) with a polypeptide comprising recombinase activity. Although an effector protein normally has nuclease activity, in some embodiments, an effector protein does not have nuclease activity. In some embodiments, an effector protein, other than the one or more alterations set forth in TABLE 1, comprising at least 60%, at least 65%, at least 70%, at least 75%, at least 80%, at least 85%, at least 90%, at least 92%, at least 95%, at least 97%, at least 98%, at least 99%, or 100% sequence identity with the sequence of SEQ ID NO: 1 is a nuclease-dead effector protein. In some embodiments, the effector protein, other than the one or more alterations set forth in TABLE 1, comprising at least 60%, at least 65%, at least 70%, at least 75%, at least 80%, at least 85%, at least 90%, at least 92%, at least 95%, at least 97%, at least 98%, at least 99%, or 100% sequence identity with the sequence of SEQ ID NO: 1 is modified or engineered to be a nuclease-dead effector protein.

In some embodiments, an effector protein comprises one or more alterations selected from 369A, 369N, 567A, 567Q, and 658N, wherein the effector protein comprises an amino acid sequence that is at least 60%, at least 65%, at least 70%, at least 75%, at least 80%, at least 85%, at least 90%, at least 92%, at least 95%, at least 97%, at least 98%, at least 99%, or 100% identical to SEQ ID NO: 1, and wherein the effector protein is a nuclease-dead effector protein. In some embodiments, an effector protein comprises one or more alterations selected from 369A, 369N, 567A, 567Q, and 658N, wherein the effector protein comprises an amino acid sequence that is at least 60%, at least 65%, at least 70%, at least 75%, at least 80%, at least 85%, at least 90%, at least 92%, at least 95%, at least 97%, at least 98%, at least 99%, or 100% similar to SEQ ID NO: 1, and wherein the effector protein is a nuclease-dead effector protein.

In some embodiments, an effector protein comprises E567A substitution, wherein the effector protein comprises an amino acid sequence that is at least 60%, at least 65%, at least 70%, at least 75%, at least 80%, at least 85%, at least 90%, at least 92%, at least 95%, at least 97%, at least 98%, at least 99%, or 100% identical to SEQ ID NO: 1, and wherein the effector protein is a nuclease-dead effector protein. In some embodiments, an effector protein comprises E567A substitution, wherein the effector protein comprises an amino acid sequence that is at least 60%, at least 65%, at least 70%, at least 75%, at least 80%, at least 85%, at least 90%, at least 92%, at least 95%, at least 97%, at least 98%, at least 99%, or 100% similar to SEQ ID NO: 1, and wherein the effector protein is a nuclease-dead effector protein.

In some embodiments, an effector protein comprises E567Q substitution, wherein the effector protein comprises an amino acid sequence that is at least 60%, at least 65%, at least 70%, at least 75%, at least 80%, at least 85%, at least 90%, at least 92%, at least 95%, at least 97%, at least 98%, at least 99%, or 100% identical to SEQ ID NO: 1, and wherein the effector protein is a nuclease-dead effector protein. In some embodiments, an effector protein comprises E567Q substitution, wherein the effector protein comprises an amino acid sequence that is at least 60%, at least 65%, at least 70%, at least 75%, at least 80%, at least 85%, at least 90%, at least 92%, at least 95%, at least 97%, at least 98%, at least 99%, or 100% similar to SEQ ID NO: 1, and wherein the effector protein is a nuclease-dead effector protein.

The effector protein can comprise a modified form of a wild type counterpart. The modified form of the wild type counterpart can comprise an amino acid change (e.g., deletion, insertion, or substitution) that reduces the nucleic acid-cleaving activity of the effector protein. For example, a nuclease domain (e.g., HEPN domain) of an effector polypeptide can be deleted or mutated so that it is no longer functional or comprises reduced nuclease activity. The modified form of the effector protein can have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart. The modified form of an effector protein can have no substantial nucleic acid-cleaving activity. When an effector protein is a modified form that has no substantial nucleic acid-cleaving activity, it can be referred to as enzymatically inactive and/or dead. A dead effector polypeptide can bind to a target sequence but may not cleave the target nucleic acid. A dead effector polypeptide can associate with a guide nucleic acid to activate or repress transcription of a target nucleic acid.

### Multimeric Complexes

Compositions, systems, and methods of the present disclosure may comprise a multimeric complex or uses thereof, wherein the multimeric complex comprises multiple effector proteins that non-covalently interact with one another. A multimeric complex may comprise enhanced activity relative to the activity of any one of its effector proteins alone. For example, a multimeric complex comprising two effector proteins (e.g., in dimeric form) may comprise greater nucleic acid binding affinity, nuclease activity (e.g., cis-cleavage activity, and/or transcollateral cleavage activity) than that of either of the effector proteins provided in monomeric form. A multimeric complex may comprise one or more heterologous proteins fused to one or more effector proteins, wherein the fusion proteins are capable of different activity than that of the one or more effector proteins. In another example, a multimeric complex comprising an effector protein and a partner protein comprising an effector partner (e.g., a fusion partner), wherein the multimeric complex may comprise greater nucleic acid binding affinity and/or nuclease activity than that of either of the effector protein or effector partner (e.g., a fusion partner) provided in monomeric form.

A multimeric complex may have an affinity for a target region of a target nucleic acid and is capable of catalytic activity (e.g., cleaving, nicking, inserting or otherwise modifying the nucleic acid) at or near the target sequence. A multimeric complex may have an affinity for a donor nucleic acid and is capable of catalytic activity (e.g., cleaving, nicking, editing or otherwise modifying the nucleic acid by creating cuts) at or near one or more ends of the donor nucleic acid. Multimeric complexes may be activated when complexed with a guide nucleic acid. Multimeric complexes may be activated when complexed with a guide nucleic acid and a target nucleic acid. In some embodiments, the multimeric complex cleaves the target nucleic acid. In some embodiments, the multimeric complex nicks the target nucleic acid.

Various aspects of the present disclosure include compositions and methods comprising multiple effector proteins, and uses thereof, respectively. An effector protein, other than the one or more alterations set forth in TABLE 1, comprising at least 65%, at least 70%, at least 75%, at least 80%, at least 85%, at least 90%, at least 95%, at least 97%, at least 98%, or at least 99% sequence identity to the sequence of SEQ ID NO: 1 may be provided with a second effector protein. An effector protein, other than the one or more alterations set forth in TABLE 1, comprising at least 65%, at least 70%, at least 75%, at least 80%, at least 85%, at least 90%, at least 95%, at least 97%, at least 98%, or at least 99% sequence similarity to the sequence of SEQ ID NO: 1 may be provided with a second effector protein. Two effector proteins may target different nucleic acid sequences. Two effector proteins may target different types of nucleic acids (e.g., a first effector protein may target double- and single-stranded nucleic acids, and a second effector protein may only target single-stranded nucleic acids). It is understood that when discussing the use of more than one effector protein in compositions, systems, and methods provided herein, the multimeric complex form is also described.

In some embodiments, multimeric complexes comprise at least one effector protein, or a fusion protein thereof, other than the one or more alterations set forth in TABLE 1, comprising an amino acid sequence with at least 65%, at least 70%, at least 75%, at least 80%, at least 85%, at least 90%, at least 95% or 100% identity to the sequence of SED ID NO: 1. In some embodiments, multimeric complexes comprise at least one effector protein, or a fusion protein thereof, other than the one or more alterations set forth in TABLE 1, comprising an amino acid sequence with at least 65%, at least 70%, at least 75%, at least 80%, at least 85%, at least 90%, at least 95% or 100% similarity to the sequence of SED ID NO: 1. In some embodiments, multimeric complexes comprise at least one effector protein or a fusion protein thereof, wherein the amino acid sequence of the effector protein, other than the one or more alterations set forth in TABLE 1, is at least 65%, at least 70%, at least 75%, at least 80%, at least 85%, at least 90%, at least 95% or 100% identical to the sequence of SEQ ID NO: 1. In some embodiments, multimeric complexes comprise at least one effector protein or a fusion protein thereof, wherein the amino acid sequence of the effector protein, other than the one or more alterations set forth in TABLE 1, is at least 65%, at least 70%, at least 75%, at least 80%, at least 85%, at least 90%, at least 95% or 100% similar to the sequence of SEQ ID NO: 1.

In some embodiments, the multimeric complex is a dimer comprising two effector proteins of identical amino acid sequences. In some embodiments, the multimeric complex comprises a first effector protein and a second effector protein, wherein the amino acid sequence of the first effector protein is at least 90%, at least 92%, at least 94%, at least 96%, at least 98% identical, or at least 99% identical to the amino acid sequence of the second effector protein.

In some embodiments, the multimeric complex is a dimer comprising two effector proteins of similar amino acid sequences. In some embodiments, the multimeric complex comprises a first effector protein and a second effector protein, wherein the amino acid sequence of the first effector protein is at least 90%, at least 92%, at least 94%, at least 96%, at least 98% identical, or at least 99% similar to the amino acid sequence of the second effector protein.

In some embodiments, the multimeric complex is a heterodimeric complex comprising at least two effector proteins of different amino acid sequences. In some embodiments, the multimeric complex is a heterodimeric complex comprising a first effector protein and a second effector protein, wherein the amino acid sequence of the first effector protein is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% identical to the amino acid sequence of the second effector protein. In some embodiments, the multimeric complex is a heterodimeric complex comprising a first effector protein and a second effector protein, wherein the amino acid sequence of the first effector protein is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% similar to the amino acid sequence of the second effector protein.

In some embodiments, a multimeric complex comprises at least two effector proteins. In some embodiments, a multimeric complex comprises more than two effector proteins. In some embodiments, a multimeric complex comprises two, three or four effector proteins. In some embodiments, at least one effector protein of the multimeric complex comprises an amino acid sequence, other than the one or more alterations set forth in TABLE 1, with at least 65%, at least 70%, at least 75%, at least 80%, at least 85%, at least 90%, at least 95% or 100% identity to the sequence of SEQ ID NO: 1. In some embodiments, at least one effector protein of the multimeric complex comprises an amino acid sequence, other than the one or more alterations set forth in TABLE 1, with at least 65%, at least 70%, at least 75%, at least 80%, at least 85%, at least 90%, at least 95% or 100% similarity to the sequence of SEQ ID NO: 1. In some embodiments, each effector protein of the multimeric complex comprises an amino acid sequence, other than the one or more alterations set forth in TABLE 1, with at least 65%, at least 70%, at least 75%, at least 80%, at least 85%, at least 90%, at least 95% or 100% identity to the sequence of SEQ ID NO: 1. In some embodiments, each effector protein of the multimeric complex comprises an amino acid sequence, other than the one or more alterations set forth in TABLE 1, with at least 65%, at least 70%, at least 75%, at least 80%, at least 85%, at least 90%, at least 95% or 100% similarity to the sequence of SEQ ID NO: 1.

In some embodiments, the multimeric complex described herein is capable of targeting polyA signals, splice site acceptors, and start codons. In some embodiments, the multimeric complex cannot create stop codons for knock-down. In some embodiments, the multimeric complex is a dimer comprising fusion protein described herein. In some embodiments, the fusion protein comprises the effector protein described herein and the effector partner (e.g., fusion partner) described herein. In some embodiments, the dimer is formed due to non-covalent interactions between the effector proteins of monomers. In some embodiments, N- and C-termini of “formerly active” monomer is closer to 5′ region of non-target strand, while the termini of the “other” monomer is closer to 3′ region, which results in a larger editing window of the multimeric complex having a larger editing window on the non-target strand. In some embodiments, the multimeric complex has a lower editing window for a target strand due to in accessibility for the effector partner (e.g., fusion partner).

### Synthesis, Isolation and Assaying of One or More Polypeptides of Variants Thereof

Any of a variety of methods can be used to generate a variant amino acid sequence of an effector protein disclosed herein. Such methods include, but are not limited to, site-directed mutagenesis, random mutagenesis, combinatorial libraries, and other mutagenesis methods described herein, rational design and de novo design (see, e.g., Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, MD (1999); Gillman et al., Directed Evolution Library Creation: Methods and Protocols (Methods in Molecular Biology) Springer, 2nd ed (2014)).

Polypeptides (e.g., effector proteins, effector partners (e.g., fusion partners), and fusion proteins) of the present disclosure may be synthesized, using any suitable method. Effector proteins of the present disclosure of the present disclosure may be synthesized, using any suitable method. Effector proteins of the present disclosure may be produced in vitro or by eukaryotic cells or by prokaryotic cells. When in vitro is described herein, it can be used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

Effector proteins can be further processed by unfolding, e.g., heat denaturation, dithiothreitol reduction, etc. and may be further refolded, using any suitable method. One non-limiting example of a method for preparing an effector protein is to express recombinant nucleic acids encoding the effector protein in a suitable microbial organism, such as a bacterial cell, a yeast cell, or other suitable cell, using methods well known in the art. In some embodiments, the nucleic acid(s) encoding the polypeptides described herein, the recombinant nucleic acid(s) described herein, the vectors described herein may be produced in vitro or in vivo by eukaryotic cells or by prokaryotic cells.

Methods of generating and assaying the effector proteins described herein are well known to one of skill in the art. Examples of such methods are described in the Examples provided herein.

In some embodiments, a polypeptide (e.g., an effector protein, an effector partner, and/or a fusion protein) provided herein is an isolated polypeptide. In some embodiments, polypeptide described herein can be isolated and purified for use in compositions, systems, and/or methods described herein. Methods described here can include the step of isolating polypeptide described herein. An isolated polypeptide provided herein can be isolated by a variety of methods well-known in the art, for example, recombinant expression systems, precipitation, gel filtration, ion-exchange, reverse-phase and affinity chromatography, and the like. Other well-known methods are described in Deutscher et al., Guide to Protein Purification: Methods in Enzymology, Vol. 182, (Academic Press, (1990)). Alternatively, the isolated polypeptides of the present disclosure can be obtained using well-known recombinant methods (see, e.g., Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999)). The methods and conditions for biochemical purification of a polypeptide described herein can be chosen by those skilled in the art, and purification monitored, for example, by a functional assay.

For example, compositions and/or systems described herein can further comprise a purification tag that can be attached to a polypeptide (e.g., an effector protein, effector partner, and/or fusion protein) or a nucleic acid encoding for a purification tag that can be attached to a nucleic acid encoding for a polypeptide as described herein. A purification tag, as used herein, can be an amino acid sequence which can attach or bind with high affinity to a separation substrate and assist in isolating the protein of interest from its environment, which can be its biological source, such as a cell lysate. Attachment of the purification tag can be at the N or C terminus of the polypeptide. Furthermore, an amino acid sequence recognized by a protease or a nucleic acid encoding for an amino acid sequence recognized by a protease, such as TEV protease or the HRV3C protease can be inserted between the purification tag and the effector protein, such that biochemical cleavage of the sequence with the protease after initial purification liberates the purification tag. Purification and/or isolation can be through high performance liquid chromatography (HPLC), exclusion chromatography, gel electrophoresis, affinity chromatography, or other purification technique. Examples of purification tags are as described herein.

In some embodiments, polypeptides (e.g., effector proteins, effector partners such as fusion partners, and/or fusion proteins) described herein are isolated from cell lysate. In some embodiments, the compositions described herein can comprise 20% or more by weight, 75% or more by weight, 95% or more by weight, or 99.5% or more by weight of a polypeptide, related to the method of preparation of compositions described herein and its purification thereof, wherein percentages can be upon total protein content in relation to contaminants. Thus, in some cases, a polypeptide described herein is at least 80% pure, at least 85% pure, at least 90% pure, at least 95% pure, at least 98% pure, or at least 99% pure (e.g., free of contaminants, non-engineered polypeptide proteins or other macromolecules, etc.).

### Protospacer Adjacent Motif (PAM) Sequences

Polypeptides (e.g., effector protein, effector partner, such as a fusion partner, and fusion protein, and dimers or multimeric complexes thereof) of the present disclosure may cleave or nick a target nucleic acid within or near a protospacer adjacent motif (PAM) sequence of the target nucleic acid. In some embodiments, the target nucleic acid is a double stranded nucleic acid comprising a target strand and a non-target strand. In some embodiments, cleavage occurs within 1, 2, 3, 4, 5, 6, 7, 8, 9, 10, 15, 20, 25, 30, 35, 40, 45, or 50 nucleotides of a 5′ or 3′ terminus of a PAM sequence. In some embodiments, polypeptide described herein recognize a PAM sequence. In some embodiments, recognizing a PAM sequence comprises interacting with a sequence adjacent to the PAM (i.e., a target sequence). In some embodiments, a target nucleic acid comprises a target sequence that is adjacent to a PAM sequence. A target nucleic acid may comprise a PAM sequence adjacent to a sequence (i.e., a target sequence) that is complementary to a guide nucleic acid spacer sequence. In some embodiments, the polypeptide does not require a PAM to bind and/or cleave a target nucleic acid.

In some embodiments, a target nucleic acid is a single stranded target nucleic acid comprising a target sequence. Accordingly, in some embodiments, the single stranded target nucleic acid comprises a PAM sequence described herein that is adjacent (e.g., within 1, 2, 3, 4, 5, 6, 7, 8, 9, 10, 15, 20, 25, 30, 35, 40, 45, or 50 nucleotides) or directly adjacent to the target sequence. In some embodiments, an RNP cleaves the single stranded target nucleic acid.

In some embodiments, a target nucleic acid is a double stranded nucleic acid comprising a target strand and a non-target strand, wherein the target strand comprises a target sequence. In some embodiments, the PAM sequence is located on the target strand. In some embodiments, the PAM sequence is located on the non-target strand. In some embodiments, the PAM sequence described herein is adjacent (e.g., within 1, 2, 3, 4, 5, 6, 7, 8, 9, 10, 15, 20, 25, 30, 35, 40, 45, or 50 nucleotides) to the target sequence on the target strand or the non-target strand. In some embodiments, the PAM sequence is located 5′ of a reverse complement of the target sequence on the non-target strand. In some embodiments, such a PAM described herein is directly adjacent to the target sequence on the target strand or the non-target strand. In some embodiments, an RNP cleaves the target strand or the non-target strand. In some embodiments, the RNP cleaves both, the target strand and the non-target strand. In some embodiments, an RNP recognizes the PAM sequence, and hybridizes to a target sequence of the target nucleic acid. In some embodiments, the RNP cleaves the target nucleic acid, wherein the RNP has recognized the PAM sequence and is hybridized to the target sequence.

In some embodiments, an effector protein described herein, or a multimeric complex thereof, recognizes a PAM on a target nucleic acid. In some embodiments, multiple effector proteins of the multimeric complex recognize a PAM on a target nucleic acid. In some embodiments, at least two of the multiple effector proteins recognize the same PAM sequence. In some embodiments, at least two of the multiple effector proteins recognize different PAM sequences. In some embodiments, only one effector protein of the multimeric complex recognizes a PAM on a target nucleic acid.

An effector protein of the present disclosure, or a multimeric complex thereof, may cleave or nick a target nucleic acid within or near a protospacer adjacent motif (PAM) sequence of the target nucleic acid. In some embodiments, cleavage occurs within 1, 2, 3, 4, 5, 6, 7, 8, 9 or 10 nucleotides of a 5′ or 3′ terminus of a PAM sequence.

In some embodiments, a PAM sequence provided herein comprises any one of the nucleotide sequences recited in TABLE 1.5. PAMs used in compositions, systems, and methods herein are further described throughout the application.

### III. Nucleic Acid Systems

A nucleic acid described herein refers to a polymer of nucleotides. A nucleic acid may comprise ribonucleotides, deoxyribonucleotides, combinations thereof, and modified versions of the same. A nucleic acid may be single-stranded or double-stranded, unless specified. Non-limiting examples of nucleic acids are double stranded DNA (dsDNA), single stranded (ssDNA), messenger RNA, genomic DNA, cDNA, DNA-RNA hybrids, and a polymer comprising purine and pyrimidine bases or other natural, chemically or biochemically modified, non-natural, or derivatized nucleotide bases. Accordingly, nucleic acids as described herein may comprise one or more mutations, one or more engineered modifications, or both.

A person of ordinary skill in the art when referring to nucleotides, nucleosides, and/or nucleobases would also understand the differences between RNA and DNA (generally the exchange of uridine for thymidine or vice versa) and the presence of nucleoside analogs, such as modified uridines, do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, NI-methyl pseudouridine, or 5-methoxyuridine, is considered 100% identical to AUG in that both are perfectly complementary to the same sequence (5′-CAU).

### Guide Nucleic Acids

The compositions, systems, and methods of the present disclosure may comprise a guide nucleic acid or a use thereof. Unless otherwise indicated, compositions, systems and methods comprising guide nucleic acids or uses thereof, as described herein and throughout, include DNA molecules, such as expression vectors, that encode a guide nucleic acid. Accordingly, compositions, systems, and methods of the present disclosure comprise a guide nucleic acid or a nucleotide sequence encoding the guide nucleic acid.

In some embodiments, when describing a guide nucleic acid, reference is made to a nucleic acid that, when in a complex with one or more polypeptides described herein (e.g., an RNP complex) can impart sequence selectivity to the complex when the complex interacts with a target nucleic acid. Guide nucleic acids are often referred to as “guide RNA” or (gRNA). However, a guide nucleic acid may comprise deoxyribonucleotides, ribonucleotides, biochemically or chemically modified nucleotides (e.g., one or more engineered modifications as described herein), or any combinations thereof. The term, “guide RNA,” as well as any components thereof (e.g., spacer sequence, repeat sequence, linker nucleotide sequence, crRNA) includes guide nucleic acids comprising DNA bases, RNA bases and chemically modified bases (e.g., one or more engineered modifications as described herein) thereof. Guide nucleic acids may comprise DNA, RNA, or a combination thereof (e.g., RNA with a thymine base). Guide nucleic acids may include a chemically modified nucleobase or phosphate backbone.

The guide RNA may be chemically synthesized or recombinantly produced. The sequence of the guide nucleic acid, or a portion thereof, may be different from the sequence of a naturally occurring nucleic acid. A guide nucleic acid may comprise a naturally occurring guide nucleic acid. A guide nucleic acid may comprise a non-naturally occurring guide nucleic acid, including a guide nucleic acid that is designed to contain a chemical or biochemical modification. Guide nucleic acids and portions thereof may be found in or identified from a CRISPR array present in the genome of a host organism.

In general, a guide nucleic acid is a nucleic acid molecule that binds to an effector protein, thereby forming a ribonucleoprotein complex (RNP). Guide nucleic acids, when complexed with an effector protein, may bring the effector protein into proximity of a target nucleic acid. Sufficient conditions for hybridization of a guide nucleic acid to a target nucleic acid and/or for binding of a guide nucleic acid to an effector protein include in vivo physiological conditions of a desired cell type or in vitro conditions sufficient for assaying catalytic activity of a protein, polypeptide or peptide described herein, such as the nuclease activity of an effector protein.

The guide nucleic acid may also form complexes as described through herein. For example, a guide nucleic acid may hybridize to another nucleic acid, such as target nucleic acid, or a portion thereof. In another example, a guide nucleic acid may complex with an effector protein. In such embodiments, a guide nucleic acid-effector protein complex may be described herein as an RNP. In some embodiments, when in a complex, at least a portion of the complex may bind, recognize, and/or hybridize to a target nucleic acid. For example, when a guide nucleic acid and an effector protein are complexed to form an RNP, at least a portion of the guide nucleic acid hybridizes to a target sequence in a target nucleic acid. Those skilled in the art in reading the below specific examples of guide nucleic acids as used in RNPs described herein, will understand that in some embodiments, a RNP may hybridize to one or more target sequences in a target nucleic acid, thereby allowing the RNP to modify and/or recognize a target nucleic acid or sequence contained therein (e.g., PAM) or to modify and/or recognize non-target sequences depending on the guide nucleic acid, and in some embodiments, the effector protein, used.

In some embodiments, a guide nucleic acid may comprise or form intramolecular secondary structure (e.g., hairpins, stem-loops, etc.). In some embodiments, a guide nucleic acid comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the guide nucleic acid comprises a pseudoknot (e.g., a secondary structure comprising a stem, at least partially, hybridized to a second stem or half-stem secondary structure). An effector protein may recognize a guide nucleic acid comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the guide nucleic acid comprises at least 2, at least 3, at least 4, or at least 5 stem regions.

In some embodiments, the guide nucleic acid comprises 10, 11, 12, 13, 14, 15, 16, 17, 18, 19, 20, 21, 22, 23, 24, 25, 26, 27, 28, 29, or 30 linked nucleotides. In general, a guide nucleic acid comprises at least linked nucleotides. In some embodiments, a guide nucleic acid comprises at least 25 linked nucleotides. A guide nucleic acid may comprise 10 to 50 linked nucleotides. In some embodiments, the guide nucleic acid comprises or consists essentially of about 12 to about 80 linked nucleotides, about 12 to about 50, about 12 to about 45, about 12 to about 40, about 12 to about 35, about 12 to about 30, about 12 to about 25, from about 12 to about 20, about 12 to about 19, about 19 to about 20, about 19 to about 25, about 19 to about 30, about 19 to about 35, about 19 to about 40, about 19 to about 45, about 19 to about 50, about 19 to about 60, about 20 to about 25, about 20 to about 30, about 20 to about 35, about 20 to about 40, about 20 to about 45, about 20 to about 50, or about 20 to about 60 linked nucleotides. In some embodiments, the guide nucleic acid has about 10 to about 60, about 20 to about 50, or about 30 to about 40 linked nucleotides. In some embodiments, when describing the length of a sequence or the length of a linked sequence, reference is made to a nucleic acid (polynucleotide) or polypeptide, may be expressed as “kilobases” (kb) or “base pairs (bp),”. Thus, a length of 1 kb refers to a length of 1000 linked nucleotides, and a length of 500 bp refers to a length of 500 linked nucleotides. Similarly, a protein having a length of 500 linked amino acids may also be simply described as having a length of 500 amino acids.

In some embodiments, the engineered guide nucleic acid comprises at least 10, 11, 12, 13, 14, 15, 16, 17, 18, 19, 20, 21, 22, 23, 24, 25, 26, 27, 28, 29, or 30 contiguous nucleotides that are complementary to a eukaryotic sequence. Such a eukaryotic sequence is a sequence of nucleotides that is present in a host eukaryotic cell. Such a sequence of nucleotides is distinguished from nucleotide sequences present in other host cells, such as prokaryotic cells, or viruses. Said sequences present in a eukaryotic cell can be located a gene, an exon, an intron, a non-coding (e.g., promoter or enhancer) region, a selectable marker, tag, signal, and the like. In some cases, the engineered guide nucleic acid comprises at least 10 contiguous nucleotides that are complementary to a eukaryotic sequence. In some cases, the engineered guide nucleic acid comprises at least 11 contiguous nucleotides that are complementary to a eukaryotic sequence. In some cases, the engineered guide nucleic acid comprises at least 12 contiguous nucleotides that are complementary to a eukaryotic sequence. In some cases, the engineered guide nucleic acid comprises at least 13 contiguous nucleotides that are complementary to a eukaryotic sequence. In some cases, the engineered guide nucleic acid comprises at least 14 contiguous nucleotides that are complementary to a eukaryotic sequence. In some cases, the engineered guide nucleic acid comprises at least 15 contiguous nucleotides that are complementary to a eukaryotic sequence. In some cases, the engineered guide nucleic acid comprises at least 16 contiguous nucleotides that are complementary to a eukaryotic sequence. In some cases, the engineered guide nucleic acid comprises at least 17 contiguous nucleotides that are complementary to a eukaryotic sequence. In some cases, the engineered guide nucleic acid comprises at least 18 contiguous nucleotides that are complementary to a eukaryotic sequence. In some cases, the engineered guide nucleic acid comprises at least 19 contiguous nucleotides that are complementary to a eukaryotic sequence. In some cases, the engineered guide nucleic acid comprises at least 20 contiguous nucleotides that are complementary to a eukaryotic sequence. In some cases, the engineered guide nucleic acid comprises at least 21 contiguous nucleotides that are complementary to a eukaryotic sequence. In some cases, the engineered guide nucleic acid comprises at least 22 contiguous nucleotides that are complementary to a eukaryotic sequence. In some cases, the engineered guide nucleic acid comprises at least 23 contiguous nucleotides that are complementary to a eukaryotic sequence. In some cases, the engineered guide nucleic acid comprises at least 24 contiguous nucleotides that are complementary to a eukaryotic sequence. In some cases, the engineered guide nucleic acid comprises at least 25 contiguous nucleotides that are complementary to a eukaryotic sequence. In some cases, the engineered guide nucleic acid comprises at least 26 contiguous nucleotides that are complementary to a eukaryotic sequence. In some cases, the engineered guide nucleic acid comprises at least 27 contiguous nucleotides that are complementary to a eukaryotic sequence. In some cases, the engineered guide nucleic acid comprises at least 28 contiguous nucleotides that are complementary to a eukaryotic sequence. In some cases, the engineered guide nucleic acid comprises at least 29 contiguous nucleotides that are complementary to a eukaryotic sequence. In some cases, the engineered guide nucleic acid comprises at least 30 or more contiguous nucleotides that are complementary to a eukaryotic sequence.

In some embodiments, the compositions, systems, and methods of the present disclosure may comprise an additional guide nucleic acid or a use thereof. An additional guide nucleic acid can target an effector protein to a different location in the target nucleic acid by binding to a different portion of the target nucleic acid from the first guide nucleic acid. For example, a guide nucleic acid can bind a portion of the target nucleic acid that is upstream or downstream of the target gene in a cell or subject as described herein, wherein the additional guide nucleic acid can bind to a portion of the target nucleic acid that is located either upstream or downstream of where the first guide nucleic acid has targeted. In such embodiments, the dual-guided compositions, systems, and methods described herein can modify the target nucleic acid in two locations. In some embodiments, the dual-guided compositions, systems, and methods described herein can cleave the target nucleic acid in the two locations targeted by the guide nucleic acids. In some embodiments, a donor nucleic acid is inserted in replacement of the deleted sequence. The modification of the target nucleic acid at two different loci is referred to herein as “dual-cutting”. Accordingly, in some embodiments, dual-guided compositions, systems, and methods can comprise two effector proteins, individually corresponding a guide nucleic acid or a single effector protein with two different guide nucleic acid to achieve dual-cutting.

In some embodiments, the compositions, systems, and methods of the present disclosure comprise two or more guide nucleic acids (e.g., 2, 3, 4, 5, 6, 7, 9, 10 or more guide nucleic acids), and/or uses thereof. Multiple guide nucleic acids may target an effector protein to different locations in the target nucleic acid by hybridizing to different target sequences. In some embodiments, a first guide nucleic acid may hybridize within a location of the target nucleic acid that is different from where a second guide nucleic acid may hybridize the target nucleic acid. In some embodiments, the first loci and the second loci of the target nucleic acid may be located at least 1, at least 10, at least 20, at least 30, at least 40, at least 50, at least 60, at least 70, at least 80, at least 90 or at least 100 nucleotides apart. In some embodiments, the first loci and the second loci of the target nucleic acid may be located between 100 and 200, 200 and 300, 300 and 400, 400 and 500, 500 and 600, 600 and 700, 700 and 800, 800 and 900 or 900 and 1000 nucleotides apart. In some embodiments, the first loci and/or the second loci of the target nucleic acid are located in an intron of a gene. In some embodiments, the first loci and/or the second loci of the target nucleic acid are located in an exon of a gene. In some embodiments, the first loci and/or the second loci of the target nucleic acid span an exon-intron junction of a gene. In some embodiments, the first portion and/or the second portion of the target nucleic acid are located on either side of an exon and cutting at both sites results in deletion of the exon. In some embodiments, composition, systems, and methods comprise a donor nucleic acid that may be inserted in replacement of a deleted or cleaved sequence of the target nucleic acid. In some embodiments, compositions, systems, and methods comprising multiple guide nucleic acids or uses thereof comprise multiple effector proteins, wherein the effector proteins may be identical, non-identical, or combinations thereof.

In some embodiments, guide nucleic acids comprise additional elements that contribute additional functionality (e.g., stability, heat resistance, etc.) to the guide nucleic acid. Such elements may be one or more nucleotide alterations, nucleotide sequences, intermolecular secondary structures, or intramolecular secondary structures (e.g., one or more hair pin regions, one or more bulges, etc.).

In some embodiments, guide nucleic acids comprise one or more linkers connecting different nucleotide sequences as described herein. A linker may comprise 1, 2, 3, 4, 5, 6, 7, 8, 9, 10, or more nucleotides. A linker may be any suitable linker, examples of which are described herein.

The guide nucleic acid may comprise a first region complementary to a target nucleic acid (FR1) and a second region that is not complementary to the target nucleic acid (FR2). In some embodiments, FR1 is located 5′ to FR2 (FR1-FR2). In some embodiments, FR2 is located 5′ to FR1 (FR2-FR1). In some embodiments, the FR comprises a repeat sequence. In some embodiments, at least a portion of the FR2 interacts or binds to an effector protein. In some embodiments, the FR1 comprises a spacer sequence, wherein the spacer sequence can interact in a sequence-specific manner with (e.g., has complementarity with, or can hybridize to a target sequence in) a target nucleic acid.

In some embodiments, the first region, the second region, or both may be about 8 nucleotides, about 10 nucleotides, about 12 nucleotides, about 14 nucleotides, about 16 nucleotides, about 18 nucleotides, about 20 nucleotides, about 22 nucleotides, about 24 nucleotides, about 26 nucleotides, about 28 nucleotides, about 30 nucleotides, about 32 nucleotides, about 34 nucleotides, about 36 nucleotides, about 38 nucleotides, about 40 nucleotides, about 42 nucleotides, about 44 nucleotides, about 46 nucleotides, about 48 nucleotides, or about 50 nucleotides long.

In some embodiments, the first region, the second region, or both may be from about 8 to about 12, from about 8 to about 16, from about 8 to about 20, from about 8 to about 24, from about 8 to about 28, from about 8 to about 30, from about 8 to about 32, from about 8 to about 34, from about 8 to about 36, from about 8 to about 38, from about 8 to about 40, from about 8 to about 42, from about 8 to about 44, from about 8 to about 48, or from about 8 to about 50 nucleotides long.

In some embodiments, the first region, the second region, or both may have a melting temperature of about 38° C., about 40° C., about 42° C., about 44° C., about 46° C., about 48° C., about 50° C., about 52° C., about 54° C., about 56° C., about 58° C., about 60° C., about 62° C., about 64° C., about 66° C., about 68° C., about 70° C., about 72° C., about 74° C., about 76° C., about 78° C., about 80° C., about 82° C., about 84° C., about 86° C., about 88° C., about 90° C., or about 92° C. In some embodiments, the first region, the second region, or both may have a melting temperature of from about 35° C. to about 40° C., from about 35° C. to about 45° C., from about 35° C. to about 50° C., from about 35° C. to about 55° C., from about 35° C. to about 60° C., from about 35° C. to about 65° C., from about 35° C. to about 70° C., from about 35° C. to about 75° C., from about 35° C. to about 80° C., or from about 35° C. to about 85° C.

In some embodiments, the guide nucleic acid comprises a nucleotide sequence that is capable of hybridizing to a target sequence in a target nucleic acid, wherein the target nucleic acid is any one of: a naturally occurring eukaryotic sequence, a naturally occurring prokaryotic sequence, a naturally occurring viral sequence, a naturally occurring bacterial sequence, a naturally occurring fungal sequence, an engineered eukaryotic sequence, an engineered prokaryotic sequence, an engineered viral sequence, an engineered bacterial sequence, an engineered fungal sequence, a fragment of a naturally occurring sequence, a fragment of an engineered sequence, and combinations thereof.

In some embodiments, the guide nucleic acid is isolated from any one of: a naturally occurring cell, a eukaryotic cell, a prokaryotic cell, a plant cell, a fungal cell, an animal cell, cell of an invertebrate, a fly cell, a cell of a vertebrate, a mammalian cell, a primate cell, a non-human primate cell, a human cell, a living cell, a non-living cell, a modified cell, a derived cell, and a non-naturally occurring cell.

In some embodiments, the guide nucleic acid or a nucleic acid encoding the guide nucleic acid comprises a nucleotide sequence as described herein (e.g., TABLE 3, TABLE 4, or TABLE 5). Such nucleotide sequences described herein (e.g., TABLE 3, TABLE 4, or TABLE 5) may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that encodes a guide nucleic acid, such as a nucleotide sequence described herein for a vector. Similarly, disclosure of the nucleotide sequences described herein (e.g., TABLE 3, TABLE 4, or TABLE 5) also discloses the complementary nucleotide sequence, the reverse nucleotide sequence, and the reverse complement nucleotide sequence, any one of which can be a nucleotide sequence for use in a guide nucleic acid as described herein.

In some embodiments, the guide nucleic acid or a nucleic acid encoding the guide nucleic acid comprises a sequence that is at least 70%, at least 80%, at least 85%, at least 90%, at least 95%, or 100% identical to any one of the sequences recited in TABLE 3 or TABLE 4, or both. In some embodiments, the guide nucleic acid or a nucleic acid encoding the guide nucleic acid comprises a sequence that is at least 70%, at least 80%, at least 85%, at least 90%, at least 95%, or 100% identical to any one of the sequences recited in TABLE 5.

### Repeat Sequence

Guide nucleic acids described herein may comprise one or more repeat sequences. In some embodiments, a repeat sequence comprises a nucleotide sequence that is not complementary to a target sequence of a target nucleic acid. In some embodiments, a repeat sequence comprises a nucleotide sequence that may interact with an effector protein. In some embodiments, a repeat sequence is connected to another sequence of a guide nucleic acid, such as an intermediary sequence, that is capable of non-covalently interacting with an effector protein. In some embodiments, a repeat sequence includes a nucleotide sequence that is capable of forming a guide nucleic acid-effector protein complex (e.g., a RNP complex).

In some embodiments, the repeat sequence is between 10 and 50, 12 and 48, 14 and 46, 16 and 44, and 18 and 42 nucleotides in length.

In some embodiments, a repeat sequence is adjacent to a spacer sequence. In some embodiments, a repeat sequence is followed by a spacer sequence in the 5′ to 3′ direction. In some embodiments, a repeat sequence is preceded by a spacer sequence in the 5′ to 3′ direction. In some embodiments, a repeat sequence is adjacent to an intermediary sequence. In some embodiments, a repeat sequence is 3′ to an intermediary sequence. In some embodiments, an intermediary sequence is followed by a repeat sequence, which is followed by a spacer sequence in the 5′ to 3′ direction. In some embodiments, a repeat sequence is linked to a spacer sequence and/or an intermediary sequence. In some embodiments, a guide nucleic acid comprises a repeat sequence linked to a spacer sequence and/or to an intermediary sequence, which may be a direct link or by any suitable linker, examples of which are described herein.

In some embodiments, guide nucleic acids comprise more than one repeat sequence (e.g., two or more, three or more, or four or more repeat sequences). In some embodiments, a guide nucleic acid comprises more than one repeat sequence separated by another sequence of the guide nucleic acid. For example, in some embodiments, a guide nucleic acid comprises two repeat sequences, wherein the first repeat sequence is followed by a spacer sequence, and the spacer sequence is followed by a second repeat sequence in the 5′ to 3′ direction. In some embodiments, the more than one repeat sequences are identical. In some embodiments, the more than one repeat sequences are not identical.

In some embodiments, the repeat sequence comprises two sequences that are complementary to each other and hybridize to form a double stranded RNA duplex (dsRNA duplex). In some embodiments, the two sequences are not directly linked and hybridize to form a stem loop structure. In some embodiments, the dsRNA duplex comprises 5, 10, 15, 20 or 25 base pairs (bp). In some embodiments, not all nucleotides of the dsRNA duplex are paired, and therefore the duplex forming sequence may include a bulge. In some embodiments, the repeat sequence comprises a hairpin or stem-loop structure, optionally at the 5′ portion of the repeat sequence. In some embodiments, a strand of the stem portion comprises a sequence and the other strand of the stem portion comprises a sequence that is, at least partially, complementary. In some embodiments, such sequences may have 65% to 100% complementarity (e.g., 65%, 70%, 75%, 80%, 85%, 90%, 95%, or 100% complementarity). In some embodiments, a guide nucleic acid comprises nucleotide sequence that when involved in hybridization events may hybridize over one or more segments such that intervening or adjacent segments are not involved in the hybridization event (e.g., a bulge, a loop structure or hairpin structure, etc.).

TABLE 3 provides illustrative repeat sequences for use with the compositions, systems and methods of the disclosure. In some embodiments, the repeat sequence comprises a sequence that is at least 70%, at least 80%, at least 90%, at least 92%, at least 95%, at least 97%, or at least 99%, or 100% identical to any one of the sequences recited in TABLE 3. In some embodiments, the repeat sequence comprises one or more nucleotide alterations at one or more positions in any one of the sequences of TABLE 3. Alternative nucleotides can be any one or more of A, C, G, T or U, or a deletion, or an insertion.

### Spacer Sequence

Guide nucleic acids described herein may comprise one or more spacer sequences. In some embodiments, a spacer sequence is capable of hybridizing to a target sequence of a target nucleic acid. In some embodiments, a spacer sequence comprises a nucleotide sequence that is, at least partially, hybridizable to an equal length of a sequence (e.g., a target sequence) of a target nucleic acid. Exemplary hybridization conditions are described herein. For example, when describing hybridization, hybridizable, and grammatical equivalents thereof, reference is made to a nucleotide sequence that is able to noncovalently interact, i.e. form Watson-Crick base pairs and/or G/U base pairs, or anneal, to another nucleotide sequence in a sequence-specific, antiparallel, manner (i.e., a nucleotide sequence specifically interacts to a complementary nucleotide sequence) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) for both DNA and RNA. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, a guanine (G) can be considered complementary to both an uracil (U) and to an adenine (A). Accordingly, when a G/U base-pair can be made at a given nucleotide position, the position is not considered to be non-complementary, but is instead considered to be complementary. While hybridization typically occurs between two nucleotide sequences that are complementary, mismatches between bases are possible. It is understood that two nucleotide sequences need not be 100% complementary to be specifically hybridizable, hybridizable, partially hybridizable, or for hybridization to occur. Moreover, a nucleotide sequence may hybridize over one or more segments such that intervening or adjacent segments are not involved in the hybridization event (e.g., a bulge, a loop structure or hairpin structure, etc.). The conditions appropriate for hybridization between two nucleotide sequences depend on the length of the sequence and the degree of complementarity, variables which are well known in the art. For hybridizations between nucleic acids with short stretches of complementarity (e.g., complementarity over 35 or less, 30 or less, 25 or less, 22 or less, 20 or less, or 18 or less nucleotides) the position of mismatches may become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). Any suitable in vitro assay may be utilized to assess whether two sequences hybridize. One such assay is a melting point analysis where the greater the degree of complementarity between two nucleotide sequences, the greater the value of the melting temperature (Tm) for hybrids of nucleic acids having those sequences. The conditions of temperature and ionic strength determine the “stringency” of the hybridization. Temperature, wash solution salt concentration, and other conditions may be adjusted as necessary according to factors such as length of the region of complementation and the degree of complementation. Hybridization and washing conditions are well known and exemplified in Sambrook, J. and Russell, W., Molecular Cloning: A Laboratory Manual, Third Edition, Cold Spring Harbor Laboratory Press, Cold Spring Harbor (2001); and in Green, M. and Sambrook, J., Molecular Cloning: A Laboratory Manual, Fourth Edition, Cold Spring Harbor Laboratory Press, Cold Spring Harbor (2012).

In some embodiments, the spacer sequence may function to direct an RNP complex comprising the guide nucleic acid to the target nucleic acid for detection and/or modification. The spacer sequence may function to direct a RNP to the target nucleic acid for detection and/or modification. A spacer sequence may be complementary to a target sequence that is adjacent to a PAM that is recognizable by an effector protein described herein. The spacer may comprise complementarity with (e.g., hybridize to) a target sequence of a target nucleic acid.

In some embodiments, a spacer sequence comprises at least 5 to about 50 contiguous nucleotides that are complementary to a target sequence in a target nucleic acid. In some embodiments, a spacer sequence comprises at least 5 to about 50 linked nucleotides. In some embodiments, a spacer sequence comprises at least 5 to about 50, at least 5 to about 25, at least about 10 to about 25, or at least 15 to about 25 linked nucleotides. In some embodiments, the spacer sequence comprises 15-28 linked nucleotides. In some embodiments, a spacer sequence comprises 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleotides. In some embodiments, the spacer sequence is 15-28 linked nucleotides in length. In some embodiments, the spacer sequence is 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleotides in length. In some embodiments, the spacer sequence is 18-24 linked nucleotides in length. In some embodiments, the spacer sequence is at least 15 linked nucleotides in length. In some embodiments, the spacer sequence is at least 16, 18, 20, or 22 linked nucleotides in length. In some embodiments, the spacer sequence is at least 20 linked nucleotides in length. In some embodiments, the spacer sequence is at least 80%, at least 85%, at least 90%, at least 95% or 100% complementary to a target sequence of the target nucleic acid. In some embodiments, the spacer sequence is 100% complementary to the target sequence of the target nucleic acid. In some embodiments, the spacer sequence comprises at least 15 contiguous nucleotides that are complementary to the target nucleic acid. In some embodiments, the spacer sequence comprises at least 10, 11, 12, 13, 14, 15, 16, 17, 18, 19, 20, 21, 22, 23, 24, 25, 26, 27, 28, 29, or 30 nucleotides. In some embodiments, the spacer sequence is at least 17 linked nucleotides in length. In some embodiments, the spacer sequence is at least 18 linked nucleotides in length. In some embodiments, the spacer sequence comprises 10, 11, 12, 13, 14, 15, 16, 17, 18, 19, 20, 21, 22, 23, 24, 25, 26, 27, 28, 29, 30, or more nucleotides.

In some embodiments, a spacer sequence is adjacent to a repeat sequence. In some embodiments, a spacer sequence follows a repeat sequence in a 5′ to 3′ direction. In some embodiments, a spacer sequence precedes a repeat sequence in a 5′ to 3′ direction. In some embodiments, the spacer sequence(s) and the repeat sequence(s) of the guide nucleic acid are present within the same molecule. In some embodiments, the spacer(s) and repeat sequence(s) are linked directly to one another. In some embodiments, a linker is present between the spacer(s) and repeat sequences. Linkers may be any suitable linker. In some embodiments, the spacer sequence(s) and the repeat sequence(s) of the guide nucleic acid are present in separate molecules, which are joined to one another by base pairing interactions.

In some embodiments, a spacer sequence comprises a nucleotide sequence that is at least 65%, at least 70%, at least 75%, at least 80%, at least 85%, at least 90%, at least 95% or 100% complementary to a target sequence of a target nucleic acid. A spacer sequence is capable of hybridizing to an equal length portion of a target nucleic acid (e.g., a target sequence). In some embodiments, a target nucleic acid, such as DNA or RNA, may be a cancer gene or gene associated with a genetic disorder, or an amplicon thereof, as described herein. In some embodiments, a target nucleic acid is a gene selected from TABLE 6. In some embodiments, a spacer sequence comprises a nucleotide sequence that is at least 65%, at least 70%, at least 75%, at least 80%, at least 85%, at least 90%, at least 95% or 100% complementary to a target sequence of a target nucleic acid selected from TABLE 6. In some embodiments, a target nucleic acid is a nucleic acid associated with a disease or syndrome set forth in TABLE 6.1. In some embodiments, a spacer sequence comprises a nucleotide sequence that is at least 65%, at least 70%, at least 75%, at least 80%, at least 85%, at least 90%, at least 95% or 100% complementary to a target sequence of a target nucleic acid associated with a disease or syndrome set forth in TABLE 6.1. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are capable of hybridizing to the target sequence. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are complementary to the target sequence.

In some embodiments, when describing percent complementary and grammatical equivalents thereof, of two or more nucleic acid molecules, refer to the percent of nucleotides in two nucleotide sequences in said nucleic acid molecules of equal length that can undergo cumulative base pairing at two or more individual corresponding positions in an antiparallel orientation. Accordingly, also described are nucleic acid sequences that are not completely complementary over their entire length, which indicates that the two or more nucleic acid molecules include one or more mismatches. A mismatch is present at any position in the two opposed nucleotides that are not complementary. The % complementary is calculated by dividing the total number of the complementary residues by the total number of the nucleotides in one of the equal length sequences, and multiplying by 100. Complete or total complementarity describes nucleotide sequences in 100% of the residues of a nucleotide sequence are complementary to residues in a reference nucleotide sequence. In some embodiments, when describing something that is partially complementarity, reference is made to nucleotide sequences in which at least 20%, but less than 100%, of the residues of a nucleotide sequence are complementary to residues in a reference nucleotide sequence. In some instances, at least 50%, but less than 100%, of the residues of a nucleotide sequence are complementary to residues in a reference nucleotide sequence. In some instances, at least 70%, 80%, 90% or 95%, but less than 100%, of the residues of a nucleotide sequence are complementary to residues in a reference nucleotide sequence. In some embodiments, when describing something that is noncomplementary, reference is made to nucleotide sequences in which less than 20% of the residues of a nucleotide sequence are complementary to residues in a reference nucleotide sequence.

It is understood that the spacer sequence of a spacer sequence need not be 100% complementary to that of a target sequence of a target nucleic acid to hybridize or hybridize specifically to the target sequence. For example, the spacer sequence may comprise at least one alteration, such as a substituted or modified nucleotide, that is not complementary to the corresponding nucleotide of the target sequence. The guide nucleic acid may comprise at least one uracil between nucleic acid residues 5 to 20 of the spacer region that is not complementary to the corresponding nucleotide of the target sequence. The guide nucleic acid may comprise at least one uracil between nucleic acid residues 5 to 9, 10 to 14, or 15 to 20 of the spacer region that is not complementary to the corresponding nucleotide of the target sequence. In some embodiments, the region of the target nucleic acid that is complementary to the spacer region comprises an epigenetic modification or a post-transcriptional modification. In some embodiments, the epigenetic modification comprises an acetylation, methylation, or thiol modification. Spacer sequences are further described throughout herein, for example, in the Examples section.

TABLE 4 provides illustrative spacer sequences for use with the compositions, systems and methods of the disclosure. In some embodiments, the spacer sequence comprises a sequence that is at least 70%, at least 80%, at least 90%, at least 92%, at least 95%, at least 97%, or at least 99%, or 100% identical to any one of the sequences recited in TABLE 4. In some embodiments, the spacer sequence comprises one or more nucleotide alterations at one or more positions in any one of the sequences of TABLE 4. Alternative nucleotides can be any one or more of A, C, G, T or U, or a deletion, or an insertion.

In some embodiments, the guide nucleic acid or a nucleic acid encoding the guide nucleic acid comprises a spacer sequence and/or a repeat sequence. In some embodiments, the guide nucleic acid comprises a repeat sequence that is at least 70%, at least 75%, at least 80%, at least 85%, at least 90%, at least 95%, or 100% identical to any one of the sequences of TABLE 3 and a spacer sequence that is at least 70%, at least 75%, at least 80%, at least 85%, at least 90%, at least 95%, or 100% identical to any one of the sequences of TABLE 4.

### Linker for Nucleic Acids

In some embodiments, a guide nucleic acid for use with compositions, systems, and methods described herein comprises one or more linkers, or a nucleic acid encoding one or more linkers. In some embodiments, the guide nucleic acid comprises at least one, at least two, at least three, at least four, at least five, at least six, at least seven, at least eight, at least nine, or at least ten linkers. In some embodiments, the guide nucleic acid comprises one, two, three, four, five, six, seven, eight, nine, or ten linkers. In some embodiments, the guide nucleic acid comprises more than one linker. In some embodiments, at least two of the more than one linker are the same. In some embodiments, at least two of the more than one linker are not same.

In some embodiments, a linker comprises one to ten, one to seven, one to five, one to three, two to ten, two to eight, two to six, two to four, three to ten, three to seven, three to five, four to ten, four to eight, four to six, five to ten, five to seven, six to ten, six to eight, seven to ten, or eight to ten linked nucleotides. In some embodiments, the linker comprises one, two, three, four, five, six, seven, eight, nine, or ten linked nucleotides. In some embodiments, a linker comprises a nucleotide sequence of 5′-GAAA-3′.

In some embodiments, a guide nucleic acid comprises one or more linkers connecting one or more repeat sequences. In some embodiments, the guide nucleic acid comprises one or more linkers connecting one or more repeat sequences and one or more spacer sequences. In some embodiments, the guide nucleic acid comprises at least two repeat sequences connected by a linker.

### A Single Nucleic Acid System

In some embodiments, compositions, systems and methods described herein comprise a single nucleic acid system comprising a guide nucleic acid or a nucleotide sequence encoding the guide nucleic acid, and one or more effector proteins or a nucleotide sequence encoding the one or more effector proteins.

In some embodiments, when describing a single nucleic acid system, reference is made to a system that uses a guide nucleic acid complexed with one or more polypeptides described herein, wherein the complex is capable of interacting with a target nucleic acid in a sequence specific manner, and wherein the guide nucleic acid is capable of non-covalently interacting with the one or more polypeptides described herein, and wherein the guide nucleic acid is capable of hybridizing with a target sequence of the target nucleic acid. A single nucleic acid system lacks a duplex of a guide nucleic acid as hybridized to a second nucleic acid, wherein in such a duplex the second nucleic acid, and not the guide nucleic acid, is capable of interacting with the effector protein

In some embodiments, a second region of the guide nucleic acid non-covalently interacts with the one or more polypeptides described herein. In some embodiments, a first region of the guide nucleic acid hybridizes with a target sequence of the target nucleic acid. In the single nucleic acid system having a complex of the guide nucleic acid and the effector protein, the effector protein is not transactivated by the guide nucleic acid. In other words, activity of effector protein does not require binding to a second non-target nucleic acid molecule. An exemplary guide nucleic acid for a single nucleic acid system is a crRNA.

crRNA

In some embodiments, a guide nucleic acid may comprise a CRISPR RNA (crRNA). In some embodiments, guide nucleic acid can generally comprise a crRNA, at least a portion of which is complementary to a target sequence of a target nucleic acid. A crRNA may be the product of processing of a longer precursor CRISPR RNA (pre-crRNA) transcribed from the CRISPR array by cleavage of the pre-crRNA within each direct repeat sequence to afford shorter, mature crRNAs. A crRNA may be generated by a variety of mechanisms, including the use of dedicated endonucleases (e.g., Cas6 or Cas5d in Type I and III systems), coupling of a host endonuclease (e.g., RNase III) with tracrRNA (Type II systems), or a ribonuclease activity endogenous to the effector protein itself (e.g., Cpf1, from Type V systems). A crRNA may also be specifically generated outside of processing of a pre-crRNA and individually contacted to an effector protein in vivo or in vitro.

In some embodiments, the guide nucleic acid comprises a portion of, or all of a repeat sequence that interacts with the effector protein. In general, a crRNA can comprise a spacer region that hybridizes to a target sequence of a target nucleic acid, and in some embodiments can further comprise, a repeat region that interacts with the effector protein. In some embodiments, a crRNA comprises a first region and a second region, wherein the second region of the crRNA comprises a repeat sequence, and the first region of the crRNA comprises a spacer sequence. In some embodiments, the repeat sequence and the spacer sequences are directly connected to each other (e.g., covalent bond (phosphodiester bond)). In some embodiments, the repeat sequence and the spacer sequence are connected by a linker.

In some embodiments, a crRNA is useful as a single nucleic acid system for compositions, methods, and systems described herein or as part of a single nucleic acid system for compositions, methods, and systems described herein. In some embodiments, a crRNA is useful as part of a single nucleic acid system for compositions, methods, and systems described herein. In such embodiments, a single nucleic acid system comprises a guide nucleic acid comprising a crRNA wherein, a repeat sequence of a crRNA is capable of connecting a crRNA to an effector protein. In some embodiments, a single nucleic acid system comprises a guide nucleic acid comprising a crRNA linked to another nucleotide sequence that is capable of being non-covalently bond by an effector protein. In such embodiments, a repeat sequence of a crRNA can be linked to an intermediary sequence. In some embodiments, a single nucleic acid system comprises a guide nucleic acid comprising a crRNA and an intermediary sequence.

A crRNA may include deoxyribonucleosides, ribonucleosides, chemically modified nucleosides, or any combination thereof. In some embodiments, a crRNA comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In some embodiments, a crRNA comprises at least: 10, 15, 20, 25, 30, 35, 40, 45, 50, 55, 60 linked nucleotides. In some embodiments, the length of the crRNA is about 20 to about 120 linked nucleotides. In some embodiments, the length of a crRNA is about 20 to about 100, about 30 to about 100, about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a crRNA is about 20, about 25, about 30, about 35, about 40, about 45, about 50, about 55, about 60, about 65, about 70 or about 75 linked nucleotides.

TABLE 5 provides illustrative crRNA sequences for use with the compositions, systems and methods of the disclosure. In some embodiments, the crRNA sequence comprises at least 65%, at least 70%, at least 80%, at least 90%, at least 92%, at least 95%, at least 97%, or at least 99%, or 100% sequence identity to a sequence as set forth in TABLE 5. In some embodiments, the crRNA sequence comprises one or more nucleotide alterations at one or more positions in any one of the sequences of TABLE 5. Alternative nucleotides can be any one or more of A, C, G, T or U, or a deletion, or an insertion.

### IV. Modifications

Polypeptides (e.g., effector proteins) and nucleic acids (e.g., engineered guide nucleic acids) described herein can be further modified as described throughout and as further described herein. Examples are modifications of interest that do not alter the primary sequence of the polypeptides or nucleic acids, including chemical derivatization of polypeptides, e.g., acylation, acetylation, carboxylation, amidation, etc, or other modifications that do not alter the primary sequence of the polypeptide of nucleic acid. Also included are modifications of glycosylation, e.g., those made by modifying the glycosylation patterns of a polypeptide during its synthesis and processing or in further processing steps; e.g., by exposing the polypeptide to enzymes which affect glycosylation, such as mammalian glycosylating or deglycosylating enzymes. Also embraced are polypeptide sequences that have phosphorylated amino acid residues, e.g., phosphotyrosine, phosphoserine, or phosphothreonine.

Modifications disclosed herein can also include modification of described polypeptides and/or engineered guide nucleic acids through any suitable method, such as molecular biological techniques and/or synthetic chemistry, to improve their resistance to proteolytic degradation, to change the target sequence specificity, to optimize solubility properties, to alter protein activity (e.g., transcription modulatory activity, enzymatic activity, etc.) or to render them more suitable for their intended purpose (e.g., in vivo administration, in vitro methods, or ex vivo applications). Analogs of such polypeptides include those containing residues other than naturally occurring L-amino acids, e.g., D-amino acids or non-naturally occurring synthetic amino acids. D-amino acids may be substituted for some or all of the amino acid residues. Modifications can also include modifications with non-naturally occurring unnatural amino acids. The particular sequence and the manner of preparation will be determined by convenience, economics, purity required, and the like.

Modifications can further include the introduction of various groups to polypeptides and/or engineered guide nucleic acids described herein. For example, groups can be introduced during synthesis or during expression of a polypeptide (e.g., an effector protein), which allow for linking to other molecules or to a surface. Thus, e.g., cysteines can be used to make thioethers, histidines for linking to a metal ion complex, carboxyl groups for forming amides or esters, amino groups for forming amides, and the like.

Modifications can further include modification of nucleic acids described herein (e.g., nucleic acids encoding effector proteins, engineered guide nucleic acids, or nucleic acids encoding engineered guide nucleic acids) to provide the nucleic acid with a new or enhanced feature, such as improved stability. Such modifications of a nucleic acid include a base editing, a base modification, a backbone modification, a sugar modification, or combinations thereof, of one or more nucleotides, nucleosides, or nucleobases in a nucleic acid.

In some embodiments, nucleic acids (e.g., engineered guide nucleic acids) described herein comprise one or more modifications comprising: 2′O-methyl modified nucleotides (e.g., 2′-O-Methyl (2′OMe) sugar modifications); 2′ Fluoro modified nucleotides (e.g., 2′-fluoro (2′-F) sugar modifications); locked nucleic acid (LNA) modified nucleotides; peptide nucleic acid (PNA) modified nucleotides; nucleotides with phosphorothioate linkages; a 5′ cap (e.g., a 7-methylguanylate cap (m7G)), phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkyl phosphoramidates, phosphorodiamidates, thionophosphor amidates, thionoalkylphosphonates, thionoalkylphosphotriesters, selenophosphates and boranophosphates having normal 3′-5′ linkages, 2′-5′ linked analogs of these, and those having inverted polarity wherein one or more intemucleotide linkages is a 3′ to 3′, 5′ to 5′ or 2′ to 2′ linkage; phosphorothioate and/or heteroatom internucleoside linkages, such as —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—); morpholino linkages (formed in part from the sugar portion of a nucleoside); morpholino backbones; phosphorodiamidate or other non-phosphodiester internucleoside linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; other backbone modifications having mixed N, O, S and CH₂ component parts; and combinations thereof.

Methods of modifying a nucleic acid or amino acid sequence are known. One of ordinary skill in the art will appreciate that the engineered modification(s) may be located at any position(s) of a nucleic acid such that the function of the nucleic acid, protein, composition or system is not substantially decreased. Nucleic acids provided herein can be prepared according to any available technique including, but not limited to chemical synthesis, enzymatic synthesis, which is generally termed in vitro-transcription, cloning, enzymatic, or chemical cleavage, etc. In some instances, the nucleic acids provided herein are not uniformly modified along the entire length of the molecule. Different nucleotide modifications and/or backbone structures can exist at various positions within the nucleic acid.

### V. Vectors and Multiplexed Expression Vectors

Compositions, methods, and systems described herein comprise a vector or a use thereof. A vector can comprise a nucleic acid of interest. In some embodiments, the nucleic acid of interest comprises one or more components of a composition or system described herein. In some embodiments, the nucleic acid of interest comprises a nucleotide sequence that encodes one or more components of the composition or system described herein. In some embodiments, one or more components comprises a polypeptide(s) (e.g., effector protein(s), effector partner(s) such as fusion partner(s), fusion protein(s), or combinations thereof), guide nucleic acid(s), target nucleic acid(s), and donor nucleic acid(s). In some embodiments, the component comprises a nucleic acid encoding a polypeptide (e.g., effector protein(s), effector partner(s) such as a fusion partner(s), fusion protein(s), or combinations thereof), a donor nucleic acid, and a guide nucleic acid or a nucleic acid encoding the guide nucleic acid. In some embodiments, a vector encoding a donor nucleic acid further encodes a target nucleic acid.

In some embodiments, a vector may be part of a vector system. The vector system may comprise a library of vectors each encoding one or more component of a composition or system described herein. In some embodiments, components described herein (e.g., an effector protein, a guide nucleic acid, and/or a donor nucleic acid) are encoded by the same vector. In some embodiments, components described herein (e.g., an effector protein, a guide nucleic acid, and/or a donor nucleic acid) are each encoded by different vectors of the system. In some instances, compositions, methods and systems provided herein comprise a vector system encoding a polypeptide (e.g., an effector protein) described herein. In some instances, compositions and systems provided herein comprise a vector system encoding a guide nucleic acid (e.g., crRNA) described herein. In some instances, compositions and systems provided herein comprise a multi-vector system encoding an effector protein and a guide nucleic acid described herein, wherein the guide nucleic acid and the effector protein are encoded by the same or different vectors. In some instances, the engineered guide and the engineered effector protein are encoded by different vectors of the system. In some embodiments, a nucleic acid encoding a polypeptide (e.g., an effector protein) comprises an expression vector. In some embodiments, a nucleic acid encoding a polypeptide is a messenger RNA. In some embodiments, an expression vector comprises or encodes an engineered guide nucleic acid. In some cases, the expression vector encodes the crRNA.

In some embodiments, a vector comprises a nucleotide sequence encoding one or more polypeptides (e.g., effector proteins, effector partners such as fusion partners, fusion proteins, or combinations thereof as described herein. In some embodiments, the one or more polypeptides comprise at least two polypeptides. In some embodiments, the at least two polypeptides are the same. In some embodiments, the at least two polypeptides are different from each other. In some embodiments, the nucleotide sequence is operably linked to a promoter that is operable in a target cell, such as a eukaryotic cell. In some embodiments, the vector comprises the nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more polypeptides.

In some embodiments, a vector may encode one or more of any system components, including but not limited to polypeptides (e.g., effector proteins, effector partners such as fusion partners, fusion proteins, or combinations thereof), guide nucleic acids, and target nucleic acids as described herein. In some embodiments, a system component encoding sequence is operably linked to a promoter that is operable in a target cell, such as a eukaryotic cell. In some embodiments, a vector may encode 1, 2, 3, 4 or more of any system components. For example, a vector may encode two or more guide nucleic acids, wherein each guide nucleic acid comprises a different sequence. A vector may encode the polypeptide and the guide nucleic acid. A vector may encode the polypeptide, a guide nucleic acid, a donor nucleic acid, or combinations thereof.

In some instances, a vector may encode one or more engineered effector proteins. In some instances, a vector may encode 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 engineered effector proteins. In some instances, a vector can encode one or more engineered effector proteins comprising any one or more of the alterations set forth in TABLE 1.

In some instances, a vector may encode one or more guide nucleic acids. In some embodiments, a vector comprises one or more guide nucleic acids, or a nucleotide sequence encoding the one or more guide nucleic acids as described herein. In some embodiments, the one or more guide nucleic acids comprise at least two guide nucleic acids. In some embodiments, the at least two guide nucleic acids are the same. In some embodiments, the at least two guide nucleic acids are different from each other. In some embodiments, the guide nucleic acid or the nucleotide sequence encoding the guide nucleic acid is operably linked to a promoter that is operable in a target cell, such as a eukaryotic cell. In some embodiments, the vector comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids. In some embodiments, the vector comprises a nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids. In some instances, a vector may encode 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 different guide nucleic acids. In some instances, a vector can encode one or more guide nucleic acids comprising a sequence set forth in TABLE 3, TABLE 4, or both. In some instances, a vector can encode one or more guide nucleic acids comprising a crRNA sequence of any one of any one of the sequences set forth in TABLE 5.

In some embodiments, a vector comprises one or more donor nucleic acids as described herein. In some embodiments, the one or more donor nucleic acids comprise at least two donor nucleic acids. In some embodiments, the at least two donor nucleic acids are the same. In some embodiments, the at least two donor nucleic acids are different from each other. In some embodiments, the vector comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more donor nucleic acids.

In some embodiments, a vector may comprise or encode one or more regulatory elements. Regulatory elements may refer to transcriptional and translational control sequences, such as promoters, enhancers, polyadenylation signals, terminators, protein degradation signals, and the like, that provide for and/or regulate transcription of a non-coding sequence or a coding sequence and/or regulate translation of an encoded polypeptide. In some embodiments, a vector may comprise or encode for one or more additional elements, such as, for example, replication origins, antibiotic resistance (or a nucleic acid encoding the same), a tag (or a nucleic acid encoding the same), selectable markers, and the like. In some embodiments, a vector comprises or encodes for one or more elements, such as, for example, ribosome binding sites, and RNA splice sites.

Vectors described herein can encode a promoter—a regulatory region on a nucleic acid, such as a DNA sequence, capable of initiating transcription of a downstream (3′ direction) coding or non-coding sequence. A promoter can be linked at its 3′ terminus to a nucleic acid, the expression or transcription of which is desired, and extends upstream (5′ direction) to include bases or elements necessary to initiate transcription or induce expression, which could be measured at a detectable level. A promoter can comprise a nucleotide sequence, referred to herein as a “promoter sequence”. The promoter sequence can include a transcription initiation site, and one or more protein binding domains responsible for the binding of transcription machinery, such as RNA polymerase. When eukaryotic promoters are used, such promoters can contain “TATA” boxes and “CAT” boxes. Various promoters, including inducible promoters, may be used to drive expression, i.e., transcriptional activation, of the nucleic acid of interest. Accordingly, in some embodiments, the nucleic acid of interest can be operably linked to a promoter.

Promotors may be any suitable type of promoter envisioned for the compositions, systems, and methods described herein. Examples include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc. Suitable promoters include, but are not limited to: SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, and a human Hl promoter (Hl). By transcriptional activation, it is intended that transcription will be increased above basal levels in the target cell by 2 fold, 5 fold, 10 fold, 50 fold, by 100 fold, 500 fold, or by 1000 fold, or more. In addition, vectors used for providing a nucleic acid that, when transcribed, produces a guide nucleic acid and/or a nucleic acid that encodes a polypeptide (e.g., an effector protein, an effector partner such as a fusion partner, a fusion protein, or a combination thereof) to a cell may include nucleic acid sequences that encode for selectable markers in the target cells, so as to identify cells that have taken up the guide nucleic acid and/or the polypeptide.

In general, vectors provided herein comprise at least one promotor or a combination of promoters driving expression or transcription of one or more genome editing tools described herein. In some embodiments, the vector comprises a nucleotide sequence of a promoter. In some embodiments, the vector comprises two promoters. In some embodiments, the vector comprises three promoters. In some embodiments, a length of the promoter is less than about 500, less than about 400, less than about 300, or less than about 200 linked nucleotides. In some embodiments, a length of the promoter is at least 100, at least 200, at least 300, at least 400, or at least 500 linked nucleotides. Non-limiting examples of promoters include CMV, 7SK, EF1a, RPBSA, hPGK, EFS, SV40, PGK1, Ubc, human beta actin, TRE, UAS, Ac5, Polyhedrin, CaMKIIa, GAL1-10, H1, TEF1, GDS, ADH1, CaMV35S, HSV TK, Ubi, U6, MNDU3, MSCV, MND[, if target specific, insert specific promoters here] and CAG.

In some embodiments, some promoters (e.g., U6, enhanced U6, Hl and 7SK) prefers the nucleic acid being transcribed having “g” nucleotide at the 5′ end of the coding sequence. Accordingly, when such coding sequence is expressed, it comprises an additional “g” nucleotide at 5′ end. In some embodiments, vectors provided herein comprise a promotor driving expression or transcription of any one of the guide nucleic acids described herein (e.g., TABLE 4, TABLE 5, TABLE 6, TABLE 7, and TABLE 8) further comprises “g” nucleotide at 5′ end of the guide nucleic acid, wherein the promotor is selected from U6, enhanced U6, Hl and 7SK.

In some embodiments, the promoter is a constitutive promoter. In some embodiments, the promoter is an inducible promoter. In some embodiments, the inducible promoter only drives expression of its corresponding coding sequence (e.g., polypeptide or guide nucleic acid) when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter. [In some embodiments, the promoter for expressing effector protein is a muscle-specific promoter. In some embodiments, the muscle-specific promoter comprises Ck8e, SPC5-12, or Desmin promoter sequence.] In some embodiments, the promoter for expressing a polypeptide (e.g., an effector protein, an effector partner such as a fusion partner, a fusion protein, or a combination thereof) is a ubiquitous promoter. In some embodiments, the ubiquitous promoter comprises MND or CAG promoter sequence.

In some embodiments, the promoters are prokaryotic promoters (e.g., drive expression of a gene in a prokaryotic cell). In some embodiments, the promoters are eukaryotic promoters, (e.g., drive expression of a gene in a eukaryotic cell). In some embodiments, the promoter is EF1a. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, a vector described herein is a nucleic acid expression vector. In some embodiments, a vector described herein is a recombinant expression vector. In some embodiments, a vector described herein is a messenger RNA. In some embodiments, a vector comprising the recombinant nucleic acid as described herein, wherein the vector is a viral vector, an adeno associated viral (AAV) vector, a retroviral vector, or a lentiviral vector. In some embodiments, a vector described herein or a recombinant nucleic acid described herein is comprised in a cell. In some embodiments, a recombinant nucleic acid integrated into a genomic DNA sequence of the cell, wherein the cell is a eukaryotic cell or a prokaryotic cell.

In some embodiments, a vector described herein is a delivery vector. In some embodiments, the delivery vector is a eukaryotic vector, a prokaryotic vector (e.g., a bacterial vector) a viral vector, or any combination thereof. In some embodiments, the delivery vehicle is a non-viral vector. In some embodiments, the delivery vector is a plasmid. In some embodiments, the plasmid comprises DNA. In some embodiments, the plasmid comprises RNA. In some embodiments, the plasmid comprises circular double-stranded DNA. In some embodiments, the plasmid is linear. In some embodiments, the plasmid comprises one or more coding sequences of interest and one or more regulatory elements. In some embodiments, the plasmid comprises a bacterial backbone containing an origin of replication and an antibiotic resistance gene or other selectable marker for plasmid amplification in bacteria. In some embodiments, the plasmid is a minicircle plasmid. In some embodiments, the plasmid contains one or more genes that provide a selective marker to induce a target cell to retain the plasmid. In some examples, the plasmids are engineered through synthetic or other suitable means known in the art. For example, in some embodiments, the genetic elements are assembled by restriction digest of the desired genetic sequence from a donor plasmid or organism to produce ends of the DNA which is then be readily ligated to another genetic sequence.

In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo basepairs. Furthermore, enhancers are located optionally upstream or downstream of a gene region to be transcribed, and/or located within the gene, to activate the transcription. Exemplary enhancers include, but are not limited to, WPRE; CMV enhancers; the R—U5′ segment in LTR of HTLV-I.

### Administration of a Non-Viral Vector

In some embodiments, an administration of a non-viral vector comprises contacting a cell, such as a host cell, with the non-viral vector. In some embodiments, a physical method or a chemical method is employed for delivering the vector into the cell. Exemplary physical methods include electroporation, gene gun, sonoporation, magnetofection, or hydrodynamic delivery. Exemplary chemical methods include delivery of the recombinant polynucleotide by liposomes such as, cationic lipids or neutral lipids; lipofection; dendrimers; lipid nanoparticle (LNP); or cell-penetrating peptides.

In some embodiments, a vector is administered as part of a method of nucleic acid detection, editing, and/or treatment as described herein. In some embodiments, a vector is administered in a single vehicle, such as a single expression vector. In some embodiments, at least two of the three components, a nucleic acid encoding one or more polypeptides (e.g., effector proteins, effector partners such as fusion partners, fusion proteins, or combinations thereof), one or more donor nucleic acids, and one or more guide nucleic acids or a nucleic acid encoding the one or more guide nucleic acid, are provided in the single expression vector. In some embodiments, components, such as a guide nucleic acid and a polypeptide (e.g., effector protein, effector partner, such as a fusion partner, fusion protein, or combinations thereof), are encoded by the same vector. In some embodiments, a polypeptide (e.g., effector protein, effector partner, such as a fusion partner, fusion protein, or combinations thereof) (or a nucleic acid encoding same) and/or an engineered guide nucleic acid (or a nucleic acid that, when transcribed, produces same) are not co-administered with donor nucleic acid in a single vehicle. In some embodiments, a polypeptides (e.g., effector protein, effector partner, such as a fusion partner, fusion protein, or combinations thereof) (or a nucleic acid encoding same), an engineered guide nucleic acid (or a nucleic acid that, when transcribed, produces same), and/or donor nucleic acid are administered in one or more or two or more vehicles, such as one or more, or two or more expression vectors.

In some embodiments, a vector system is administered as part of a method of nucleic acid detection, editing, and/or treatment as described herein, wherein at least two vectors are co-administered. In some embodiments, the at least two vectors comprise different components. In some embodiments, the at least two vectors comprise the same component having different sequences. In some embodiments, at least one of the three components, a nucleic acid encoding one or more polypeptides (e.g., effector proteins, effector partners, such as a fusion partners, fusion proteins, or combinations thereof), one or more donor nucleic acids, and one or more guide nucleic acids or a nucleic acid encoding the one or more guide nucleic acids, or a variant thereof is provided in a different vector. In some embodiments, the nucleic acid encoding the polypeptides (e.g., effector proteins, effector partners, such as a fusion partners, fusion proteins, or combinations thereof), and a guide nucleic acid or a nucleic acid encoding the guide nucleic acid are provided in different vectors. In some embodiments, the donor nucleic acid is encoded by a different vector than the vector encoding the effector protein and the guide nucleic acid.

### Lipid Particles and Non-Viral Vectors

In some instances, compositions and systems provided herein comprise a lipid particle. In some embodiments, a lipid particle is a lipid nanoparticle (LNP). In some embodiments, a lipid or a lipid nanoparticle can encapsulate a nucleic acid (e.g., DNA or RNA) encoding one or more of the components as described herein. In some embodiments, a lipid or a lipid nanoparticle can encapsulate an expression vector. In some embodiments, a lipid or a lipid nanoparticle can encapsulate the effector protein, the sgRNA or crRNA, the nucleic acid encoding the effector protein and/or the DNA molecule encoding the sgRNA or crRNA.

LNPs are a non-viral delivery system for gene therapy. LNPs are effective for delivery of nucleic acids. Beneficial properties of LNP include ease of manufacture, low cytotoxicity and immunogenicity, high efficiency of nucleic acid encapsulation and cell transfection, multi-dosing capabilities and flexibility of design (Kulkarni et al., (2018) Nucleic Acid Therapeutics, 28(3):146-157).

In some embodiments, compositions and methods comprise a lipid, polymer, nanoparticle, or a combination thereof, or use thereof, to introduce one or more effector proteins, one or more guide nucleic acids, one or more donor nucleic acids, or any combinations thereof to a cell. Non-limiting examples of lipids and polymers are cationic polymers, cationic lipids, ionizable lipids, or bio-responsive polymers. In some embodiments, the ionizable lipids exploits chemical-physical properties of the endosomal environment (e.g., pH) offering improved delivery of nucleic acids. In some embodiments, the ionizable lipids are neutral at physiological pH. In some embodiments, the ionizable lipids are protonated under acidic pH. In some embodiments, the bio-responsive polymer exploits chemical-physical properties of the endosomal environment (e.g., pH) to preferentially release the genetic material in the intracellular space. In some cases, a method can comprise contacting a cell with an expression vector. In some cases, contacting can comprise electroporation, lipofection, or lipid nanoparticle (LNP) delivery of an expression vector.

In some embodiments, a LNP comprises an outer shell and an inner core. In some embodiments, the outer shell comprises lipids. In some embodiments, the lipids comprise modified lipids. In some embodiments, the modified lipids comprise pegylated lipids. In some embodiments, the lipids comprise one or more of cationic lipids, anionic lipids, ionizable lipids, and non-ionic lipids. In some embodiments, the LNP comprises one or more of N1,N3,N5-tris(3-(didodecylamino)propyl)benzene-1,3,5-tricarboxamide (TT3), 2-dioleoyl-sn-glycero-3-phosphoethanolamine (DOPE), 1-palmitoyl-2-oleoylsn-glycero-3-phosphoethanolamine (POPE), 1,2-distearoyl-sn-glycero-3-phosphocholine (DSPC), cholesterol (Chol), 1,2-dimyristoyl-sn-glycerol, and methoxypolyethylene glycol (DMG-PEChooo), derivatives, analogs, or variants thereof. In some embodiments, the LNP has a negative net overall charge prior to complexation with one or more of a guide nucleic acid, a nucleic acid encoding the one or more guide nucleic acid, a nucleic acid encoding a polypeptide (e.g., effector protein, effector partner, such as a fusion partner, fusion protein, or combinations thereof), and/or a donor nucleic acid. In some embodiments, the inner core is a hydrophobic core. In some embodiments, the one or more of a guide nucleic acid, the nucleic acid encoding the one or more guide nucleic acid, the nucleic acid encoding the polypeptide, and/or the donor nucleic acid forms a complex with one or more of the cationic lipids and the ionizable lipids. In some embodiments, the nucleic acid encoding the polypeptide or the nucleic acid encoding the guide nucleic acid is self-replicating.

In some embodiments, a LNP comprises one or more of cationic lipids, ionizable lipids, and modified versions thereof. In some embodiments, the ionizable lipid comprises TT3 or a derivative thereof. Accordingly, in some embodiments, the LNP comprises one or more of TT3 and pegylated TT3. The publication WO2016187531 is hereby incorporated by reference in its entirety, which describes representative LNP formulations in Table 2 and Table 3, and representative methods of delivering LNP formulations in Example 7.

In some embodiments, a LNP comprises a lipid composition targeting to a specific organ. In some embodiments, the lipid composition comprises lipids having a specific alkyl chain length that controls accumulation of the LNP in the specific organ (e.g., liver or spleen). In some embodiments, the lipid composition comprises a biomimetic lipid that controls accumulation of the LNP in the specific organ (e.g., brain). In some embodiments, the lipid composition comprises lipid derivatives (e.g., cholesterol derivatives) that controls accumulation of the LNP in a specific cell (e.g., liver endothelial cells, Kupffer cells, hepatocytes).

In some embodiments, the LNP described herein comprises nucleic acids (e.g., DNA or RNA) encoding an effector protein described herein, an effector partner (e.g., a fusion partner) described herein, a fusion protein described herein, a guide nucleic acid described herein, or combinations thereof. In some embodiments, the LNP comprises an mRNA that produces an effector protein described herein, an effector partner (e.g., a fusion partner) described herein, or a fusion protein described herein when translated. In some embodiments, the LNP comprises chemically modified guide nucleic acids.

### Viral Vectors

An expression vector can be a viral vector. In some embodiments, a viral vector comprises a nucleic acid to be delivered into a host cell via a recombinantly produced virus or viral particle. The nucleic acid may be single-stranded or double stranded, linear or circular, segmented or non-segmented. The nucleic acid may comprise DNA, RNA, or a combination thereof. In some embodiments, the expression vector is an adeno-associated viral vector. There are a variety of viral vectors that are associated with various types of viruses, including but not limited to retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. In some embodiments, the vector is an adeno-associated viral (AAV) vector. In some embodiments, the viral vector is a recombinant viral vector. In some embodiments, the vector is a retroviral vector. In some embodiments, the retroviral vector is a lentiviral vector. In some embodiments, the retroviral vector comprises gamma-retroviral vector. A viral vector provided herein can be derived from or based on any such virus. For example, in some embodiments, the gamma-retroviral vector is derived from a Moloney Murine Leukemia Virus (MoMLV, MMLV, MuLV, or MLV) or a Murine Stem cell Virus (MSCV) genome. In some embodiments, the lentiviral vector is derived from the human immunodeficiency virus (HIV) genome. In some embodiments, the viral vector is a chimeric viral vector. In some embodiments, the chimeric viral vector comprises viral portions from two or more viruses. In some embodiments, the viral vector corresponds to a virus of a specific serotype.

Often the viral vectors provided herein are an adeno-associated viral vector (AAV vector). In some embodiments, a viral particle that delivers a viral vector described herein is an AAV. In some embodiments, the AAV comprises any AAV known in the art. In some embodiments, the viral vector corresponds to a virus of a specific AAV serotype. In some embodiments, the AAV serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, an AAV12 serotype, an AAV-rh10 serotype, and any combination, derivative, or variant thereof. In some embodiments, the AAV vector is a recombinant vector, a hybrid AAV vector, a chimeric AAV vector, a self-complementary AAV (scAAV) vector, a single-stranded AAV, or any combination thereof. scAAV genomes are generally known in the art and contain both DNA strands which can anneal together to form double-stranded DNA.

In some embodiments, an AAV vector described herein is a chimeric AAV vector. In some embodiments, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

Generally, an AAV vector has two inverted terminal repeats (ITRs). According, in some embodiments, the viral vector provided herein comprises two inverted terminal repeats of AAV. The nucleotide (e.g., DNA) sequence in between the ITRs of an AAV vector provided herein may be referred to herein as the sequence encoding the genome editing tools. These genome editing tools can include, but are not limited to, a nucleic acid encoding one or more polypeptides (e.g., an effector protein, effector partners such as fusion partners, fusion proteins, or combinations thereof), a nucleic acid encoding the one or more polypeptides comprising a heterologous peptides (e.g., nuclear localization signal (NLS), polyA tail), one or more guide nucleic acid(s), a nucleic acid encoding the one or more guide nucleic acids, respective promoter(s), and one or more donor nucleic acid, or combinations thereof. In some embodiments, a nuclear localization signal comprises an entity (e.g., peptide) that facilitates localization of a nucleic acid, protein, or small molecule to the nucleus, when present in a cell that contains a nuclear compartment.

In some embodiments, viral vectors provided herein comprise at least one promotor or a combination of promoters driving expression or transcription of one or more genome editing tools described herein. In some embodiments, a coding region of the AAV vector forms an intramolecular double-stranded DNA template thereby generating the AAV vector that is a self-complementary AAV (scAAV) vector. In some embodiments, the scAAV vector comprises the sequence encoding genome editing tools that has a length of about 2 kb to about 3 kb. In some embodiments, the AAV vector provided herein is a self-inactivating AAV vector. In some embodiments, the AAV vector provided herein comprises a modification, such as an insertion, deletion, chemical alteration, or synthetic modification, relative to a wild-type AAV vector.

In general, viral vectors provided herein comprise at least one promotor or a combination of promoters driving expression or transcription of one or more genome editing tools described herein. In some embodiments, the length of the promoter is less than about 500, less than about 400, or less than about 300 linked nucleotides. In some embodiments, the length of the promoter is at least 100 linked nucleotides. Non-limiting examples of promoters include CMV, EF1a, RPBSA, hPGK, EFS, SV40, PGK1, Ubc, human beta actin promoter, CAG, TRE, UAS, Ac5, Polyhedrin, CaMKIIa, GAL1, H1, TEF1, GDS, ADH1, CaMV35S, Ubi, U6, MNDU3, and MSCV. In some embodiments, the promoter is an inducible promoter that only drives expression of its corresponding gene when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter, as described further in Kulemzin et al., (2019), BMC Med Genomics, 12:44.

In some embodiments, the coding region of the AAV vector forms an intramolecular double-stranded DNA template thereby generating an AAV vector that is a self-complementary AAV (scAAV) vector. In general, the sequence encoding the genome editing tools of an scAAV vector has a length of about 2 kb to about 3 kb. The scAAV vector can comprise nucleotide sequences encoding an effector protein, providing guide nucleic acids described herein, and a donor nucleic acid described herein. In some embodiments, the AAV vector provided herein is a self-inactivating AAV vector.

In some embodiments, an AAV vector provided herein comprises a modification, such as an insertion, deletion, chemical alteration, or synthetic modification, relative to a wild-type AAV vector.

In some embodiments, the viral particle that delivers the viral vector described herein is an AAV. AAVs are characterized by their serotype. Non-limiting examples of AAV serotypes are AAV1, AAV2, AAV3, AAV4, AAV5, AAV6, AAV7, AAV8, AAV9, AAV10, AAV11, AAV12, scAAV, AAV-rh10, chimeric or hybrid AAV, or any combination, derivative, or variant thereof.

### Producing AAV Delivery Vectors

In some embodiments, methods of producing AAV delivery vectors herein comprise packaging a nucleic acid encoding an polypeptide (e.g., effector protein, effector partner, fusion protein, or combinations thereof) and a guide nucleic acid, or a combination thereof, into an AAV vector. In some embodiments, methods of producing the delivery vector comprises, (a) contacting a cell with at least one nucleic acid encoding: (i) a guide nucleic acid; (ii) a Replication (Rep) gene; and (iii) a Capsid (Cap) gene that encodes an AAV capsid protein; (b) expressing the AAV capsid protein in the cell; (c) assembling an AAV particle; and (d) packaging the polypeptide encoding nucleic acid into the AAV particle, thereby generating an AAV delivery vector. In some embodiments, promoters, stuffer sequences, and any combination thereof may be packaged in the AAV vector. In some examples, the AAV vector may package 1, 2, 3, 4, or 5 guide nucleic acids or copies thereof. In some embodiments, the AAV vector comprises inverted terminal repeats, e.g., a 5′ inverted terminal repeat and a 3′ inverted terminal repeat. In some embodiments, the AAV vector comprises a mutated inverted terminal repeat that lacks a terminal resolution site.

In some embodiments, a hybrid AAV vector is produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV9), wherein the first and second AAV serotypes may be not the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV 2/4, AAV2/5, AAV2/8, or AAV2/9 vector.

### Producing AAV Particles

The AAV particles described herein can be referred to as recombinant AAV (rAAV). Often, rAAV particles are generated by transfecting AAV producing cells with an AAV-containing plasmid carrying the sequence encoding the genome editing tools, a plasmid that carries viral encoding regions, i.e., Rep and Cap gene regions; and a plasmid that provides the helper genes such as E1A, E1B, E2A, E40RF6 and VA. In some embodiments, the AAV producing cells are mammalian cells. In some embodiments, host cells for rAAV viral particle production are mammalian cells. In some embodiments, a mammalian cell for rAAV viral particle production is a COS cell, a HEK293T cell, a HeLa cell, a KB cell, a derivative thereof, or a combination thereof. In some embodiments, rAAV virus particles can be produced in the mammalian cell culture system by providing the rAAV plasmid to the mammalian cell. In some embodiments, producing rAAV virus particles in a mammalian cell can comprise transfecting vectors that express the rep protein, the capsid protein, and the gene-of-interest expression construct flanked by the ITR sequence on the 5′ and 3′ ends. Methods of such processes are provided in, for example, Naso et al., BioDrugs, 2017 Aug.; 31(4):317-334 and Benskey et al., (2019), Methods Mol Biol., 1937:3-26, each of which is incorporated by reference in their entireties.

In some embodiments, rAAV is produced in a non-mammalian cell. In some embodiments, rAAV is produced in an insect cell. In some embodiments, an insect cell for producing rAAV viral particles comprises a Sf9 cell. In some embodiments, production of rAAV virus particles in insect cells can comprise baculovirus. In some embodiments, production of rAAV virus particles in insect cells can comprise infecting the insect cells with three recombinant baculoviruses, one carrying the cap gene, one carrying the rep gene, and one carrying the gene-of-interest expression construct enclosed by an ITR on both the 5′ and 3′ end. In some embodiments, rAAV virus particles are produced by the One Bac system. In some embodiments, rAAV virus particles can be produced by the Two Bac system. In some embodiments, in the Two Bac system, the rep gene and the cap gene of the AAV is integrated into one baculovirus virus genome, and the ITR sequence and the gene-of-interest expression construct is integrated into another baculovirus virus genome. In some embodiments, in the One Bac system, an insect cell line that expresses both the rep protein and the capsid protein is established and infected with a baculovirus virus integrated with the ITR sequence and the gene-of-interest expression construct. Details of such processes are provided in, for example, Smith et. al., (1983), Mol. Cell. Biol., 3(12):2156-65; Urabe et al., (2002), Hum. Gene. Ther., 1; 13(16):1935-43; and Benskey et al., (2019), Methods Mol Biol., 1937:3-26, each of which is incorporated by reference in its entirety.

### VI. Target Nucleic Acids and Samples

Described herein are compositions, systems and methods for modifying or detecting a target nucleic acid, wherein the target nucleic acid is a gene, a portion thereof, a transcript thereof. In some embodiments, the target nucleic acid is a reverse transcript (e.g., a cDNA) of an mRNA transcribed from the gene, or an amplicon thereof. acid. In some instances, the target nucleic acid is a single stranded nucleic acid. Alternatively, or in combination, the target nucleic acid is a double stranded nucleic acid and is prepared into single stranded nucleic acids before or upon contacting the reagents and/or RNP. In some instances, the target nucleic acid is a double stranded nucleic acid. In some instances, the double stranded nucleic acid is DNA. The target nucleic acid may be an RNA. The RNA target nucleic acids include but are not limited to mRNA, rRNA, tRNA, non-coding RNA, long non-coding RNA, single stranded RNA (ssRNA), and microRNA (miRNA). In some instances, the target nucleic acid is complementary DNA (cDNA) synthesized from a single-stranded RNA template in a reaction catalyzed by a reverse transcriptase. In some cases, the target nucleic acid is single-stranded RNA (ssRNA) or mRNA. In some embodiments, the target nucleic acid comprises an RNA, a DNA, or combination thereof. In some embodiments, guide nucleic acids described herein hybridize to a portion of the target nucleic acid. In some cases, the target nucleic acid is from a virus, a parasite, or a bacterium described herein. As another non-limiting example, the target nucleic acid may be responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides).

In certain embodiments, the target nucleic acid is a double stranded nucleic acid comprising a target strand and a non-target strand, wherein the target strand comprises a target sequence. In some embodiments, where a target strand comprises a target sequence, at least a portion of the engineered guide nucleic acid is complementary to the target sequence on the target strand. In some embodiments, where the target nucleic acid is a double stranded nucleic acid comprising a target strand and a non-target strand, and wherein the target strand comprises a target sequence, at least a portion of the engineered guide nucleic acid is complementary to the target sequence on the target strand.

In some embodiments, a target nucleic acid comprising a target sequence comprises a PAM sequence. In some embodiments, the PAM sequence is adjacent to the target sequence. In some embodiments, a target nucleic acid comprises a PAM as described herein that is located on the non-target strand. In some embodiments, the PAM sequence is 5′ to the target sequence. In some embodiments, the PAM sequenced is directly 5′ to the target sequence. Such a PAM described herein, in some embodiments, is adjacent (e.g., within 1, 2, 3, 4 or 5 nucleotides) to the 5′ end of the target sequence on the non-target strand of the double stranded DNA molecule. In certain embodiments, such a PAM described herein is directly adjacent to the 5′ end of a target sequence on the non-target strand of the double stranded DNA molecule. In some embodiments, the PAM sequence is adjacent to the target sequence. In some embodiments, the PAM sequence is 3′ to the target sequence. In some embodiments, the PAM sequence is directly 3′ to the target sequence. In some instances, the target nucleic acid as described in the methods herein does not initially comprise a PAM sequence. However, any target nucleic acid of interest may be generated using the methods described herein to comprise a PAM sequence, and thus be a PAM target nucleic acid. A PAM target nucleic acid, as used herein, refers to a target nucleic acid that has been amplified to insert a PAM sequence that is recognized by an effector protein system described herein.

An effector protein of the present disclosure, a dimer thereof, or a multimeric complex thereof may cleave or nick a target nucleic acid within or near a protospacer adjacent motif (PAM) sequence of the target nucleic acid. In some instances, cleavage occurs within 1, 2, 3, 4, 5, 6, 7, 8, 9 or 10 nucleotides of a 5′ or 3′ terminus of a PAM sequence. A target nucleic acid may comprise a PAM sequence adjacent to a sequence that is complementary to a guide nucleic acid spacer region. In some cases, an effector protein (e.g., a CasPhi.12 variant) or a multimeric complex thereof recognizes a PAM on a target nucleic acid. In some cases, multiple effector proteins of the multimeric complex recognize a PAM on a target nucleic acid. In some cases, only one effector protein of the multimeric complex recognizes a PAM on a target nucleic acid. In some cases, the PAM is 3′ to the spacer region of the crRNA. In some cases, the PAM is directly 3′ to the spacer region of the crRNA.

In some cases, the target nucleic acid comprises 5 to 100, 5 to 90, 5 to 80, 5 to 70, 5 to 60, 5 to 50, 5 to 40, 5 to 30, 5 to 25, 5 to 20, 5 to 15, or 5 to 10 linked nucleotides. In some cases, the target nucleic acid comprises 10 to 90, 20 to 80, 30 to 70, or 40 to 60 linked nucleotides. In some cases, the target nucleic acid comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 linked nucleotides. In some instances, the target nucleic acid comprises at least 10, at least 20, at least 30, at least 40, at least 50, at least 60, at least 70, at least 80, at least 90, or at least 100 linked nucleotides. In some embodiments, the target sequence in the target nucleic acid comprises at least 10 contiguous nucleotides that are complementary to the guide nucleic acid or engineered guide nucleic acid.

In some embodiments, compositions, systems, and methods described herein comprise a target nucleic acid may be responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides). In some embodiments, the target nucleic acid has undergone a modification (e.g., an editing) after contacting with an RNP. In some embodiments, the editing is a change in the sequence of the target nucleic acid. In some embodiments, the change comprises an insertion, deletion, or substitution of one or more nucleotides compared to the target nucleic acid that has not undergone any modification.

In some embodiments, the target nucleic acid comprises a portion or a specific region of a nucleic acid from a genomic locus, any DNA amplicon of, a reverse transcribed mRNA, or a cDNA from a gene described herein. In some embodiments, the target nucleic acid is an amplicon of at least a portion of a gene. Non-limiting examples of genes are set forth in TABLE 6. Nucleic acid sequences of target nucleic acids and/or corresponding genes are readily available in public databases as known and used in the art. In some cases, the target nucleic acid is selected from the target nucleic acids listed in TABLE 6.

In some cases, the target nucleic acid comprises a target locus. In certain embodiments, the target nucleic acid comprises more than one target loci.

In some cases, the target nucleic acid is B2M. In some cases, the B2M target nucleic acid comprises one or more target loci. In some cases, the B2M target nucleic acid comprises two target loci. In some cases, the B2M target locus comprises B2M2 or B2M4. In some cases, the target nucleic acid is FUT8.

In some cases, the target nucleic acid is B2M, TRAC, CIITA, PCSK9, NGCG_B2M, or any combination thereof. In some cases, the B2M, TRAC, CIITA, PCSK9 or NGCG_B2M target nucleic acid comprises one or more target loci. In some cases, the B2M, TRAC, CIITA, PCSK9, or NGCG_B2M target nucleic acid comprises two target loci.

In some embodiments, a target nucleic acid is the KRAS gene or a fragment thereof. KRAS or (Kirsten ras) gene encodes a protein that is a member of the small GTPase superfamily and which is involved in checkpoints for cell proliferation. The KRAS gene contains 7 exons and is located on chromosome 12, at cytogenetic location 12p12.1. A sequence representing a human wildtype allele of KRAS may be found in the NCBI database with gene accession ID: NC_000012.12. A sequence representing human wildtype KRAS mRNA (also a sense strand of human KRAS cDNA) may be found in the Ensembl database with accession number ENST00000311936.8.

Further description of editing or detecting a target nucleic acid in the foregoing genes can be found in more detail in Kim et al., “Enhancement of target specificity of CRISPR-Cas12a by using a chimeric DNA-RNA guide”, Nucleic Acids Res. 2020 Sep. 4; 48(15):8601-8616; Wang et al., “Specificity profiling of CRISPR system reveals greatly enhanced off-target gene editing”, Scientific Reports volume 10, Article number: 2269 (2020); Tuladhar et al., “CRISPR-Cas9-based mutagenesis frequently provokes on-target mRNA misregulation”, Nature Communications volume 10, Article number: 4056 (2019); Dong et al., “Genome-Wide Off-Target Analysis in CRISPR-Cas9 Modified Mice and Their Offspring”, G3, Volume 9, Issue 11, 1 Nov. 2019, Pages 3645-3651; Winter et al., “Genome-wide CRISPR screen reveals novel host factors required for Staphylococcus aureus α-hemolysin-mediated toxicity”, Scientific Reports volume 6, Article number: 24242 (2016); and Ma et al., “A CRISPR-Based Screen Identifies Genes Essential for West-Nile-Virus-Induced Cell Death”, Cell Rep. 2015 Jul. 28; 12(4):673-83, which are hereby incorporated by reference in their entirety.

In some instances, target nucleic acids comprise at least one nucleic acid comprising at least 50% sequence identity to the target nucleic acid or a portion thereof. Sometimes, the at least one nucleic acid comprises a nucleotide sequence that is at least 60%, 70%, 75%, 80%, 85%, 90%, 91%, 92%, 93%, 94%, 95%, 96%, 97%, 98%, or 99% identical to an equal length portion of the target nucleic acid. Sometimes, the at least one nucleic acid comprises a nucleotide sequence that is 100% identical to an equal length portion of the target nucleic acid. Sometimes, the nucleotide sequence of the at least one nucleic acid is at least 60%, 70%, 75%, 80%, 85%, 90%, 91%, 92%, 93%, 94%, 95%, 96%, 97%, 98%, or 99% identical to the target nucleic acid. Sometimes, the target nucleic acid comprises a nucleotide sequence that is less than 60%, 70%, 75%, 80%, 85%, 90%, 91%, 92%, 93%, 94%, 95%, 96%, 97%, 98%, or 99% identical to an equal length portion of the at least one nucleic acid.

In some instances, the target nucleic acid comprises a nucleic acid sequence from a pathogen responsible for a disease. Non-limiting examples of pathogens are bacteria, a virus and a fungus. In some embodiments, the target sequence is a portion of a nucleic acid from a virus or bacterium or other agents responsible for a disease in a sample. The target nucleic acid, in some cases, is a portion of a nucleic acid from a sexually transmitted infection or a contagious disease in a sample. The target sequence, in some embodiments, is a portion of a nucleic acid from an upper respiratory tract infection, a lower respiratory tract infection, or a contagious disease, in the sample. The target sequence, in some embodiments, is a portion of a nucleic acid from a hospital acquired infection or a contagious disease, in the sample. The target sequence, in some embodiments, is a portion of a nucleic acid from sepsis, in the sample.

In some cases, the target nucleic acid is a portion of a nucleic acid from a genomic locus, or any DNA amplicon, such as a reverse transcribed mRNA or a cDNA from a gene locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. A pathogenic virus can be a DNA virus or an RNA virus. Pathogenic viruses include but are not limited to respiratory viruses; adenoviruses; parainfluenza viruses; severe acute respiratory syndrome (SARS); coronavirus (e.g., SARS-CoV); MERS; gastrointestinal viruses (e.g., noroviruses, rotaviruses, some adenoviruses, astroviruses); exanthematous viruses (e.g., the virus that causes measles, the virus that causes rubella, the virus that causes chickenpox/shingles, the virus that causes roseola, the virus that causes smallpox, the virus that causes fifth disease, chikungunya virus infection), hepatic viral diseases (e.g., hepatitis A, B, C, D, E), cutaneous viral diseases (e.g., warts (including genital, anal), herpes (including oral, genital, anal), molluscum contagiosum); hemmorhagic viral diseases (e.g., Ebola, Lassa fever, dengue fever, yellow fever, Marburg hemorrhagic fever, Crimean-Congo hemorrhagic fever); neurologic viruses (e.g., polio, viral meningitis, viral encephalitis, rabies); sexually transmitted viruses (e.g., HIV, HPV, and the like), Adenovirus, coronavirus (i.e., a virus that causes COVID-19), Coronavirus HKU1, Coronavirus NL63, Coronavirus 229E, Coronavirus OC43, Severe Acute Respiratory Syndrome Coronavirus 2 (SARS-CoV-2), Human Metapneumovirus (hMPV), Human Rhinovirus/Enterovirus, influenza virus, Influenza A, Influenza A/H1, Influenza A/H3, Influenza A/H1-2009, Influenza B, Influenza C, Parainfluenza Virus 1, Parainfluenza Virus 2, Parainfluenza Virus 3, Parainfluenza Virus 4, Respiratory Syncytial Virus), human immunodeficiency virus (e.g., HIV); influenza virus; dengue; human papillomavirus (e.g., HPV); chlamydia; gonorrhea; syphilis; trichomoniasis; sexually transmitted infection; malaria; Dengue fever; Ebola; chikungunya; leishmaniasis; Orthopoxvirus (e.g., monkeypox virus, cowpox virus, camelpox virus, horsepox virus, vaccinia virus, and variola virus); West Nile virus; herpes virus; yellow fever virus; Hepatitis Virus C; Hepatitis Virus A; Hepatitis Virus B; papillomavirus; and the like. Pathogens include, e.g., HIV virus, Bordetella parapertussis, Bordetella pertussis, Chlamydia pneumoniae, Mycoplasma pneumoniae, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium, Enterobacter cloacae, Kiebsiella aerogenes, Proteus vulgaris, Serratia macesens, Enterococcus faecalis, Enterococcus faecium, Streptococcus intermdius, Streptococcus pneumoniae, Streptococcus pyogenes, and M. pneumoniae. In some cases, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

In some embodiments, the target sequence is comprised in a sample. In some embodiments, the sample used for genetic disorder testing, cancer testing, or cancer risk testing can comprise at least one target sequence or target nucleic acid segment that can bind to a guide nucleic acid of the reagents described herein. In some embodiments, the sample used comprises a target sequence or target nucleic acid of a gene recited in TABLE 6.

In some embodiments, the sample used for phenotyping testing can comprise at least one target nucleic acid segment that can bind to a guide nucleic acid of the reagents described herein. The target nucleic acid segment, in some cases, is a portion of a nucleic acid from a gene associated with a phenotypic trait.

In some embodiments, the sample used for genotyping testing can comprise at least one target nucleic acid segment that can bind to a guide nucleic acid of the reagents described herein. The target nucleic acid segment, in some cases, is a portion of a nucleic acid from a gene associated with a genotype.

In some instances, the target nucleic acid comprises a nucleic acid sequence of a virus, a bacterium, or other pathogen responsible for a disease in a plant (e.g., a crop). Methods and compositions of the disclosure may be used to treat or detect a disease in a plant. For example, the methods of the disclosure may be used to target a viral nucleic acid sequence in a plant. An effector protein of the disclosure (e.g., a CasPhi.12 variant) may cleave the viral nucleic acid. In some instances, the target nucleic acid comprises a nucleic acid sequence of a virus or a bacterium or other agents (e.g., any pathogen) responsible for a disease in the plant (e.g., a crop). In some instances, the target nucleic acid comprises RNA. The target nucleic acid, in some cases, is a portion of a nucleic acid from a virus or a bacterium or other agents responsible for a disease in the plant (e.g., a crop). In some cases, the target nucleic acid is a portion of a nucleic acid from a genomic locus, or any NA amplicon, such as a reverse transcribed mRNA or a cDNA from a gene locus, a transcribed mRNA, or a reverse transcribed cDNA from a gene locus in at a virus or a bacterium or other agents (e.g., any pathogen) responsible for a disease in the plant (e.g., a crop). A virus infecting the plant may be an RNA virus. A virus infecting the plant may be a DNA virus. Non-limiting examples of viruses that may be targeted with the disclosure include Tobacco mosaic virus (TMV), Tomato spotted wilt virus (TSWV), Cucumber mosaic virus (CMV), Potato virus Y (PVY), Cauliflower mosaic virus (CaMV) (RT virus), Plum pox virus (PPV), Brome mosaic virus (BMV) and Potato virus X (PVX).

In some embodiments, a target nucleic acid comprises a portion or a specific region of a nucleic acid from a genomic locus, any DNA amplicon of, a reverse transcribed mRNA, or a cDNA from a gene described herein. In some embodiments, the target nucleic acid is an amplicon of at least a portion of a gene. Non-limiting examples of genes are recited in TABLE 6. Nucleic acid sequences of target nucleic acids and/or corresponding genes are readily available in public databases as known and used in the art. In some embodiments, the target nucleic acid is selected from TABLE 6. In some embodiments, the target nucleic acid comprises one or more target sequences. In some embodiments, the one or more target sequence is within any one of the target nucleic acids set forth in TABLE 6.

In some embodiments, the target nucleic acid is any one of: a naturally occurring eukaryotic sequence, a naturally occurring prokaryotic sequence, a naturally occurring viral sequence, a naturally occurring bacterial sequence, a naturally occurring fungal sequence, an engineered eukaryotic sequence, an engineered prokaryotic sequence, an engineered viral sequence, an engineered bacterial sequence, an engineered fungal sequence, a fragment of a naturally occurring sequence, a fragment of an engineered sequence, and combinations thereof.

In some embodiments, the target nucleic acid is isolated from any one of: a naturally occurring cell, a eukaryotic cell, a prokaryotic cell, a plant cell, a fungal cell, an animal cell, cell of an invertebrate, a fly cell, a cell of a vertebrate, a mammalian cell, a primate cell, a non-human primate cell, a human cell, a living cell, a non-living cell, a modified cell, a derived cell, and a non-naturally occurring cell. In some embodiments, the target nucleic acid is isolated from a population of cells.

Nucleic acids, such as DNA and pre-mRNA, described herein can contain at least one intron and at least one exon, wherein as read in the 5′ to the 3′ direction of a nucleic acid strand, the 3′ end of an intron can be adjacent to the 5′ end of an exon, and wherein said intron and exon correspond for transcription purposes. If a nucleic acid strand contains more than one intron and exon, the 5′ end of the second intron is adjacent to the 3′ end of the first exon, and 5′ end of the second exon is adjacent to the 3′ end of the second intron. The junction between an intron and an exon can be referred to herein as a splice junction, wherein a 5′ splice site (SS) can refer to the +1/+2 position at the 5′ end of intron and a 3′SS can refer to the last two positions at the 3′ end of an intron. Alternatively, a 5′ SS can refer to the 5′ end of an exon and a 3′SS can refer to the 3′ end of an exon. In some embodiments, nucleic acids can contain one or more elements that act as a signal during transcription, splicing, and/or translation. In some embodiments, signaling elements include a 5′SS, a 3′SS, a premature stop codon, U1 and/or U2 binding sequences, and cis acting elements such as branch site (BS), polypyridine tract (PYT), exonic and intronic splicing enhancers (ESEs and ISEs) or silencers (ESSs and ISSs). In some embodiments, nucleic acids may also comprise an untranslated region (UTR), such as a 5′ UTR or a 3′ UTR. In some embodiments, the start of an exon or intron is referred to interchangeably herein as the 5′ end of an exon or intron, respectively. Likewise, in some embodiments, the end of an exon or intron is referred to interchangeably herein as the 3′ end of an exon or intron, respectively.

In some embodiments, at least a portion of at least one target sequence is within 1, about 5 or more, about 10 or more, about 15 or more, about 20 or more, about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, about 100 or more, about 105 or more, about 110 or more, about 115 or more, about 120 or more, about 125 or more, about 130 or more, about 135 or more, about 140 or more, about 145 or more, or about 150 to about 300 nucleotides adjacent to: the 5′ end of an exon; the 3′ end of an exon; the 5′ end of an intron; the 3′ end of an intron; one or more signaling element comprising a 5′SS, a 3′SS, a premature stop codon, U1 binding sequence, U2 binding sequence, a BS, a PYT, ESE, an ISE, an ESS, an ISS; a 5′ UTR; a 3′ UTR; more than one of the foregoing, or any combination thereof. In some embodiments, the target nucleic acid comprises a target locus. In some embodiments, the target nucleic acid comprises more than one target loci. In some embodiments, the target nucleic acid comprises two target loci. Accordingly, in some embodiments, the target nucleic acid can comprise one or more target sequences.

In some embodiments, compositions, systems, and methods described herein comprise a modified target nucleic acid which can describe a target nucleic acid wherein the target nucleic acid has undergone a modification, for example, after contact with an effector protein. In some cases, the modification is an alteration in the sequence of the target nucleic acid. In some cases, the modified target nucleic acid comprises an insertion, deletion, or replacement of one or more nucleotides compared to the unmodified target nucleic acid.

In some embodiments, compositions, systems, and methods described herein comprise an edited target nucleic acid which can describe a target nucleic acid wherein the target nucleic acid has undergone a change, for example, after contact with a polypeptide (e.g., an effector protein, an effector partner such as a fusion partner, a fusion protein, or a combination thereof). In some embodiments, the editing is an alteration in the sequence of the target nucleic acid. In some embodiments, the edited target nucleic acid comprises a nicked target strand or a nicked non-target strand. In some embodiments, the edited target nucleic acid comprises an insertion, deletion, or replacement of one or more nucleotides compared to the unedited target nucleic acid. In some embodiments, the editing is a mutation.

### Mutations

In some instances, target nucleic acids comprise a mutation. In some embodiments, a composition, system or method described herein can be used to modify a target nucleic acid comprising a mutation such that the mutation is modified to be a wild-type nucleotide or nucleotide sequence. In some embodiments, a composition, system or method described herein can be used to detect a target nucleic acid comprising a mutation. In some instances, a sequence comprising a mutation may be modified to a wildtype sequence with a composition, system or method described herein. In some instances, a sequence comprising a mutation may be detected with a composition, system or method described herein.

The mutation may be a mutation of 1, 2, 3, 4, 5, 6, 7, 8, 9, 10, 11, 12, 13, 14, 15, 16, 17, 18, 19, 20 or more nucleotides. The mutation may comprise a deletion of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation may comprise a deletion of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides. Non-limiting examples of mutations are insertion-deletion (indel), single nucleotide polymorphism (SNP), and frameshift mutations. In some instances, guide nucleic acids described herein hybridize to a region of the target nucleic acid comprising the mutation. The mutation may be located in a non-coding region or a coding region of a gene.

A mutation may be in an open reading frame of a target nucleic acid. A mutation may result in the insertion of at least one amino acid in a protein encoded by the target nucleic acid. A mutation may result in the deletion of at least one amino acid in a protein encoded by the target nucleic acid. A mutation may result in the substitution of at least one amino acid in a protein encoded by the target nucleic acid. A mutation that results in the deletion, insertion, or substitution of one or more amino acids of a protein encoded by the target nucleic acid may result in misfolding of a protein encoded by the target nucleic acid. A mutation may result in a premature stop codon, thereby resulting in a truncation of the encoded protein.

In some embodiments, a mutation comprises a point mutation or single nucleotide polymorphism (SNP), a chromosomal mutation, a copy number mutation, a copy number variation, a frameshift mutation, an insertion-deletion (indel), or any combination thereof. A point mutation optionally comprises a substitution, insertion, or deletion. In some embodiments, a mutation comprises a chromosomal mutation. A chromosomal mutations can comprise an inversion, a deletion, a duplication, or a translocation of one or more nucleotides. In some embodiments, a mutation comprises a copy number variation. A copy number variation can comprise a gene amplification or an expanding trinucleotide repeat. In some embodiments, guide nucleic acids described herein hybridize to a target sequence of a target nucleic acid comprising the mutation. In some embodiments, mutations are located in a non-coding region of a gene. In some embodiments, an indel mutation is an insertion or deletion of one or more nucleotides. In some embodiments, a frameshift mutation occurs when the number of nucleotides in the insertion/deletion is not divisible by three, and it occurs in a protein coding region. In some embodiments, an SNP is associated with a phenotype of the sample or a phenotype of the organism from which the sample was taken. In some embodiments, an SNP is associated with altered phenotype from wild type phenotype. In some embodiments, the SNP is a synonymous substitution or a nonsynonymous substitution. In some embodiments, the nonsynonymous substitution is a missense substitution or a nonsense point mutation. In some embodiments, the synonymous substitution is a silent substitution.

In some embodiments, a target nucleic acid described herein comprises a mutation of one or more nucleotides. In some embodiments, the one or more nucleotides comprises 1, 2, 3, 4, 5, 6, 7, 8, 9, 10, 11, 12, 13, 14, 15, 16, 17, 18, 19, 20 or more nucleotides. In some embodiments, the mutation comprises a deletion, insertion, and/or substitution of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. In some embodiments, the mutation comprises a deletion, insertion, and/or substitution of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides. The mutation may be located in a non-coding region or a coding region of a gene, wherein the gene is a target nucleic acid. A mutation may be in an open reading frame of a target nucleic acid. In some embodiments, guide nucleic acids described herein hybridize to a portion of the target nucleic acid comprising or adjacent to the mutation.

In some embodiments, the target nucleic acid comprises one or more mutations. In some embodiments, the target nucleic acid comprises one or more, two or more, three or more, four or more, five or more, six or more, seven or more, eight or more, nine or more, or ten or more mutations as compared to the unmutated target nucleic acid. In some embodiments, the target nucleic acid comprises a sequence comprising one or more, two or more, three or more, four or more, five or more, six or more, seven or more, eight or more, nine or more, or ten or more mutations as compared to the wildtype sequence. In some embodiments, the target nucleic acid comprises a mutation associated with a disease or disorder.

In some instances, target nucleic acids comprise a mutation, wherein the mutation is a SNP. The single nucleotide mutation or SNP may be associated with a phenotype of the sample or a phenotype of the organism from which the sample was taken. The SNP, in some cases, is associated with altered phenotype from wild type phenotype. In some embodiments, a single nucleotide mutation, SNP, or deletion described herein is associated with a disease, such as a genetic disease. The SNP may be a synonymous substitution or a nonsynonymous substitution. The nonsynonymous substitution may be a missense substitution or a nonsense point mutation. The synonymous substitution may be a silent substitution. The mutation may be a deletion of one or more nucleotides. Often, the single nucleotide mutation, SNP, or deletion is associated with a disease such as cancer or a genetic disorder. The mutation, such as a single nucleotide mutation, a SNP, or a deletion, may be encoded in the sequence of a target nucleic acid from the germline of an organism or may be encoded in a target nucleic acid from a diseased cell, such as a cancer cell.

In some embodiments, the mutation is associated with a disease, such as a genetic disorder. In some embodiments, the mutation may be encoded in the sequence of a target nucleic acid from the germline of an organism or may be encoded in a target nucleic acid from a diseased cell. In some embodiments, the target nucleic acid comprises a mutation associated with a disease. In some examples, a mutation associated with a disease refers to a mutation whose presence in a subject indicates that the subject is susceptible to or suffers from, a disease, disorder, condition, or syndrome. In some examples, a mutation associated with a disease refers to a mutation which causes, contributes to the development of, or indicates the existence of the disease, disorder, condition, or syndrome. A mutation associated with a disease may also refer to any mutation which generates transcription or translation products at an abnormal level, or in an abnormal form, in cells affected by a disease relative to a control without the disease. In some examples, a mutation associated with a disease refers to a mutation whose presence in a subject indicates that the subject is susceptible to, or suffers from, a disease, disorder, or pathological state. In some embodiments, a mutation associated with a disease, comprises the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation.

In some instances, target nucleic acids comprise a mutation, wherein the mutation is a deletion of 1, 2, 3, 4, 5, 6, 7, 8, 9, 10, 11, 12, 13, 14, 15, 16, 17, 18, 19, 20 or more nucleotides. In some embodiments, a target nucleic acid comprises a mutation of 1, 2, 3, 4, 5, 6, 7, 8, 9, 10, 11, 12, 13, 14, 15, 16, 17, 18, 19, 20 or more nucleotides. The mutation may be a deletion of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation may be a deletion of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides.

In some instances, target nucleic acids comprise a mutation, wherein the mutation is a substitution of 1, 2, 3, 4, 5, 6, 7, 8, 9, 10, 11, 12, 13, 14, 15, 16, 17, 18, 19, 20 or more nucleotides. In some embodiments, a target nucleic acid comprises a mutation of 1, 2, 3, 4, 5, 6, 7, 8, 9, 10, 11, 12, 13, 14, 15, 16, 17, 18, 19, 20 or more nucleotides. The mutation may be a substitution of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation may be a substitution of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides.

In some instances, target nucleic acids comprise a mutation, wherein the mutation is an insertion of 1, 2, 3, 4, 5, 6, 7, 8, 9, 10, 11, 12, 13, 14, 15, 16, 17, 18, 19, 20 or more nucleotides. In some embodiments, a target nucleic acid comprises a mutation of 1, 2, 3, 4, 5, 6, 7, 8, 9, 10, 11, 12, 13, 14, 15, 16, 17, 18, 19, 20 or more nucleotides. The mutation may be an insertion of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation may be an insertion of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides.

In some embodiments, a target nucleic acid is any target nucleic acid described herein, wherein the target nucleic acid comprises a mutation. In some embodiments, a target nucleic acid is any nucleic acid selected from TABLE 6, wherein the target nucleic acid comprises a mutation.

In some embodiments, a target nucleic acid is KRAS, wherein the target nucleic acid comprises a mutation. In some embodiments, a KRAS mutation may result in the uninhibited proliferation of cells and accumulation of mutation. In some embodiments, a KRAS mutation may be associated with a disease, such as a cancer. In some embodiments, a KRAS mutation may be an SNP. In some embodiments, a KRAS mutation may be in any one of the exon coding regions of the KRAS gene. In some embodiments, a KRAS mutation may be in exon 1, exon 2, exon 3, exon 4, exon 5, exon 6, or exon 7 of a KRAS gene. In some embodiments, a KRAS mutation may be in exon 2 of a KRAS gene. Exemplary KRAS mutations include, but are not limited, to KRAS p.G 12C-c.34G>T; KRAS p.G 12D-c.35G>A; and KRAS p.G 12V-c.35G>T.

### Detection of Target Nucleic Acids

Described herein are systems and methods for detecting the presence of a target nucleic acid in a sample. In some embodiments, a target nucleic acid described herein comprises a mutation associated with a disease, wherein the target nucleic acid is any one of the target nucleic acids set forth in TABLE 6. In some embodiments, a target nucleic acid described herein comprises a mutation associated with a disease, wherein the disease is any one of the diseases set forth in TABLE 6.1.

In some embodiments, the target nucleic acid is in a cell. In general, the cell is a human cell. In some embodiments, the human cell is a: blood cell, myeloid cell, lymphoid cell, hemopoietic stem or progenitor cell, myeloid common progenitor cell, megakaryocytes-erythrocyte progenitor cell, granulocytes-macrophages progenitor cell, monocytic-dendritic progenitor cell, lymphoid common progenitor cell, muscle cell, cardiac cell, visceral cell, cardiac muscle cell, smooth muscle cell, cardiomyocyte, nodal cardiac muscle cell, smooth muscle cell, visceral muscle cell, skeletal muscle cell, myocyte, red (or slow) skeletal muscle cell, white (fast) skeletal muscle cell, intermediate skeletal muscle, muscle satellite cell, muscle stem cell, myoblast, muscle progenitor cell, induced pluripotent stem cell (iPS), or a cell derived from an iPS cell, modified to have its gene edited and differentiated into myoblasts, muscle progenitor cells, muscle satellite cells, muscle stem cells, skeletal muscle cells, cardiac muscle cells or smooth muscle cells. In some embodiments, the human cell is derived from peripheral blood mononuclear cells, umbilical cord blood cells, bone marrow, lymph nodes, spleen, muscle, skin, or the like. In some instances, the target nucleic acid is in a cell. In some instances, the cell is a single-cell eukaryotic organism; a plant cell an algal cell; a fungal cell; an animal cell; a cell an invertebrate animal; a cell a vertebrate animal such as fish, amphibian, reptile, bird, and mammal; or a cell a mammal such as a human, a non-human primate, an ungulate, a feline, a bovine, an ovine, and a caprine. In preferred embodiments, the cell is a eukaryotic cell. In preferred embodiments, the cell is a mammalian cell, a human cell, or a plant cell.

An effector protein-guide nucleic acid complex may comprise high selectivity for a target sequence. In some cases, a ribonucleoprotein complex (RNP complex) may comprise a selectivity of at least 200:1, 100:1, 50:1, 20:1, 10:1, or 5:1 for a target nucleic acid over a single nucleotide variant of the target nucleic acid. In some cases, a ribonucleoprotein complex (RNP complex) may comprise a selectivity of at least 5:1 for a target nucleic acid over a single nucleotide variant of the target nucleic acid.

By leveraging such effector protein selectivity, some methods described herein may detect a target nucleic acid present in the sample in various concentrations or amounts as a target nucleic acid population. In some embodiments, the method detects at least 2 target nucleic acid populations. In some embodiments, the method detects at least 3, 4, 5, 6, 7, 8, 9, 10, 20, 30, 40, or 50 target nucleic acid populations. In some embodiments, the method detects 3 to 50, 5 to 40, or 10 to 25 target nucleic acid populations. In some embodiments, the method detects at least 2 individual target nucleic acids. In some embodiments, the method detects at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 individual target nucleic acids. In some embodiments, the method detects 1 to 10,000, 100 to 8000, 400 to 6000, 500 to 5000, 1000 to 4000, or 2000 to 3000 individual target nucleic acids.

Leveraging effector protein selectivity, some methods described herein may detect a target nucleic acid present in the sample in various concentrations or amounts as a target nucleic acid population. In some cases, the sample has at least 2 target nucleic acids. In some cases, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acids. In some cases, the sample comprises 1 to 10,000, 100 to 8000, 400 to 6000, 500 to 5000, 1000 to 4000, or 2000 to 3000 target nucleic acids. In some cases, the method detects target nucleic acid present at least at one copy per 10 non-target nucleic acids, 10² non-target nucleic acids, 103 non-target nucleic acids, 104 non-target nucleic acids, 105 non-target nucleic acids, 10⁶ non-target nucleic acids, 107 non-target nucleic acids, 10′ non-target nucleic acids, 109 non-target nucleic acids, or 10¹⁰ non-target nucleic acids.

In some embodiments, compositions described herein exhibit indiscriminate trans cleavage of a nucleic acid (e.g., a ssDNA and ssRNA), enabling their use for detection of a nucleic acid (e.g., DNA and RNA) in samples. In some embodiments, target nucleic acids are generated from many nucleic acid templates (e.g., RNA) in order to achieve cleavage of a reporter (e.g., a FQ reporter) in a device (e.g., a DETECTR platform). Certain effector proteins may be activated by a nucleic acid (e.g., a ssDNA and ssRNA), upon which they may exhibit trans cleavage of the nucleic acid (e.g., ssDNA and ssRNA) and may, thereby, be used to cleave the reporter molecules (e.g., ssDNA and ssRNA FQ reporter molecules) in a device (e.g., a DETECTR system). These effector proteins may target nucleic acids present in the sample or nucleic acids generated and/or amplified from any number of nucleic acid templates (e.g., RNA). Described herein are reagents comprising a single stranded reporter nucleic acid comprising a detection moiety, wherein the reporter nucleic acid (e.g., a ssDNA-FQ reporter described herein) is capable of being cleaved by the effector protein, upon generation (e.g., cDNA) and amplification of nucleic acids from a nucleic acid template (e.g., ssRNA) using the methods disclosed herein, thereby generating a first detectable signal. While DNA is used as an exemplary reporter in the foregoing, any suitable reporter may be used.

A target nucleic acid may be an amplified nucleic acid of interest. The nucleic acid of interest may be any nucleic acid disclosed herein or from any sample as disclosed herein. The nucleic acid of interest may be an RNA that is reverse transcribed before amplification. The nucleic acid of interest may be amplified then the amplicons may be transcribed into RNA. In some embodiments, the nucleic acid of interest is DNA. In some embodiments, the nucleic acid of interest is an RNA. In some embodiments, the nucleic acid of interest is an RNA that is reverse transcribed before amplification. In some embodiments, the target nucleic acid is an amplicon of a target nucleic acid (DNA or RNA) generated via amplification (with or without reverse transcription). In some embodiments, the target nucleic acid is an amplicon of a target nucleic acid (DNA or RNA) generated via amplification that is reverse transcribed before amplification.

In some instances, target nucleic acids may activate an effector protein to initiate sequence-independent cleavage of a nucleic acid-based reporter (e.g., a reporter comprising an RNA sequence, or a reporter comprising DNA and RNA). For example, an effector protein of the present disclosure is activated by a target nucleic acid to cleave reporters having an RNA (also referred to herein as an “RNA reporter”). Alternatively, an effector protein of the present disclosure is activated by a target nucleic acid to cleave reporters having an RNA. Alternatively, an effector protein of the present disclosure is activated by a target RNA to cleave reporters having an RNA (also referred to herein as a “RNA reporter”). The RNA reporter may comprise a single-stranded RNA labelled with a detection moiety or may be any RNA reporter as disclosed herein.

### Samples

Various sample types comprising a target nucleic acid of interest are consistent with the present disclosure. These samples may comprise a target nucleic acid for detection. In some instances, the detection of the target nucleic indicates an ailment, such as a disease, cancer, or genetic disorder, or genetic information, such as for phenotyping, genotyping, or determining ancestry and are compatible with the reagents and support mediums as described herein. Generally, a sample from an individual or an animal or an environmental sample may be obtained to test for presence of a disease, cancer, genetic disorder, or any mutation of interest.

Often, the target nucleic acid may be from 0.05% to 20% of total nucleic acids in the sample. Sometimes, the target nucleic acid is 0.1% to 10% of the total nucleic acids in the sample. The target nucleic acid, in some cases, is 0.1% to 5% of the total nucleic acids in the sample. The target nucleic acid may also be 0.1% to 1% of the total nucleic acids in the sample. The target nucleic acid may be DNA or RNA. The target nucleic acid may be any amount less than 100% of the total nucleic acids in the sample. The target nucleic acid may be 100% of the total nucleic acids in the sample.

The target nucleic acid may be 0.05% to 20% of total nucleic acids in the sample. Sometimes, the target nucleic acid is 0.1% to 10% of the total nucleic acids in the sample. The target nucleic acid, in some cases, is 0.1% to 5% of the total nucleic acids in the sample. Often, a sample comprises the segment of the target nucleic acid and at least one nucleic acid comprising less than 100% sequence identity to the segment of the target nucleic acid but no less than 50% sequence identity to the segment of the target nucleic acid. For example, the segment of the target nucleic acid comprises a mutation as compared to at least one nucleic acid comprising less than 100% sequence identity to the segment of the target nucleic acid but no less than 50% sequence identity to the segment of the target nucleic acid. Often, the segment of the target nucleic acid comprises a single nucleotide mutation as compared to at least one nucleic acid comprising less than 100% sequence identity to the segment of the target nucleic acid but no less than 50% sequence identity to the segment of the target nucleic acid.

A number of target nucleic acid populations are consistent with the methods and compositions disclosed herein. Some methods described herein may detect two or more target nucleic acid populations present in the sample in various concentrations or amounts. In some cases, the sample has at least 2 target nucleic acid populations. In some cases, the sample has at least 3, 4, 5, 6, 7, 8, 9, 10, 20, 30, 40, or 50 target nucleic acid populations. In some cases, the sample has 3 to 50, 5 to 40, or 10 to 25 target nucleic acid populations.

In some cases, the method detects target nucleic acid populations that are present at least at one copy per 101 non-target nucleic acids, 102 non-target nucleic acids, 10³ non-target nucleic acids, 104 non-target nucleic acids, 105 non-target nucleic acids, 106 non-target nucleic acids, 107 non-target nucleic acids, 10′ non-target nucleic acids, 109 non-target nucleic acids, or 10¹⁰ non-target nucleic acids. The target nucleic acid populations may be present at different concentrations or amounts in the sample.

In some instances, samples comprise a target nucleic acid at a concentration of less than 1 nM, less than 2 nM, less than 3 nM, less than 4 nM, less than 5 nM, less than 6 nM, less than 7 nM, less than 8 nM, less than 9 nM, less than 10 nM, less than 20 nM, less than 30 nM, less than 40 nM, less than 50 nM, less than 60 nM, less than 70 nM, less than 80 nM, less than 90 nM, less than 100 nM, less than 200 nM, less than 300 nM, less than 400 nM, less than 500 nM, less than 600 nM, less than 700 nM, less than 800 nM, less than 900 nM, less than 1 μM, less than 2 μM, less than 3 μM, less than 4 μM, less than 5 μM, less than 6 μM, less than 7 μM, less than 8 μM, less than 9 μM, less than 10 μM, less than 100 μM, or less than 1 mM. In some instances, the sample comprises a target nucleic acid at a concentration of 1 nM to 2 nM, 2 nM to 3 nM, 3 nM to 4 nM, 4 nM to 5 nM, 5 nM to 6 nM, 6 nM to 7 nM, 7 nM to 8 nM, 8 nM to 9 nM, 9 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1 μM, 1 μM to 2 μM, 2 μM to 3 μM, 3 μM to 4 μM, 4 μM to 5 μM, 5 μM to 6 μM, 6 μM to 7 μM, 7 μM to 8 μM, 8 μM to 9 μM, 9 μM to 10 μM, 10 μM to 100 PM, 100 μM to 1 mM, 1 nM to 10 nM, 1 nM to 100 nM, 1 nM to 1 μM, 1 nM to 10 μM, 1 nM to 100 μM, 1 nM to 1 mM, 10 nM to 100 nM, 10 nM to 1 μM, 10 nM to 10 μM, 10 nM to 100 μM, 10 nM to 1 mM, 100 nM to 1 μM, 100 nM to 10 μM, 100 nM to 100 μM, 100 nM to 1 mM, 1 μM to 10 μM, 1 μM to 100 μM, 1 μM to 1 mM, 10 μM to 100 μM, 10 μM to 1 mM, or 100 μM to 1 mM. In some instances, the sample comprises a target nucleic acid at a concentration of 20 nM to 200 μM, 50 nM to 100 μM, 200 nM to 50 μM, 500 nM to 20 μM, or 2 μM to 10 μM. In some instances, the target nucleic acid is not present in the sample.

In some instances, samples comprise fewer than 10 copies, fewer than 100 copies, fewer than 1000 copies, fewer than 10,000 copies, fewer than 100,000 copies, or fewer than 1,000,000 copies of a target nucleic acid. In some instances, the sample comprises 10 copies to 100 copies, 100 copies to 1000 copies, 1000 copies to 10,000 copies, 10,000 copies to 100,000 copies, 100,000 copies to 1,000,000 copies, 10 copies to 1000 copies, 10 copies to 10,000 copies, 10 copies to 100,000 copies, 10 copies to 1,000,000 copies, 100 copies to 10,000 copies, 100 copies to 100,000 copies, 100 copies to 1,000,000 copies, 1,000 copies to 100,000 copies, or 1,000 copies to 1,000,000 copies of a target nucleic acid. In some instances, the sample comprises 10 copies to 500,000 copies, 200 copies to 200,000 copies, 500 copies to 100,000 copies, 1000 copies to 50,000 copies, 2000 copies to 20,000 copies, 3000 copies to 10,000 copies, or 4000 copies to 8000 copies. In some instances, the target nucleic acid is not present in the sample.

In some instances, the sample is a biological sample, an environmental sample, or a combination thereof. Non-limiting examples of biological samples are blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, and a tissue sample (e.g., a biopsy sample). A tissue sample from a subject may be dissociated or liquified prior to application to detection system of the present disclosure. Non-limiting examples of environmental samples are soil, air, or water. In some instances, an environmental sample is taken as a swab from a surface of interest or taken directly from the surface of interest.

In some instances, the sample is a raw (unprocessed, unmodified) sample. Raw samples may be applied to a system for detecting or modifying a target nucleic acid, such as those described herein. In some instances, the sample is diluted with a buffer or a fluid or concentrated prior to its application to the system or be applied neat to the detection system. Sometimes, the sample contains no more 20 μl of buffer or fluid. The sample, in some cases, is contained in no more than 1, 5, 10, 15, 20, 25, 30, 35 40, 45, 50, 55, 60, 65, 70, 75, 80, 90, 100, 200, 300, 400, 500 μl, or any of value 1 μl to 500 μl, preferably 10 μL to 200 μL, or more preferably 50 μL to 100 μL of buffer or fluid. Sometimes, the sample is contained in more than 500 μl.

In some instances, the sample is taken from a single-cell eukaryotic organism; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some instances, the sample is taken from nematodes, protozoans, helminths, or malarial parasites. In some cases, the sample comprises nucleic acids from a cell lysate from a eukaryotic cell, a mammalian cell, a human cell, a prokaryotic cell, or a plant cell. In some cases, the sample comprises nucleic acids expressed from a cell.

In some instances, samples are used for diagnosing a disease. In some instances the disease is cancer. The sample used for cancer testing may comprise at least one target nucleic acid that may bind to a guide nucleic acid of the reagents described herein. The target nucleic acid, in some cases, comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes a cancer biomarker, such as a prostate cancer biomarker or non-small cell lung cancer. In some cases, the assay may be used to detect “hotspots” in target nucleic acids that may be predictive of lung cancer. In some cases, the target nucleic acid comprises a portion of a nucleic acid that is associated with a blood fever. In some cases, the target nucleic acid is a portion of a nucleic acid from a genomic locus, any DNA amplicon of, a reverse transcribed mRNA, or a cDNA from a locus of at least one of a gene set forth in TABLE 6. Any region of the aforementioned gene loci may be probed for a mutation or deletion using the compositions, systems and methods disclosed herein. For example, in the EGFR gene locus, the compositions, systems and methods for detection disclosed herein may be used to detect a single nucleotide polymorphism or a deletion.

In some instances, samples are used to diagnose a genetic disorder, also referred to as genetic disorder testing. The sample used for genetic disorder testing may comprise at least one target nucleic acid that may bind to a guide nucleic acid of the reagents described herein. In some instances, the genetic disorder is hemophilia, sickle cell anemia, β-thalassemia, Duchene muscular dystrophy, severe combined immunodeficiency, Huntington's disease, or cystic fibrosis. The target nucleic acid, in some cases, is from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some cases, the target nucleic acid is a nucleic acid from a genomic locus, a transcribed mRNA, or a reverse transcribed mRNA, a DNA amplicon of or a cDNA from a locus of at least one of a gene set forth in TABLE 6.

The sample used for phenotyping testing may comprise at least one target nucleic acid that may bind to a guide nucleic acid of the reagents described herein. The target nucleic acid, in some cases, is a nucleic acid encoding a sequence associated with a phenotypic trait. The sample used for genotyping testing may comprise at least one target nucleic acid that may bind to a guide nucleic acid of the reagents described herein. The target nucleic acid, in some cases, is a nucleic acid encoding a sequence associated with a genotype of interest. The sample used for ancestral testing may comprise at least one target nucleic acid that may bind to a guide nucleic acid of the reagents described herein. The target nucleic acid, in some cases, is a nucleic acid encoding a sequence associated with a geographic region of origin or ethnic group. The sample may be used for identifying a disease status. For example, a sample is any sample described herein, and is obtained from a subject for use in identifying a disease status of a subject. The disease may be a cancer or genetic disorder. Sometimes, a method comprises obtaining a serum sample from a subject; and identifying a disease status of the subject. Often, the disease status is prostate disease status, but the status of any disease may be assessed.

Any of the above disclosed samples are consistent with the methods, compositions, reagents, enzymes, and systems disclosed herein.

### VII. Compositions

Disclosed herein are compositions comprising one or more polypeptides (e.g., effector proteins, effector partners such as a fusion partners, fusion proteins, or combinations thereof) described herein or nucleic acids encoding the one or more polypeptides, one or more guide nucleic acids described herein or nucleic acids encoding the one or more guide nucleic acids described herein, or combinations thereof. In some embodiments, repeat sequences of the one or more guide nucleic acids are capable of interacting with the one or more of the effector proteins. In some embodiments, spacer sequences of the one or more guide nucleic acids hybridizes with a target sequence of a target nucleic acid. In some embodiments, the compositions comprise one or more donor nucleic acids described herein. In some embodiments, the compositions are capable of editing a target nucleic acid in a cell or a subject. In some embodiments, the compositions are capable of editing a target nucleic acid or the expression thereof in a cell, in a tissue, in an organ, in vitro, in vivo, or ex vivo. In some embodiments, the compositions are capable of editing a target nucleic acid in a sample comprising the target nucleic.

In some embodiments, compositions described herein comprise plasmids described herein, viral vectors described herein, non-viral vectors described herein, or combinations thereof. In some embodiments, compositions described herein comprise the viral vectors. In some embodiments, compositions described herein comprise an AAV. In some embodiments, compositions described herein comprise liposomes (e.g., cationic lipids or neutral lipids), dendrimers, lipid nanoparticle (LNP), or cell-penetrating peptides. In some embodiments, compositions described herein comprise an LNP.

### Pharmaceutical Compositions

Disclosed herein, in some aspects, are pharmaceutical compositions for modifying a target nucleic acid in a cell or a subject, comprising any one of the polypeptides (e.g., effector proteins, engineered effector proteins or variant effector proteins), effector partners (e.g., fusion partners), fusion effector proteins (i.e., fusion proteins)), or guide nucleic acids as described herein and any combination thereof. Also disclosed herein, in some aspects, are pharmaceutical compositions comprising a nucleic acid encoding any one of the effector proteins, engineered effector proteins, fusion effector proteins, or guide nucleic acids as described herein and any combination thereof. In some embodiments, pharmaceutical compositions comprise a plurality of guide nucleic acids. Pharmaceutical compositions may be used to modify a target nucleic acid or the expression thereof in a cell in vitro, in vivo or ex vivo.

Also described herein are formulations of introducing compositions or components of a system described herein to a host. In some embodiments, compositions described herein are pharmaceutical compositions. In some embodiments, the pharmaceutical compositions comprise compositions described herein or systems described herein. In some embodiments, the pharmaceutical composition comprises a pharmaceutically acceptable salt, one or more of a vehicle, adjuvant, excipient, diluent, or carrier, such as a filler, disintegrant, a surfactant, a binder, a lubricant, or combinations thereof.

In some embodiments, when describing a component as pharmaceutically acceptable, such as a pharmaceutically acceptable excipient, carrier or diluent, reference is made to any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by suitable methods (see, e.g., Remington's Pharmaceutical Sciences, 18th edition, A. Gennaro, ed., Mack Publishing Co., Easton, Pa., 1990; and Remington, The Science and Practice of Pharmacy 21st Ed. Mack Publishing, 2005).

Non-limiting examples of pharmaceutically acceptable carriers and diluents suitable for the pharmaceutical compositions disclosed herein include buffers (e.g., neutral buffered saline, phosphate buffered saline); carbohydrates (e.g., glucose, mannose, sucrose, dextran, mannitol); polypeptides or amino acids (e.g., glycine); antioxidants; chelating agents (e.g., EDTA, glutathione); adjuvants (e.g., aluminum hydroxide); surfactants (Polysorbate 80, Polysorbate 20, or Pluronic F68); glycerol; sorbitol; mannitol; polyethyleneglycol; and preservatives. In some embodiments, the vector is formulated for delivery through injection by a needle carrying syringe. In some embodiments, the composition is formulated for delivery by electroporation. In some embodiments, the composition is formulated for delivery by chemical method. In some embodiments, the pharmaceutical compositions comprise a virus vector or a non-viral vector, examples of both of which are described herein throughout.

Pharmaceutical compositions described herein may comprise a salt. In some embodiments, the salt is a sodium salt. In some embodiments, the salt is a potassium salt. In some embodiments, the salt is a magnesium salt. In some embodiments, the salt is NaCl. In some embodiments, the salt is KNO3. In some embodiments, the salt is Mg²⁺SO₄²⁻.

Pharmaceutical compositions described herein are in the form of a solution (e.g., a liquid). In some embodiments, the solution is formulated for injection, e.g., intravenous or subcutaneous injection. In some embodiments, the pH of the solution is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some cases, the pH of the solution is less than 7. In some cases, the pH is greater than 7.

In some embodiments, pharmaceutical compositions comprise one or more nucleic acids encoding an effector protein, fusion effector protein, fusion partner, a guide nucleic acid, or a combination thereof; and a pharmaceutically acceptable carrier or diluent. The effector protein, fusion effector protein, fusion partner protein, or combination thereof may be any one of those described herein. The one or more nucleic acids may comprise a plasmid. The one or more nucleic acids may comprise a nucleic acid expression vector. The one or more nucleic acids may comprise a viral vector. In some embodiments, the viral vector is a lentiviral vector. In some embodiments, the vector is an adeno-associated viral (AAV) vector. In some embodiments, compositions, including pharmaceutical compositions, comprise a viral vector encoding a fusion effector protein and a guide nucleic acid, wherein at least a portion of the guide nucleic acid binds to the effector protein of the fusion effector protein.

In some embodiments, pharmaceutical compositions comprise a virus comprising a viral vector encoding a fusion effector protein, an effector protein, a fusion partner, a guide nucleic acid, or a combination thereof; and a pharmaceutically acceptable carrier or diluent. The virus may be a lentivirus. The virus may be an adenovirus. The virus may be a non-replicating virus. The virus may be an adeno-associated virus (AAV). The viral vector may be a retroviral vector. Retroviral vectors may include gamma-retroviral vectors such as vectors derived from the Moloney Murine Leukemia Virus (MoMLV, MMLV, MuLV, or MLV) or the Murine Stem cell Virus (MSCV) genome. Retroviral vectors may include lentiviral vectors such as those derived from the human immunodeficiency virus (HIV) genome. In some embodiments, the viral vector is a chimeric viral vector, comprising viral portions from two or more viruses. In some embodiments, the viral vector is a recombinant viral vector.

In some embodiments, the viral vector is an AAV. The AAV may be any AAV known in the art. In some embodiments, the viral vector corresponds to a virus of a specific serotype. In some examples, the serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, and an AAV12 serotype. In some embodiments the AAV vector is a recombinant vector, a hybrid AAV vector, a chimeric AAV vector, a self-complementary AAV (scAAV) vector, a single-stranded AAV or any combination thereof. scAAV genomes are generally known in the art and contain both DNA strands which can anneal together to form double-stranded DNA.

In some embodiments, methods of producing delivery vectors herein comprise packaging a nucleic acid encoding an effector protein and a guide nucleic acid, or a combination thereof, into an AAV vector. In some embodiments, methods of producing the delivery vector comprises, (a) contacting a cell with at least one nucleic acid encoding: (i) a guide nucleic acid; (ii) a Replication (Rep) gene; and (iii) a Capsid (Cap) gene that encodes an AAV capsid protein; (b) expressing the AAV capsid protein in the cell; (c) assembling an AAV particle; and (d) packaging a Cas effector encoding nucleic acid into the AAV particle, thereby generating an AAV delivery vector. In some embodiments, promoters, stuffer sequences, and any combination thereof may be packaged in the AAV vector. In some examples, the AAV vector can package 1, 2, 3, 4, or 5 guide nucleic acids or copies thereof. In some embodiments, the AAV vector comprises inverted terminal repeats, e.g., a 5′ inverted terminal repeat and a 3′ inverted terminal repeat. In some embodiments, the AAV vector comprises a mutated inverted terminal repeat that lacks a terminal resolution site.

In some embodiments, a hybrid AAV vector is produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV9), wherein the first and second AAV serotypes may be not the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV 2/4, AAV2/5, AAV2/8, or AAV2/9 vector.

In some embodiments, the AAV vector may be a chimeric AAV vector. In some embodiments, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

In some examples, the delivery vector may be a eukaryotic vector, a prokaryotic vector (e.g., a bacterial vector) a viral vector, or any combination thereof. In some embodiments, the delivery vehicle may be a non-viral vector. In some embodiments, the delivery vehicle may be a plasmid. In some embodiments, the plasmid comprises DNA. In some embodiments, the plasmid comprises RNA. In some examples, the plasmid comprises circular double-stranded DNA. In some examples, the plasmid may be linear. In some examples, the plasmid comprises one or more genes of interest and one or more regulatory elements. In some examples, the plasmid comprises a bacterial backbone containing an origin of replication and an antibiotic resistance gene or other selectable marker for plasmid amplification in bacteria. In some examples, the plasmid may be a minicircle plasmid. In some examples, the plasmid contains one or more genes that provide a selective marker to induce a target cell to retain the plasmid. In some examples, the plasmid may be formulated for delivery through injection by a needle carrying syringe. In some examples, the plasmid may be formulated for delivery via electroporation. In some examples, the plasmids may be engineered through synthetic or other suitable means known in the art. For example, in some embodiments, the genetic elements may be assembled by restriction digest of the desired genetic sequence from a donor plasmid or organism to produce ends of the DNA which may then be readily ligated to another genetic sequence.

In some embodiments, the vector is a non-viral vector, and a physical method or a chemical method is employed for delivery into the somatic cell. Exemplary physical methods include electroporation, gene gun, sonoporation, magnetofection, or hydrodynamic delivery. Exemplary chemical methods include delivery of the recombinant polynucleotide via liposomes such as, cationic lipids or neutral lipids; dendrimers; nanoparticles; or cell-penetrating peptides.

In some embodiments, a fusion effector protein as described herein is inserted into a vector. In some embodiments, the vector comprises one or more promoters, enhancers, ribosome binding sites, RNA splice sites, polyadenylation sites, a replication origin, and/or transcriptional terminator sequences.

In general, plasmids and vectors described herein comprise at least one promoter. In some embodiments, the promoters are constitutive promoters. In other embodiments, the promoters are inducible promoters. In additional embodiments, the promoters are prokaryotic promoters (e.g., drive expression of a gene in a prokaryotic cell). In some embodiments, the promoters are eukaryotic promoters, (e.g., drive expression of a gene in a eukaryotic cell). Exemplary promoters include, but are not limited to, CMV, EF1a, SV40, PGK1, Ubc, human beta actin, CAG, TRE, UAS, Ac5, polyhedron, CaMKIIa, GAL1-10, TEF1, GDS, ADH1, CaMV35S, Ubi, H1, U6, CaMV35S, SV40, CMV, and HSV TK promoter. In some embodiments, the promoter is CMV. In some embodiments, the promoter is EF1a. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo basepairs. Furthermore, enhancers are located optionally upstream or downstream of a gene region to be transcribed, and/or located within the gene, to activate the transcription. Exemplary enhancers include, but are not limited to, WPRE; CMV enhancers; the R—U5′ segment in LTR of HTLV-I (Mol. Cell. Biol., Vol. 8(1), p. 466-472, 1988); SV40 enhancer; the intron sequence between exons 2 and 3 of rabbit β-globin (Proc. Natl. Acad. Sci. USA., Vol. 78(3), p. 1527-31, 1981); and the genome region of human growth hormone (J Immunol., Vol. 155(3), p. 1286-95, 1995).

In some embodiments, pharmaceutical compositions comprise an: effector protein, fusion effector protein, fusion partner, a guide nucleic acid, or a combination thereof; and a pharmaceutically acceptable carrier or diluent. In some embodiments, pharmaceutical compositions comprise one or more nucleic acids encoding an: effector protein, fusion effector protein, fusion partner, a guide nucleic acid, or a combination thereof; and a pharmaceutically acceptable carrier or diluent. In some embodiments, guide nucleic acid can be a plurality of guide nucleic acids. In some embodiments, the effector protein comprises a sequence that, other than one or more alterations set forth in TABLE 1, is at least 65%, at least 70%, at least 75%, at least 80%, at least 85%, at least 90%, at least 95%, at least 97%, or at least 98%, at least 99%, or 100% identical to the sequence of SEQ ID NO: 1. In some embodiments, the effector protein comprises a sequence that, other than one or more alterations set forth in TABLE 1, is at least 65%, at least 70%, at least 75%, at least 80%, at least 85%, at least 90%, at least 95%, at least 97%, or at least 98%, at least 99%, or 100% similar to the sequence of SEQ ID NO: 1. In some embodiments, the guide nucleic acid comprises a nucleotide sequence of any one of the sequences of TABLE 3, TABLE 4, TABLE 5, or any combination thereof.

In combination with a pharmaceutically acceptable carrier or diluent, each row in TABLE 4 or TABLE 5 can represent an exemplary pharmaceutical composition comprising an effector protein as described in TABLE 1 and a guide nucleic acid wherein the guide nucleic acid can further comprise a sequence of TABLE 3.

### VIII. Systems

Disclosed herein, in some aspects, are systems for detecting, modifying, or editing a target nucleic acid, comprising any one of the polypeptides described herein (e.g., effector proteins, effector partners (e.g., fusion partners), and/or fusion proteins described herein), or a multimeric complex thereof. Systems may be used to detect, modify, or edit a target nucleic acid. Systems may be used to modify the activity or expression of a target nucleic acid.

In some embodiments, systems comprise one or more components comprising a guide nucleic acid described herein. In some embodiments, systems comprise one or more components comprising a guide nucleic acid and an additional nucleic acid. In some embodiments, systems comprise one or more components comprising an effector protein described herein, one or more guide nucleic acids, an additional nucleic acid, a reagent, a support medium, or combinations thereof. In some embodiments, systems comprise one or more components comprising compositions, a solution, a buffer, a reagent, a support medium, or combinations thereof. In some embodiments, systems further comprise one or more components comprising a donor nucleic acid as disclosed herein. In some embodiments, systems comprise one or more components comprising an effector protein described herein, a reagent, support medium, or a combination thereof. In some embodiments, systems comprise one or more components comprising an effector protein described herein, a guide nucleic acid described herein, a reagent, support medium, or a combination thereof. In some embodiments, the effector protein comprises an effector protein, or a fusion protein thereof, described herein. In some embodiments, systems or system components described herein are comprised in a single composition.

In some embodiments, solutions, compositions, systems, and methods comprise 0.01 μL, 0.02 μL, 0.03 μL, 0.04 μL, 0.05 μL, 0.06 μL, 0.07 μL, 0.08 μL, 0.09 μL, 0.1 μL, 0.2 μL, 0.3 μL, 0.4 μL, 0.5 μL, 0.6 μL, 0.7 μL, 0.8 μL, 0.9 μL, 1 μL, 2 μL, 3 μL, 4 μL, 5 μL, 6 μL, 7 μL, 8 μL, 9 μL, 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, 500 μL, or more, effector proteins, or nucleic acids encoding the effector proteins, as described herein. In some embodiments, solutions, compositions, systems, and methods comprise 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, or more, effector proteins, or nucleic acids encoding the effector proteins, as described herein. In some embodiments, solutions, compositions, systems, and methods, comprise 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 150 μM, 200 μM, 250 μM, 300 μM, 350 μM, 400 μM, 450 μM, 500 μM, or more, effector proteins, or nucleic acids encoding the effector proteins, as described herein. In some embodiments, solutions, compositions, systems, and methods comprise 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or more, effector proteins, or nucleic acids encoding the effector proteins, as described herein.

In some embodiments, solutions, compositions, systems, and methods comprise 0.01 μL, 0.02 μL, 0.03 μL, 0.04 μL, 0.05 μL, 0.06 μL, 0.07 μL, 0.08 μL, 0.09 μL, 0.1 μL, 0.2 μL, 0.3 μL, 0.4 μL, 0.5 μL, 0.6 μL, 0.7 μL, 0.8 μL, 0.9 μL, 1 μL, 2 μL, 3 μL, 4 μL, 5 μL, 6 μL, 7 μL, 8 μL, 9 μL, 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, 500 μL, or more, guide nucleic acids, or nucleic acids encoding the guide nucleic acids, as described herein. In some embodiments, solutions, compositions, systems, and methods comprise 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, or more, guide nucleic acids, or nucleic acids encoding the guide nucleic acids, as described herein. In some embodiments, solutions, compositions, systems, and methods comprise 1 μM, 2 μM, 3 μM, 4 μM, 5 μM, 6 PM, 7 PM, 8 μM, 9 μM, 10 μM, 20 μM, 30 μM, 40 μM, 50 μM, 60 μM, 70 μM, 80 μM, 90 μM, 100 PM, 150 PM, 200 μM, 250 μM, 300 μM, 350 μM, 400 μM, 450 μM, 500 μM, or more, guide nucleic acids, or nucleic acids encoding the guide nucleic acids, as described herein. In some embodiments, solutions, compositions, systems, and methods comprise 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or more, guide nucleic acids, or nucleic acids encoding the guide nucleic acids, as described herein.

Systems may be used for detecting the presence or the absence of a target nucleic acid as described herein. Systems may be used for detecting the presence or the absence of a target nucleic acid associated with or causative of a disease or disorder, such as cancer, a genetic disorder, or an infection. Systems may be used for detecting the presence or the absence of a target nucleic acid associated with or causative of a disease or disorder as described herein. In some embodiments, systems are useful for phenotyping, genotyping, or determining ancestry. Unless specified otherwise, systems include kits and may be referred to as kits. Unless specified otherwise, systems include devices and may also be referred to as devices. Systems described herein may be provided in the form of a companion diagnostic assay or device, a point-of-care assay or device, or an over-the-counter diagnostic assay/device. Unless specified otherwise, systems described herein may be used in methods for detecting the presence of a target nucleic acid.

Reagents and effector proteins of various systems may be provided in a reagent chamber or on a support medium. Alternatively, the reagent and/or effector protein may be contacted with the reagent chamber or the support medium by the individual using the system. An exemplary reagent chamber is a test well or container. The opening of the reagent chamber may be large enough to accommodate the support medium. Optionally, the system comprises a buffer and a dropper. The buffer may be provided in a dropper bottle for ease of dispensing. The dropper may be disposable and transfer a fixed volume. The dropper may be used to place a sample into the reagent chamber or on the support medium.

### System Solutions

In general, system components comprise a solution in which the activity of an effector protein occurs. Often, the solution comprises or consists essentially of a buffer. The solution or buffer may comprise a buffering agent, a salt, a crowding agent, a detergent, a reducing agent, a competitor, or a combination thereof. Often the buffer is the primary component or the basis for the solution in which the activity occurs. Thus, concentrations for components of buffers described herein (e.g., buffering agents, salts, crowding agents, detergents, reducing agents, and competitors) are the same or essentially the same as the concentration of these components in the solution in which the activity occurs. In some embodiments, a buffer is required for cell lysis activity or viral lysis activity.

In some embodiments, systems comprise a buffer, wherein the buffer comprise at least one buffering agent. Exemplary buffering agents include HEPES, TRIS, MES, ADA, PIPES, ACES, MOPSO, BIS-TRIS propane, BES, MOPS, TES, DISO, Trizma, TRICINE, GLY-GLY, HEPPS, BICINE, TAPS, A MPD, A MPSO, CHES, CAPSO, AMP, CAPS, IB1, TCEP, EGTA, Tween 20, KCl, KOH, MgCl2, glycerol, or any combination thereof. In some embodiments, a buffer may comprise Tris-HCl pH 8.8, VLB, EGTA, CH3COOH, TCEP, IsoAmp®, (NH4)2SO4, KCl, MgSO4, Tween20, KOAc, MgOAc, BSA, phosphate, citrate, acetate, imidazole, or any combination thereof. In some embodiments, the concentration of the buffering agent in the buffer is 1 mM to 200 mM. A buffer compatible with an effector protein may comprise a buffering agent at a concentration of 10 mM to 30 mM. A buffer compatible with an effector protein may comprise a buffering agent at a concentration of about 20 mM. A buffering agent may provide a pH for the buffer or the solution in which the activity of the effector protein occurs. The pH may be 3 to 4, 3.5 to 4.5, 4 to 5, 4.5 to 5.5, 5 to 6, 5.5 to 6.5, 6 to 7, 6.5 to 7.5, 7 to 8, 7.5 to 8.5, 8 to 9, 8.5 to 9.5, 9 to 10, or 9.5 to 10.5.

In some embodiments, systems comprise a solution, wherein the solution comprises at least one salt. Accordingly, in some embodiments, the at least one salt may be selected from a magnesium salt, a zinc salt, a potassium salt, a calcium salt, and a sodium salt. In some embodiments, the at least one salt is selected from potassium acetate, magnesium acetate, sodium chloride, potassium chloride, magnesium chloride, calcium chloride, and any combination thereof. In some embodiments, the salt is a combination of two or more salts. For example, in some embodiments, the salt is a combination of two or more salts selected from a magnesium salt, a zinc salt, a potassium salt, a calcium salt and a sodium salt. In some embodiments, the salt is magnesium acetate. In some embodiments, the salt is magnesium chloride. In some embodiments, the salt is potassium acetate. In some embodiments, the salt is potassium nitrate. In some embodiments, the salt is zinc chloride. In embodiments, the salt is sodium chloride. In some embodiments, the salt is potassium chloride.

In some embodiments, the concentration of the one or more salt in the solution is about 0.001 mM to about 500 mM. In some embodiments, the concentration of the salt is about 0.001 mM to about 400 mM. In some embodiments, the concentration of the salt is about 0.001 mM to about 300 mM. In some embodiments, the concentration of the salt is about 0.001 mM to about 200 mM. In some embodiments, the concentration of the salt is about 0.001 mM to about 100 mM. In some embodiments, the concentration of the salt is about 0.001 mM to about 10 mM. In some embodiments, the concentration of the salt is about 0.01 mM to about 500 mM. In some embodiments, the concentration of the salt is about 0.01 mM to about 400 mM. In some embodiments, the concentration of the salt is about 0.01 mM to about 300 mM. In some embodiments, the concentration of the salt is about 0.01 mM to about 200 mM. In some embodiments, the concentration of the salt is about 0.01 mM to about 100 mM. In some embodiments, the concentration of the salt is about 0.01 mM to about 10 mM. In some embodiments, the concentration of the salt is about 0.1 mM to about 500 mM. In some embodiments, the concentration of the salt is about 0.1 mM to about 400 mM. In some embodiments, the concentration of the salt is about 0.1 mM to about 300 mM. In some embodiments, the concentration of the salt is about 0.1 mM to about 200 mM. In some embodiments, the concentration of the salt is about 0.1 mM to about 100 mM. In some embodiments, the concentration of the salt is about 0.1 mM to about 10 mM. In some embodiments, the concentration of the salt is about 1 mM to about 500 mM. In some embodiments, the concentration of the salt is about 1 mM to about 400 mM. In some embodiments, the concentration of the salt is about 1 mM to about 300 mM. In some embodiments, the concentration of the salt is about 1 mM to about 200 mM. In some embodiments, the concentration of the salt is about 1 mM to about 100 mM. In some embodiments, the concentration of the salt is about 1 mM to about 10 mM. In some embodiments, the concentration of the salt is about 10 mM to about 500 mM. In some embodiments, the concentration of the salt is about 10 mM to about 400 mM. In some embodiments, the concentration of the salt is about 10 mM to about 300 mM. In some embodiments, the concentration of the salt is about 10 mM to about 200 mM. In some embodiments, the concentration of the salt is about 10 mM to about 100 mM. In some embodiments, the concentration of the salt is about 100 mM to about 500 mM. In some embodiments, the concentration of the salt is about 100 mM to about 400 mM. In some embodiments, the concentration of the salt is about 100 mM to about 300 mM. In some embodiments, the concentration of the salt is about 100 mM to about 200 mM. In some embodiments, the salt is potassium acetate and the concentration of salt in the solution is about 100 mM. In some embodiments, the salt is potassium acetate or sodium chloride and the concentration of salt in the solution is about 200 mM. In some embodiments, the salt is potassium acetate or sodium chloride and the concentration of salt in the solution is about 100 mM to about 200 mM. In some embodiments, the concentration of the at least one salt in the solution is 5 mM to 100 mM, 5 mM to 10 mM, 1 mM to 60 mM, or 1 mM to 10 mM. In some embodiments, the concentration of the at least one salt is about 105 mM. In some embodiments, the concentration of the at least one salt is about 55 mM. In some embodiments, the concentration of the at least one salt is about 7 mM. In some embodiments, the solution comprises potassium acetate and magnesium acetate. In some embodiments, the solution comprises sodium chloride and magnesium chloride. In some embodiments, the solution comprises potassium chloride and magnesium chloride. In some embodiments, the salt is a magnesium salt and the concentration of magnesium in the solution is at least 5 mM, 7 mM, at least 9 mM, at least 11 mM, at least 13 mM, or at least 15 mM. In some embodiments, the concentration of magnesium is less than 20 mM, less than 18 mM, or less than 16 mM.

In some embodiments, systems comprise a solution, wherein the solution comprises at least one crowding agent. A crowding agent may reduce the volume of solvent available for other molecules in the solution, thereby increasing the effective concentrations of said molecules. Exemplary crowding agents include glycerol and bovine serum albumin. In some embodiments, the crowding agent is glycerol. In some embodiments, the concentration of the crowding agent in the solution is 0.01% (v/v) to 10% (v/v). In some embodiments, the concentration of the crowding agent in the solution is 0.5% (v/v) to 10% (v/v).

In some embodiments, systems comprise a solution, wherein the solution comprises at least one detergent. Exemplary detergents include Tween, Triton-X, and IGEPAL. A solution may comprise Tween, Triton-X, or any combination thereof. A solution may comprise Triton-X. A solution may comprise IGEPAL CA-630. In some embodiments, the concentration of the detergent in the solution is 2% (v/v) or less. In some embodiments, the concentration of the detergent in the solution is 1% (v/v) or less. In some embodiments, the concentration of the detergent in the solution is 0.00001% (v/v) to 0.01% (v/v). In some embodiments, the concentration of the detergent in the solution is about 0.01% (v/v).

In some embodiments, systems comprise a solution, wherein the solution comprises at least one reducing agent. Exemplary reducing agents comprise dithiothreitol (DTT), β-mercaptoethanol (BME), or tris(2-carboxyethyl) phosphine (TCEP). In some embodiments, the reducing agent is DTT. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is 0.5 mM to 2 mM. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is about 1 mM.

In some embodiments, systems comprise a solution, wherein the solution comprises a competitor. In general, competitors compete with the target nucleic acid or the reporter nucleic acid for cleavage by the effector protein or a dimer thereof. Exemplary competitors include heparin, and imidazole, and salmon sperm DNA. In some embodiments, the concentration of the competitor in the solution is 1 μg/mL to 100 μg/mL. In some embodiments, the concentration of the competitor in the solution is 40 μg/mL to 60 μg/mL.

In some embodiments, systems comprise a solution, wherein the solution comprises a co-factor. In some embodiments, the co-factor allows an effector protein or a multimeric complex thereof to perform a function, including pre-crRNA processing and/or target nucleic acid cleavage. The suitability of a cofactor for an effector protein or a multimeric complex thereof may be assessed, such as by methods based on those described by Sundaresan et al. (Cell Rep. 2017 Dec. 26; 21(13): 3728-3739). In some embodiments, an effector or a multimeric complex thereof forms a complex with a co-factor. In some embodiments, the co-factor is a divalent metal ion. In some embodiments, the divalent metal ion is selected from Mg2+, Mn2+, Zn2+, Ca2+, Cu2+. In some embodiments, the divalent metal ion is Mg2+. In some embodiments, the co-factor is Mg2+.

In some embodiments, systems, and compositions for use with systems comprise a catalytic reagent for signal improvement or enhancement. In some embodiments, the catalytic reagent enhances signal generation via hydrolysis of inorganic pyrophosphates. In some embodiments, catalytic reagents enhance signal generation via enhancement of DNA replication. In some embodiments, catalytic reagents enhance signal amplification via revival of ions (e.g., Mg2+) in a buffer, thereby enhancing the function of an effector protein. In some embodiments, the catalytic reagent for signal improvement may be an enzyme. In some embodiments, the catalytic reagent for signal improvement are particularly useful in amplification and/or detection reactions as described herein. Other exemplary reagents useful for amplification and/or detection reactions (i.e., amplification and detection reagents, respectively) are described throughout herein.

Any of the systems, methods, or compositions described herein may comprise a catalytic reagent or the use thereof. In some embodiments, compositions comprise about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 enzyme unit (U) of a catalytic reagent per 10 μL of solution. In some embodiments, a catalytic reagent is present in a composition at a concentration of 0.125 Units, 0.5 Units, 0.25 Units, 1.0 Units, 2.0 Units, 2.5 Units, or 4 Units per discrete reaction volume. In some embodiments, a catalytic reagent is provided in a system separately from a buffer provided in the system. In some embodiments, systems comprise a buffer, wherein a catalytic reagent is provided in the buffer.

In some embodiments, a catalytic reagent improves the signal to noise ratio of an effector protein-based detection reaction. In some embodiments, a catalytic reagent improves overall signal (e.g., fluorescence of a cleaved reporter). A catalytic reagent may improve signal by a factor, wherein the signal is indicative of the presence of a target nucleic acid. In some embodiments, the factor may be at least about 1.1, at least about 2, at least about 3, at least about 4, at least about 5, at least about 6, at least about 7, at least about 8, at least about 9, or at least about 10.

Also provided herein are reagents for: detection reactions, nuclease purification, cell lysis, in vitro transcription reactions, amplification reactions, reverse transcription reactions, and the like. In some embodiments, systems, compositions, and/or solutions described herein comprise one or more of: detection reagents, nuclease purification reagents, cell lysis reagents, in vitro transcription reagents, amplification reagents, reverse transcription reagents, and combinations thereof. In some embodiments, any such reagents suitable with the solutions, compositions, systems, and/or methods described herein may be used to achieve one or more of the foregoing described reactions. Reagents provided herein may be used with any other solution components described herein, including buffers, amino acids or derivatives thereof, chaotropes, chelators, cyclodextrins, inhibitors, ionic liquids, linkers, metals, non-detergent sulfobetaines, organic acids, osmolytes, peptides, polyamides, polymers, polyols, polyols and salts, salts, or combinations thereof.

### Detection Reagents/Components and Reporters

In some embodiments, systems disclosed herein comprise detection reagents to facilitate detection of nucleic acids as described herein. Non-limiting examples of detection reagents include a reporter nucleic acid, a detection moiety, and additional polypeptides. In some embodiments, the detection reagent is operably linked to an effector protein described herein such that a detection event occurs upon contacting the detection reagent and effector protein with a target nucleic acid. Upon the occurrence of the detection event, a signal (e.g., a detectable signal or detectable product) can be generated thereby indicating detection of the target nucleic acid. When a detection event is described in reference to a microfluidic device, reference is made to a moment in which compositions within the detection region of a microfluidic device exhibit binding of an effector protein to a guide nucleic acid, binding of a guide nucleic acid to a target nucleic acid or target amplicon, and/or access to and cleavage of a reporter by an activated effector protein, in accordance to the assay(s) being performed. A detection event may produce a detectable product or a detectable signal. When a detectable product is described herein, reference is made to a unit produced after the cleavage of a reporter that is capable of being discovered, identified, perceived or noticed. A detectable product can comprise a detectable label and/or moiety that emits a detectable signal. A detectable product may include other components that are not capable of being readily discovered, identified, perceived or noticed at the same time as the detectable signal. For example, a detectable product may comprise remnants of the reporter. Accordingly, in some instances, the detectable product comprises RNA and/or DNA.

Any suitable detection reagent may be used, including: a nucleic acid (which may be referred to herein as a detection or reporter nucleic acid), a detection moiety, an additional polypeptide, or a combination thereof. Other detection reagents include buffers, reverse transcriptase mix, a catalytic reagent, a stain, and the like. Any reagents suitable with the detection reactions, events, and signals described herein are useful as detection reagents for the solutions, compositions, systems, and methods provided herein. In some embodiments, detection reagents are capable of detecting a nucleic acid in a sample.

In some embodiments, solutions, compositions, systems, and methods comprise 0.01 μL, 0.02 μL, 0.03 μL, 0.04 μL, 0.05 μL, 0.06 μL, 0.07 μL, 0.08 μL, 0.09 μL, 0.1 μL, 0.2 μL, 0.3 μL, 0.4 μL, 0.5 μL, 0.6 μL, 0.7 μL, 0.8 μL, 0.9 μL, 1 μL, 2 μL, 3 μL, 4 μL, 5 μL, 6 μL, 7 μL, 8 μL, 9 μL, 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, 500 μL, or more of each detection reagent as described herein. In some embodiments, solutions, compositions, systems, and methods comprise 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, or more of each detection reagent as described herein. In some embodiments, solutions, compositions, systems, and methods comprise 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 150 μM, 200 μM, 250 μM, 300 μM, 350 μM, 400 μM, 450 μM, 500 μM, or more of each detection reagent as described herein. In some embodiments, solutions, compositions, systems, and methods comprise 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or more of each detection reagent as described herein.

In some embodiments, detection reagents are capable of detecting a nucleic acid in a sample. Nucleic acid amplification of the target nucleic acid may improve at least one of sensitivity, specificity, or accuracy of the assay in detecting the target nucleic acid. Accordingly, in some embodiments, nucleic acid detection involves PCR or isothermal nucleic acid amplification, providing improved sensitive, specific, or rapid detection. The reagents or components for nucleic acid detection may comprise recombinases, primers, polypeptides, buffers, and signal reagents suitable for a detection reaction.

In some embodiments, systems described herein comprise a PCR tube, a PCR well or a PCR plate. In some embodiments, the wells of the PCR plate may be pre-aliquoted with the reagent for detecting a nucleic acid, as well as a guide nucleic acid, an effector protein, a multimeric complex, an amplification reagent, or any combination thereof. In some embodiments, the pre-aliquoted guide nucleic acid targeting a target sequence, and an effector protein capable of being activated when complexed with the guide nucleic acid and the target sequence. A user may thus add a sample of interest to a well of the pre-aliquoted PCR plate.

In some embodiments, nucleic acid detection is performed in a nucleic acid detection region on a support medium, or sample interface. Alternatively, or in combination, the nucleic acid detection is performed in a reagent chamber, and the resulting sample is applied to the support medium, sample interface, or surface within a reagent chamber.

In some embodiments, the reporter nucleic acid is capable of being cleaved by the activated nuclease, thereby generating a detectable signal. A user may thus add a sample of interest to a well of the pre-aliquoted PCR plate and measure for the detectable signal with a fluorescent light reader or a visible light reader.

In some embodiments, detection reaction of nucleic acid as described herein is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value 1 to 60 minutes. In some embodiments, the detection reaction is performed for 1 to 60, 5 to 55, 10 to 50, 15 to 45, 20 to 40, or 25 to 35 minutes. In some embodiments, the detection reaction is performed at a temperature of around 20-45° C. In some embodiments, the detection reaction is performed at a temperature no greater than 20° C., 25° C., 30° C., 35° C., 37° C., 40° C., 45° C., or any value 20° C. to 45° C. In some embodiments, the detection reaction is performed at a temperature of at least 20° C., 25° C., 30° C., 35° C., 37° C., 40° C., or 45° C., or any value 20° C. to 45° C. In some embodiments, the detection reaction is performed at a temperature of 20° C. to 45° C., 25° C. to 40° C., 30° C. to 40° C., or 35° C. to 40° C.

These reagents are compatible with the samples, solutions, compositions, systems, methods of detection, and support mediums as described herein for detection of an ailment, such as a disease, cancer, or genetic disorder, or genetic information, such as for phenotyping, genotyping, or determining ancestry. The reagents described herein for detecting a disease, cancer, or genetic disorder comprise a guide nucleic acid targeting the target nucleic acid segment indicative of a disease, cancer, or genetic disorder.

In some embodiments, systems disclosed herein comprise a reporter. By way of non-limiting and illustrative example, a reporter may comprise a single stranded nucleic acid and a detection moiety (e.g., a labeled single stranded RNA reporter), wherein the nucleic acid is capable of being cleaved by an effector protein (e.g., a CRISPR/Cas protein as disclosed herein) or a multimeric complex thereof, releasing the detection moiety, and generating a detectable signal or a detectable product comprising a detectable moiety or a detectable signal. As used herein, “reporter” is used interchangeably with “reporter nucleic acid” or “reporter molecule”. The effector proteins disclosed herein, activated upon hybridization of a guide nucleic acid to a target nucleic acid, may cleave the reporter. Cleaving the “reporter” may be referred to herein as cleaving the “reporter nucleic acid,” the “reporter molecule,” or the “nucleic acid of the reporter.” Cleavage of a reporter may produce different types of signals (e.g., a detectable signal). In some cases, cleavage of the reporter can produce a calorimetric signal, a potentiometric signal, an amperometric signal, an optical signal, or a piezo-electric signal. Various devices and/or sensors can be used to detect these different types of signals, which indicate whether a target nucleic acid, is present in the sample. The sensors usable to detect such signals can include, for example, optical sensors (e.g., imaging devices for detecting fluorescence or optical signals with various wavelengths and frequencies), electric potential sensors, surface plasmon resonance (SPR) sensors, interferometric sensors, or any other type of sensor suitable for detecting calorimetric signals, potentiometric signals, amperometric signals, optical signals, or piezo-electric signals.

Reporters may comprise RNA. Reporters may comprise DNA. Reporters may be double-stranded. Reporters may be single-stranded. In some embodiments, reporters comprise a protein capable of generating a signal. In some embodiments, a reporter may comprise a protein capable of generating a detectable signal or signal. In some embodiments, a reporter may be operably linked to the protein capable of generating a signal. A signal may be a calorimetric, potentiometric, amperometric, optical (e.g., fluorescent, colorimetric, etc.), or piezo-electric signal. In some embodiments, the reporter comprises a detection moiety. In some embodiments, the reporter is configured to release a detection moiety or generate a signal indicative of a presence or absence of the target nucleic acid. For example, the signal can indicate a presence of the target nucleic acid in the sample, and an absence of the signal can indicate an absence of the target nucleic acid in the sample. Suitable detectable labels and/or moieties that may provide a signal include, but are not limited to, an enzyme, a radioisotope, a member of a specific binding pair; a fluorophore; a fluorescent protein; a quantum dot; and the like.

In some embodiments, the reporter comprises a detection moiety and a quenching moiety. In some embodiments, the reporter comprises a cleavage site, wherein the detection moiety is located at a first site on the reporter and the quenching moiety is located at a second site on the reporter, wherein the first site and the second site are separated by the cleavage site. Sometimes the quenching moiety is a fluorescence quenching moiety. In some embodiments, the quenching moiety is 5′ to the cleavage site and the detection moiety is 3′ to the cleavage site. In some embodiments, the detection moiety is 5′ to the cleavage site and the quenching moiety is 3′ to the cleavage site. Sometimes the quenching moiety is at the 5′ terminus of the nucleic acid of a reporter. Sometimes the detection moiety is at the 3′ terminus of the nucleic acid of a reporter. In some embodiments, the detection moiety is at the 5′ terminus of the nucleic acid of a reporter. In some embodiments, the quenching moiety is at the 3′ terminus of the nucleic acid of a reporter.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Suitable enzymes include, but are not limited to, horseradish peroxidase (HRP), alkaline phosphatase (AP), beta-galactosidase (GAL), glucose-6-phosphate dehydrogenase, beta-N-acetylglucosaminidase, (3-glucuronidase, invertase, Xanthine Oxidase, firefly luciferase, and glucose oxidase (GO).

In some embodiments, the detection moiety comprises an invertase. The substrate of the invertase may be sucrose. A DNS reagent may be included in the system to produce a colorimetric change when the invertase converts sucrose to glucose. In some embodiments, the reporter nucleic acid and invertase are conjugated using a heterobifunctional linker via sulfo-SMCC chemistry.

Suitable fluorophores may provide a detectable fluorescence signal in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO™ 633 (NHS Ester) (Integrated DNA Technologies). Non-limiting examples of fluorophores are fluorescein amidite, 6-Fluorescein, IRDye 700, TYE 665, Alex Fluor 594, or ATTO™ 633 (NHS Ester). The fluorophore may be an infrared fluorophore. The fluorophore may emit fluorescence in the range of 500 nm and 720 nm. In some embodiments, the fluorophore emits fluorescence at a wavelength of 700 nm or higher. In other embodiments, the fluorophore emits fluorescence at about 665 nm. In some embodiments, the fluorophore emits fluorescence in the range of 500 nm to 520 nm, 500 nm to 540 nm, 500 nm to 590 nm, 590 nm to 600 nm, 600 nm to 610 nm, 610 nm to 620 nm, 620 nm to 630 nm, 630 nm to 640 nm, 640 nm to 650 nm, 650 nm to 660 nm, 660 nm to 670 nm, 670 nm to 680 nm, 690 nm to 690 nm, 690 nm to 700 nm, 700 nm to 710 nm, 710 nm to 720 nm, or 720 nm to 730 nm. In some embodiments, the fluorophore emits fluorescence in the range 450 nm to 750 nm, 500 nm to 650 nm, or 550 to 650 nm.

Systems may comprise a quenching moiety. A quenching moiety may be chosen based on its ability to quench the detection moiety. A quenching moiety may be a non-fluorescent fluorescence quencher. A quenching moiety may quench a detection moiety that emits fluorescence in the range of 500 nm and 720 nm. A quenching moiety may quench a detection moiety that emits fluorescence in the range of 500 nm and 720 nm. In some embodiments, the quenching moiety quenches a detection moiety that emits fluorescence at a wavelength of 700 nm or higher. In other embodiments, the quenching moiety quenches a detection moiety that emits fluorescence at about 660 nm or about 670 nm. In some embodiments, the quenching moiety quenches a detection moiety that emits fluorescence in the range of 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some embodiments, the quenching moiety quenches a detection moiety that emits fluorescence in the range 450 nm to 750 nm, 500 nm to 650 nm, or 550 to 650 nm. A quenching moiety may quench fluorescein amidite, 6-Fluorescein, IRDye 700, TYE 665, Alex Fluor 594, or ATTO™ 633 (NHS Ester). A quenching moiety may be Iowa Black RQ, Iowa Black FQ or IRDye QC-1 Quencher. A quenching moiety may quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO™ 633 (NHS Ester) (Integrated DNA Technologies). A quenching moiety may be Iowa Black RQ (Integrated DNA Technologies), Iowa Black FQ (Integrated DNA Technologies) or IRDye QC-1 Quencher (LiCor). Any of the quenching moieties described herein may be from any commercially available source, may be an alternative with a similar function, a generic, or a non-tradename of the quenching moieties listed.

The generation of the detectable product or detectable signal from the release of the detection moiety may indicate that cleavage by the effector protein has occurred and that the sample contains the target nucleic acid. In some embodiments, the detection moiety comprises a fluorescent dye. Sometimes the detection moiety comprises a fluorescence resonance energy transfer (FRET) pair. In some embodiments, the detection moiety comprises an infrared (IR) dye. In some embodiments, the detection moiety comprises an ultraviolet (UV) dye. Alternatively, or in combination, the detection moiety comprises a protein. Sometimes the detection moiety comprises an antigen. Sometimes the detection moiety comprises a biotin. Sometimes the detection moiety comprises at least one of avidin or streptavidin. In some embodiments, the detection moiety comprises a polysaccharide, a polymer, or a nanoparticle. In some embodiments, the detection moiety comprises a gold nanoparticle or a latex nanoparticle.

A detection moiety may be any moiety capable of generating a detectable product or detectable signal upon cleavage of the reporter by the effector protein. The detectable product may be a detectable unit generated from the detectable moiety and capable of emitting a detectable signal as described herein. In some embodiments, the detectable product further comprises a detectable label, a fluorophore, a reporter, or a combination thereof. In some embodiments, the detectable product comprises RNA, DNA, or both. In some embodiments, the detectable product is configured to generate a signal indicative of the presence or absence of the target nucleic acid in, for instance, a cell or a sample.

A detection moiety may be any moiety capable of generating a calorimetric, potentiometric, amperometric, optical (e.g., fluorescent, colorimetric, etc.), or piezo-electric signal. A nucleic acid of a reporter, sometimes, is protein-nucleic acid that is capable of generating a calorimetric, potentiometric, amperometric, optical (e.g., fluorescent, colorimetric, etc.), or piezo-electric signal upon cleavage of the nucleic acid. Often a calorimetric signal is heat produced after cleavage of the nucleic acids of a reporter. Sometimes, a calorimetric signal is heat absorbed after cleavage of the nucleic acids of a reporter. A potentiometric signal, for example, is electrical potential produced after cleavage of the nucleic acids of a reporter. An amperometric signal may be movement of electrons produced after the cleavage of nucleic acid of a reporter. Often, the signal is an optical signal, such as a colorimetric signal or a fluorescence signal. An optical signal is, for example, a light output produced after the cleavage of the nucleic acids of a reporter. Sometimes, an optical signal is a change in light absorbance between before and after the cleavage of nucleic acids of a reporter. Often, a piezo-electric signal is a change in mass between before and after the cleavage of the nucleic acid of a reporter.

The detectable signal may be a colorimetric signal or a signal visible by eye. In some embodiments, the detectable signal may be fluorescent, electrical, chemical, electrochemical, or magnetic. In some embodiments, the first detection signal may be generated by binding or interaction of the detection moiety to the capture molecule in the detection region, where the first detection signal indicates that the sample contained the target nucleic acid. Sometimes systems are capable of detecting more than one type of target nucleic acid, wherein the system comprises more than one type of guide nucleic acid and more than one type of reporter nucleic acid. In some embodiments, the detectable signal may be generated directly by the cleavage event. Alternatively, or in combination, the detectable signal may be generated indirectly by the signal event. Sometimes the detectable signal is not a fluorescent signal. In some embodiments, the detectable signal may be a colorimetric or color-based signal. In some embodiments, the detected target nucleic acid may be identified based on its spatial location on the detection region of the support medium. In some embodiments, the second detectable signal may be generated in a spatially distinct location than the first generated signal.

In some embodiments, the reporter nucleic acid is a single-stranded nucleic acid sequence comprising ribonucleotides. The nucleic acid of a reporter may be a single-stranded nucleic acid sequence comprising at least one ribonucleotide. In some embodiments, the nucleic acid of a reporter is a single-stranded nucleic acid comprising at least one ribonucleotide residue at an internal position that functions as a cleavage site. In some embodiments, the nucleic acid of a reporter comprises at least 2, at least 3, at least 4, at least 5, at least 6, at least 7, at least 8, at least 9, or at least 10 ribonucleotide residues at an internal position. In some embodiments, the nucleic acid of a reporter comprises from 2 to 10, from 3 to 9, from 4 to 8, or from 5 to 7 ribonucleotide residues at an internal position. Sometimes the ribonucleotide residues are continuous. Alternatively, the ribonucleotide residues are interspersed in between non-ribonucleotide residues. In some embodiments, the nucleic acid of a reporter has only ribonucleotide residues. In some embodiments, the nucleic acid of a reporter has only deoxyribonucleotide residues. In some embodiments, the nucleic acid comprises nucleotides resistant to cleavage by the effector protein described herein. In some embodiments, the nucleic acid of a reporter comprises synthetic nucleotides. In some embodiments, the nucleic acid of a reporter comprises at least one ribonucleotide residue and at least one non-ribonucleotide residue.

In some embodiments, the nucleic acid of a reporter comprises at least one uracil ribonucleotide. In some embodiments, the nucleic acid of a reporter comprises at least two uracil ribonucleotides. Sometimes the nucleic acid of a reporter has only uracil ribonucleotides. In some embodiments, the nucleic acid of a reporter comprises at least one adenine ribonucleotide. In some embodiments, the nucleic acid of a reporter comprises at least two adenine ribonucleotides. In some embodiments, the nucleic acid of a reporter has only adenine ribonucleotides. In some embodiments, the nucleic acid of a reporter comprises at least one cytosine ribonucleotide. In some embodiments, the nucleic acid of a reporter comprises at least two cytosine ribonucleotides. In some embodiments, the nucleic acid of a reporter comprises at least one guanine ribonucleotide. In some embodiments, the nucleic acid of a reporter comprises at least two guanine ribonucleotides. In some embodiments, a nucleic acid of a reporter comprises a single unmodified ribonucleotide. In some embodiments, a nucleic acid of a reporter comprises only unmodified deoxyribonucleotides.

In some embodiments, the nucleic acid of a reporter is 5 to 20, 5 to 15, 5 to 10, 7 to 20, 7 to 15, or 7 to 10 nucleotides in length. In some embodiments, the nucleic acid of a reporter is 3 to 20, 4 to 10, 5 to 10, or 5 to 8 nucleotides in length. In some embodiments, the nucleic acid of a reporter is 5 to 12 nucleotides in length. In some embodiments, the reporter nucleic acid is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides in length. In some embodiments, the reporter nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length.

In some embodiments, systems comprise a plurality of reporters. The plurality of reporters may comprise a plurality of signals. In some embodiments, systems comprise at least 2, at least 3, at least 4, at least 5, at least 6, at least 7, at least 8, at least 9, at least 10, at least 11, at least 12, at least 13, at least 14, at least 15, at least 20, at least 30, at least 40, or at least 50 reporters. In some embodiments, there are 2 to 50, 3 to 40, 4 to 30, 5 to 20, or 6 to 10 different reporters.

In some embodiments, systems comprise an effector protein and a reporter nucleic acid configured to undergo trans cleavage by the effector protein. Trans cleavage of the reporter may generate a signal from the reporter or alter a signal from the reporter. In some embodiments, the signal is an optical signal, such as a fluorescence signal or absorbance band. Trans cleavage of the reporter may alter the wavelength, intensity, or polarization of the optical signal. For example, the reporter may comprise a fluorophore and a quencher, such that trans cleavage of the reporter separates the fluorophore and the quencher thereby increasing a fluorescence signal from the fluorophore. Herein, detection of reporter cleavage to determine the presence of a target nucleic acid may be referred to as ‘DETECTR’. In some embodiments described herein is a method of assaying for a target nucleic acid in a sample comprising contacting the target nucleic acid with an effector protein, a non-naturally occurring guide nucleic acid that hybridizes to a segment of the target nucleic acid, and a reporter nucleic acid, and assaying for a change in a signal, wherein the change in the signal is produced by cleavage of the reporter nucleic acid.

In the presence of a large amount of non-target nucleic acids, an activity of an effector protein (e.g., an effector protein as disclosed herein) may be inhibited. This is because the activated effector proteins collaterally cleave any nucleic acids. If total nucleic acids are present in large amounts, they may outcompete reporters for the effector proteins. In some embodiments, systems comprise an excess of reporter(s), such that when the system is operated and a solution of the system comprising the reporter is combined with a sample comprising a target nucleic acid, the concentration of the reporter in the combined solution-sample is greater than the concentration of the target nucleic acid. In some embodiments, the sample comprises amplified target nucleic acid. In some embodiments, the sample comprises an unamplified target nucleic acid. In some embodiments, the concentration of the reporter is greater than the concentration of target nucleic acids and non-target nucleic acids. The non-target nucleic acids may be from the original sample, either lysed or unlysed. The non-target nucleic acids may comprise byproducts of amplification. In some embodiments, systems comprise a reporter wherein the concentration of the reporter in a solution 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold excess of total nucleic acids. 1.5 fold to 100 fold, 2 fold to 10 fold, 10 fold to 20 fold, 20 fold to 30 fold, 30 fold to 40 fold, 40 fold to 50 fold, 50 fold to 60 fold, 60 fold to 70 fold, 70 fold to 80 fold, 80 fold to 90 fold, 90 fold to 100 fold, 1.5 fold to 10 fold, 1.5 fold to 20 fold, 10 fold to 40 fold, 20 fold to 60 fold, or 10 fold to 80 fold excess of total nucleic acids.

### Amplification Reagents/Components

In some embodiments, systems described herein comprise a reagent or component for amplifying a nucleic acid. Non-limiting examples of reagents for amplifying a nucleic acid include polymerases, primers, and nucleotides. In some embodiments, systems comprise reagents for nucleic acid amplification of a target nucleic acid in a sample. Nucleic acid amplification of the target nucleic acid may improve at least one of sensitivity, specificity, or accuracy of the assay in detecting the target nucleic acid. In some embodiments, nucleic acid amplification is isothermal nucleic acid amplification, providing for the use of the system or system in remote regions or low resource settings without specialized equipment for amplification. In some embodiments, amplification of the target nucleic acid increases the concentration of the target nucleic acid in the sample relative to the concentration of nucleic acids that do not correspond to the target nucleic acid.

The reagents for nucleic acid amplification may comprise a recombinase, a primer, an oligonucleotide primer, an activator, a deoxynucleoside triphosphate (dNTP), a ribonucleoside triphosphate (rNTP), a single-stranded DNA binding (SSB) protein, Rnase inhibitor, water, a polymerase, reverse transcriptase mix, or a combination thereof that is suitable for an amplification reaction. Non-limiting examples of amplification reactions are transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA).

Such amplification reactions may also be used in combination with reverse transcription (RT) of an RNA of interest. Accordingly, also provided herein are reagents for both the reverse transcription and amplification of nucleic acids. In some embodiments, solutions, compositions, systems and methods comprise 0.01 μL, 0.02 μL, 0.03 μL, 0.04 μL, 0.05 μL, 0.06 μL, 0.07 μL, 0.08 μL, 0.09 μL, 0.1 μL, 0.2 μL, 0.3 μL, 0.4 μL, 0.5 μL, 0.6 μL, 0.7 μL, 0.8 μL, 0.9 μL, 1 μL, 2 μL, 3 μL, 4 μL, 5 μL, 6 μL, 7 μL, 8 μL, 9 μL, 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, 500 μL, or more of each amplification described herein. In some embodiments, solutions, compositions, systems and methods comprise 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, or more of each amplification reagent as described herein. In some embodiments, solutions, compositions, systems and methods comprise 1 μM, 2 μM, 3 μM, 4 μM, 5 μM, 6 μM, 7 μM, 8 μM, 9 μM, 10 μM, 20 μM, 30 μM, 40 μM, 50 μM, 60 PM, 70 μM, 80 μM, 90 μM, 100 μM, 150 μM, 200 μM, 250 μM, 300 μM, 350 μM, 400 μM, 450 PM, 500 PM, or more of each amplification reagent as described herein. In some embodiments, solutions, compositions, systems and methods comprise 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or more of each amplification reagent as described herein.

In some embodiments, systems comprise a PCR tube, a PCR well or a PCR plate. The wells of the PCR plate may be pre-aliquoted with the reagent for amplifying a nucleic acid, as well as a guide nucleic acid, an effector protein, a multimeric complex, or any combination thereof. The wells of the PCR plate may be pre-aliquoted with a guide nucleic acid targeting a target sequence, an effector protein capable of being activated when complexed with the guide nucleic acid and the target sequence, and at least one population of a single stranded reporter nucleic acid comprising a detection moiety. A user may thus add the biological sample of interest to a well of the pre-aliquoted PCR plate and measure for the detectable signal with a fluorescent light reader or a visible light reader.

In some embodiments, systems comprise a PCR plate; a guide nucleic acid targeting a target sequence; an effector protein capable of being activated when complexed with the guide nucleic acid and the target sequence; and a single stranded reporter nucleic acid comprising a detection moiety, wherein the reporter nucleic acid is capable of being cleaved by the activated nuclease, thereby generating a detectable signal.

In some embodiments, systems comprise a support medium; a guide nucleic acid targeting a target sequence; and an effector protein capable of being activated when complexed with the guide nucleic acid and the target sequence. In some embodiments, nucleic acid amplification is performed in a nucleic acid amplification region on the support medium. Alternatively, or in combination, the nucleic acid amplification is performed in a reagent chamber, and the resulting sample is applied to the support medium.

In some embodiments, a system for modifying a target nucleic acid comprises a PCR plate; a guide nucleic acid targeting a target sequence; and an effector protein capable of being activated when complexed with the guide nucleic acid and the target sequence. The wells of the PCR plate may be pre-aliquoted with the guide nucleic acid targeting a target sequence, and an effector protein capable of being activated when complexed with the guide nucleic acid and the target sequence. A user may thus add the biological sample of interest to a well of the pre-aliquoted PCR plate.

Often, the nucleic acid amplification is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value 1 to 60 minutes. Sometimes, the nucleic acid amplification is performed for 1 to 60, 5 to 55, 10 to 50, 15 to 45, 20 to 40, or 25 to 35 minutes. Sometimes, the nucleic acid amplification reaction is performed at a temperature of around 20-45° C. In some embodiments, the nucleic acid amplification reaction is performed at a temperature no greater than 20° C., 25° C., 30° C., 35° C., 37° C., 40° C., 45° C., 47° C., 50° C., 55° C., 57° C., 60° C., 65° C., 67° C., 70° C., 75° C., 77° C., 80° C., or any value 20° C. to 80° C. In some embodiments, the nucleic acid amplification reaction is performed at a temperature of at least 20° C., 25° C., 27° C., 30° C., 35° C., 37° C., 40° C., 45° C., 47° C., 50° C., 55° C., 57° C., 60° C., 65° C., 67° C., 70° C., 75° C., 77° C., 80° C., or any value 20° C. to 80° C. In some embodiments, the nucleic acid amplification reaction is performed at a temperature of 20° C. to 45° C., 25° C. to 40° C., 30° C. to 40° C., 35° C. to 40° C., 35° C. to 40° C., 40° C. to 45° C., 45° C. to 50° C., 50° C. to 55° C., 55° C. to 60° C., 35° C. to 40° C., 50° C. to 65° C., 65° C. to 70° C., 70° C. to 80° C., or 75° C. to 80° C.

Often, systems comprise primers for amplifying a target nucleic acid to produce an amplification product comprising the target nucleic acid and a PAM. For embodiment, at least one of the primers may comprise the PAM that is incorporated into the amplification product during amplification. The compositions for amplification of target nucleic acids and methods of use thereof, as described herein, are compatible with any of the methods disclosed herein including methods of assaying for at least one base difference (e.g., assaying for a SNP or a base mutation) in a target nucleic acid, methods of assaying for a target nucleic acid that lacks a PAM by amplifying the target nucleic acid to introduce a PAM, and compositions used in introducing a PAM via amplification into the target nucleic acid.

### Additional System Components

In some embodiments, systems include a package, carrier, or container that is compartmentalized to receive one or more containers such as vials, tubes, and the like, each of the container(s) comprising one of the separate elements to be used in a method described herein. Suitable containers include, for example, test wells, bottles, vials, syringes, and test tubes. In one embodiment, the containers are formed from a variety of materials such as glass, plastic, or polymers. The system or systems described herein contain packaging materials. Examples of packaging materials include, but are not limited to, pouches, blister packs, bottles, tubes, bags, containers, bottles, and any packaging material suitable for intended mode of use.

A system may include labels listing contents and/or instructions for use, or package inserts with instructions for use. A set of instructions will also typically be included. In one embodiment, a label is on or associated with the container. In some embodiments, a label is on a container when letters, numbers or other characters forming the label are attached, molded, or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein. After packaging the formed product and wrapping or boxing to maintain a sterile barrier, the product may be terminally sterilized by heat sterilization, gas sterilization, gamma irradiation, or by electron beam sterilization. Alternatively, the product may be prepared and packaged by aseptic processing.

In some embodiments, systems comprise a solid support. An RNP or effector protein may be attached to a solid support. The solid support may be an electrode or a bead. The bead may be a magnetic bead. Upon cleavage, the RNP is liberated from the solid support and interacts with other mixtures. For example, upon cleavage of the nucleic acid of the RNP, the effector protein of the RNP flows through a chamber into a mixture comprising a substrate. When the effector protein meets the substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

### Certain System Conditions

In some embodiments, systems and methods are employed under certain conditions that enhance an activity of the effector protein relative to alternative conditions, as measured by a detectable signal released from cleavage of a reporter in the presence of the target nucleic acid. The detectable signal may be generated at about the rate of trans cleavage of a reporter nucleic acid. In some embodiments, the reporter nucleic acid is a homopolymeric reporter nucleic acid comprising 5 to 20 consecutive adenines (SEQ ID NO: 96), 5 to 20 consecutive thymines (SEQ ID NO: 97), 5 to 20 consecutive cytosines (SEQ ID NO: 98), or 5 to 20 consecutive guanines (SEQ ID NO: 99). In some embodiments, the reporter is an RNA-FQ reporter.

In some embodiments, effector proteins disclosed herein recognize, bind, or are activated by, different target nucleic acids having different sequences, but are active toward the same reporter nucleic acid, allowing for facile multiplexing in a single assay having a single ssRNA-FQ reporter.

In some embodiments, systems are employed under certain conditions that enhance trans cleavage activity of an effector protein. In some embodiments, under certain conditions, trans cleavage occurs at a rate of at least 0.005 mmol/min, at least 0.01 mmol/min, at least 0.05 mmol/min, at least 0.1 mmol/min, at least 0.2 mmol/min, at least 0.5 mmol/min, or at least 1 mmol/min. In some embodiments, systems and methods are employed under certain conditions that enhance cis-cleavage activity of the effector protein.

Certain conditions that may enhance the activity of an effector protein include a certain salt presence or salt concentration of the solution in which the activity occurs. For example, cis-cleavage activity of an effector protein may be inhibited or halted by a high salt concentration. The salt may be a sodium salt, a potassium salt, a zinc salt, a calcium salt, a lithium salt, an ammonium salt or a magnesium salt. In some embodiments, the salt is NaCl. In some embodiments, the salt is KNO3. In some embodiments, the salt is magnesium acetate. In some embodiments, the salt is magnesium chloride. In some embodiments, the salt is potassium acetate. In some embodiments, the salt is potassium nitrate. In some embodiments, the salt is zinc chloride. In embodiments, the salt is sodium chloride. In some embodiments, the salt is potassium chloride. In some embodiments, the salt is lithium acetate. In some embodiments, the salt is ammonium sulfate. In some embodiments, the salt concentration is less than 150 mM, less than 125 mM, less than 100 mM, less than 75 mM, less than 50 mM, or less than 25 mM. In some embodiments, the salt concentration is more than 1 mM, but less than 150 mM, less than 125 mM, less than 100 mM, less than 75 mM, less than 50 mM, or less than 25 mM. In some embodiments, the salt concentration is more than 10 mM, but less than 150 mM, less than 125 mM, less than 100 mM, less than 75 mM, less than 50 mM, or less than 25 mM. In some embodiments, the salt is potassium acetate or sodium chloride and the concentration of salt in the solution is about 200 mM. In some embodiments, the salt is potassium acetate or, sodium chloride, lithium acetate, or ammonium sulfate and the concentration of salt in the solution is about 100 mM to about 200 mM.

Certain conditions that may enhance the activity of an effector protein include the pH of a solution in which the activity. For example, increasing pH may enhance trans cleavage activity. For example, the rate of trans cleavage activity may increase with increase in pH up to pH 9. In some embodiments, the pH is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some embodiments, the pH is less than 7. In some embodiments, the pH is greater than 7.

Certain conditions that may enhance the activity of an effector protein includes the temperature at which the activity is performed. In some embodiments, the temperature is about 25° C. to about 80° C. In some embodiments, the temperature is about 20° C. to about 40° C., about 30° C. to about 50° C., or about 40° C. to about 60° C. In some embodiments, the temperature is about 25° C., about 30° C., about 35° C., about 40° C., about 45° C., about 50° C., about 55° C., about 60° C., about 65° C., about 70° C., about 75° C., or about 80° C.

IX. Methods and Formulations for Introducing System Components and Compositions into a Target Cell

Disclosed herein, in some aspects, are systems and methods for introducing systems and components of such systems into a target cell. Such systems may comprise, as described herein, one or more components having any one of the polypeptides (e.g., effector proteins, effector partners such as fusion partners, fusion proteins, or combination thereof) or a nucleic acid comprising a nucleotide sequence encoding same. In some embodiments, such systems comprise, as described herein, one or more components having a guide nucleic acid or a nucleic acid comprising a nucleotide sequence encoding same. In some embodiments, systems comprise one or more components having a guide nucleic acid and an additional nucleic acid. Systems and components thereof may be used to introduce the polypeptides, guide nucleic acids, or combinations thereof into a target cell. Such methods may be used to modify or edit a target nucleic acid. In some embodiments, systems comprise the polypeptide, one or more guide nucleic acids, and a reagent for facilitating the introduction of the polypeptide and the one or more guide nucleic acids. In some embodiments, system components for the methods comprise a solution, a buffer, a reagent for facilitating the introduction of the polypeptide and the one or more guide nucleic acids, or combinations thereof. A guide nucleic acid (or a nucleic acid comprising a nucleotide sequence encoding same) and/or a polypeptide (e.g., an effector protein, effector partner such as a fusion partner, and/or fusion protein as described herein) (or a nucleic acid comprising a nucleotide sequence encoding same) can be introduced into a host cell by any of a variety of well-known methods. As a non-limiting example, a guide nucleic acid and/or polypeptide (e.g., effector protein) can be combined with a lipid. As another non-limiting example, a guide RNA and/or polypeptide (e.g., effector protein) can be combined with a particle, or formulated into a particle.

### Methods for Introducing System Components and Compositions to a Host

Described herein are methods of introducing various components described herein to a host. A host can be any suitable host, such as a host cell. When described herein, a host cell can be an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells can be, or have been, used as recipients for methods of introduction described herein, and include the progeny of the original cell which has been transformed by the methods of introduction described herein. It is understood that the progeny of a single cell may not necessarily be completely identical in morphology or in genomic or total DNA complement as the original parent, due to natural, accidental, or deliberate mutation. A host cell can be a recombinant host cell or a genetically modified host cell, if a heterologous nucleic acid, e.g., an expression vector, has been introduced into the cell.

Methods of introducing a nucleic acid and/or protein into a host cell are known in the art, and any convenient method can be used to introduce a subject nucleic acid (e.g., an expression construct/vector) into a target cell (e.g., a human cell, and the like). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al. Adv Drug Deliv Rev. 2012 Sep. 13. pii: S0169-409X(12)00283-9. doi: 10.1016/j.addr.2012.09.023), and the like. In some instances, the nucleic acid and/or protein are introduced into a disease cell comprised in a pharmaceutical composition comprising the guide nucleic acid and/or polypeptide (e.g., the effector protein) and a pharmaceutically acceptable excipient, or combinations thereof.

In certain embodiments, molecules of interest, such as nucleic acids of interest, are introduced to a host. In certain embodiments, polypeptides, such as an effector protein are introduced to a host. In certain embodiments, vectors, such as lipid particles and/or viral vectors can be introduced to a host. Introduction can be for contact with a host or for assimilation into the host, for example, introduction into a host cell.

In some instances, described herein are methods of introducing one or more nucleic acids, such as a nucleic acid encoding a polypeptide (e.g., an effector protein), a nucleic acid encoding an engineered guide nucleic acid and that when transcribed produces an engineered guide nucleic acid, and/or a donor nucleic acid, or combinations thereof, into a host cell. Any suitable method can be used to introduce a nucleic acid into a cell. Suitable methods include, for example, viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like. Further methods are described throughout.

Introducing one or more nucleic acids into a host cell can occur in any culture media and under any culture conditions that promote the survival of the cells. Introducing one or more nucleic acids into a host cell can be carried out in vivo or ex vivo. Introducing one or more nucleic acids into a host cell can be carried out in vitro.

In some embodiments, a polypeptide described herein (e.g., an effector protein, effector partner such as a fusion partner, fusion protein, or combination thereof) can be provided as RNA. The RNA can be provided by direct chemical synthesis or may be transcribed in vitro from a DNA (e.g., encoding the polypeptide). Once synthesized, the RNA may be introduced into a cell by way of any suitable technique for introducing nucleic acids into cells (e.g., microinjection, electroporation, transfection, etc.). In some embodiments, introduction of one or more nucleic acid can be through the use of a vector and/or a vector system, accordingly, in some embodiments, compositions and system described herein comprise a vector and/or a vector system.

Vectors may be introduced directly to a host. In certain embodiments, host cells can be contacted with one or more vectors as described herein, and in certain embodiments, said vectors are taken up by the cells. Methods for contacting cells with vectors include but are not limited to electroporation, calcium chloride transfection, microinjection, lipofection, micro-injection, contact with the cell or particle that comprises a molecule of interest, or a package of cells or particles that comprise molecules of interest.

Components described herein can also be introduced directly to a host. For example, an engineered guide nucleic acid can be introduced to a host, specifically introduced into a host cell. Methods of introducing nucleic acids, such as RNA into cells include, but are not limited to direct injection, transfection, or any other method used for the introduction of nucleic acids.

Polypeptides (e.g., effector proteins, effector partners such as a fusion partners, fusion proteins or combination thereof) described herein can also be introduced directly to a host. In some embodiments, polypeptides described herein can be modified to promote introduction to a host. For example, polypeptides described herein can be modified to increase the solubility of the polypeptide. Such a polypeptide may optionally be fused to a polypeptide domain that increases solubility. The domain may be linked to the polypeptide through a defined protease cleavage site, such as TEV sequence which is cleaved by TEV protease. The linker may also include one or more flexible sequences, e.g., from 1 to 10 glycine residues (SEQ ID NO: 125). In some embodiments, the cleavage of the polypeptide is performed in a buffer that maintains solubility of the product, e.g., in the presence of from 0.5 to 2 M urea, in the presence of polypeptides and/or polynucleotides that increase solubility, and the like. Domains of interest include endosomolytic domains, e.g., influenza HA domain; and other polypeptides that aid in production, e.g., IF2 domain, GST domain, GRPE domain, and the like. In another example, the polypeptide can be modified to improve stability. For example, the polypeptides may be PEGylated, where the polyethyleneoxy group provides for enhanced lifetime in the blood stream. Polypeptides can also be modified to promote uptake by a host, such as a host cell. For example, a polypeptide described herein can be fused to a polypeptide permeant domain to promote uptake by a host cell. Any suitable permeant domains can be used in the non-integrating polypeptides of the present disclosure, including peptides, peptidomimetics, and non-peptide carriers. Examples include penetratin, a permeant peptide may be derived from the third alpha helix of Drosophila melanogaster transcription factor Antennapaedia; the HIV-1 tat basic region amino acid sequence, e.g., amino acids 49-57 of a naturally-occurring tat protein; and poly-arginine motifs, for example, the region of amino acids 34-56 of HIV-1 rev protein, nonaarginine, octa-arginine, and the like. The site at which the fusion is made may be selected in order to optimize the biological activity, secretion or binding characteristics of the polypeptide. The optimal site can be determined by suitable methods.

### Formulations for Introducing Systems and Compositions to a Host

Described herein are formulations of introducing systems, system components and/or compositions described herein to a host. In some embodiments, such formulations, systems and compositions described herein comprise polypeptide described herein (e.g., an effector protein, effector partner such as a fusion partner, fusion protein, or combination thereof) and a carrier (e.g., excipient, diluent, vehicle, or filling agent).

In some aspects of the present disclosure the polypeptide (e.g., the effector protein, effector partner, such as a fusion partner, fusion protein, or combination thereof) is provided in a pharmaceutical composition comprising the effector protein and any pharmaceutically acceptable excipient, carrier, or diluent. In some embodiments, a pharmaceutically acceptable excipient, carrier or diluent can describe any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by well-known conventional methods (see, e.g., Remington's Pharmaceutical Sciences, 18th edition, A. Gennaro, ed., Mack Publishing Co., Easton, Pa., 1990; and Remington, The Science and Practice of Pharmacy 21st Ed. Mack Publishing, 2005).

In some embodiments, a pharmaceutically acceptable excipient, carrier or diluent, comprises any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by well-known conventional methods (see, e.g., Remington's Pharmaceutical Sciences, 18th edition, A. Gennaro, ed., Mack Publishing Co., Easton, Pa., 1990; and Remington, The Science and Practice of Pharmacy 21st Ed. Mack Publishing, 2005).

### X. Methods of Nucleic Acid Modifying

Provided herein are compositions, methods, and systems for modifying (e.g., editing) target nucleic acids. When modifying a target nucleic acid, a change in the physical composition of a target nucleic acid is described. In general, editing refers to modifying the nucleotide sequence of a target nucleic acid. However, compositions, methods, and systems disclosed herein may also be capable of modifying target nucleic acids, such as making epigenetic modifications of target nucleic acids, which does not change the nucleotide sequence of the target nucleic acids per se. Polypeptides (e.g., effector proteins, effector partners such as fusion partners, fusion proteins, or combinations thereof), multimeric complexes thereof, compositions and systems described herein may be used for modifying a target nucleic acid, which includes editing a target nucleic acid sequence. Modifying a target nucleic acid may comprise one or more of cleaving the target nucleic acid, deleting one or more nucleotides of the target nucleic acid, inserting one or more nucleotides into the target nucleic acid, mutating one or more nucleotides of the target nucleic acid, or otherwise changing one or more nucleotides of the target nucleic acid (e.g., methylating, demethylating, deaminating, or oxidizing) of one or more nucleotides of the target nucleic acid. In some embodiments, the target nucleic acid is selected from the group consisting of B2M gene, TRAC gene and CIITA gene. In some embodiments, the methods described herein edit the target nucleic acid without resulting in translocation or chromosomal rearrangements in a cell. Alternatively, in some embodiments, the methods described herein edit the target nucleic acid with fewer translocations or chromosomal rearrangements relative to editing of the target nucleic acid by Cas9 effector protein. In some embodiments, the methods comprise editing of more than one target sequences by the compositions or systems described herein, wherein the more than one target sequences are edited simultaneously or sequentially. In some embodiments, the more than one target sequences are within the same target nucleic acid. Alternatively, in some embodiments, at least two of the more than on target sequences are in different target nucleic acids.

The target nucleic acid may be a gene or a portion thereof. Methods, systems and compositions may modify a coding portion of a gene, a non-coding portion of a gene, or a combination thereof. Modifying at least one gene using the compositions, systems and methods described herein may reduce or increase expression of one or more genes. In some embodiments, compositions, systems and methods reduce expression of one or more genes by at least 10%, at least 20%, at least 30%, at least 40%, at least 50%, at least 60%, at least 70%, at least 80%, at least 90%, or at least 95%. In some embodiments, compositions, systems and methods described herein reduce expression of a gene wherein the gene is a mutant gene. In some embodiments, compositions, systems and methods remove all expression of a gene, also referred to as genetic knock out. In some embodiments, compositions, systems and methods increase expression of one or more genes by at least 10%, at least 20%, at least 30%, at least 40%, at least 50%, at least 60%, at least 70%, at least 80%, at least 90%, or at least 100%.

Modifying a target nucleic acid using the compositions, systems and methods described herein may reduce or increase expression of an allele of a gene. In some embodiments, compositions, systems and methods increase expression of an allele of a gene by at least 10%, at least 20%, at least 30%, at least 40%, at least 50%, at least 60%, at least 70%, at least 80%, at least 90%, at least 95% or more. In some embodiments, compositions, systems and methods reduce expression of an allele of a gene by at least 10%, at least 20%, at least 30%, at least 40%, at least 50%, at least 60%, at least 70%, at least 80%, at least 90%, at least 95% or more.

In some embodiments, compositions, systems and methods reduce expression of an allele of a gene by at least 10%, at least 20%, at least 30%, at least 40%, at least 50%, at least 60%, at least 70%, at least 80%, at least 90%, at least 95% or more relative to expression of a second allele of a gene, wherein the second allele is a WT allele or an allele that has not been contacted by a system or composition described herein.

In some embodiments, compositions, systems and methods reduce expression of an allele of a KRAS gene by at least 10%, at least 20%, at least 30%, at least 40%, at least 50%, at least 60%, at least 70%, at least 80%, at least 90%, at least 95% or more relative to expression of a second allele of a KRAS gene. In some embodiments, compositions, systems and methods reduce expression of a mutant allele of a KRAS gene by at least 10%, at least 20%, at least 30%, at least 40%, at least 50%, at least 60%, at least 70%, at least 80%, at least 90%, at least 95% or more relative to expression of a WT allele of a KRAS gene or an allele that has not been contacted by a system or composition described herein.

In some embodiments, compositions, systems and methods use effector proteins that are fused to a heterologous protein. Heterologous proteins include, but are not limited to, transcriptional activators, transcriptional repressors, deaminases, methyltransferases, acetyltransferases, and other nucleic acid modifying proteins. In some embodiments, effector proteins need not be fused to a partner protein to accomplish the required protein (expression) modification.

In some embodiments, compositions, systems and methods comprise a nucleic acid expression vector, or use thereof, to introduce a polypeptide (e.g., an effector protein, effector partner such as fusion partner, fusion protein, or combination thereof), guide nucleic acid, donor template or any combination thereof to a cell. In some embodiments, the nucleic acid expression vector is a viral vector. Viral vectors include, but are not limited to, retroviruses, adenoviruses, adeno-associated viruses, and herpes simplex viruses. In some embodiments, the viral vector is a replication-defective viral vector, comprising an insertion of a therapeutic gene inserted in genes essential to the lytic cycle, preventing the virus from replicating and exerting cytotoxic effects. In some embodiments, the viral vector is an adeno associated viral (AAV) vector. In some embodiments, the nucleic acid expression vector is a non-viral vector. In some embodiments, compositions and methods comprise a lipid, polymer, nanoparticle, or a combination thereof, or use thereof, to introduce a polypeptide, guide nucleic acid, donor template or any combination thereof to a cell. Non-limiting examples of lipids and polymers are cationic polymers, cationic lipids, or bio-responsive polymers. In some embodiments, the bio-responsive polymer exploits chemical-physical properties of the endosomal environment (e.g., pH) to preferentially release the genetic material in the intracellular space.

Methods of modifying may comprise contacting a target nucleic acid with one or more components, compositions or systems described herein. In some embodiments, a method of modifying comprises contacting a target nucleic acid with at least one of: a) one or more polypeptides (e.g., effector proteins, effector partners such as fusion partners, fusion proteins, or combination thereof), or one or more nucleic acids encoding the one or more polypeptides; or b) one or more guide nucleic acids, or one or more nucleic acids encoding one or more guide nucleic acids. In some embodiments, a method of modifying comprises contacting a target nucleic acid with a system described herein wherein the system comprises components comprising at least one of: a) one or more polypeptides (e.g., effector proteins, effector partners such as fusion partners, fusion proteins, or combination thereof), or one or more nucleic acids encoding the one or more polypeptides; or b) one or more guide nucleic acids, or one or more nucleic acids encoding one or more guide nucleic acids. In some embodiments, a method of modifying comprises contacting a target nucleic acid with a composition described herein comprising at least one of: a) one or more polypeptides (e.g., effector proteins, effector partners such as fusion partners, fusion proteins, or combination thereof), or one or more nucleic acids encoding the one or more polypeptides; or b) one or more guide nucleic acids, or one or more nucleic acids encoding one or more guide nucleic acids; in a composition. In some embodiments, a method of modifying as described herein produces a modified target nucleic acid. Methods of modifying (e.g., editing) may comprise contacting a target nucleic acid with an effector protein as described herein and a guide nucleic acid.

Editing a target nucleic acid sequence may introduce a mutation (e.g., point mutations, deletions) in a target nucleic acid relative to a corresponding wildtype nucleotide sequence. Editing a target nucleic acid sequence may remove or correct a disease-causing mutation in a nucleic acid sequence to produce a corresponding wildtype nucleotide sequence. Editing a target nucleic acid sequence may remove/correct point mutations, deletions, null mutations, or tissue-specific mutations in a target nucleic acid. Editing a target nucleic acid sequence may be used to generate gene knock-out, gene knock-in, gene editing, gene tagging, or a combination thereof. Methods of the disclosure may be targeted to any locus in a genome of a cell.

Modifying a target nucleic acid sequence may comprise single stranded cleavage, double stranded cleavage, donor nucleic acid insertion, epigenetic modification (e.g., methylation, demethylation, acetylation, or deacetylation), or a combination thereof. In some embodiments, cleavage (single-stranded or double-stranded) is site-specific, meaning cleavage occurs at a specific site in the target nucleic acid, often within the region of the target nucleic acid that hybridizes with the guide nucleic acid spacer region. In some embodiments, the effector proteins introduce a single-stranded break in a target nucleic acid to produce a cleaved nucleic acid. In some embodiments, the polypeptides (e.g., effector proteins, effector partners such as fusion partners, fusion proteins, or combination thereof) is capable of introducing a break in a single stranded RNA (ssRNA). The polypeptide (e.g., effector protein, effector partner such as fusion partners, fusion protein, or combination thereof) may be coupled to a guide nucleic acid that targets a particular region of interest in the ssRNA. In some embodiments, the target nucleic acid, and the resulting cleaved nucleic acid is contacted with a nucleic acid for homologous recombination (e.g., homology directed repair (HDR)) or non-homologous end joining (NHEJ). In some embodiments, a double-stranded break in the target nucleic acid may be repaired (e.g., by NHEJ or HDR) without insertion of a donor template, such that the repair results in an indel in the target nucleic acid at or near the site of the double-stranded break. In some embodiments, an indel, sometimes referred to as an insertion-deletion or indel mutation, is a type of genetic mutation that results from the insertion and/or deletion of one or more nucleotide in a target nucleic acid. An indel can vary in length (e.g., 1 to 1,000 nucleotides in length) and be detected using methods well known in the art, including sequencing. If the number of nucleotides in the insertion/deletion is not divisible by three, and it occurs in a protein coding region, it is also a frameshift mutation. Indel percentage is the percentage of sequencing reads that show at least one nucleotide has been mutation that results from the insertion and/or deletion of nucleotides regardless of the size of insertion or deletion, or number of nucleotides mutated. For example, if there is at least one nucleotide deletion detected in a given target nucleic acid, it counts towards the percent indel value. As another example, if one copy of the target nucleic acid has one nucleotide deleted, and another copy of the target nucleic acid has 10 nucleotides deleted, they are counted the same. This number reflects the percentage of target nucleic acids that are modified by a given polypeptide (e.g., effector protein, effector partner such as fusion partners, fusion protein, or combination thereof).

In some embodiments, methods of modifying described herein cleave a target nucleic acid at one or more locations to generate a cleaved target nucleic acid. In some embodiments, the cleaved target nucleic acid undergoes recombination (e.g., NHEJ or HDR). In some embodiments, cleavage in the target nucleic acid may be repaired (e.g., by NHEJ or HDR) without insertion of a donor nucleic acid, such that the repair results in an indel in the target nucleic acid at or near the site of the cleavage site. In some embodiments, cleavage in the target nucleic acid may be repaired (e.g., by NHEJ or HDR) with insertion of a donor nucleic acid, such that the repair results in an indel in the target nucleic acid at or near the site of the cleavage site.

In some embodiments, wherein the compositions, systems, and methods of the present disclosure comprise an additional guide nucleic acid or a use thereof, the dual-guided compositions, systems, and methods described herein can modify the target nucleic acid in two locations. In some embodiments, dual-guided editing can comprise cleavage of the target nucleic acid in the two locations targeted by the guide nucleic acids. In certain embodiments, upon removal of the sequence between the guide nucleic acids, the wild-type reading frame is restored. A wild-type reading frame can be a reading frame that produces at least a partially, or fully, functional protein. A non-wild-type reading frame can be a reading frame that produces a non-functional or partially non-functional protein. In some embodiments, when describing a functional protein reference is made to a protein that retains at least some if not all activity relative to the wildtype protein. A functional protein can also include a protein having enhanced activity relative to the wildtype protein. Assays are known and available for detecting and quantifying protein activity, e.g., colorimetric and fluorescent assays. In some instances, a functional protein is a wildtype protein. In some instances, a functional protein is a functional portion of a wildtype protein.

Accordingly, in some embodiments, compositions, systems, and methods described herein can edit 1 to 1,000 nucleotides or any integer in between, in a target nucleic acid. In certain embodiments, 1 to 1,000, 2 to 900, 3 to 800, 4 to 700, 5 to 600, 6 to 500, 7 to 400, 8 to 300, 9 to 200, or 10 to 100 nucleotides, or any integer in between, can be edited by the compositions, systems, and methods described herein. In some embodiments, 1, 2, 3, 4, 5, 6, 7, 8, 9, 10, or more nucleotides can be edited by the compositions, systems, and methods described herein. In some embodiments, 10, 20, 30, 40, 50, 60, 70, 80 90, 100 or more nucleotides, or any integer in between, can be edited by the compositions, systems, and methods described herein. In some embodiments, 100, 200, 300, 400, 500, 600, 700, 800, 900 or more nucleotides, or any integer in between, can be edited by the compositions, systems, and methods described herein.

In some embodiments, the polypeptide (e.g., effector protein, effector partner such as a fusion partner, fusion protein, or combination thereof) is fused to a chromatin-modifying enzyme. In some embodiments, the fusion protein chemically modifies the target nucleic acid, for example by methylating, demethylating, or acetylating the target nucleic acid in a sequence specific or non-specific manner.

Methods may comprise use of two or more polypeptides (e.g., effector proteins, effector partners such as fusion partners, fusion proteins, or combination thereof). An illustrative method for introducing a break in a target nucleic acid comprises contacting the target nucleic acid with: (a) a first engineered guide nucleic acid comprising a region that binds to a first effector protein; and (b) a second engineered guide nucleic acid comprising a region that binds to a second effector protein, wherein the first engineered guide nucleic acid comprises an additional region that binds to the target nucleic acid and wherein the second engineered guide nucleic acid comprises an additional region that binds to the target nucleic acid. In some embodiments, the first and second effector protein are identical. In some embodiments, the first and second effector protein are not identical.

In some embodiments, editing a target nucleic acid comprises genome editing. Genome editing may comprise modifying a genome, chromosome, plasmid, or other genetic material of a cell or organism. In some embodiments, the genome, chromosome, plasmid, or other genetic material of the cell or organism is modified in vivo. In some embodiments, the genome, chromosome, plasmid, or other genetic material of the cell or organism is modified in a cell. In some embodiments, the genome, chromosome, plasmid, or other genetic material of the cell or organism is modified in vitro. For example, a plasmid may be modified in vitro using a composition described herein and introduced into a cell or organism. In some embodiments, modifying a target nucleic acid may comprise deleting a sequence from a target nucleic acid. For example, a mutated sequence or a sequence associated with a disease may be removed from a target nucleic acid. In some embodiments, modifying a target nucleic acid may comprise replacing a sequence in a target nucleic acid with a second sequence. For example, a mutated sequence or a sequence associated with a disease may be replaced with a second sequence lacking the mutation or that is not associated with the disease. In some embodiments, editing a target nucleic acid may comprise deleting or replacing a sequence comprising markers associated with a disease or disorder. In some embodiments, modifying a target nucleic acid may comprise introducing a sequence into a target nucleic acid. For example, a beneficial sequence or a sequence that may reduce or eliminate a disease may be inserted into the target nucleic acid.

In some embodiments, methods comprise inserting a donor nucleic acid into a cleaved target nucleic acid. The donor nucleic acid may be inserted at a specified (e.g., effector protein targeted) point within the target nucleic acid. In some embodiments, the cleaved target nucleic acid is cleaved at a single location. In some embodiments, methods comprise contacting a target nucleic acid with an effector protein, thereby introducing a single-stranded break in the target nucleic acid; contacting the target nucleic acid with a second effector protein, to generate a second cleavage site in the target nucleic acid, ligating the regions flanking the first and second cleavage site, optionally through NHEJ or single-strand annealing, thereby resulting in the excision of a portion of the target nucleic acid between the first and second cleavage sites from the target nucleic acid; and contacting the target nucleic acid with a donor nucleic acid for homologous recombination, optionally via HDR or NHEJ, thereby introducing a new sequence into the target nucleic acid (e.g., at a cleavage site or in between two cleavage sites).

In some embodiments, the cleaved target nucleic acid is cleaved at two locations. In some embodiments, methods comprise contacting a target nucleic acid with an effector protein, thereby introducing a single-stranded break in the target nucleic acid; contacting the target nucleic acid with a second effector protein, to generate a second cleavage site in the target nucleic acid, ligating the regions flanking the first and second cleavage site, optionally through NHEJ or single-strand annealing, thereby resulting in the excision of a portion of the target nucleic acid between the first and second cleavage sites from the target nucleic acid; and contacting the target nucleic acid with a donor nucleic acid for homologous recombination, optionally via HDR or NHEJ, thereby introducing a new sequence into the target nucleic acid (e.g., at a cleavage site or in between two cleavage sites).

In some embodiments, methods comprise editing a target nucleic acid with two or more polypeptides (e.g., effector proteins, effector partners such as fusion partners, fusion proteins, or combinations thereof). Editing a target nucleic acid may comprise introducing a two or more single-stranded breaks in a target nucleic acid. In some embodiments, a break may be introduced by contacting a target nucleic acid with a polypeptide (e.g., an effector protein, effector partner such as a fusion partner, fusion protein, or combination thereof) and a guide nucleic acid. The guide nucleic acid may bind to the polypeptide (e.g., effector protein, effector partner such as a fusion partner, fusion protein, or combination thereof) and hybridize to a region of the target nucleic acid, thereby recruiting the polypeptide to the region of the target nucleic acid. Binding of the polypeptide (e.g., effector protein, effector partner such as a fusion partner, fusion protein, or combination thereof) to the guide nucleic acid and the region of the target nucleic acid may activate the polypeptide, and the polypeptide may introduce a break (e.g., a single stranded break) in the region of the target nucleic acid. In some embodiments, modifying a target nucleic acid may comprise introducing a first break in a first region of the target nucleic acid and a second break in a second region of the target nucleic acid. For example, modifying a target nucleic acid may comprise contacting a target nucleic acid with a first guide nucleic acid that binds to a first effector protein and hybridizes to a first region of the target nucleic acid and a second guide nucleic acid that binds to a second effector protein or programmable nickase and hybridizes to a second region of the target nucleic acid. The first effector protein may introduce a first break in a first strand at the first region of the target nucleic acid, and the second effector protein may introduce a second break in a second strand at the second region of the target nucleic acid. In some embodiments, a segment of the target nucleic acid between the first break and the second break may be removed, thereby modifying the target nucleic acid. In some embodiments, a segment of the target nucleic acid between the first break and the second break may be replaced (e.g., with donor nucleic acid), thereby modifying the target nucleic acid.

In some embodiments, editing is achieved by fusing an effector protein to a heterologous sequence. The heterologous sequence may be a suitable fusion partner, e.g., a protein that provides recombinase activity by acting on the target nucleic acid. In some embodiments, the fusion protein comprises an effector protein fused to a heterologous sequence by a linker. The heterologous sequence or fusion partner may be a base editing domain. The base editing domain may be an ADAR1/2 or any functional variant thereof. The heterologous sequence or fusion partner may be fused to the C-terminus, N-terminus, or an internal portion (e.g., a portion other than the N- or C-terminus) of the effector protein.

Methods, systems and compositions described herein can edit or modify a target nucleic acid wherein such editing or modification can effect one or more indels. In some embodiments, where compositions, systems, and/or methods described herein effect one or more indels, then in certain embodiments, the impact on the transcription and/or translation of the target nucleic acid can be predicted depending on: 1) the amount of indels generated; and 2) the location of the indel on the target nucleic acid. For example, as described herein, in certain embodiments, if the amount of indels is not divisible by three, and the indels occur within or along a protein coding region, then the modification or mutation can be a frameshift mutation.

In certain embodiments, if the amount of indels is divisible by three, then a frameshift mutation may not be effected, but a splicing disruption mutation and/or sequence skip mutation may be effected, such as an exon skip mutation. In certain embodiments, if the amount of indels is not evenly divisible by three, then a frameshift mutation may be effected.

Methods, systems and compositions described herein can edit or modify a target nucleic acid wherein such editing or modification can be measured by indel activity. Indel activity measures the amount of change in a target nucleic acid (e.g., nucleotide deletion(s) and/or insertion(s)) compared to a target nucleic acid that has not been contacted by a polypeptide described in compositions, systems, and methods described herein. For example, indel activity can be detected by next generation sequencing of one or more target loci of a target nucleic acid where indel percentage is calculated as the fraction of sequencing reads containing insertions or deletions relative to an unedited reference sequence. In certain instances, methods, systems, and compositions comprising a polypeptide (e.g., effector protein, effector partner such as a fusion partner, fusion protein, or combination thereof) and guide nucleic acid described herein can exhibit about 0.0001% to about 65% or more indel activity upon contact to a target nucleic acid compared to a target nucleic acid non-contacted with compositions, systems, or by methods described herein. For example, methods, systems, and compositions comprising a polypeptide (e.g., effector protein, effector partner such as a fusion partner, fusion protein, or combination thereof) and guide nucleic acid described herein can exhibit about 0.0001%, about 0.001%, about 0.01%, about 0.1%, about 1%, about 5%, about 10%, about 15%, about 20%, about 25%, about 30%, about 35%, about 40%, about 45%, about 50%, about 55%, about 60%, about 65% or more indel activity.

In some embodiments, editing or modifications of a target nucleic acid as described herein effects one or more mutations comprising splicing disruption mutations, frameshift mutations (e.g., 1+ or 2+ frameshift mutation), sequence deletion, sequence skipping, sequence refraining, sequence knock-in, or any combination thereof.

A splicing disruption can be a modification that disrupts the splicing of a target nucleic acid or splicing of a sequence that is transcribed from a target nucleic acid relative to a target nucleic acid without the splicing disruption.

A frameshift mutation can be a modification that alters the reading frame of a target nucleic acid relative to a target nucleic acid without the frameshift mutation. In certain embodiments, a frameshift mutation can be a +2 frameshift mutation wherein a reading frame is modified by 2 bases. In certain embodiments, a frameshift mutation can be a +1 frameshift mutation wherein a reading frame is modified by 1 base. In certain embodiments, a frameshift mutation is a modification that alters the number of bases in a target nucleic acid so that it is not divisible by three. In some embodiments, a frameshift mutation can be a modification that is not a splicing disruption.

A sequence as described in reference to a sequence deletion, sequence skipping, sequence refraining, and sequence knock-in can be a DNA sequence, a RNA sequence, a modified DNA or RNA sequence, a mutated sequence, a wild-type sequence, a coding sequence, a non-coding sequence, an exonic sequence (exon), an intronic sequence (intron), or any combination thereof. Such a sequence can be a sequence that is associated with a disease as described herein, such as cancer, or a genetic disease (e.g., sickle cell disease, sickle cell anemia, and/or β-thalassemia).

In certain embodiments, sequence deletion is a modification where one or more sequences in a target nucleic acid are deleted relative to a target nucleic acid without the sequence deletion. In certain embodiments, a sequence deletion can result in or effect a splicing disruption or a frameshift mutation. In certain embodiments, a sequence deletion result in or effect a splicing disruption.

In certain embodiments, sequence skipping is a modification where one or more sequences in a target nucleic acid are skipped upon transcription or translation of the target nucleic acid relative to a target nucleic acid without the sequence skipping. In certain embodiments, sequence skipping can result in or effect a splicing disruption or a frameshift mutation. In certain embodiments, sequence skipping can result in or effect a splicing disruption.

In certain embodiments, sequence reframing is a modification where one or more bases in a target are modified so that the reading frame of the sequence is reframed relative to a target nucleic acid without the sequence reframing. In certain embodiments, sequence reframing can result in or effect a splicing disruption or a frameshift mutation. In certain embodiments, sequence reframing can result in or effect a frameshift mutation.

In certain embodiments, sequence knock-in is a modification where one or more sequences is inserted into a target nucleic acid relative to a target nucleic acid without the sequence knock-in. In certain embodiments, sequence knock-in can result in or effect a splicing disruption or a frameshift mutation. In certain embodiments, sequence knock-in can result in or effect a splicing disruption.

In certain embodiments, editing or modification of a target nucleic acid can be locus specific, wherein compositions, systems, and methods described herein can edit or modify a target nucleic acid at one or more specific loci to effect one or more specific mutations comprising splicing disruption mutations, frameshift mutations, sequence deletion, sequence skipping, sequence reframing, sequence knock-in, or any combination thereof. For example, editing or modification of a specific locus can effect any one of a splicing disruption, frameshift (e.g., 1+ or 2+ frameshift), sequence deletion, sequence skipping, sequence reframing, sequence knock-in, or any combination thereof. In certain embodiments, editing or modification of a target nucleic acid can be locus specific, modification specific, or both. In certain embodiments, editing or modification of a target nucleic acid can be locus specific, modification specific, or both, wherein compositions, systems, and methods described herein comprise a polypeptide (e.g., effector protein, effector partner such as a fusion partner, fusion protein, or combination thereof) described herein and a guide nucleic acid described herein.

Methods of editing a target nucleic acid or modulating the expression of a target nucleic acid may be performed in vivo. Methods of editing a target nucleic acid or modulating the expression of a target nucleic acid may be performed in vitro. For example, a plasmid may be modified in vitro using a composition described herein and introduced into a cell or organism. Methods of editing a target nucleic acid or modulating the expression of a target nucleic acid may be performed ex vivo. For example, methods may comprise obtaining a cell from a subject, modifying a target nucleic acid in the cell with methods described herein, and returning the cell to the subject.

In some embodiments, methods of modifying described herein comprise contacting a target nucleic acid with one or more components, compositions or systems described herein. In some embodiments, the one or more components, compositions or systems described herein comprise at least one of: a) one or more polypeptides (e.g., effector proteins, effector partners such as fusion partners, fusion proteins, or combination thereof), or one or more nucleic acids encoding the one or more polypeptides; and b) one or more guide nucleic acids, or one or more nucleic acids encoding the one or more guide nucleic acids. In some embodiments, the one or more effector proteins introduce a single-stranded break or a double-stranded break in the target nucleic acid. In some embodiments, methods of modifying described herein produce a modified target nucleic acid comprising an engineered nucleic acid sequence that expresses polypeptide having new activity as compared to an unmodified target nucleic acid, or alters expression of an endogenous polypeptide as compared to an unmodified target nucleic acid.

In some embodiments, methods of modifying described herein comprise using one or more guide nucleic acids or uses thereof, wherein the methods modify a target nucleic acid at a single location. In some embodiments, the methods comprise contacting an RNP comprising polypeptides (e.g., effector proteins, effector partners such as fusion partners, fusion proteins, or combination thereof) and a guide nucleic acid to the target nucleic acid. In some embodiments, the methods introduce a mutation (e.g., point mutations, deletions) in the target nucleic acid relative to a corresponding wildtype nucleotide sequence. In some embodiments, the methods remove or correct a disease-causing mutation in a nucleic acid sequence to produce a corresponding wildtype nucleotide sequence. In some embodiments, the methods remove/correct point mutations, deletions, null mutations, or tissue-specific mutations in a target nucleic acid. In some embodiments, the methods introduce a single stranded cleavage, a nick, a deletion of one or two nucleotides, an insertion of one or two nucleotides, a substitution of one or two nucleotides, an epigenetic modification (e.g., methylation, demethylation, acetylation, or deacetylation), or a combination thereof to the target nucleic acid. In some embodiments, the methods comprise using an effector protein and two guide nucleic acids, wherein two RNPs cleave the target nucleic acid at the same location, wherein a first RNP comprises the effector protein and a first guide nucleic acid, and wherein a second RNP comprises the effector protein and a second guide nucleic acid. In some embodiments, methods comprising using two effector protein and two guide nucleic acids, wherein both RNPs cleave the target nucleic acid at the same location, wherein a first RNP comprises a first effector protein and a first target nucleic acid, and wherein a second RNP comprises a second effector protein and a second target nucleic acid.

In some embodiments, methods of modifying described herein comprise using one or more guide nucleic acids or uses thereof, wherein the methods modify a target nucleic acid at two different locations. In some embodiments, the methods introduce two cleavage sites in the target nucleic acid, wherein a first cleavage site and a second cleavage site comprise one or more nucleotides therebetween. In some embodiments, the methods cause deletion of the one or more nucleotides. In some embodiments, the deletion restores a wild-type reading frame. In some embodiments, the wild-type reading frame produces at least a partially functional protein. In some embodiments, the deletion causes a non-wild-type reading frame. In some embodiments, a non-wild-type reading frame produces a partially functional protein or non-functional protein. In some embodiments, the at least partially functional protein has at least 50%, at least 60%, at least 70%, at least 80%, at least 90%, at least 100%, at least 110%, at least 120%, at least 130%, at least 140%, at least 150%, at least 180%, at least 200%, at least 300%, at least 400% activity compared to a corresponding wildtype protein. In some embodiments, the methods comprise using an effector protein and two guide nucleic acids, wherein two RNPs cleave the target nucleic acid at different locations, wherein a first RNP comprises the effector protein and a first guide nucleic acid, and wherein a second RNP comprises the effector protein and a second guide nucleic acid. In some embodiments, methods comprising using two effector protein and two guide nucleic acids, wherein both RNPs cleave the target nucleic acid at the same location, wherein a first RNP comprises a first effector protein and a first target nucleic acid, and wherein a second RNP comprises a second effector protein and a second target nucleic acid.

In some embodiments, methods of editing described herein comprise inserting a donor nucleic acid into a cleaved target nucleic acid. In some embodiments, the cleaved target nucleic acid formed by introducing a single-stranded break into a target nucleic acid. The donor nucleic acid may be inserted at a specified (e.g., effector protein targeted) point within the target nucleic acid. In some embodiments, the cleaved target nucleic acid is cleaved at a single location. In such embodiments, the methods comprise contacting a target nucleic acid with an effector protein described herein, thereby introducing a single-stranded break in the target nucleic acid; and contacting the target nucleic acid with a donor nucleic acid for homologous recombination, optionally by HDR or NHEJ, thereby introducing a new sequence into the target nucleic acid (e.g., at a cleavage site). In some embodiments, the cleaved target nucleic acid is cleaved at two locations. In such embodiments, the methods comprise contacting a target nucleic acid with an effector protein described herein, thereby introducing a single-stranded break in the target nucleic acid; contacting the target nucleic acid with a second effector protein described herein, to generate a second cleavage site in the target nucleic acid, ligating the regions flanking the first and second cleavage site, optionally through NHEJ or single-strand annealing, thereby resulting in the excision of a portion of the target nucleic acid between the first and second cleavage sites from the target nucleic acid; and contacting the target nucleic acid with a donor nucleic acid for homologous recombination, optionally by HDR or NHEJ, thereby introducing a new sequence into the target nucleic acid (e.g., in between two cleavage sites).

### Donor Nucleic Acids

In some embodiments, a donor nucleic acid comprises a nucleic acid that is incorporated into a target nucleic acid or genome. A donor nucleic acid may comprise one or more donor nucleotides. In some embodiments, a donor nucleic acid comprises a sequence that is derived from a plant, bacteria, fungi, virus, or an animal. In some embodiments, the animal is a non-human animal, such as, by way of non-limiting example, a mouse, rat, hamster, rabbit, pig, bovine, deer, sheep, goat, chicken, cat, dog, ferret, a bird, non-human primate (e.g., marmoset, rhesus monkey). In some embodiments, the non-human animal is a domesticated mammal or an agricultural mammal. In some embodiments, the animal is a human. In some embodiments, the sequence comprises a human wild-type (WT) gene or a portion thereof. In some embodiments, the human WT gene or the portion thereof comprises a nucleotide sequence that is at least 70%, at least 80%, at least 90%, at least 92%, at least 95%, at least 97%, at least 99%, or 100% identical to an equal length portion of the WT sequence of any one of the sequences recited in TABLE 6. In some embodiments, the donor nucleic acid is incorporated into an insertion site of a target nucleic acid.

In some embodiments, the donor nucleic acid comprises single-stranded DNA or linear double-stranded DNA. In some embodiments, the donor nucleic acid comprises a nucleotide sequence encoding a functional polypeptide and/or wherein the donor nucleic acid comprises a wildtype sequence. In some embodiments, the donor nucleic acid comprises a protein coding sequence, a gene, a gene fragment, an exon, an intron, an exon fragment, an intron fragment, a gene regulatory fragment, a gene regulatory region fragment, coding sequences thereof, or combinations thereof. In some embodiments, the donor nucleic acid comprises a naturally occurring sequence. In some embodiments, the naturally occurring sequence does not contain a mutation.

In some embodiments, the donor nucleic acid comprises a gene fragment, an exon fragment, an intron fragment, a gene regulatory region fragment, or combinations thereof. In some embodiments, the fragment is at least 10, at least 20, at least 30, at least 40, at least 50, at least 60, at least 70, or at least 80 contiguous nucleotides.

Donor nucleic acids of any suitable size may be integrated into a target nucleic acid or genome. In some embodiments, the donor polynucleotide integrated into a genome is less than 3, about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 kilobases in length. In some embodiments, donor nucleic acids are more than 500 kilobases (kb) in length.

In reference to a viral vector, the term, “donor nucleic acid,” refers to a sequence of nucleotides that will be or has been introduced into a cell following transfection of the viral vector. The donor nucleic acid may be introduced into the cell by any mechanism of the transfecting viral vector, including, but not limited to, integration into the genome of the cell or introduction of an episomal plasmid or viral genome.

As another example, when used in reference to the activity of an effector protein, the term, “donor nucleic acid,” refers to a sequence of nucleotides that will be or has been inserted at the site of cleavage by the effector protein (cleaving (hydrolysis of a phosphodiester bond) of a nucleic acid resulting in a nick or double strand break-nuclease activity).

As yet another example, when used in reference to homologous recombination, the term, “donor nucleic acid,” refers to a sequence of DNA that serves as a template in the process of homologous recombination, which may carry the alteration that is to be or has been introduced into the target nucleic acid. By using this donor nucleic acid as a template, the genetic information, including the modification, is copied into the target nucleic acid by way of homologous recombination.

The donor nucleic acid may comprise a sequence that is derived from a plant, bacteria, virus or an animal. The animal may be human. The animal may be a non-human animal, such as, by way of non-limiting example, a mouse, rat, hamster, rabbit, pig, bovine, deer, sheep, goat, chicken, cat, dog, ferret, a bird, non-human primate (e.g., marmoset, rhesus monkey). The non-human animal may be a domesticated mammal or an agricultural mammal.

### Genetically Modified Cells and Organisms

Methods of editing described herein may be employed to generate a genetically modified cell. The cell may be a eukaryotic cell (e.g., a mammalian cell) or a prokaryotic cell (e.g., an archaeal cell). The cell may be derived from a multicellular organism and cultured as a unicellular entity. The cell may comprise a heritable genetic modification, such that progeny cells derived therefrom comprise the heritable genetic mutation. The cell may be progeny of a genetically modified cell comprising a genetic modification of the genetically modified parent cell. A genetically modified cell may comprise a deletion, insertion, mutation, or non-native sequence relative to a wild-type version of the cell or the organism from which the cell was derived.

Methods of the disclosure may be performed in a cell. A cell may be in vitro. A cell may be in vivo. A cell may be ex vivo. A cell may be in vitro. A cell may be an isolated cell. A cell may be a cell inside of an organism. A cell may be an organism. A cell may be a cell in a cell culture. A cell may be one of a collection of cells. A cell may be a mammalian cell or derived from a mammalian cell. A cell may be a rodent cell or derived from a rodent cell. A cell may be a human cell or derived from a human cell. In some embodiments, the human cell is a T cell. A cell may be a eukaryotic cell or derived from a eukaryotic cell. A cell may be a pluripotent stem cell. A cell may be a plant cell or derived from a plant cell. A cell may be an animal cell or derived from an animal cell. A cell may be an invertebrate cell or derived from an invertebrate cell. A cell may be a vertebrate cell or derived from a vertebrate cell. A cell may be a progenitor cell or derived therefrom.

A cell may be from a specific organ or tissue. In some embodiments, the cell is a hepatocyte. In some embodiments, the tissue is a subject's blood, bone marrow, or cord blood. In some embodiments, the tissue is a heterologous donor blood, cord blood, or bone marrow. In some embodiments, the tissue is an allogenic blood, cord blood, or bone marrow. In some embodiments, the tissue is spleen, lymph, or pancreatic tissue. The tissue may be muscle. The muscle may be skeletal muscle. In some embodiments, the cell is a myocyte. In some embodiments, the cell is a muscle cell. In some embodiments, the muscle cell is a skeletal muscle cell. In some embodiments, the skeletal muscle cell is a red (slow) skeletal muscle cell, a white (fast) skeletal muscle cell or an intermediate skeletal muscle cell.

The tissue may be the subject's blood, bone marrow, or cord blood. The tissue may be heterologous donor blood, cord blood, or bone marrow. The tissue may be allogenic blood, cord blood, or bone marrow. In some embodiments, the cell is a: a stem cell, muscle satellite cell, muscle stem cell, myoblast, muscle progenitor cell, a pluripotent stem cell or a cell derived from a pluripotent stem cell.

Methods may comprise contacting a cell with a nucleic acid (e.g., a plasmid or mRNA) comprising a nucleotide sequence encoding a polypeptide (e.g., an effector protein, effector partner such as a fusion partner, fusion protein, or combination thereof). Methods may comprise contacting cells with a nucleic acid (e.g., a plasmid or mRNA) comprising a nucleotide sequence encoding a guide nucleic acid, or a crRNA. In some embodiments, the methods comprise contacting the cells with a nucleic acid encoding the effector protein at a dose of 3 μg to 10 μg (e.g., 3 μg, 6 μg, 9 μg or 10 μg), and a nucleic acid encoding the guide nucleic acid at a dose of 500 μM. Contacting may comprise electroporation, acoustic poration, optoporation, viral vector-based delivery, iTOP, nanoparticle delivery (e.g., lipid or gold nanoparticle delivery), cell-penetrating peptide (CPP) delivery, DNA nanostructure delivery, or any combination thereof. Methods may comprise contacting a cell with a polypeptide (e.g., an effector protein, effector partner such as a fusion partner, fusion protein, or combination thereof) or a multimeric complex thereof.

Methods of the disclosure may be performed in a subject. Compositions or systems of the disclosure may be administered to a subject. A subject may be a human. A subject may be a mammal (e.g., rat, mouse, cow, dog, pig, sheep, horse). A subject may be a vertebrate or an invertebrate. A subject may be a laboratory animal. A subject may be a patient. A subject may be at risk of developing, suffering from, or displaying symptoms a disease or disorder as set forth in herein. The subject may have a mutation associated with a gene described herein. The subject may display symptoms associated with a mutation of a gene described herein. In some embodiments, a mutation comprises a point mutation or single nucleotide polymorphism (SNP), a chromosomal mutation, a copy number mutation, or any combination thereof. A point mutation optionally comprises a substitution, insertion, or deletion. In some embodiments, a mutation comprises a chromosomal mutation. A chromosomal mutation can comprise an inversion, a deletion, a duplication, or a translocation. In some embodiments, a mutation comprises a copy number variation. A copy number variation can comprise a gene amplification or an expanding trinucleotide repeat. In some embodiments, mutations may be as described herein.

### XI. Methods of Detecting a Target Nucleic Acid

Provided herein are methods of detecting target nucleic acids. Methods may comprise detecting target nucleic acids with compositions or systems described herein. Methods may comprise detecting a target nucleic acid in a sample, e.g., a cell lysate, a biological fluid, or environmental sample. Methods may comprise detecting a target nucleic acid in a cell. In some embodiments, methods of detecting a target nucleic acid in a sample or cell comprises contacting the sample or cell with an effector protein or a multimeric complex thereof, a guide nucleic acid, wherein at least a portion of the guide nucleic acid is complementary to at least a portion of the target nucleic acid, and a reporter nucleic acid that is cleaved in the presence of the effector protein, the guide nucleic acid, and the target nucleic acid, and detecting a signal produced by cleavage of the reporter nucleic acid, thereby detecting the target nucleic acid in the sample. In some embodiments, methods result in trans cleavage of the reporter nucleic acid. In some embodiments, methods result in cis cleavage of the reporter nucleic acid. In some embodiments, methods of detecting a target nucleic acid include a reporter nucleic acid comprising a detectable moiety that produces a detectable signal in the presence of the target nucleic acid, the effector protein, and the guide nucleic acid.

In some embodiments, methods of detecting comprise contacting a target nucleic acid, a cell comprising the target nucleic acid, or a sample comprising a target nucleic acid with a polypeptide (e.g., an effector protein, effector partner such as a fusion partner, fusion protein, or combination thereof). In some embodiments, methods of detecting comprise contacting a target nucleic acid, a cell comprising the target nucleic acid, or a sample comprising a target nucleic acid with polypeptide (e.g., an effector protein, effector partner such as a fusion partner, fusion protein, or combination thereof) and an engineered guide nucleic acid. In some embodiments, the methods of detecting a target nucleic acid comprising: a) contacting the target nucleic acid with a composition comprising an effector protein as described herein, a guide nucleic acid as described herein, and a reporter nucleic acid that is cleaved in the presence of the effector protein, the guide nucleic acid, and the target nucleic acid; and b) detecting a signal produced by cleavage of the reporter nucleic acid, thereby detecting the target nucleic acid in the sample. In some embodiments, the methods result in trans cleavage of the reporter nucleic acid. In some embodiments, the methods result in cis cleavage of the reporter nucleic acid. In some embodiments, the reporter nucleic acid is a single stranded nucleic acid. In some embodiments, the reporter comprises a detection moiety. In some embodiments, the reporter nucleic acid is capable of being cleaved by the effector protein. In some embodiments, a cleaved reporter nucleic acid generates a detectable product or a first detectable signal. In some embodiments, the first detectable signal is a change in color. In some embodiments, the change is color is measured indicating presence of the target nucleic acid. In some embodiments, the first detectable signal is measured on a support medium.

Methods may comprise contacting the sample to a complex comprising a guide nucleic acid comprising a segment that is reverse complementary to a segment of the target nucleic acid and an effector protein that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample.

Methods may comprise contacting the sample comprising the target nucleic acid with a guide nucleic acid targeting a target nucleic acid segment, an effector protein capable of being activated when complexed with the guide nucleic acid and the target nucleic acid segment, a single stranded nucleic acid of a reporter comprising a detection moiety, wherein the nucleic acid of a reporter is capable of being cleaved by the activated effector protein, thereby generating a first detectable signal, cleaving the single stranded nucleic acid of a reporter using the effector protein that cleaves as measured by a change in color, and measuring the first detectable signal on the support medium.

Methods may comprise contacting the sample or cell with an effector protein or a multimeric complex thereof and a guide nucleic acid at a temperature of at least about 25° C., at least about 30° C., at least about 35° C., at least about 37° C., at least about 40° C., at least about 50° C., at least about 60° C., at least about 65° C., at least about 70° C., or at least about 75° C. In some embodiments, the temperature is not greater than 80° C. In some embodiments, the temperature is about 25° C., about 30° C., about 35° C., at least about 37° C., about 40° C., about 45° C., about 50° C., about 55° C., about 60° C., about 65° C., about 70° C., about 75° C., about 80° C., about 85° C. In some embodiments, the temperature is about 25° C. to about 45° C., about 35° C. to about 55° C., about 37° C. to about 60° C., or about 55° C. to about 65° C. In some embodiments, the temperature is about 37° C. to about 45° C., about 37° C. to about 50° C., about 37° C. to about 55° C., about 37° C. to about 60° C., or about 37° C. to about 65° C.

Methods may comprise contacting the sample or cell with an effector protein or a multimeric complex thereof and a guide nucleic acid in the presence of salts (e.g., compositions comprising salts). In some embodiments, the method may comprise a solution, wherein the solution comprises one or more salt. Accordingly, in some embodiments, the salt may be one or more salt selected from a magnesium salt, a zinc salt, a potassium salt, a calcium salt, and a sodium salt. In some embodiments, the salt is a combination of two or more salts. For example, in some embodiments, the salt is a combination of two or more salts selected from a magnesium salt, a zinc salt, a potassium salt, a calcium salt and a sodium salt. In some embodiments, the salt is magnesium acetate. In some embodiments, the salt is magnesium chloride. In some embodiments, the salt is potassium acetate. In some embodiments, the salt is potassium nitrate. In some embodiments, the salt is zinc chloride. In embodiments, the salt is sodium chloride. In some embodiments, the salt is potassium chloride. In some embodiments, the concentration of the one or more salt in the solution is about 0.001 mM to about 500 mM. In some embodiments, the concentration of the salt is about 0.001 mM to about 400 mM. In some embodiments, the concentration of the salt is about 0.001 mM to about 300 mM. In some embodiments, the concentration of the salt is about 0.001 mM to about 200 mM. In some embodiments, the concentration of the salt is about 0.001 mM to about 100 mM. In some embodiments, the concentration of the salt is about 0.001 mM to about 10 mM. In some embodiments, the concentration of the salt is about 0.01 mM to about 500 mM. In some embodiments, the concentration of the salt is about 0.01 mM to about 400 mM. In some embodiments, the concentration of the salt is about 0.01 mM to about 300 mM. In some embodiments, the concentration of the salt is about 0.01 mM to about 200 mM. In some embodiments, the concentration of the salt is about 0.01 mM to about 100 mM. In some embodiments, the concentration of the salt is about 0.01 mM to about 10 mM. In some embodiments, the concentration of the salt is about 0.1 mM to about 500 mM. In some embodiments, the concentration of the salt is about 0.1 mM to about 400 mM. In some embodiments, the concentration of the salt is about 0.1 mM to about 300 mM. In some embodiments, the concentration of the salt is about 0.1 mM to about 200 mM. In some embodiments, the concentration of the salt is about 0.1 mM to about 100 mM. In some embodiments, the concentration of the salt is about 0.1 mM to about 10 mM. In some embodiments, the concentration of the salt is about 1 mM to about 500 mM. In some embodiments, the concentration of the salt is about 1 mM to about 400 mM. In some embodiments, the concentration of the salt is about 1 mM to about 300 mM. In some embodiments, the concentration of the salt is about 1 mM to about 200 mM. In some embodiments, the concentration of the salt is about 1 mM to about 100 mM. In some embodiments, the concentration of the salt is about 1 mM to about 10 mM. In some embodiments, the concentration of the salt is about 10 mM to about 500 mM. In some embodiments, the concentration of the salt is about 10 mM to about 400 mM. In some embodiments, the concentration of the salt is about 10 mM to about 300 mM. In some embodiments, the concentration of the salt is about 10 mM to about 200 mM. In some embodiments, the concentration of the salt is about 10 mM to about 100 mM. In some embodiments, the concentration of the salt is about 100 mM to about 500 mM. In some embodiments, the concentration of the salt is about 100 mM to about 400 mM. In some embodiments, the concentration of the salt is about 100 mM to about 300 mM. In some embodiments, the concentration of the salt is about 100 mM to about 200 mM. In some embodiments, the salt is potassium acetate and the concentration of salt in the solution is about 100 mM. In some embodiments, the salt is potassium acetate or sodium chloride and the concentration of salt in the solution is about 200 mM. In some embodiments, the salt is potassium acetate or sodium chloride and the salt of potassium in the solution is about 100 mM to about 200 mM.

In some embodiments, methods of detecting a target nucleic acid by a cleavage assay. In some embodiments, the target nucleic acid is a single-stranded target nucleic acid. In some embodiments, the cleavage assay comprises: a) contacting the target nucleic acid with a composition comprising an effector protein as described; and b) cleaving the target nucleic acid. In some embodiments, the cleavage assay comprises an assay designed to visualize, quantitate or identify cleavage of a nucleic acid. In some embodiments, the method is an in vitro trans cleavage assay. In some embodiments, a cleavage activity is a trans cleavage activity. In some embodiments, the method is an in vitro cis cleavage assay. In some embodiments, a cleavage activity is a cis cleavage activity. In some embodiments, the cleavage assay follows a procedure comprising: (i) providing a composition comprising an equimolar amounts of an effector protein as described herein, and a guide nucleic acid described herein, under conditions to form an RNP complex; (ii) adding a plasmid comprising a target nucleic acid, wherein the target nucleic acid is a linear dsDNA, wherein the target nucleic acid comprises a target sequence and a PAM (iii) incubating the mixture under conditions to enable cleavage of the plasmid; (iv) quenching the reaction with EDTA and a protease; and (v) analyzing the reaction products (e.g., viewing the cleaved and uncleaved linear dsDNA with gel electrophoresis).

Methods may comprise cleaving a strand of a single-stranded target nucleic acid with an effector protein or a multimeric complex thereof, as assessed with an in vitro cis-cleavage assay. A cleavage assay can comprise an assay designed to visualize, quantitate or identify cleavage of a nucleic acid. In some cases, the cleavage activity may be cis-cleavage activity. In some cases, the cleavage activity may be trans-cleavage activity. An example of such an assay (an in vitro cis-cleavage assay). An example of such an assay may follow a procedure comprising: (i) providing equimolar amounts of an effector protein and a guide nucleic acid under conditions to form a ribonucleoprotein complex; (ii) adding linear dsDNA comprising a nucleic acid sequence targeted by the guide nucleic acid and adjacent to a PAM (iii) incubating the mixture under conditions to enable cleavage of the plasmid; (iv) quenching the reaction with EDTA and a protease; and (v) analyzing the reaction products (e.g., viewing the cleaved and uncleaved linear dsDNA with gel electrophoresis).

Methods may comprise cleaving a strand of a single-stranded target nucleic acid with an effector protein or a multimeric complex thereof, as assessed with an in vitro cis-cleavage assay. A cleavage assay can comprise an assay designed to visualize, quantitate or identify cleavage of a nucleic acid. In some cases, the cleavage activity may be cis-cleavage activity. In some cases, the cleavage activity may be trans-cleavage activity. An example of such an assay (an in vitro cis-cleavage assay). An example of such an assay may follow a procedure comprising: (i) providing equimolar amounts of an effector protein and a guide nucleic acid under conditions to form a ribonucleoprotein complex; (ii) adding linear dsDNA comprising a nucleic acid sequence targeted by the guide nucleic acid and adjacent to a PAM (iii) incubating the mixture under conditions to enable cleavage of the plasmid; (iv) quenching the reaction with EDTA and a protease; and (v) analyzing the reaction products (e.g., viewing the cleaved and uncleaved linear dsDNA with gel electrophoresis).

In some cases, there is a threshold of detection for methods of detecting target nucleic acids. In some instances, methods are not capable of detecting target nucleic acids that are present in a sample or solution at a concentration less than or equal to 10 nM. The term, “threshold of detection,” is used herein to describe the minimal amount of target nucleic acid that must be present in a sample in order for detection to occur. For example, when a threshold of detection is 10 nM, then a signal can be detected when a target nucleic acid is present in the sample at a concentration of 10 nM or more. In some cases, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 μM, 1 μM, 500 fM, 250 fM, 100 fM, 50 fM, 10 fM, 5 fM, 1 fM, 500 attomole (aM), 100 aM, 50 aM, 10 aM, or 1 aM. In some cases, the threshold of detection is in a range of from 1 aM to 1 nM, 1 aM to 500 μM, 1 aM to 200 μM, 1 aM to 100 μM, 1 aM to 10 μM, 1 aM to 1 μM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 μM, 10 aM to 200 μM, 10 aM to 100 μM, 10 aM to 10 μM, 10 aM to 1 μM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 μM, 100 μM to 200 μM, 100 aM to 100 μM, 100 aM to 10 μM, 100 aM to 1 μM, 100 aM to 500 fM, 100 aM to 100 fM, 100 aM to 1 fM, 100 aM to 500 aM, 500 aM to 1 nM, 500 aM to 500 μM, 500 aM to 200 μM, 500 aM to 100 μM, 500 aM to 10 μM, 500 aM to 1 μM, 500 aM to 500 fM, 500 aM to 100 fM, 500 aM to 1 fM, 1 fM to 1 nM, 1 fM to 500 μM, 1 fM to 200 μM, 1 fM to 100 μM, 1 fM to 10 μM, 1 fM to 1 μM, 10 fM to 1 nM, 10 fM to 500 μM, 10 fM to 200 μM, 10 fM to 100 μM, 10 fM to 10 μM, 10 fM to 1 μM, 500 fM to 1 nM, 500 fM to 500 μM, 500 fM to 200 μM, 500 fM to 100 μM, 500 fM to 10 μM, 500 fM to 1 μM, 800 fM to 1 nM, 800 fM to 500 μM, 800 fM to 200 μM, 800 fM to 100 μM, 800 fM to 10 μM, 800 fM to 1 μM, 1 μM to 1 nM, 1 μM to 500 μM, 1 μM to 200 μM, 1 μM to 100 μM, or 1 μM to 10 μM. In some cases, the threshold of detection in a range of from 800 fM to 100 μM, 1 μM to 10 μM, 10 fM to 500 fM, 10 fM to 50 fM, 50 fM to 100 fM, 100 fM to 250 fM, or 250 fM to 500 fM. In some cases, the threshold of detection is in a range of from 2 aM to 100 μM, from 20 aM to 50 μM, from 50 aM to 20 μM, from 200 aM to 5 μM, or from 500 aM to 2 μM. In some cases, the minimum concentration at which a target nucleic acid is detected in a sample is in a range of from 1 aM to 1 nM, 10 aM to 1 nM, 100 aM to 1 nM, 500 aM to 1 nM, 1 fM to 1 nM, 1 fM to 500 μM, 1 fM to 200 μM, 1 fM to 100 μM, 1 fM to 10 μM, 1 fM to 1 μM, 10 fM to 1 nM, 10 fM to 500 μM, 10 fM to 200 μM, 10 fM to 100 μM, 10 fM to 10 μM, 10 fM to 1 μM, 500 fM to 1 nM, 500 fM to 500 μM, 500 fM to 200 μM, 500 fM to 100 μM, 500 fM to 10 μM, 500 fM to 1 μM, 800 fM to 1 nM, 800 fM to 500 μM, 800 fM to 200 μM, 800 fM to 100 μM, 800 fM to 10 μM, 800 fM to 1 μM, 1 μM to 1 nM, 1 μM to 500 μM, from 1 μM to 200 μM, 1 μM to 100 μM, or 1 μM to 10 μM. In some cases, the minimum concentration at which a target nucleic acid is detected in a sample is in a range of from 2 aM to 100 μM, from 20 aM to 50 μM, from 50 aM to 20 μM, from 200 aM to 5 μM, or from 500 aM to 2 μM. In some cases, the minimum concentration at which a single stranded target nucleic acid can be detected in a sample is in a range of from 1 aM to 100 μM. In some cases, the minimum concentration at which a target nucleic acid can be detected in a sample is in a range of from 1 fM to 100 μM. In some cases, the minimum concentration at which a single stranded target nucleic acid can be detected in a sample is in a range of from 10 fM to 100 μM. In some cases, the minimum concentration at which a single stranded target nucleic acid can be detected in a sample is in a range of from 800 fM to 100 μM. In some cases, the minimum concentration at which a single stranded target nucleic acid can be detected in a sample is in a range of from 1 μM to 10 μM. In some cases, the devices, systems, fluidic devices, kits, and methods described herein detect a target single-stranded nucleic acid in a sample comprising a plurality of nucleic acids such as a plurality of non-target nucleic acids, where the target single-stranded nucleic acid is present at a concentration as low as 1 aM, 10 aM, 100 aM, 500 aM, 1 fM, 10 fM, 500 fM, 800 fM, 1 μM, 10 μM, 100 μM, or 1 μM.

In some instances, the target nucleic acid is present in a cleavage reaction at a concentration of about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 10 μM, or about 100 μM. In some instances, the target nucleic acid is present in the cleavage reaction at a concentration of from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 10 μM, from 10 μM to 100 μM, from 10 nM to 100 nM, from 10 nM to 1 μM, from 10 nM to 10 μM, from 10 nM to 100 μM, from 100 nM to 1 μM, from 100 nM to 10 μM, from 100 nM to 100 μM, or from 1 μM to 100 μM. In some instances, the target nucleic acid is present in the cleavage reaction at a concentration of from 20 nM to 50 μM, from 50 nM to 20 μM, or from 200 nM to 5 μM.

In some cases, methods detect a target nucleic acid in less than 60 minutes. In some cases, methods detect a target nucleic acid in less than about 120 minutes, less than about 110 minutes, less than about 100 minutes, less than about 90 minutes, less than about 80 minutes, less than about 70 minutes, less than about 60 minutes, less than about 55 minutes, less than about 50 minutes, less than about 45 minutes, less than about 40 minutes, less than about 35 minutes, less than about 30 minutes, less than about 25 minutes, less than about 20 minutes, less than about 15 minutes, less than about 10 minutes, less than about 5 minutes, less than about 4 minutes, less than about 3 minutes, less than about 2 minutes, or less than about 1 minute.

In some cases, methods require at least about 120 minutes, at least about 110 minutes, at least about 100 minutes, at least about 90 minutes, at least about 80 minutes, at least about 70 minutes, at least about 60 minutes, at least about 55 minutes, at least about 50 minutes, at least about 45 minutes, at least about 40 minutes, at least about 35 minutes, at least about 30 minutes, at least about 25 minutes, at least about 20 minutes, at least about 15 minutes, at least about 10 minutes, or at least about 5 minutes to detect a target nucleic acid. In some cases, the sample is contacted with the reagents for from 5 minutes to 120 minutes, from 5 minutes to 100 minutes, from 10 minutes to 90 minutes, from 15 minutes to 45 minutes, or from 20 minutes to 35 minutes.

In some cases, methods of detecting are performed in less than 10 hours, less than 9 hours, less than 8 hours, less than 7 hours, less than 6 hours, less than 5 hours, less than 4 hours, less than 3 hours, less than 2 hours, less than 1 hour, less than 50 minutes, less than 45 minutes, less than 40 minutes, less than 35 minutes, less than 30 minutes, less than 25 minutes, less than 20 minutes, less than 15 minutes, less than 10 minutes, less than 9 minutes, less than 8 minutes, less than 7 minutes, less than 6 minutes, or less than 5 minutes. In some cases, methods of detecting are performed in about 5 minutes to about 10 hours, about 10 minutes to about 8 hours, about 15 minutes to about 6 hours, about 20 minutes to about 5 hours, about 30 minutes to about 2 hours, or about 45 minutes to about 1 hour.

Methods may comprise detecting a detectable signal within 5 minutes of contacting the sample and/or the target nucleic acid with the guide nucleic acid and/or the effector protein. In some cases, detecting occurs within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the target nucleic acid. In some instances, detecting occurs within 1 to 120, 5 to 100, 10 to 90, 15 to 80, 20 to 60, or 30 to 45 minutes of contacting the target nucleic acid.

In some embodiments, methods of detecting as disclosed herein are compatible with methods for diagnosis of a disease or disorder.

### Amplification of a Target Nucleic Acid

Methods of detecting may comprise amplifying a target nucleic acid for detection using any of the compositions or systems described herein. Amplifying may comprise changing the temperature of the amplification reaction, also known as thermal amplification (e.g., PCR). Amplifying may be performed at essentially one temperature, also known as isothermal amplification. Amplifying may improve at least one of sensitivity, specificity, or accuracy of the detection of the target nucleic acid.

Amplifying may comprise subjecting a target nucleic acid to an amplification reaction selected from transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA), or any of the amplification methods described herein.

In some instances, amplification of the target nucleic acid comprises modifying the sequence of the target nucleic acid. For example, amplification may be used to insert a PAM sequence into a target nucleic acid that lacks a PAM sequence. In some cases, amplification may be used to increase the homogeneity of a target nucleic acid in a sample. For example, amplification may be used to remove a nucleic acid variation that is not of interest in the target nucleic acid.

Amplifying may take 1, 2, 3, 4, 5, 6, 7, 8, 9, 10, 11, 12, 13, 14, 15, 16, 17, 18, 19, 20, 25, 30, 40, 50, or 60 minutes. Amplifying may be performed at a temperature of around 20-45° C. Amplifying may be performed at a temperature of less than about 20° C., less than about 25° C., less than about 30° C., less than about 35° C., less than about 37° C., less than about 40° C., or less than about 45° C. The nucleic acid amplification reaction may be performed at a temperature of at least about 20° C., at least about 25° C., at least about 30° C., at least about 35° C., at least about 37° C., at least about 40° C., or at least about 45° C.

### Detection of a Target Nucleic Acid

Described herein are various methods of sample amplification and detection in a single reaction volume. In some embodiments, methods include simultaneous amplification and detection in the same volume and/or in the same reaction. In some embodiments, methods include sequential amplification and detection in the same volume. In some embodiments, amplification and detection may occur in a single reaction, where reverse transcription, amplification, in vitro transcription, or any combination thereof, and detection are carried out in a single volume. Any suitable method of reverse transcription, amplification, in vitro transcription, and detection can be used in such a reaction, such as methods of reverse transcription, amplification, in vitro transcription, and detection described herein.

In some embodiments, a DETECTR reaction may be used to detect the presence of a specific target gene in the same. The DETECTR reaction may produce a detectable signal, as described elsewhere herein, in the presence of a target nucleic acid sequence comprising a target gene. The DETECTR reaction may not produce a signal in the absence of the target nucleic acid or in the presence of a nucleic acid sequence that does not comprise the specific mutation or comprises a different mutation. In some embodiments the mutation is a SNP. In some embodiments, a DETECTR reaction may comprise a guide RNA reverse complementary to a portion of a target nucleic acid sequence comprising a specific SNP. The guide RNA and the target nucleic acid comprising the specific SNP may bind to and activate a effector protein, thereby producing a detectable signal as described elsewhere herein. The guide RNA and a nucleic acid sequence that does not comprise the specific SNP may not bind to or activate the effector protein and may not produce a detectable signal. In some embodiments, a target nucleic acid sequence that may or may not comprise a specific SNP may be amplified using any amplification method disclosed herein. In some embodiments, the amplification reaction may be combined with a reverse transcription reaction, a DETECTR reaction, or both. In some embodiments, the target nucleic acid sequence can comprise a SNP. In some embodiments, the target nucleic acid sequence can comprise a sequence indicative of a human disease.

A DETECTR reaction, as described elsewhere herein, may produce a detectable signal specifically in the presence of a target nucleic acid sequence comprising a target gene. In some embodiments, the target nucleic acid sequence can comprise a sequence indicative of a human disease. In some embodiments, the detectable signal produced in the DETECTR reaction may be higher in the presence of a target nucleic acid comprising target nucleic acid than in the presence of a nucleic acid that does not comprise the target nucleic acid. In some embodiments, the DETECTR reaction may produce a detectable signal that is at least 1-fold, at least 2-fold, at least 3-fold, at least 4-fold, at least 5-fold, at least 10-fold, at least 20-fold, at least 30-fold, at least 40-fold, at least 50-fold, at least 100-fold, at least 200-fold, at least 300-fold, at last 400-fold, at least 500-fold, at least 1000-fold, at least 2000-fold, at least 3000-fold, at least 4000-fold, at least 5000-fold, at least 6000-fold, at least 7000-fold, at least 8000-fold, at least 9000-fold, at least 10000-fold, at least 50000-fold, at least 100000-fold, at least 500000-fold, or at least 1000000-fold greater in the presence of a target nucleic acid comprising a target nucleic acid than in the presence of a nucleic acid that does not comprise the target nucleic acid. In some embodiments, the DETECTR reaction may produce a detectable signal that is from 1-fold to 2-fold, from 2-fold to 3-fold, from 3-fold to 4-fold, from 4-fold to 5-fold, from 5-fold to 10-fold, from 10-fold to 20-fold, from 20-fold to 30-fold, from 30-fold to 40-fold, from 40-fold to 50-fold, from 50-fold to 100-fold, from 100-fold to 500-fold, from 500-fold to 1000-fold, from 1000-fold to 10,000-fold, from 10,000-fold to 100,000-fold, or from 100,000-fold to 1,000,000-fold greater in the presence of a target nucleic acid comprising a specific mutation or SNP than in the presence of a nucleic acid that does not comprise the specific mutation or SNP. In some embodiments, the target nucleic acid sequence can comprise a SNP. In some embodiments, the target nucleic acid sequence can comprise a sequence indicative of a human disease.

A DETECTR reaction may be used to detect the presence of a target nucleic acid associated with a disease or a condition in a nucleic acid sample. The DETECTR reaction may reach signal saturation within about 30 seconds, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 75 minutes, about 80 minutes, or about 85 minutes and be used to detect the presence of a target gene associated with an increased likelihood of developing a disease or a condition in a nucleic acid sample. The DETECTR reaction may be used to detect the presence of a target gene associated with a phenotype in a nucleic acid sample. For example, a DETECTR reaction may be used to detect a target nucleic acid, such as a gene or exon, or a mutation of a target nucleic acid, such as a SNP, as set forth in TABLE 6. In another example, a DETECTR reaction may be used to detect target nucleic acid or a mutation of a target nucleic acid associated with any one of the diseases or disorders recited in TABLE 6.1. A DETECTR reaction may be used to detect a SNP associated with a phenotype, for example, eye color, hair color, height, skin color, race, alcohol flush reaction, caffeine consumption, deep sleep, genetic weight, lactose intolerance, muscle composition, saturated fat and weight, or sleep movement. A DETECTR reaction may also be used to detect the presence of a pathological organism. In some embodiments, the pathological organism is a prokaryote, eukaryote, or a protozoa. In some embodiments, the pathological organism is a virus, an opportunistic pathogen, a parasite, a bacterium, or any combination thereof. In some embodiments, the pathological organism is SARS-CoV-2 or Streptococcus pyogenes.

### XII. Method of Treating a Disease or Disorder

Described herein are compositions, systems and methods for treating a disease in a subject by modifying a target nucleic acid associated with a gene or expression of a gene related to the disease. In some embodiments, described herein are methods for treating a disease in a subject by contacting a target nucleic acid with a composition or system described herein, wherein the target nucleic acid is associated with a gene or expression of a gene related to the disease. In some embodiments, methods comprise treating, preventing, or inhibiting a disease or disorder associated with a mutation or aberrant expression of a gene. In some embodiments, methods for treating a disease or disorder comprise methods of editing a nucleic acid described herein.

Methods may comprise administration of a composition(s) or component(s) of a system described herein. In some embodiments, the composition(s) or component(s) of the system comprises use of a recombinant nucleic acid (DNA or RNA), administered for the purpose to edit a nucleic acid. In some embodiments, the composition or component of the system comprises use of a vector to introduce a functional gene or transgene. In some embodiments, vectors comprise nonviral vectors, including cationic polymers, cationic lipids, or bio-responsive polymers. In some embodiments, the bio-responsive polymer exploits chemical-physical properties of the endosomal environment (e.g., pH) to preferentially release the genetic material in the intracellular space. In some embodiments, vectors comprise viral vectors, including retroviruses, adenoviruses, adeno-associated viruses, and herpes simplex viruses. In some embodiments, the vector comprises a replication-defective viral vector, comprising an insertion of a therapeutic gene inserted in genes essential to the lytic cycle, preventing the virus from replicating and exerting cytotoxic effects. By way of non-limiting example, the composition(s) may comprise pharmaceutical compositions described herein. Methods of gene therapy that are applicable to the compositions and systems described herein are described in more detail in Ingusci et al., “Gene Therapy Tools for Brain Diseases”, Front. Pharmacol. 10:724 (2019), which is hereby incorporated by reference in its entirety.

In some embodiments, treating, preventing, or inhibiting disease or disorder in a subject may comprise contacting a target nucleic acid associated with a particular ailment with a composition described herein. In some aspects, the methods of treating, preventing, or inhibiting a disease or disorder may involve removing, editing, modifying, replacing, transposing, or affecting the regulation of a genomic sequence of a patient in need thereof. In some embodiments, the methods of treating, preventing, or inhibiting a disease or disorder may involve modulating gene expression.

In some embodiments, the compositions and systems described herein are for use in therapy. In some embodiments, the compositions and systems described herein are for use in treating a disease or condition described herein. Also provided is the use of the compositions described herein in the manufacture of a medicament. Also provided is the use of the compositions described herein in the manufacture of a medicament for therapeutic and/or prophylactic treatment of a disease or condition described herein.

In some embodiments, the polypeptides (e.g., effector proteins, effector partners such as fusion partners, fusion proteins, or combination thereof) described herein are for use in therapy. In some embodiments, the polypeptides described herein are for use in treating a disease or condition described herein. Also provided is the use of the polypeptides described herein in the manufacture of a medicament. Also provided is the use of the polypeptides described herein in the manufacture of a medicament for therapeutic and/or prophylactic treatment of a disease or condition described herein.

In some embodiments, the guide nucleic acids described herein are for use in therapy. In some embodiments, the guide nucleic acids described herein are for use in treating a disease or condition described herein. Also provided is the use of the guide nucleic acids described herein in the manufacture of a medicament. Also provided is the use of the guide nucleic acids described herein in the manufacture of a medicament for therapeutic and/or prophylactic treatment of a disease or condition described herein.

Described herein are compositions, systems and methods for treating a disease in a subject by editing a target nucleic acid associated with a gene or expression of a gene related to the disease. For example, in some embodiments, the editing comprises knock-out of a gene comprising the target nucleic acid. In some embodiments, the compositions, systems and methods comprise LNPs, wherein the LNPs comprise the effector proteins described herein or nucleic acids encoding the effector proteins, the effector partners (e.g., fusion partners) described herein or nucleic acids encoding the effector partners (e.g., fusion partners), the fusion proteins described herein or nucleic acids encoding the fusion proteins, or combinations thereof. In some embodiments, the LNPs comprise chemically modified guide nucleic acids. In some embodiments, the LNPs described herein are used for delivering the compositions, or one or more components of the systems described herein to a specific organ (e.g., liver). Alternatively, in some embodiments, the compositions, systems and methods comprise AAV particles, wherein the AAV particles comprise nucleic acids encoding the effector proteins described herein, the effector partners (e.g., fusion partners) described herein, the fusion proteins described herein, or combinations thereof. In some embodiments, the AAV particles comprise nucleic acids encoding guide nucleic acids described herein. In some embodiments, the AAV particles described herein are used for delivering the compositions, or one or more components of the systems described herein to a specific cells (e.g., nerve cells or muscle cells).

In some embodiments, methods comprise administering a composition or cell described herein to a subject. By way of non-limiting example, the disease may be a cancer, an ophthalmological disorder, a neurological disorder, a neurodegenerative disease, a blood disorder, or a metabolic disorder, or a combination thereof. The disease may be an inherited disorder, also referred to as a genetic disorder. The disease may be the result of an infection or associated with an infection. Also by way of non-limiting example, the compositions are pharmaceutical compositions described herein.

The compositions and methods described herein may be used to treat, prevent, or inhibit a disease or syndrome in a subject. In some embodiments, the disease is a liver disease, a lung disease, an eye disease, or a muscle disease. Exemplary diseases and syndromes include, but are not limited to the disease and syndromes listed in TABLE 6.1.

In some embodiments, compositions and methods modify at least one gene associated with the disease or the expression thereof. In some embodiments, the disease is Alzheimer's disease and the gene is selected from APP, BACE-1, PSD95, MAPT, PSEN1, PSEN2, and APOEE4. In some embodiments, the disease is congenital muscular dystrophy 1A (MDC1A) and the gene is LAMA1 or LAMA2. In some embodiments, the disease is Ullrich Congenital Muscular Dystrophy (UCMD) and the gene is selected from COL6A1, COL6A2 and COL6A3. In some embodiments, the disease is Limb Girdle Muscular Dystrophies (LGMD1B, LGMD2A, LGMD2B) and the gene is selected from LMNA, DYSF, and CAPN3. In some embodiments, the disease is Nemaline Myopathy and the gene is selected from ACTA1, NEB, TPM2, TPM3, TNNT1, TNNT3, TNNI2 and LMOD3. In some embodiments, the disease is Parkinson's disease and the gene is selected from SNCA, GDNF, and LRRK2. In some embodiments, the disease comprises Centronuclear myopathy and the gene is DNM2. In some embodiments, the disease is Huntington's disease and the gene is HTT. In some embodiments, the disease is Alpha-1 antitrypsin deficiency (AATD) and the gene is SERPINA1. In some embodiments, the disease is amyotrophic lateral sclerosis (ALS) and the gene is selected from SOD1, FUS, C90RF72, ATXN2, TARDBP, and CHCHD10. In some embodiments, the disease comprises Alexander Disease and the gene is GFAP. In some embodiments, the disease comprises anaplastic large cell lymphoma and the gene is CD30. In some embodiments, the disease comprises Angelman Syndrome and the gene is UBE3A. In some embodiments, the disease comprises calcific aortic stenosis and the gene is Apo(a). In some embodiments, the disease comprises CD3Z-associated primary T-cell immunodeficiency and the gene is CD3Z or CD247. In some embodiments, the disease comprises CD18 deficiency and the gene is ITGB2. In some embodiments, the disease comprises CD40L deficiency and the gene is CD40L. In some embodiments, the disease is congenital adrenal hyperplasia and the gene is CAH1. In some embodiments, the disease comprises CNS trauma and the gene is VEGF. In some embodiments, the disease comprises coronary heart disease and the gene is selected from FGA, FGB, and FGG. In some embodiments, the disease comprises MECP2 Duplication syndrome and Rett syndrome and the gene is MECP2. In some embodiments, the disease comprises a bleeding disorder (coagulation) and the gene is FXI. In some embodiments, the disease comprises fragile X syndrome and the gene is FMR1. In some embodiments, the disease comprises Fuchs corneal dystrophy and the gene is selected from ZEB1, SLC4A11, and LOXHD1. In some embodiments, the disease comprises GM2-Gangliosidoses (e.g., Tay Sachs Disease, Sandhoff disease) and the gene is selected from HEXA and HEXB. In some embodiments, the disease comprises Hearing loss disorders and the gene is DFNA36. In some embodiments, the disease is Pompe disease, including infantile onset Pompe disease (IOPD) and late onset Pompe disease (LOPD) and the gene is GAA. In some embodiments, the disease is Retinitis pigmentosa and the gene is selected from PDE6B, RHO, RP1, RP2, RPGR, PRPH2, IMPDH1, PRPF31, CRB1, PRPF8, TULP1, CA4, HPRPF3, ABCA4, EYS, CERKL, FSCN2, TOPORS, SNRNP200, PRCD, NR2E3, MERTK, USH2A, PROM1, KLHL7, CNGB1, TTC8, ARL6, DHDDS, BEST1, LRAT, SPARA7, CRX, CLRN1, RPE65, and WDR19. In some embodiments, the disease comprises Leber Congenital Amaurosis Type 10 and the gene is CEP290. In some embodiments, the disease is cardiovascular disease and/or lipodystrophies and the gene is selected from ABCG5, ABCG8, AGT, ANGPTL3, APOCIII, APOA1, APOL1, ARH, CDKN2B, CFB, CXCL12, FXI, FXII, GATA-4, MIA3, MKL2, MTHFD1L, MYH7, NKX2-5, NOTCH1, PKK, PCSK9, PSRC1, SMAD3, and TTR. In some embodiments, the disease is cardiovascular disease and/or lipodystrophies and the gene is ANGPTL3. In some embodiments, the disease is cardiovascular disease and/or lipodystrophies and the gene is PCSK9. In some embodiments, the disease is cardiovascular disease and/or lipodystrophies and the gene is TTR. In some embodiments, the disease is severe hypertriglyceridemia (SHTG) and the gene is APOCIII or ANGPTL4. In some embodiments, the disease comprises acromegaly and the gene is GHR. In some embodiments, the disease comprises acute myeloid leukemia and the gene is CD22. In some embodiments, the disease is diabetes and the gene is GCGR. In some embodiments, the disease is NAFLD/NASH and the gene is selected from HSD17B13, PSD3, GPAM, CIDEB, DGAT2 and PNPLA3. In some embodiments, the disease is NASH/cirrhosis and the gene is MARC1. In some embodiments, the disease is cancer and the gene is selected from STAT3, YAP1, FOXP3, AR (Prostate cancer), and IRF4 (multiple myeloma). In some embodiments, the disease is cystic fibrosis and the gene is CFTR. In some embodiments, the disease is Duchenne muscular dystrophy and the gene is DMD. In some embodiments, the disease is ornithine transcarbamylase deficiency (OTCD) and the gene is OTC. In some embodiments, the disease is congenital adrenal hyperplasia (CAH) and the gene is CYP21A2. In some embodiments, the disease is atherosclerotic cardiovascular disease (ASCVD) and the gene is LPA. In some embodiments, the disease is hepatitis B virus infection (CHB) and the gene is HBV covalently closed circular DNA (cccDNA). In some embodiments, the disease is citrullinemia type I and the gene is ASS1. In some embodiments, the disease is citrullinemia type I and the gene is SLC25A13. In some embodiments, the disease is citrullinemia type I and the gene is ASS1. In some embodiments, the disease is arginase-1 deficiency and the gene is ARG1. In some embodiments, the disease is carbamoyl phosphate synthetase I deficiency and the gene is CPS1. In some embodiments, the disease is argininosuccinic aciduria and the gene is ASL. In some embodiments, the disease comprises angioedema and the gene is PKK. In some embodiments, the disease comprises thalassemia and the gene is TMPRSS6. In some embodiments, the disease comprises achondroplasia and the gene is FGFR3. In some embodiments, the disease comprises Cri du chat syndrome and the gene is selected from CTNND2. In some embodiments, the disease comprises Alagille Syndrome and the gene is selected from JAG1 and NOTCH2. In some embodiments, the disease comprises Charcot Marie Tooth disease and the gene is selected from PMP22 and MFN2. In some embodiments, the disease comprises Crouzon syndrome and the gene is selected from FGFR2, FGFR3, and FGFR3. In some embodiments, the disease comprises Dravet Syndrome and the gene is selected from SCN1A and SCN2A. In some embodiments, the disease comprises Emery-Dreifuss syndrome and the gene is selected from EMD, LMNA, SYNE1, SYNE2, FHL1, and TMEM43. In some embodiments, the disease comprises Factor V Leiden thrombophilia and the gene is F5. In some embodiments, the disease is fabry disease and the gene is GLA. In some embodiments, the disease is facioscapulohumeral muscular dystrophy and the gene is FSHD1. In some embodiments, the disease comprises Fanconi anemia and the gene is selected from FANCA, FANCB, FANCC, FANCD1, FANCD2, FANCE, FANCF, FANCG, FANCI, FANCJ, FANCL, FANCM, FANCN, FANCP, FANCS, RAD51C, and XPF. In some embodiments, the disease comprises Familial Creutzfeld-Jakob disease and the gene is PRNP. In some embodiments, the disease comprises Familial Mediterranean Fever and the gene is MEFV. In some embodiments, the disease comprises Friedreich's ataxia and the gene is FXN. In some embodiments, the disease comprises Gaucher disease and the gene is GBA. In some embodiments, the disease comprises human papilloma virus (HPV) infection and the gene is HPV E7. In some embodiments, the disease comprises hemochromatosis and the gene is HFE, optionally comprising a C282Y mutation. In some embodiments, the disease comprises Hemophilia A and the gene is FVIII. In some embodiments, the disease is hereditary angioedema and the gene is SERPING1 or KLKB1. In some embodiments, the disease comprises histiocytosis and the gene is CD1. In some embodiments, the disease comprises immunodeficiency 17 and the gene is CD3D. In some embodiments, the disease comprises immunodeficiency 13 and the gene is CD4. In some embodiments, the disease comprises Common Variable Immunodeficiency and the gene is selected from CD19 and CD81. In some embodiments, the disease comprises Joubert syndrome and the gene is selected from INPP5E, TMEM216, AHI1, NPHP1, CEP290, TMEM67, RPGRIP1L, ARL13B, CC2D2A, OFD1, TMEM138, TCTN3, ZNF423, and AMRC9. In some embodiments, the disease comprises leukocyte adhesion deficiency and the gene is CD18. In some embodiments, the disease comprises Li-Fraumeni syndrome and the gene is TP53. In some embodiments, the disease comprises lymphoproliferative syndrome and the gene is CD27. In some embodiments, the disease comprises Lynch syndrome and the gene is selected from MSH2, MLH1, MSH6, PMS2, PMS1, TGFBR2, and MLH3. In some embodiments, the disease comprises mantle cell lymphoma and the gene is CD5. In some embodiments, the disease comprises Marfan syndrome and the gene is FBN1. In some embodiments, the disease comprises mastocytosis and the gene is CD2. In some embodiments, the disease comprises methylmalonic acidemia and the gene is selected from MMAA, MMAB, and MUT. In some embodiments, the disease is mycosis fungoides and the gene is CD7. In some embodiments, the disease is myotonic dystrophy and the gene is selected from CNBP and DMPK. In some embodiments, the disease comprises neurofibromatosis and the gene is selected from NF1, and NF2. In some embodiments, the disease comprises osteogenesis imperfecta and the gene is selected from COL1A1, COL1A2, and IFITM5. In some embodiments, the disease is non-small cell lung cancer and the gene is selected from KRAS, EGFR, ALK, METex14, BRAF V600E, ROS1, RET, and NTRK. In some embodiments, the disease comprises Peutz-Jeghers syndrome and the gene is STK11. In some embodiments, the disease comprises polycystic kidney disease and the gene is selected from PKD1 and PKD2. In some embodiments, the disease comprises Severe Combined Immune Deficiency and the gene is selected from IL7R, RAG1, and JAK3. In some embodiments, the disease comprises PRKAG2 cardiac syndrome and the gene is PRKAG2. In some embodiments, the disease comprises spinocerebellar ataxia and the gene is selected from ATXN1, ATXN2, ATXN3, PLEKHG4, SPTBN2, CACNA1A, ATXN7, ATXN8OS, ATXN10, TTBK2, PPP2R2B, KCNC3, PRKCG, ITPR1, TBP, KCND3, and FGF14. In some embodiments, the disease is thrombophilia due to antithrombin III deficiency and the gene is SERPINC1. In some embodiments the disease is spinal muscular atrophy and the gene is SMN1. In some embodiments, the disease comprises Usher Syndrome and the gene is selected from MYO7A, USH1C, CDH23, PCDH15, USH1G, USH2A, GPR98, DFNB31, and CLRN1.

In some embodiments, the disease comprises von Willebrand disease and the gene is VWF. In some embodiments, the disease comprises Waardenburg syndrome and the gene is selected from PAX3, MITF, WS2B, WS2C, SNAI2, EDNRB, EDN3, and SOX10. In some embodiments, the disease comprises Wiskott-Aldrich Syndrome and the gene is WAS. In some embodiments, the disease comprises von Hippel-Lindau disease and the gene is VHL. In some embodiments, the disease comprises Wilson disease and the gene is ATP7B. In some embodiments, the disease comprises Zellweger syndrome and the gene is selected from PEX1, PEX2, PEX3, PEX5, PEX6, PEX10, PEX12, PEX13, PEX14, PEX16, PEX19, and PEX26. In some embodiments, the disease comprises infantile myofibromatosis and the gene is CD34. In some embodiments, the disease comprises platelet glycoprotein IV deficiency and the gene is CD36. In some embodiments, the disease comprises immunodeficiency with hyper-IgM type 3 and the gene is CD40. In some embodiments, the disease comprises hemolytic uremic syndrome and the gene is CD46.

In some embodiments, the disease comprises complement hyperactivation, angiopathic thrombosis, or protein-losing enteropathy and the gene is CD55. In some embodiments, the disease comprises hemolytic anemia and the gene is CD59. In some embodiments, the disease comprises calcification of joints and arteries and the gene is CD73. In some embodiments, the disease comprises immunoglobulin alpha deficiency and the gene is CD79A. In some embodiments, the disease comprises C syndrome and the gene is CD96. In some embodiments, the disease comprises hairy cell leukemia and the gene is CD123. In some embodiments, the disease comprises histiocytic sarcoma and the gene is CD163. In some embodiments, the disease comprises autosomal dominant deafness and the gene is CD164. In some embodiments, the disease comprises immunodeficiency 25 and the gene is CD247. In some embodiments, the disease comprises methymalonic acidemia due to transcobalamin receptor defect and the gene is CD320.

### Cancer

In some embodiments, the disease is cancer. In some embodiments, the cancer is a solid cancer (i.e., a tumor). In some embodiments, the cancer is selected from a blood cell cancer, a leukemia, and a lymphoma. The cancer can be a leukemia, such as, by way of non-limiting example, acute myeloid (or myelogenous) leukemia (AML), chronic myeloid (or myelogenous) leukemia (CML), acute lymphocytic (or lymphoblastic) leukemia (ALL), and chronic lymphocytic leukemia (CLL). Non-limiting examples of cancers include: acute lymphoblastic leukemia; acute lymphoblastic lymphoma; acute lymphocytic leukemia; acute myelogenous leukemia; acute myeloid leukemia (adult/childhood); adrenocortical carcinoma; AIDS-related cancers; AIDS-related lymphoma; anal cancer; appendix cancer; astrocytoma; atypical teratoid/rhabdoid tumor; basal-cell carcinoma; bile duct cancer; extrahepatic (cholangiocarcinoma); bladder cancer; bone osteosarcoma/malignant fibrous histiocytoma; brain cancer (adult/childhood); brain tumor; cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; Ewing sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver cancer); Hodgkin's lymphoma; hypopharyngeal cancer; hypothalamic and visual pathway glioma; intraocular melanoma; islet cell carcinoma (endocrine pancreas); Kaposi Sarcoma; kidney cancer (renal cell cancer); Langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenström; male breast cancer; malignant fibrous histiocytoma of bone/osteosarcoma; medulloblastoma; medulloepithelioma; melanoma; melanoma, intraocular (eye); Merkel cell cancer; Merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-Hodgkin's lymphoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pituitary tumor, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituitary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; NUT midline carcinoma; retinoblastoma; rhabdomyosarcoma, childhood; salivary gland cancer; sarcoma, Ewing family of tumors; Sézary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sézary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor.

In some embodiments, the cancer is any one of colon cancer, rectal cancer, renal-cell carcinoma, liver cancer, bladder cancer, cancer of the kidney or ureter, lung cancer, non-small cell lung cancer, cancer of the small intestine, esophageal cancer, melanoma, bone cancer, pancreatic cancer, skin cancer, brain cancer (e.g., glioblastoma), cancer of the head or neck, melanoma, uterine cancer, ovarian cancer, breast cancer, testicular cancer, cervical cancer, stomach cancer, Hodgkin's Disease, non-Hodgkin's lymphoma, and thyroid cancer.

In some embodiments, mutations are associated with cancer or are causative of cancer. The target nucleic acid, in some embodiments, comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, a gene associated with cell cycle, or a combination thereof. Non-limiting examples of genes comprising a mutation associated with cancer are ABL, ACE, AF4/HRX, AKT-2, ALK, ALK/NPM, AML1, AML1/MTG8, APC, ATM, AXIN2, AXL, BAP1, BARD1, BCL-2, BCL-3, BCL-6, BCR/ABL, BLM, BMPR1A, BRCA1, BRCA2, BRIP1, c-MYC, CASR, CCR5, CDC73, CDH1, CDK4, CDKN1B, CDKN1C, CDKN2A, CEBPA, CHEK2, CREBBP, CTNNA1, DBL, DEK/CAN, DICER1, DIS3L2, E2A/PBX1, EGFR, ENL/HRX, EPCAM, ERG/TLS, ERBB, ERBB-2, ETS-1, EWS/FLI-1, FH, FKRP, FLCN, FMS, FOS, FPS, GATA2, GCG, GLI, GPC3, GPGSP, GREM1, HER2/neu, HOX11, HOXB13, HRAS, HST, IL-3, INT-2, JAKI, JUN, KIT, KS3, K-SAM, LBC, LCK, LMO1, LMO2, L-MYC, LYL-1, LYT-10, LYT-10/Cα1, MAS, MAX, MDM-2, MEN1, MET, MITF, MLH1, MLL, MOS, MSH1, MSH2, MSH3, MSH6, MTG8/AML1, MUTYH, MYB, MYH11/CBFB, NBN, NEU, NF1, NF2, N-MYC, NTHL1, OST, PALB2, PAX-5, PBX1/E2A, PCDC1, PDGFRA, PHOX2B, PIM-1, PMS2, POLD1, POLE, POT1, PPARG, PRAD-1, PRKAR1A, PTCH1, PTEN, RAD50, RAD51C, RAD51D, RAF, RAR/PML, RAS-H, RAS-K, RAS-N, RB1, RECQL4, REL/NRG, RET, RHOM1, RHOM2, ROS, RUNX1, SDHA, SDHAF, SDHAF2, SDHB, SDHC, SDHD, SET/CAN, SIS, SKI, SMAD4, SMARCA4, SMARCB1, SMARCE1, SRC, STK11, SUFU, TAL1, TAL2, TAN-1, TIAM1, TERC, TERT, TIMP3, TMEM127, TNF, TP53, TRAC, TSC1, TSC2, TRK, VHL, WRN, and WT1. Non-limiting examples of oncogenes are KRAS, NRAS, BRAF, MYC, CTNNB1, and EGFR. In some instances, the oncogene is a gene that encodes a cyclin dependent kinase (CDK). Non-limiting examples of CDKs are Cdk1, Cdk4, Cdk5, Cdk7, Cdk8, Cdk9, Cdk11 and CDK20. Non-limiting examples of tumor suppressor genes are TP53, RB1, and PTEN.

In some embodiments, the target nucleic acid comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, a gene associated with cell cycle, or a combination thereof, wherein the gene is a KRAS gene. In some embodiments, the target nucleic acid comprises a portion of a KRAS gene wherein the compositions, systems, and methods described herein may be used to selectively reduce the growth, reduce the viability, induce cell death or arrest the cell cycle of at least a portion of cells in a population of cells, wherein the at least a portion of cells comprises a mutant KRAS allele. In some embodiments, the target nucleic acid comprises a portion of a KRAS gene wherein the compositions, systems, and methods described herein are useful for treating cancer. In some embodiments, the cancer is pancreatic cancer, colon cancer or lung cancer.

### Infections

Described herein are compositions, systems and methods for treating an infection in a subject. Infections may be caused by a pathogen, e.g., bacteria, viruses, fungi, and parasites. Compositions, systems and methods may modify a target nucleic acid associated with the pathogen or parasite causing the infection. In some embodiments, the target nucleic acid may be in the pathogen or parasite itself or in a cell, tissue or organ of the subject that the pathogen or parasite infects. In some embodiments, the methods described herein include treating an infection caused by one or more bacterial pathogens. Non-limiting examples of bacterial pathogens include Acholeplasma laidlawii, Brucella abortus, Chlamydia psittaci, Chlamydia trachomatis, Cryptococcus neoformans, Escherichia coli, Legionella pneumophila, Lyme disease spirochetes, methicillin-resistant Staphylococcus aureus, Mycobacterium leprae, Mycobacterium tuberculosis, Mycoplasma arginini, Mycoplasma arthritidis, Mycoplasma genitalium, Mycoplasma hyorhinis, Mycoplasma orale, Mycoplasma pneumoniae, Mycoplasma salivarium, Neisseria gonorrhoeae, Neisseria meningitidis, Pneumococcus, Pseudomonas aeruginosa, sexually transmitted infection, Streptococcus agalactiae, Streptococcus pyogenes, and Treponema pallidum.

In some embodiments, methods, compositions, or systems described herein include treating an infection caused by one or more viral pathogens. Non-limiting examples of viral pathogens include adenovirus, blue tongue virus, chikungunya, coronavirus (e.g., SARS-CoV-2), cytomegalovirus, Dengue virus, Ebola, Epstein-Barr virus, feline leukemia virus, Hemophilus influenzae B, Hepatitis virus A, Hepatitis virus B, Hepatitis virus C, herpes simplex virus I, herpes simplex virus II, human papillomavirus (HPV) including HPV16 and HPV18, human serum parvo-like virus, human T-cell leukemia viruses, immunodeficiency virus (e.g., HIV), influenza virus, lymphocytic choriomeningitis virus, measles virus, mouse mammary tumor virus, mumps virus, murine leukemia virus, polio virus, rabies virus, Reovirus, respiratory syncytial virus (RSV), rubella virus, Sendai virus, simian virus 40, Sindbis virus, varicella-zoster virus, vesicular stomatitis virus, wart virus, West Nile virus, yellow fever virus, or any combination thereof.

In some embodiments, methods described herein include treating an infection caused by one or more parasites. Non-limiting examples of parasites include helminths, annelids, platyhelminthes, nematodes, and thorny-headed worms. In some embodiments, parasitic pathogens comprise, without limitation, Babesia bovis, Echinococcus granulosus, Eimeria tenella, Leishmania tropica, Mesocestoides corti, Onchocerca volvulus, Plasmodium falciparum, Plasmodium vivax, Schistosoma japonicum, Schistosoma mansoni, Schistosoma spp., Taenia hydatigena, Taenia ovis, Taenia saginata, Theileria parva, Toxoplasma gondii, Toxoplasma spp., Trichinella spiralis, Trichomonas vaginalis, Trypanosoma brucei, Trypanosoma cruzi, Trypanosoma rangeli, Trypanosoma rhodesiense, Balantidium coli, Entamoeba histolytica, Giardia spp., Isospora spp., Trichomonas spp., or any combination thereof.

### SEQUENCES AND TABLES

TABLES 1-1.3 provides illustrative amino acid alterations relative to SEQ ID NO: 1 as described herein.

TABLE 1
EXEMPLARY AMINO ACID ALTERATIONS RELATIVE TO SEQ ID NO: 1
Amino Acid Alterations

I2R; I2H; I2K; T5R; T5H; T5K; T11R; G13R; G13H; G13K; F14R; F14H; F14K; K15R; K15H;
L16R; L16H; L16K; L16F; I17R; I17H; I17K; R18H; R18K; N19R; N19H; N19K; N19V; H20R;
H20K; S21R; S21H; S21K; R22H; R22K; T23R; T23H; T23K; A24R; A24H; A24K; G25R; G25H;
G25K; L26R; L26H; L26K; L28R; L28H; L28K; K29R; K29H; N30R; N30H; N30K; E31R; E31H;
E31K; G32R; G32H; G32K; E33R; E33H; E33K; E34R; E34H; E34K; A35R; A35H; A35K; C36R;
C36H; C36K; K37R; K37H; K38R; K38H; F39R; F39H; F39K; V40R; V40H; V40K; R41H; R41K;
E42R; E42H; E42K; N43R; N43H; N43K; N43A; E44R; E44H; E44K; P46S; D48Y; C50K; C50P;
P51R; P51H; P51K; N52R; N52H; N52K; F53R; F53H; F53K; Q54R; Q54H; Q54K; G55R; G55H;
G55K; G56R; G56H; G56K; P57R; P57H; P57K; A58W; A58F; I59K; I59R; I62K; K65W; K65F;
E68P; T70E; E71K; E73W; E73F; E73P; I74W; I74F; Y75W; Y75F; S77V; S78K; S78D; L79R;
A80K; E83K; V84Y; V84K; T87G; T87D; P89T; P89K; K90E; K92E; P94E; P94Q; P94K; E95K;
P96K; I97R; K99R; K99H; K99S; E100K; E101K; W102T; W106N; W106K; L107F; S108R; S108H;
S108K; E109R; E109H; E109K; E109A; H110R; H110K; H110T; G111R; G111H; G111K; L112R;
L112H; L112K; L112E; D113R; D113H; D113K; T114R; T114H; T114K; V115R; V115H; V115K;
P116R; P116H; P116K; P116G; Y117R; Y117H; Y117K; K118R; K118H; E119R; E119H; E119K;
E119S; A120R; A120H; A120K; A121R; A121H; A121K; A121Q; G122R; G122H; G122K; L123R;
L123H; L123K; N124R; N124H; N124K; L125R; L125H; L125K; I126R; I126H; I126K; I127R;
I127H; I127K; K128R; K128H; N129R; N129H; N129K; A130R; A130H; A130K; V131R; V131H;
V131K; N132R; N132H; N132K; T133R; T133H; T133K; Y134R; Y134H; Y134K; K135R; K135H;
G136R; G136H; G136K; V137R; V137H; V137K; Q138R; Q138H; Q138K; V139R; V139H; V139K;
K146N; N147K; N148E; L149R; L149H; L149K; A150K; K151E; N153R; E157R; I158K; A159K;
A159N; K160D; G163E; S168W; F169F; E171K; F175K; G179R; G179H; G179K; Y180R; Y180H;
Y180K; L181R; L181H; L181K; L182R; L182H; L182K; Q183R; Q183H; Q183K; K184R; K184H;
K184P; P185R; P185H; P185K; S186H; S186K; S186G; S186E; S186R; P187R; P187H; P187K;
P187I; N188R; N188H; N188K; K189R; K189H; K189P; S190R; S190H; S190K; S190V; I191R;
I191H; I191K; Y192R; Y192H; Y192K; C193R; C193H; C193K; Y194R; Y194H; Y194K; Q195R;
Q195H; Q195K; S196R; S196H; S196K; V197R; V197H; V197K; S198R; S198H; S198K; S198E;
P199R; P199H; P199K; K200R; K200H; P201R; P201H; P201K; F202R; F202H; F202K; I203R;
I203H; I203K; I203G; T204R; T204H; T204K; S205R; S205H; S205K; K206R; K206H; Y207R;
Y207H; Y207K; H208R; H208K; N209R; N209H; N209K; N209D; N209W; N209F; V210R; V210H;
V210K; G218S; G218K; Y220S; Y220K; S223P; S223K; S223V; E225K; I227R; V228R; S229R;
P230R; Y231K; Y231G; Y231R; Q232R; F233R; D234R; L236R; I238R; P239R; I240K; G241R;
E242R; P243R; G244R; Y245R; V246K; V246R; P247R; K248E; K248R; Q250N; Q250R; T252D;
T252R; F253R; L254R; S255R; K256E; K256R; K257R; E258K; E258R; N259R; K260R; R262D;
L264R; S265R; K266R; I268R; S272Q; P273A; G276V; C279R; I280K; K281R; K281H; C285V;
V286K; T295N; T295F; T295W; H297A; H297R; W298L; Y301L; H302F; H302W; P304E; P304K;
D306K; D306F; L311T; F312L; F315M; F315K; T316R; T316H; T316K; V328N; R329T; M334E;
I338S; N340S; N340F; K348R; K348H; N355R; N355H; N355K; I356W; I356F; C357L; C357E;
C357K; N360K; G361R; C363V; A366V; V368K; D369A; D369N; V370L; K384T; L390H; T391W;
K392A; K392E; T393K; T393E; I395Q; R397K; P399F; P399Q; T400L; C405L; C405R; N406K;
K407E; K435Q; F445S; I471T; S472R; K480L; V483G; G497K; G497R; D501K; M503K; W509A;
Q511R; Q511H; Q511K; D512R; D512H; D512K; Y513R; Y513H; Y513K; K514R; K514H; P515R;
P515H; P515K; K516R; K516H; L517R; L517H; L517K; V521T; D523K; S526N; E529K; E529L;
R531E; S536A; N540R; N540H; N540K; K541R; K541H; L542R; L542H; L542K; S543R; S543H;
S543K; K544R; K544H; S545R; S545H; S545K; R546H; R546K; D549L; E567A; E567Q; N568D;
G577H; S579R; G585R; Y590R; Y590H; Y590K; K591R; K591H; P592R; P592H; P592K; K593R;
K593H; K594R; K594H; E595R; E595H; E595K; N596R; N596H; N596K; W599F; A602R; A602H;
A602K; I603R; I603H; I603K; H604R; H604K; K605R; K605H; A606R; A606H; A606K; L607R;
L607H; L607K; L607F; T608R; T608H; T608K; Q612R; R617Y; L620E; M624A; K634G; S638K;
K639E; I653A; D658N; A673G; A673R; Q674R; Q674K; K678R; P679R; E682R; S684R; G685R;
A696R; P699R; F701R; D703R; P707R; P707H; P707K; Y709R; E715R; A716R

TABLE 1.1
EXEMPLARY COMBINATIONS OF TWO AMINO
ACID ALTERATIONS RELATIVE TO SEQ ID NO: 1
Combinations of Two Amino Acid Alterations

L26R, E109R; L26R, H208R; L26R, K184R; L26R, K38R; L26R, L182R; L26R, Q183R; L26R,
S108R; L26R, S198R; L26R, T114R; E109R, H208R; E109R, K184R; E109R, K38R; E109R, L182R;
E109R, Q183R; E109R, S108R; E109R, S198R; E109R, T114R; H208R, K184R; H208R, K38R;
H208R, L182R; H208R, Q183R; H208R, S108R; H208R, S198R; H208R, T114R; K184R, K38R;
K184R, L182R; K184R, Q183R; K184R, S108R; K184R, S198R; K184R, T114R; K38R, L182R;
K38R, Q183R; K38R, S108R; K38R, S198R; K38R, T114R; L182R, Q183R; L182R, S108R; L182R,
S198R; L182R, T114R; Q183R, S108R; Q183R, S198R; Q183R, T114R; S108R, S198R; S108R,
T114R; S198R, T114R; L26R, K99R; L26R, P707R; L26R, L149R; L26R, N30R; L26R, N355R;
L26R, K281R; L26R, S108R; L26R, K348R; T5R, V139R; I2R, V139R; K99R, S186R; L26R,
A673G; L26R, Q674R; S579R, L26K; F701R, E258K; T5R, L26K; L26R, K435Q; L26R, G685R;
L26R, Q674K; L26R, P699R; L26R, T70E; L26R, Q232R; L26R, T252R; L26R, P679R; L26R, E83K;
L26R, E73P; L26R, K248E; T5R, S223P; S579R, S223P; L26R, S223P; T5R, A121Q; L26R, A696R;
S198R, I471T; L26R, N153R; L26R, E682R; L26R, D703R; Q612R, L26K; L26R, I471T; K348R,
L26K; S579R, I471T; L26R, V228R; T5R, S638K; S579R, K189P; S579R, E258K; L26R, K260R;
L26R, S638K; S579R, Y220S; T5R, I471T; L26R, F233R; L26R, V521T; F701R, A121Q; L26R,
G361R; S198R, E258K; L26R, S472R; T5R, Y220S; L26R, A150K; L26R, S684R; L26R, E157R;
L26R, K248R; F701R, L26K; S198R, N406K; S198R, Y220S; S198R, S638K; S198R, V521T; S579R,
A121Q; K348R, Y220S; S198R, K189P; L26R, E242R; L26R, K678R; T5R, N406K; L26R, I158K;
T5R, V521T; L26R, N259R; L26R, K257R; L26R, K256R; T5R, K189P; L26R, C405R; S579R,
V521T; S579R, N406K; T5R, K92E; T5R, E258K; L26R, I97R; S579R, S638K; T5R, K435Q; F701R,
S638K; L26R, L236R; F701R, I471T; Q612R, S223P; F701R, S223P; S198R, E119S; S579R, K92E;
L26R, E715R; Q612R, I471T; F701R, Y220S; S198R, S223P; L26R, K266R; S198R, K92E; L26R,
I268R; L26R, G244R; F701R, K189P; Q612R, E119S; F701R, N406K; L26R, E109K; F701R, K435Q;
L26R, Y220S; L26R, G57R; L26R, A716R; S579R, E119S; K348R, V521T; Q612R, S638K; K118R,
K189P; F701R, V521T; K118R, K92E; Q612R, A121Q; F701R, E119S; L26R, P239R; L26R, Y231R;
K348R, S223P; L26R, C279R; K348R, E119S; L26R, E100K; K348R, N406K; Q612R, E258K; L26R,
S255R; F701R, K92E; Q612R, N406K; K348R, K189P; S186R, E258K; Q612R, Y220S; L26R,
N568D; L26R, G241R; F701R, N568D; Q612R, K435Q; T5R, N568D; Q612R, V521T; S186R,
V521T; K348R, A121Q; L26R, D234R; K348R, K92E; S579R, N568D; L26R, A59R; Q612R, K92E;
L26R, H297R; L26R, E258R; K348R, K435Q; L26R, P94K; K348R, I471T; L26R, E119S; S579R,
K435Q; L26R, Y709R; S198R, N568D; S186R, N568D; L26R, G497R; Q612R, N568D; K118R,
N568D; L26R, L254R; K348R, E258K; L26R, P96K; L26R, Y245R; L26R, I238R; L26R, P243R;
L26R, L16F; L26R, P230R; L26R, P199R; L26R, G585R; K348R, N568D; L26R, A673R; L26R,
V246R; L26R, S229R; L26R, S265R; L26R, C50P; L26R, F253R; L26R, L264R; L26R, I227R; L26R,
P247R; L26R, Q250R; L26R, F253R; L26R, L264R; L26R, I227R; L26R, P247R; L26R, Q250R

TABLE 1.2
EXEMPLARY COMBINATIONS OF THREE AMINO
ACID ALTERATIONS RELATIVE TO SEQ ID NO: 1
Combinations of Three Amino Acid Alterations

L26R, E109R, H208R; L26R, E109R, K184R; L26R, E109R, K38R; L26R, E109R, L182R; L26R,
E109R, Q183R; L26R, E109R, S108R; L26R, E109R, S198R; L26R, E109R, T114R; L26R, H208R,
K184R; L26R, H208R, K38R; L26R, H208R, L182R; L26R, H208R, Q183R; L26R, H208R, S108R;
L26R, H208R, S198R; L26R, H208R, T114R; L26R, K184R, K38R; L26R, K184R, L182R; L26R,
K184R, Q183R; L26R, K184R, S108R; L26R, K184R, S198R; L26R, K184R, T114R; L26R, K38R,
L182R; L26R, K38R, Q183R; L26R, K38R, S108R; L26R, K38R, S198R; L26R, K38R, T114R;
L26R, L182R, Q183R; L26R, L182R, S108R; L26R, L182R, S198R; L26R, L182R, T114R; L26R,
Q183R, S108R; L26R, Q183R, S198R; L26R, Q183R, T114R; L26R, S108R, S198R; L26R, S108R,
T114R; L26R, S198R, T114R; E109R, H208R, K184R; E109R, H208R, K38R; E109R, H208R,
L182R; E109R, H208R, Q183R; E109R, H208R, S108R; E109R, H208R, S198R; E109R, H208R,
T114R; E109R, K184R, K38R; E109R, K184R, L182R; E109R, K184R, Q183R; E109R, K184R,
S108R; E109R, K184R, S198R; E109R, K184R, T114R; E109R, K38R, L182R; E109R, K38R,
Q183R; E109R, K38R, S108R; E109R, K38R, S198R; E109R, K38R, T114R; E109R, L182R, Q183R;
E109R, L182R, S108R; E109R, L182R, S198R; E109R, L182R, T114R; E109R, Q183R, S108R;
E109R, Q183R, S198R; E109R, Q183R, T114R; E109R, S108R, S198R; E109R, S108R, T114R;
E109R, S198R, T114R; H208R, K184R, K38R; H208R, K184R, L182R; H208R, K184R, Q183R;
H208R, K184R, S108R; H208R, K184R, S198R; H208R, K184R, T114R; H208R, K38R, L182R;
H208R, K38R, Q183R; H208R, K38R, S108R; H208R, K38R, S198R; H208R, K38R, T114R; H208R,
L182R, Q183R; H208R, L182R, S108R; H208R, L182R, S198R; H208R, L182R, T114R; H208R,
Q183R, S108R; H208R, Q183R, S198R; H208R, Q183R, T114R; H208R, S108R, S198R; H208R,
S108R, T114R; H208R, S198R, T114R; K184R, K38R, L182R; K184R, K38R, Q183R; K184R,
K38R, S108R; K184R, K38R, S198R; K184R, K38R, T114R; K184R, L182R, Q183R; K184R,
L182R, S108R; K184R, L182R, S198R; K184R, L182R, T114R; K184R, Q183R, S108R; K184R,
Q183R, S198R; K184R, Q183R, T114R; K184R, S108R, S198R; K184R, S108R, T114R; K184R,
S198R, T114R; K38R, L182R, Q183R; K38R, L182R, S108R; K38R, L182R, S198R; K38R, L182R,
T114R; K38R, Q183R, S108R; K38R, Q183R, S198R; K38R, Q183R, T114R; K38R, S108R, S198R;
K38R, S108R, T114R; K38R, S198R, T114R; L182R, Q183R, S108R; L182R, Q183R, S198R;
L182R, Q183R, T114R; L182R, S108R, S198R; L182R, S108R, T114R; L182R, S198R, T114R;
Q183R, S108R, S198R; Q183R, S108R, T114R; Q183R, S198R, T114R; S108R, S198R, T114R

TABLE 1.3
EXEMPLARY COMBINATIONS OF FOUR AMINO
ACID ALTERATIONS RELATIVE TO SEQ ID NO: 1
Combinations of Four Amino Acid Alterations

L26R, E109R, H208R, K184R; L26R, E109R, H208R, K38R; L26R, E109R, H208R, L182R; L26R,
E109R, H208R, Q183R; L26R, E109R, H208R, S108R; L26R, E109R, H208R, S198R; L26R, E109R,
H208R, T114R; L26R, E109R, K184R, K38R; L26R, E109R, K184R, L182R; L26R, E109R, K184R,
Q183R; L26R, E109R, K184R, S108R; L26R, E109R, K184R, S198R; L26R, E109R, K184R, T114R;
L26R, E109R, K38R, L182R; L26R, E109R, K38R, Q183R; L26R, E109R, K38R, S108R; L26R,
E109R, K38R, S198R; L26R, E109R, K38R, T114R; L26R, E109R, L182R, Q183R; L26R, E109R,
L182R, S108R; L26R, E109R, L182R, S198R; L26R, E109R, L182R, T114R; L26R, E109R, Q183R,
S108R; L26R, E109R, Q183R, S198R; L26R, E109R, Q183R, T114R; L26R, E109R, S108R, S198R;
L26R, E109R, S108R, T114R; L26R, E109R, S198R, T114R; L26R, H208R, K184R, K38R; L26R,
H208R, K184R, L182R; L26R, H208R, K184R, Q183R; L26R, H208R, K184R, S108R; L26R,
H208R, K184R, S198R; L26R, H208R, K184R, T114R; L26R, H208R, K38R, L182R; L26R, H208R,
K38R, Q183R; L26R, H208R, K38R, S108R; L26R, H208R, K38R, S198R; L26R, H208R, K38R,
T114R; L26R, H208R, L182R, Q183R; L26R, H208R, L182R, S108R; L26R, H208R, L182R, S198R;
L26R, H208R, L182R, T114R; L26R, H208R, Q183R, S108R; L26R, H208R, Q183R, S198R; L26R,
H208R, Q183R, T114R; L26R, H208R, S108R, S198R; L26R, H208R, S108R, T114R; L26R, H208R,
S198R, T114R; L26R, K184R, K38R, L182R; L26R, K184R, K38R, Q183R; L26R, K184R, K38R,
S108R; L26R, K184R, K38R, S198R; L26R, K184R, K38R, T114R; L26R, K184R, L182R, Q183R;
L26R, K184R, L182R, S108R; L26R, K184R, L182R, S198R; L26R, K184R, L182R, T114R; L26R,
K184R, Q183R, S108R; L26R, K184R, Q183R, S198R; L26R, K184R, Q183R, T114R; L26R, K184R,
S108R, S198R; L26R, K184R, S108R, T114R; L26R, K184R, S198R, T114R; L26R, K38R, L182R,
Q183R; L26R, K38R, L182R, S108R; L26R, K38R, L182R, S198R; L26R, K38R, L182R, T114R;
L26R, K38R, Q183R, S108R; L26R, K38R, Q183R, S198R; L26R, K38R, Q183R, T114R; L26R,
K38R, S108R, S198R; L26R, K38R, S108R, T114R; L26R, K38R, S198R, T114R; L26R, L182R,
Q183R, S108R; L26R, L182R, Q183R, S198R; L26R, L182R, Q183R, T114R; L26R, L182R, S108R,
S198R; L26R, L182R, S108R, T114R; L26R, L182R, S198R, T114R; L26R, Q183R, S108R, S198R;
L26R, Q183R, S108R, T114R; L26R, Q183R, S198R, T114R; L26R, S108R, S198R, T114R; E109R,
H208R, K184R, K38R; E109R, H208R, K184R, L182R; E109R, H208R, K184R, Q183R; E109R,
H208R, K184R, S108R; E109R, H208R, K184R, S198R; E109R, H208R, K184R, T114R; E109R,
H208R, K38R, L182R; E109R, H208R, K38R, Q183R; E109R, H208R, K38R, S108R; E109R,
H208R, K38R, S198R; E109R, H208R, K38R, T114R; E109R, H208R, L182R, Q183R; E109R,
H208R, L182R, S108R; E109R, H208R, L182R, S198R; E109R, H208R, L182R, T114R; E109R,
H208R, Q183R, S108R; E109R, H208R, Q183R, S198R; E109R, H208R, Q183R, T114R; E109R,
H208R, S108R, S198R; E109R, H208R, S108R, T114R; E109R, H208R, S198R, T114R; E109R,
K184R, K38R, L182R; E109R, K184R, K38R, Q183R; E109R, K184R, K38R, S108R; E109R,
K184R, K38R, S198R; E109R, K184R, K38R, T114R; E109R, K184R, L182R, Q183R; E109R,
K184R, L182R, S108R; E109R, K184R, L182R, S198R; E109R, K184R, L182R, T114R; E109R,
K184R, Q183R, S108R; E109R, K184R, Q183R, S198R; E109R, K184R, Q183R, T114R; E109R,
K184R, S108R, S198R; E109R, K184R, S108R, T114R; E109R, K184R, S198R, T114R; E109R,
K38R, L182R, Q183R; E109R, K38R, L182R, S108R; E109R, K38R, L182R, S198R; E109R, K38R,
L182R, T114R; E109R, K38R, Q183R, S108R; E109R, K38R, Q183R, S198R; E109R, K38R, Q183R,
T114R; E109R, K38R, S108R, S198R; E109R, K38R, S108R, T114R; E109R, K38R, S198R, T114R;
E109R, L182R, Q183R, S108R; E109R, L182R, Q183R, S198R; E109R, L182R, Q183R, T114R;
E109R, L182R, S108R, S198R; E109R, L182R, S108R, T114R; E109R, L182R, S198R, T114R;
E109R, Q183R, S108R, S198R; E109R, Q183R, S108R, T114R; E109R, Q183R, S198R, T114R;
E109R, S108R, S198R, T114R; H208R, K184R, K38R, L182R; H208R, K184R, K38R, Q183R;
H208R, K184R, K38R, S108R; H208R, K184R, K38R, S198R; H208R, K184R, K38R, T114R;
H208R, K184R, L182R, Q183R; H208R, K184R, L182R, S108R; H208R, K184R, L182R, S198R;
H208R, K184R, L182R, T114R; H208R, K184R, Q183R, S108R; H208R, K184R, Q183R, S198R;
H208R, K184R, Q183R, T114R; H208R, K184R, S108R, S198R; H208R, K184R, S108R, T114R;
H208R, K184R, S198R, T114R; H208R, K38R, L182R, Q183R; H208R, K38R, L182R, S108R;
H208R, K38R, L182R, S198R; H208R, K38R, L182R, T114R; H208R, K38R, Q183R, S108R;
H208R, K38R, Q183R, S198R; H208R, K38R, Q183R, T114R; H208R, K38R, S108R, S198R;
H208R, K38R, S108R, T114R; H208R, K38R, S198R, T114R; H208R, L182R, Q183R, S108R;
H208R, L182R, Q183R, S198R; H208R, L182R, Q183R, T114R; H208R, L182R, S108R, S198R;
H208R, L182R, S108R, T114R; H208R, L182R, S198R, T114R; H208R, Q183R, S108R, S198R;
H208R, Q183R, S108R, T114R; H208R, Q183R, S198R, T114R; H208R, S108R, S198R, T114R;
K184R, K38R, L182R, Q183R; K184R, K38R, L182R, S108R; K184R, K38R, L182R, S198R;
K184R, K38R, L182R, T114R; K184R, K38R, Q183R, S108R; K184R, K38R, Q183R, S198R;
K184R, K38R, Q183R, T114R; K184R, K38R, S108R, S198R; K184R, K38R, S108R, T114R;
K184R, K38R, S198R, T114R; K184R, L182R, Q183R, S108R; K184R, L182R, Q183R, S198R;
K184R, L182R, Q183R, T114R; K184R, L182R, S108R, S198R; K184R, L182R, S108R, T114R;
K184R, L182R, S198R, T114R; K184R, Q183R, S108R, S198R; K184R, Q183R, S108R, T114R;
K184R, Q183R, S198R, T114R; K184R, S108R, S198R, T114R; K38R, L182R, Q183R, S108R;
K38R, L182R, Q183R, S198R; K38R, L182R, Q183R, T114R; K38R, L182R, S108R, S198R; K38R,
L182R, S108R, T114R; K38R, L182R, S198R, T114R; K38R, Q183R, S108R, S198R; K38R, Q183R,
S108R, T114R; K38R, Q183R, S198R, T114R; K38R, S108R, S198R, T114R; L182R, Q183R, S108R,
S198R; L182R, Q183R, S108R, T114R; L182R, Q183R, S198R, T114R; L182R, S108R, S198R,
T114R; Q183R, S108R, S198R, T114R

TABLE 1.4 provides illustrative variant effector protein sequences that are useful in the compositions, systems and methods described herein.

TABLE 1.4
EXEMPLARY VARIANT EFFECTOR PROTEIN AMINO ACID SEQUENCES
RELATIVE TO SEQ ID NO: 1

| Effector | | |
|---|---|---|
| Protein | | |
| SEQ ID | | |
| NO: | Descr | Sequence |
| 2 | L26R | MIKPTVSQFLTPGFKLIRNHSRTAGRKLKNEGEEACKKFVRENEIPKDECPN |
| | Variant | FQGGPAIANIIAKSREFTEWEIYQSSLAIQEVIFTLPKDKLPEPILKEEWRAQ |
| | | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSGDAKKPVRARKAKAPEFHDKLAPSYTVVLREA |
| | | V |
| 3 | L26H | MIKPTVSQFLTPGFKLIRNHSRTAGHKLKNEGEEACKKFVRENEIPKDECPN |
| | Variant | FQGGPAIANIIAKSREFTEWEIYQSSLAIQEVIFTLPKDKLPEPILKEEWRAQ |
| | | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSGDAKKPVRARKAKAPEFHDKLAPSYTVVLREA |
| | | V |
| 4 | L26K | MIKPTVSQFLTPGFKLIRNHSRTAGKKLKNEGEEACKKFVRENEIPKDECPN |
| | Variant | FQGGPAIANIIAKSREFTEWEIYQSSLAIQEVIFTLPKDKLPEPILKEEWRAQ |
| | | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSGDAKKPVRARKAKAPEFHDKLAPSYTVVLREA |
| | | V |
| 31 | L26R, | MIKPTVSQFLTPGFKLIRNHSRTAGRKLKNEGEEACKKFVRENEIPKDECPN |
| | K184R | FQGGPAIANIIAKSREFTEWEIYQSSLAIQEVIFTLPKDKLPEPILKEEWRAQ |
| | Variant | WLSEHGLDTVPYKEAAGLNLIIKNAVNTYKGVQVKVDNKNKNNLAKINR |
| | | KNEIAKLNGEQEISFEEIKAFDDKGYLLQRPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSGDAKKPVRARKAKAPEFHDKLAPSYTVVLREA |
| | | V |
| 32 | L26R, | MIKPTVSQFLTPGFKLIRNHSRTAGRKLKNEGEEACKKFVRENEIPKDECPN |
| | K184R, | FQGGPAIANIIAKSREFTEWEIYQSSLAIQEVIFTLPKDKLPEPILKEEWRAQ |
| | H208R | WLSEHGLDTVPYKEAAGLNLIIKNAVNTYKGVQVKVDNKNKNNLAKINR |
| | Variant | KNEIAKLNGEQEISFEEIKAFDDKGYLLQRPSPNKSIYCYQSVSPKPFITSKY |
| | | RNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSGDAKKPVRARKAKAPEFHDKLAPSYTVVLREA |
| | | V |
| 33 | L26R, | MIKPTVSQFLTPGFKLIRNHSRTAGRKLKNEGEEACKKFVRENEIPKDECPN |
| | Q183R, | FQGGPAIANIIAKSREFTEWEIYQSSLAIQEVIFTLPKDKLPEPILKEEWRAQ |
| | K184R, | WLSEHGLDRVPYKEAAGLNLIIKNAVNTYKGVQVKVDNKNKNNLAKINR |
| | T114R | KNEIAKLNGEQEISFEEIKAFDDKGYLLRRPSPNKSIYCYQSVSPKPFITSKY |
| | Variant | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSGDAKKPVRARKAKAPEFHDKLAPSYTVVLREA |
| | | V |
| 100 | T5R | MIKPRVSQFLTPGFKLIRNHSRTAGLKLKNEGEEACKKFVRENEIPKDECPN |
| | Variant | FQGGPAIANIIAKSREFTEWEIYQSSLAIQEVIFTLPKDKLPEPILKEEWRAQ |
| | | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSGDAKKPVRARKAKAPEFHDKLAPSYTVVLREA |
| | | V |
| 101 | I471T | MIKPTVSQFLTPGFKLIRNHSRTAGLKLKNEGEEACKKFVRENEIPKDECPN |
| | Variant | FQGGPAIANIIAKSREFTEWEIYQSSLAIQEVIFTLPKDKLPEPILKEEWRAQ |
| | | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMTSGTHFISEKAQVSNKSEIYFTSTDKGKTKDVMKSDYKWFQDY |
| | | KPKLSKEVRDALSDIEWRLRRESLEFNKLSKSREQDARQLANWISSMCDVI |
| | | GIENLVKKNNFFGGSGKREPGWDNFYKPKKENRWWINAIHKALTELSQNK |
| | | GKRVILLPAMRTSITCPKCKYCDSKNRNGEKFNCLKCGIELNADIDVATEN |
| | | LATVAITAQSMPKPTCERSGDAKKPVRARKAKAPEFHDKLAPSYTVVLRE |
| | | AV |
| 102 | S579R | MIKPTVSQFLTPGFKLIRNHSRTAGLKLKNEGEEACKKFVRENEIPKDECPN |
| | Variant | FQGGPAIANIIAKSREFTEWEIYQSSLAIQEVIFTLPKDKLPEPILKEEWRAQ |
| | | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGRGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSGDAKKPVRARKAKAPEFHDKLAPSYTVVLREA |
| | | V |
| 103 | F701R | MIKPTVSQFLTPGFKLIRNHSRTAGLKLKNEGEEACKKFVRENEIPKDECPN |
| | Variant | FQGGPAIANIIAKSREFTEWEIYQSSLAIQEVIFTLPKDKLPEPILKEEWRAQ |
| | | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSGDAKKPVRARKAKAPERHDKLAPSYTVVLREA |
| | | V |
| 104 | S223P | MIKPTVSQFLTPGFKLIRNHSRTAGLKLKNEGEEACKKFVRENEIPKDECPN |
| | Variant | FQGGPAIANIIAKSREFTEWEIYQSSLAIQEVIFTLPKDKLPEPILKEEWRAQ |
| | | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKPNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSGDAKKPVRARKAKAPEFHDKLAPSYTVVLREA |
| | | V |
| 105 | A121Q | MIKPTVSQFLTPGFKLIRNHSRTAGLKLKNEGEEACKKFVRENEIPKDECPN |
| | Variant | FQGGPAIANIIAKSREFTEWEIYQSSLAIQEVIFTLPKDKLPEPILKEEWRAQ |
| | | WLSEHGLDTVPYKEAQGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSGDAKKPVRARKAKAPEFHDKLAPSYTVVLREA |
| | | V |
| 106 | E258K | MIKPTVSQFLTPGFKLIRNHSRTAGLKLKNEGEEACKKFVRENEIPKDECPN |
| | Variant | FQGGPAIANIIAKSREFTEWEIYQSSLAIQEVIFTLPKDKLPEPILKEEWRAQ |
| | | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKKN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSGDAKKPVRARKAKAPEFHDKLAPSYTVVLREA |
| | | V |
| 107 | S198R | MIKPTVSQFLTPGFKLIRNHSRTAGLKLKNEGEEACKKFVRENEIPKDECPN |
| | Variant | FQGGPAIANIIAKSREFTEWEIYQSSLAIQEVIFTLPKDKLPEPILKEEWRAQ |
| | | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVRPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSGDAKKPVRARKAKAPEFHDKLAPSYTVVLREA |
| | | V |
| 108 | L26R, | MIKPTVSQFLTPGFKLIRNHSRTAGRKLKNEGEEACKKFVRENEIPKDECPN |
| | A673G | FQGGPAIANIIAKSREFTEWEIYQSSLAIQEVIFTLPKDKLPEPILKEEWRAQ |
| | Variant | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITGQSMPKPTCERSGDAKKPVRARKAKAPEFHDKLAPSYTVVLREA |
| | | V |
| 109 | L26R, | MIKPTVSQFLTPGFKLIRNHSRTAGRKLKNEGEEACKKFVRENEIPKDECPN |
| | Q674R | FQGGPAIANIIAKSREFTEWEIYQSSLAIQEVIFTLPKDKLPEPILKEEWRAQ |
| | Variant | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITARSMPKPTCERSGDAKKPVRARKAKAPEFHDKLAPSYTVVLREA |
| | | V |
| 110 | S579R, | MIKPTVSQFLTPGFKLIRNHSRTAGKKLKNEGEEACKKFVRENEIPKDECPN |
| | L26K | FQGGPAIANIIAKSREFTEWEIYQSSLAIQEVIFTLPKDKLPEPILKEEWRAQ |
| | Variant | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGRGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSGDAKKPVRARKAKAPEFHDKLAPSYTVVLREA |
| | | V |
| 111 | F701R, | MIKPTVSQFLTPGFKLIRNHSRTAGLKLKNEGEEACKKFVRENEIPKDECPN |
| | E258K | FQGGPAIANIIAKSREFTEWEIYQSSLAIQEVIFTLPKDKLPEPILKEEWRAQ |
| | Variant | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKKN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSGDAKKPVRARKAKAPERHDKLAPSYTVVLREA |
| | | V |
| 112 | T5R, | MIKPRVSQFLTPGFKLIRNHSRTAGKKLKNEGEEACKKFVRENEIPKDECPN |
| | L26K | FQGGPAIANIIAKSREFTEWEIYQSSLAIQEVIFTLPKDKLPEPILKEEWRAQ |
| | Variant | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSGDAKKPVRARKAKAPEFHDKLAPSYTVVLREA |
| | | V |
| 113 | L26R, | MIKPTVSQFLTPGFKLIRNHSRTAGRKLKNEGEEACKKFVRENEIPKDECPN |
| | K435Q | FQGGPAIANIIAKSREFTEWEIYQSSLAIQEVIFTLPKDKLPEPILKEEWRAQ |
| | Variant | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQQIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSGDAKKPVRARKAKAPEFHDKLAPSYTVVLREA |
| | | V |
| 114 | L26R, | MIKPTVSQFLTPGFKLIRNHSRTAGRKLKNEGEEACKKFVRENEIPKDECPN |
| | G685R | FQGGPAIANIIAKSREFTEWEIYQSSLAIQEVIFTLPKDKLPEPILKEEWRAQ |
| | Variant | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSRDAKKPVRARKAKAPEFHDKLAPSYTVVLREA |
| | | V |
| 115 | L26R, | MIKPTVSQFLTPGFKLIRNHSRTAGRKLKNEGEEACKKFVRENEIPKDECPN |
| | Q674K | FQGGPAIANIIAKSREFTEWEIYQSSLAIQEVIFTLPKDKLPEPILKEEWRAQ |
| | Variant | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAKSMPKPTCERSGDAKKPVRARKAKAPEFHDKLAPSYTVVLREA |
| | | V |
| 116 | L26R, | MIKPTVSQFLTPGFKLIRNHSRTAGRKLKNEGEEACKKFVRENEIPKDECPN |
| | P699R | FQGGPAIANIIAKSREFTEWEIYQSSLAIQEVIFTLPKDKLPEPILKEEWRAQ |
| | Variant | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSGDAKKPVRARKAKAREFHDKLAPSYTVVLREA |
| | | V |
| 117 | L26R, | MIKPTVSQFLTPGFKLIRNHSRTAGRKLKNEGEEACKKFVRENEIPKDECPN |
| | T252R | FQGGPAIANIIAKSREFTEWEIYQSSLAIQEVIFTLPKDKLPEPILKEEWRAQ |
| | Variant | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYRFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSGDAKKPVRARKAKAPEFHDKLAPSYTVVLREA |
| | | V |
| 118 | L26R, | MIKPTVSQFLTPGFKLIRNHSRTAGRKLKNEGEEACKKFVRENEIPKDECPN |
| | P679R | FQGGPAIANIIAKSREFTEWEIYQSSLAIQEVIFTLPKDKLPEPILKEEWRAQ |
| | Variant | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKRTCERSGDAKKPVRARKAKAPEFHDKLAPSYTVVLREA |
| | | V |
| 119 | L26R, | MIKPTVSQFLTPGFKLIRNHSRTAGRKLKNEGEEACKKFVRENEIPKDECPN |
| | S223P | FQGGPAIANIIAKSREFTEWEIYQSSLAIQEVIFTLPKDKLPEPILKEEWRAQ |
| | Variant | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKPNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSGDAKKPVRARKAKAPEFHDKLAPSYTVVLREA |
| | | V |
| 120 | S198R, | MIKPTVSQFLTPGFKLIRNHSRTAGLKLKNEGEEACKKFVRENEIPKDECPN |
| | I471T | FQGGPAIANIIAKSREFTEWEIYQSSLAIQEVIFTLPKDKLPEPILKEEWRAQ |
| | Variant | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVRPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMTSGTHFISEKAQVSNKSEIYFTSTDKGKTKDVMKSDYKWFQDY |
| | | KPKLSKEVRDALSDIEWRLRRESLEFNKLSKSREQDARQLANWISSMCDVI |
| | | GIENLVKKNNFFGGSGKREPGWDNFYKPKKENRWWINAIHKALTELSQNK |
| | | GKRVILLPAMRTSITCPKCKYCDSKNRNGEKFNCLKCGIELNADIDVATEN |
| | | LATVAITAQSMPKPTCERSGDAKKPVRARKAKAPEFHDKLAPSYTVVLRE |
| | | AV |
| 121 | L26R, | MIKPTVSQFLTPGFKLIRNHSRTAGRKLKNEGEEACKKFVRENEIPKDECPN |
| | E682R | FQGGPAIANIIAKSREFTEWEIYQSSLAIQEVIFTLPKDKLPEPILKEEWRAQ |
| | Variant | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCRRSGDAKKPVRARKAKAPEFHDKLAPSYTVVLREA |
| | | V |
| 122 | L26R, | MIKPTVSQFLTPGFKLIRNHSRTAGRKLKNEGEEACKKFVRENEIPKDECPN |
| | I471T | FQGGPAIANIIAKSREFTEWEIYQSSLAIQEVIFTLPKDKLPEPILKEEWRAQ |
| | Variant | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMTSGTHFISEKAQVSNKSEIYFTSTDKGKTKDVMKSDYKWFQDY |
| | | KPKLSKEVRDALSDIEWRLRRESLEFNKLSKSREQDARQLANWISSMCDVI |
| | | GIENLVKKNNFFGGSGKREPGWDNFYKPKKENRWWINAIHKALTELSQNK |
| | | GKRVILLPAMRTSITCPKCKYCDSKNRNGEKFNCLKCGIELNADIDVATEN |
| | | LATVAITAQSMPKPTCERSGDAKKPVRARKAKAPEFHDKLAPSYTVVLRE |
| | | AV |
| 123 | L26R, | MIKPTVSQFLTPGFKLIRNHSRTAGRKLKNEGEEACKKFVRENEIPKDECPN |
| | S638K | FQGGPAIANIIAKSREFTEWEIYQSSLAIQEVIFTLPKDKLPEPILKEEWRAQ |
| | Variant | WLSEHGLDTVPYKEAAGLNLIIKNAVNTYKGVQVKVDNKNKNNLAKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDKKNRNGEKFNCLKCGIELNADIDVATENL |
| | | ATVAITAQSMPKPTCERSGDAKKPVRARKAKAPEFHDKLAPSYTVVLREA |
| | | V |
| 124 | L26R, | MIKPTVSQFLTPGFKLIRNHSRTAGRKLKNEGEEACKKFVRENEIPKDECPN |
| | A150K | FQGGPAIANIIAKSREFTEWEIYQSSLAIQEVIFTLPKDKLPEPILKEEWRAQ |
| | Variant | WLSEHGLDTVPYKEAAGLNLIIKNAVNTYKGVQVKVDNKNKNNLKKINR |
| | | KNEIAKLNGEQEISFEEIKAFDDKGYLLQKPSPNKSIYCYQSVSPKPFITSKY |
| | | HNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSIND |
| | | LFDYFTGDPVIDTKANVVRFRYKMENGIVNYKPVREKKGKELLENICDQN |
| | | GSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPND |
| | | LPWDKMISGTHFISEKAQVSNKSEIYFTSTDKGKTKDVMKSDYKWFQDYK |
| | | PKLSKEVRDALSDIEWRLRRESLEFNKLSKSREQDARQLANWISSMCDVIGI |
| | | ENLVKKNNFFGGSGKREPGWDNFYKPKKENRWWINAIHKALTELSQNKG |
| | | KRVILLPAMRTSITCPKCKYCDSKNRNGEKFNCLKCGIELNADIDVATENL |
| | | ATVAITAQSMPKPTCERSGDAKKPVRARKAKAPEFHDKLAPSYTVVLREA |
| | | V |

TABLE 1.5 provides illustrative PAM sequences that are useful in the compositions, systems and methods described herein.

TABLE 1.5
EXEMPLARY PAM SEQUENCES

| Effector Protein | PAM Sequence* | PAM SEQ |
|---|---|---|
| SEQ ID NO: | (5′→3′) | ID NO: |
| 1 and variants thereof | NTTN | 14 |
| 1 and variants thereof | TTN | 57 |
| 1 and variants thereof | GTTC | 58 |
| 1 and variants thereof | TTTG | 59 |

*wherein each N is independently any one of A, G, C, or T

TABLE 2 provides illustrative sequences of exemplary heterologous polypeptide modifications of effector protein(s) that are useful in the compositions, systems and methods described herein.

TABLE 2
SEQUENCES OF EXEMPLARY HETEROLOGOUS POLYPEPTIDE
MODIFICATIONS OF EFFECTOR PROTEIN(S)

| SEQ | | |
|---|---|---|
| ID | | |
| NO: | Description | Sequence* |
| 15 | NLS | KR(K/R)R or K(K/R)RK |
| 16 | NLS | (P/R)XXKR(\hat{ }D/E )(K/R) |
| 17 | NLS | KRX(W/F/Y)XXAF |
| 18 | NLS | (R/P)XXKR(K/R)(\hat{ }D/E) |
| 19 | NLS | LGKR(K/R)(W/F/Y) |
| 20 | NLS | KRX10-12 (K/R)(K/R) or KRX10- |
| | | 12K (K/R)X(K/R) |
| 60 | EEP | GLFXALLXLLXSLWXLLLXA |
| 61 | NLS | K(K/R)RK |
| 62 | NLS | KRX₁₁K(K/R)(K/R) |
| 63 | NLS | KRX₁₂K(K/R)(K/R) |
| 64 | NLS | KRX₁₀K(K/R)X(K/R) |
| 65 | NLS | KRX₁₁K(K/R)X(K/R) |
| 66 | NLS | KRX₁₂K(K/R)X(K/R) |
| 67 | EEP | GLFXALLXLLXSLWXLLLXA |
| 21 | NLS | APKKKRKVGIHGVPAA |
| 22 | NLS | KRPAATKKAGQAKKKKEF |
| 23 | 3X FLAG tag | DYKDHDGDYKDHDIDYKDDDDK |

*wherein X is any naturally occurring amino acid; and \hat{ }D/E is any naturally occurring amino acid except Asp or Glu

TABLE 2.1 provides illustrative sequences of exemplary heterologous polypeptide modifications and effector proteins as described herein.

TABLE 2.1
EXEMPLARY SEQUENCES OF EFFECTOR PROTEINS AND MODIFICATIONS

| SEQ | | |
|---|---|---|
| ID | | |
| NO: | Name | Sequence |
| 5 | L26R | MAPKKKRKVGIHGVPAAMIKPTVSQFLTPGFKLIRNHSRTAGRKLKNEGEEA |
| | Variant | CKKFVRENEIPKDECPNFQGGPAIANIIAKSREFTEWEIYQSSLAIQEVIFTLPKD |
| | (N- & | KLPEPILKEEWRAQWLSEHGLDTVPYKEAAGLNLIIKNAVNTYKGVQVKVDN |
| | C- | KNKNNLAKINRKNEIAKLNGEQEISFEEIKAFDDKGYLLQKPSPNKSIYCYQSV |
| | term | SPKPFITSKYHNVNLPEEYIGYYRKSNEPIVSPYQFDRLRIPIGEPGYVPKWQYT |
| | NLS) | FLSKKENKRRKLSKRIKNVSPILGIICIKKDWCVFDMRGLLRTNHWKKYHKPT |
| | | DSINDLFDYFTGDPVIDTKANVVRFRYKMENGIVNYKPVREKKGKELLENICD |
| | | QNGSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPNDLP |
| | | WDKMISGTHFISEKAQVSNKSEIYFTSTDKGKTKDVMKSDYKWFQDYKPKLS |
| | | KEVRDALSDIEWRLRRESLEFNKLSKSREQDARQLANWISSMCDVIGIENLVK |
| | | KNNFFGGSGKREPGWDNFYKPKKENRWWINAIHKALTELSQNKGKRVILLPA |
| | | MRTSITCPKCKYCDSKNRNGEKFNCLKCGIELNADIDVATENLATVAITAQSM |
| | | PKPTCERSGDAKKPVRARKAKAPEFHDKLAPSYTVVLREAVKRPAATKKAG |
| | | QAKKKKEF |
| 6 | L26H | MAPKKKRKVGIHGVPAAMIKPTVSQFLTPGFKLIRNHSRTAGHKLKNEGEEA |
| | Variant | CKKFVRENEIPKDECPNFQGGPAIANIIAKSREFTEWEIYQSSLAIQEVIFTLPKD |
| | (N- & | KLPEPILKEEWRAQWLSEHGLDTVPYKEAAGLNLIIKNAVNTYKGVQVKVDN |
| | C- | KNKNNLAKINRKNEIAKLNGEQEISFEEIKAFDDKGYLLQKPSPNKSIYCYQSV |
| | term | SPKPFITSKYHNVNLPEEYIGYYRKSNEPIVSPYQFDRLRIPIGEPGYVPKWQYT |
| | NLS) | FLSKKENKRRKLSKRIKNVSPILGIICIKKDWCVFDMRGLLRTNHWKKYHKPT |
| | | DSINDLFDYFTGDPVIDTKANVVRFRYKMENGIVNYKPVREKKGKELLENICD |
| | | QNGSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPNDLP |
| | | WDKMISGTHFISEKAQVSNKSEIYFTSTDKGKTKDVMKSDYKWFQDYKPKLS |
| | | KEVRDALSDIEWRLRRESLEFNKLSKSREQDARQLANWISSMCDVIGIENLVK |
| | | KNNFFGGSGKREPGWDNFYKPKKENRWWINAIHKALTELSQNKGKRVILLPA |
| | | MRTSITCPKCKYCDSKNRNGEKFNCLKCGIELNADIDVATENLATVAITAQSM |
| | | PKPTCERSGDAKKPVRARKAKAPEFHDKLAPSYTVVLREAVKRPAATKKAG |
| | | QAKKKKEF |
| 7 | L26K | MAPKKKRKVGIHGVPAAMIKPTVSQFLTPGFKLIRNHSRTAGKKLKNEGEEA |
| | Variant | CKKFVRENEIPKDECPNFQGGPAIANIIAKSREFTEWEIYQSSLAIQEVIFTLPKD |
| | (N- & | KLPEPILKEEWRAQWLSEHGLDTVPYKEAAGLNLIIKNAVNTYKGVQVKVDN |
| | C- | KNKNNLAKINRKNEIAKLNGEQEISFEEIKAFDDKGYLLQKPSPNKSIYCYQSV |
| | term | SPKPFITSKYHNVNLPEEYIGYYRKSNEPIVSPYQFDRLRIPIGEPGYVPKWQYT |
| | NLS) | FLSKKENKRRKLSKRIKNVSPILGIICIKKDWCVFDMRGLLRTNHWKKYHKPT |
| | | DSINDLFDYFTGDPVIDTKANVVRFRYKMENGIVNYKPVREKKGKELLENICD |
| | | QNGSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPNDLP |
| | | WDKMISGTHFISEKAQVSNKSEIYFTSTDKGKTKDVMKSDYKWFQDYKPKLS |
| | | KEVRDALSDIEWRLRRESLEFNKLSKSREQDARQLANWISSMCDVIGIENLVK |
| | | KNNFFGGSGKREPGWDNFYKPKKENRWWINAIHKALTELSQNKGKRVILLPA |
| | | MRTSITCPKCKYCDSKNRNGEKFNCLKCGIELNADIDVATENLATVAITAQSM |
| | | PKPTCERSGDAKKPVRARKAKAPEFHDKLAPSYTVVLREAVKRPAATKKAG |
| | | QAKKKKEF |
| 8 | L26R | MAPKKKRKVGIHGVPAAMIKPTVSQFLTPGFKLIRNHSRTAGRKLKNEGEEA |
| | Variant | CKKFVRENEIPKDECPNFQGGPAIANIIAKSREFTEWEIYQSSLAIQEVIFTLPKD |
| | (N- & | KLPEPILKEEWRAQWLSEHGLDTVPYKEAAGLNLIIKNAVNTYKGVQVKVDN |
| | C- | KNKNNLAKINRKNEIAKLNGEQEISFEEIKAFDDKGYLLQKPSPNKSIYCYQSV |
| | term | SPKPFITSKYHNVNLPEEYIGYYRKSNEPIVSPYQFDRLRIPIGEPGYVPKWQYT |
| | NLS) | FLSKKENKRRKLSKRIKNVSPILGIICIKKDWCVFDMRGLLRTNHWKKYHKPT |
| | | DSINDLFDYFTGDPVIDTKANVVRFRYKMENGIVNYKPVREKKGKELLENICD |
| | | QNGSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPNDLP |
| | | WDKMISGTHFISEKAQVSNKSEIYFTSTDKGKTKDVMKSDYKWFQDYKPKLS |
| | | KEVRDALSDIEWRLRRESLEFNKLSKSREQDARQLANWISSMCDVIGIENLVK |
| | | KNNFFGGSGKREPGWDNFYKPKKENRWWINAIHKALTELSQNKGKRVILLPA |
| | | MRTSITCPKCKYCDSKNRNGEKFNCLKCGIELNADIDVATENLATVAITAQSM |
| | | PKPTCERSGDAKKPVRARKAKAPEFHDKLAPSYTVVLREAV |
| 9 | L26H | MAPKKKRKVGIHGVPAAMIKPTVSQFLTPGFKLIRNHSRTAGHKLKNEGEEA |
| | Variant | CKKFVRENEIPKDECPNFQGGPAIANIIAKSREFTEWEIYQSSLAIQEVIFTLPKD |
| | (N- | KLPEPILKEEWRAQWLSEHGLDTVPYKEAAGLNLIIKNAVNTYKGVQVKVDN |
| | term | KNKNNLAKINRKNEIAKLNGEQEISFEEIKAFDDKGYLLQKPSPNKSIYCYQSV |
| | NLS) | SPKPFITSKYHNVNLPEEYIGYYRKSNEPIVSPYQFDRLRIPIGEPGYVPKWQYT |
| | | FLSKKENKRRKLSKRIKNVSPILGIICIKKDWCVFDMRGLLRTNHWKKYHKPT |
| | | DSINDLFDYFTGDPVIDTKANVVRFRYKMENGIVNYKPVREKKGKELLENICD |
| | | QNGSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPNDLP |
| | | WDKMISGTHFISEKAQVSNKSEIYFTSTDKGKTKDVMKSDYKWFQDYKPKLS |
| | | KEVRDALSDIEWRLRRESLEFNKLSKSREQDARQLANWISSMCDVIGIENLVK |
| | | KNNFFGGSGKREPGWDNFYKPKKENRWWINAIHKALTELSQNKGKRVILLPA |
| | | MRTSITCPKCKYCDSKNRNGEKFNCLKCGIELNADIDVATENLATVAITAQSM |
| | | PKPTCERSGDAKKPVRARKAKAPEFHDKLAPSYTVVLREAV |
| 10 | L26K | MAPKKKRKVGIHGVPAAMIKPTVSQFLTPGFKLIRNHSRTAGKKLKNEGEEA |
| | Variant | CKKFVRENEIPKDECPNFQGGPAIANIIAKSREFTEWEIYQSSLAIQEVIFTLPKD |
| | (N- | KLPEPILKEEWRAQWLSEHGLDTVPYKEAAGLNLIIKNAVNTYKGVQVKVDN |
| | term | KNKNNLAKINRKNEIAKLNGEQEISFEEIKAFDDKGYLLQKPSPNKSIYCYQSV |
| | NLS) | SPKPFITSKYHNVNLPEEYIGYYRKSNEPIVSPYQFDRLRIPIGEPGYVPKWQYT |
| | | FLSKKENKRRKLSKRIKNVSPILGIICIKKDWCVFDMRGLLRTNHWKKYHKPT |
| | | DSINDLFDYFTGDPVIDTKANVVRFRYKMENGIVNYKPVREKKGKELLENICD |
| | | QNGSCKLATVDVGQNNPVAIGLFELKKVNGELTKTLISRHPTPIDFCNKITAYR |
| | | ERYDKLESSIKLDAIKQLTSEQKIEVDNYNNNFTPQNTKQIVCSKLNINPNDLP |
| | | WDKMISGTHFISEKAQVSNKSEIYFTSTDKGKTKDVMKSDYKWFQDYKPKLS |
| | | KEVRDALSDIEWRLRRESLEFNKLSKSREQDARQLANWISSMCDVIGIENLVK |
| | | KNNFFGGSGKREPGWDNFYKPKKENRWWINAIHKALTELSQNKGKRVILLPA |
| | | MRTSITCPKCKYCDSKNRNGEKFNCLKCGIELNADIDVATENLATVAITAQSM |
| | | PKPTCERSGDAKKPVRARKAKAPEFHDKLAPSYTVVLREAV |
| 11 | L26R | MIKPTVSQFLTPGFKLIRNHSRTAGRKLKNEGEEACKKFVRENEIPKDECPNFQ |
| | Variant | GGPAIANIIAKSREFTEWEIYQSSLAIQEVIFTLPKDKLPEPILKEEWRAQWLSE |
| | (C- | HGLDTVPYKEAAGLNLIIKNAVNTYKGVQVKVDNKNKNNLAKINRKNEIAKL |
| | term | NGEQEISFEEIKAFDDKGYLLQKPSPNKSIYCYQSVSPKPFITSKYHNVNLPEEY |
| | NLS) | IGYYRKSNEPIVSPYQFDRLRIPIGEPGYVPKWQYTFLSKKENKRRKLSKRIKN |
| | | VSPILGIICIKKDWCVFDMRGLLRTNHWKKYHKPTDSINDLFDYFTGDPVIDT |
| | | KANVVRFRYKMENGIVNYKPVREKKGKELLENICDQNGSCKLATVDVGQNN |
| | | PVAIGLFELKKVNGELTKTLISRHPTPIDFCNKITAYRERYDKLESSIKLDAIKQ |
| | | LTSEQKIEVDNYNNNFTPQNTKQIVCSKLNINPNDLPWDKMISGTHFISEKAQ |
| | | VSNKSEIYFTSTDKGKTKDVMKSDYKWFQDYKPKLSKEVRDALSDIEWRLRR |
| | | ESLEFNKLSKSREQDARQLANWISSMCDVIGIENLVKKNNFFGGSGKREPGW |
| | | DNFYKPKKENRWWINAIHKALTELSQNKGKRVILLPAMRTSITCPKCKYCDS |
| | | KNRNGEKFNCLKCGIELNADIDVATENLATVAITAQSMPKPTCERSGDAKKP |
| | | VRARKAKAPEFHDKLAPSYTVVLREAVKRPAATKKAGQAKKKKEF |
| 12 | L26H | MIKPTVSQFLTPGFKLIRNHSRTAGHKLKNEGEEACKKFVRENEIPKDECPNFQ |
| | Variant | GGPAIANIIAKSREFTEWEIYQSSLAIQEVIFTLPKDKLPEPILKEEWRAQWLSE |
| | (C- | HGLDTVPYKEAAGLNLIIKNAVNTYKGVQVKVDNKNKNNLAKINRKNEIAKL |
| | term | NGEQEISFEEIKAFDDKGYLLQKPSPNKSIYCYQSVSPKPFITSKYHNVNLPEEY |
| | NLS) | IGYYRKSNEPIVSPYQFDRLRIPIGEPGYVPKWQYTFLSKKENKRRKLSKRIKN |
| | | VSPILGIICIKKDWCVFDMRGLLRTNHWKKYHKPTDSINDLFDYFTGDPVIDT |
| | | KANVVRFRYKMENGIVNYKPVREKKGKELLENICDQNGSCKLATVDVGQNN |
| | | PVAIGLFELKKVNGELTKTLISRHPTPIDFCNKITAYRERYDKLESSIKLDAIKQ |
| | | LTSEQKIEVDNYNNNFTPQNTKQIVCSKLNINPNDLPWDKMISGTHFISEKAQ |
| | | VSNKSEIYFTSTDKGKTKDVMKSDYKWFQDYKPKLSKEVRDALSDIEWRLRR |
| | | ESLEFNKLSKSREQDARQLANWISSMCDVIGIENLVKKNNFFGGSGKREPGW |
| | | DNFYKPKKENRWWINAIHKALTELSQNKGKRVILLPAMRTSITCPKCKYCDS |
| | | KNRNGEKFNCLKCGIELNADIDVATENLATVAITAQSMPKPTCERSGDAKKP |
| | | VRARKAKAPEFHDKLAPSYTVVLREAVKRPAATKKAGQAKKKKEF |
| 13 | L26K | MIKPTVSQFLTPGFKLIRNHSRTAGKKLKNEGEEACKKFVRENEIPKDECPNFQ |
| | Variant | GGPAIANIIAKSREFTEWEIYQSSLAIQEVIFTLPKDKLPEPILKEEWRAQWLSE |
| | (C- | HGLDTVPYKEAAGLNLIIKNAVNTYKGVQVKVDNKNKNNLAKINRKNEIAKL |
| | term | NGEQEISFEEIKAFDDKGYLLQKPSPNKSIYCYQSVSPKPFITSKYHNVNLPEEY |
| | NLS) | IGYYRKSNEPIVSPYQFDRLRIPIGEPGYVPKWQYTFLSKKENKRRKLSKRIKN |
| | | VSPILGIICIKKDWCVFDMRGLLRTNHWKKYHKPTDSINDLFDYFTGDPVIDT |
| | | KANVVRFRYKMENGIVNYKPVREKKGKELLENICDQNGSCKLATVDVGQNN |
| | | PVAIGLFELKKVNGELTKTLISRHPTPIDFCNKITAYRERYDKLESSIKLDAIKQ |
| | | LTSEQKIEVDNYNNNFTPQNTKQIVCSKLNINPNDLPWDKMISGTHFISEKAQ |
| | | VSNKSEIYFTSTDKGKTKDVMKSDYKWFQDYKPKLSKEVRDALSDIEWRLRR |
| | | ESLEFNKLSKSREQDARQLANWISSMCDVIGIENLVKKNNFFGGSGKREPGW |
| | | DNFYKPKKENRWWINAIHKALTELSQNKGKRVILLPAMRTSITCPKCKYCDS |
| | | KNRNGEKFNCLKCGIELNADIDVATENLATVAITAQSMPKPTCERSGDAKKP |
| | | VRARKAKAPEFHDKLAPSYTVVLREAVKRPAATKKAGQAKKKKEF |

TABLE 2.2
SEQUENCES OF EXEMPLARY EXONUCLEASE FUSION PARTNER
SEQUENCES

| SEQ | | |
|---|---|---|
| ID | | |
| NO: | Description | Sequence* |
| 43 | dem1 | MLGRALINKYGFLIHPRRFVHLNDKSLDGTFILPSKKNHMYDVPTNDPSG |
| | having | ILNASDIDRINNLPFFDNTSPTKETNTKEGALLSEKLASVKELFGEDPENPS |
| | 5′→3′ | FINYRFPRGLENPYFDIQVNQLKKKRLSVTQLCTTQNWCELRNFYDFYSQ |
| | exonuclease | NLSNQLLNLKFQVQKGKKIHKSLEDETHPELNQYKSFTHNFLALTKLSM |
| | activity | DIDNDMDALLDNWFNSINRLVSLFTKGDGHAREIVCHGFINLEDGKLVEH |
| | for | LLNSDSKTKENVIISGVIDHLTLRNRHNHQVQKGAAHLDTEYQSWGNILT |
| | SSDNA | NLLSNLKELKSNNEIVISDIKTRSVPKIPSIESVIESSKLQTMYYKFFFSHLS |
| | | QDMTQTYHSFLINAQRRGLDVDAPINPTKILTFILTNPLFANDVKNLLYGL |
| | | PINHSAFDNDAKGSNTFDMTAFNDLLDRGPTSFNVPIEQDEDSSESTKCVS |
| | | LRDYGHFYTKWKTPLTLKYFAARLSQIYFIVGNLVSNDLMIEYYYHNDN |
| | | FHNIIFPYDPLKLGTHAHDSAMVWFGGRDMHPIEPTQKNFNTYCKFCDY |
| | | RHVCSWKNKNELKLIDLGKELKKIILESSMK |
| 44 | exo1 | MGIQGLLPQLKPIQNPVSLRRYEGEVLAIDGYAWLHRAACSCAYELAMG |
| | having | KPTDKYLQFFIKRFSLLKTFKVEPYLVFDGDAIPVKKSTESKRRDKRKEN |
| | 5′→3′ | KAIAERLWACGEKKNAMDYFQKCVDITPEMAKCIICYCKLNGIRYIVAPF |
| | exonuclease | EADSQMVYLEQKNIVQGIISEDSDLLVFGCRRLITKLNDYGECLEICRDNF |
| | activity | IKLPKKFPLGSLTNEEIITMVCLSGCDYTNGIPKVGLITAMKLVRRFNTIER |
| | for | IILSIQREGKLMIPDTYINEYEAAVLAFQFQRVFCPIRKKIVSLNEIPLYLKD |
| | dsDNA | TESKRKRLYACIGFVIHRETQKKQIVHFDDDIDHHLHLKIAQGDLNPYDF |
| | | HQPLANREHKLQLASKSNIEFGKTNTTNSEAKVKPIESFFQKMTKLDHNP |
| | | KVANNIHSLRQAEDKLTMAIKRRKLSNANVVQETLKDTRSKFFNKPSMT |
| | | VVENFKEKGDSIQDFKEDTNSQSLEEPVSESQLSTQIPSSFITTNLEDDDNL |
| | | SEEVSEVVSDIEEDRKNSEGKTIGNEIYNTDDDGDGDTSEDYSETAESRVP |
| | | TSSTTSFPGSSQRSISGCTKVLQKFRYSSSFSGVNANRQPLFPRHVNQKSR |
| | | GMVYVNQNRDDDCDDNDGKNQITQRPSLRKSLIGARSQRIVIDMKSVDE |
| | | RKSFNSSPILHEESKKRDIETTKSSQARPAVRSISLLSQFVYKGK |
| 45 | exo5 | MSKSWGKFIEEEEAEMASRRNLMIVDGTNLGFRFKHNNSKKPFASSYVS |
| | having | TIQSLAKSYSARTTIVLGDKGKSVFRLEHLPEYKGNRDEKYAQRTEEEKA |
| | 5′→3′ | LDEQFFEYLKDAFELCKTTFPTFTIRGVEADDMAAYIVKLIGHLYDHVWL |
| | exonuclease | ISTDGDWDTLLTDKVSRFSFTTRREYHLRDMYEHHNVDDVEQFISLKAIM |
| | activity | GDLGDNIRGVEGIGAKRGYNIIREFGNVLDIIDQLPLPGKQKYIQNLNASE |
| | for | ELLFRNLILVDLPTYCVDAIAAVGQDVLDKFTKDILEIAEQ |
| | SSDNA | |
| | and | |
| | dsDNA | |
| 46 | FenA | MTAPILLLDGASMWFRSYFGVPSSIKAPDGRPVNAVRGFIDAISTLVTREK |
| | having | PRRLVVCRDDDWRPQWRVDLIPSYKAHRVAEPEPDGVPDIEEVPDDLTP |
| | 5′→3′ | QVNMILELLDAFGIPTAGAAGFEADDVLGTLSAREERDPVVVVSGDRDL |
| | exonuclease | LQLVRDEPAPQVRVLYLGRGLAKATKWGPAEVAEQYGVPLDRAGTAYA |
| | activity | ELALLRGDPSDGLPGVAGIGEKTAASLLAKHGSLQNILDAAHDPKSGLSK |
| | for | AHRTKLLGAVDYIAAAETVVRVATDAPVTFSTPTDTLPLAAGDPARVAE |
| | SSDNA | LAAAYGVSSSISRLQTALDQLPD |
| | and | |
| | dsDNA | |
| 47 | nurA | MIDKAYEELIKMKEKIMRDSYTIHKELSESVESILNELWINYSPESVEHSK |
| | having | KLLAIDGGMWVKETRQGVIFIVNAKAIVFEGINEINSEGKVLVHIFSPGNY |
| | 5′→3′ | AKERIELLMQLLELQLALKLVENVDYVLLDGSFSKKLGRHKSELKVDLL |
| | exonuclease | DDIVSIDKILSLEEKDEDNMLRFLIAENQLVLSELVSRYKDKLLFISKNSKS |
| | activity | SDLFKQAYSDITILELFTQNCGYSKILEKKIDENYILSRKASKLLSGLNYYF |
| | for | TNLRLEPSERLFRLDFFNADKIFEYLKVLKPVSLKGYPYPLIKVHKDVRVG |
| | SSDNA | KEDRERIYSILEMKRKDISWWPSQFY |
| | and | |
| | dsDNA | |
| 48 | recJ | MKKLIKRREIPIGNSVSNHPLLDRLYRARHIQNTKELDRTLKSMLNPNQL |
| | having | YGIEQAVNLLVEAYQPQQKIVIVGDFDADGATSTALSVLALRQLGFSDVD |
| | 5′→3′ | YLVPNRFEQGYGLSIPVAEMAIEKGVQLLMTVDNGVSSFDGVAFLKEKGI |
| | exonuclease | RVLVTDHHLPPETLPPADAIVNPNLSQCGFPSKSLAGVGVAFYLMLAVRA |
| | activity | KFRELGIFTAETQPNFTDLLDLVALGTIADVVPLDQNNRILAYQGLMRIRA |
| | for | RHCRLGIIALAEVANRNVEQFTSSDLGFCIGPRLNAAGRLDNMSIGVELLL |
| | SSDNA | ANEMSKARELALDLDQLNQTRKEIEAGMKLEAIKICQNLTALFKELPYGI |
| | | TLYQPDWHQGVLGIVSSRIKDQYHRPVIAFAQDSEGILKGSARSIEGLHM |
| | | RDVLERIHSQHPNMILKFGGHAMAAGLSIREEHFADFQHIFNQTVADWL |
| | | DEEHLQGVIWTDGELNSNEFNLETAELIKSVGTWGQGFPEPCFDGEFKIL |
| | | DQRAIGQNKNHLKMLLEPKQGGVLLDAIAFNINTRLYPDLSIKQARLAYK |
| | | LEINEFRGNRSLQLLVDYIEPIDE |
| 49 | sbcB | MMNDGKQQSTFLFHDYETFGTHPALDRPAQFAAIRTDSEFNVIGEPEVFY |
| | having | CKPADDYLPQPGAVLITGITPQEARAKGENEAAFAARIHSLFTVPKTCILG |
| | 5′→3′ | YNNVRFDDEVTRNIFYRNFYDPYAWSWQHDNSRWDLLDVMRACYALR |
| | exonuclease | PEGINWPENDDGLPSFRLEHLTKANGIEHSNAHDAMADVYATIAMAKLV |
| | activity | KTRQPRLFDYLFTHRNKHKLMALIDVPQMKPLVHVSGMFGAWRGNTSW |
| | for | VAPLAWHPENRNAVIMVDLAGDISPLLELDSDTLRERLYTAKTDLGDNA |
| | SSDNA | AVPVKLVHINKCPVLAQANTLRPEDADRLGINRQHCLDNLKILRENPQVR |
| | | EKVVAIFAEAEPFTPSDNVDAQLYNGFFSDADRAAMKIVLETEPRNLPAL |
| | | DITFVDKRIEKLLFNYRARNFPGTLDYAEQQRWLEHRRQVFTPEFLQGYA |
| | | DELQMLVQQYADDKEKVALLKALWQYAEEIV |
| 50 | snm1 | MSRKSIVQIRRSEVKRKRSSTASSTSEGKTLHKNTHTSSKRQRTLTEFNIPT |
| | having | SSNLPVRSSSYSFSRFSCSTSNKNTEPVIINDDDHNSICLEDTAKVEITIDTD |
| | 5′→3′ | EEELVSLHDNEVSAIENRTEDRIVTELEEQVNVKVSTEVIQCPICLENLSHL |
| | exonuclease | ELYERETHCDTCIGSDPSNMGTPKKNIRSFISNPSSPAKTKRDIATSKKPTR |
| | activity | VKLVLPSFKIIKFNNGHEIVVDGFNYKASETISQYFLSHFHSDHYIGLKKS |
| | for | WNNPDENPIKKTLYCSKITAILVNLKFKIPMDEIQILPMNKRFWITDTISVV |
| | SSDNA | TLDANHCPGAIIMLFQEFLANSYDKPIRQILHTGDFRSNAKMIETIQKWLA |
| | and | ETANETIDQVYLDTTYMTMGYNFPSQHSVCETVADFTLRLIKHGKNKTF |
| | dsDNA | GDSQRNLFHFQRKKTLTTHRYRVLFLVGTYTIGKEKLAIKICEFLKTKLFV |
| | | MPNSVKFSMMLTVLQNNENQNDMWDESLLTSNLHESSVHLVPIRVLKSQ |
| | | ETIEAYLKSLKELETDYVKDIEDVVGFIPTGWSHNFGLKYQKKNDDDENE |
| | | MSGNTEYCLELMKNDRDNDDENGFEISSILRQYKKYNKFQVFNVPYSEH |
| | | SSFNDLVKFGCKLKCSEVIPTVNLNNLWKVRYMTNWFQCWENVRKTRA |
| | | AK |
| 51 | TatD | MFDIGVNLTSSQFVKDRDDVVTRALAAGVSGMLLTGTNLHESQQAQKL |
| | having | AQRYACCWSTAGVHPHDSSQWQSETEDAIVALARQPDVVAIGECGLDFN |
| | 5′→3′ | RNFSTPQEQERAFQAQLRIAAELQMPVFMHCRDAHARFLALLEPWLDKL |
| | exonuclease | PGAVLHCFTGTREEMQECIDRGLYIGITGWVCDERRGLELRELLPFIPAEK |
| | activity | LLIETDAPYLLPRDLTPKPASRRNEPAHLAHILARVAHWRGEDPQWLAAT |
| | for | TDANVKTLFGIAF |
| | SSDNA | |
| | and RNA | |
| 52 | TREX1 | MGSQALPPGPMQTLIFFDMEATGLPFSQPKVTELCLLAVHRCALESPPTS |
| | having | QGPPPTVPPPPRVVDKLSLCVAPGKACSPAASEITGLSTAVLAAHGRQCF |
| | 5′→3′ | DDNLANLLLAFLRRQPQPWCLVAHNGDRYDFPLLQAELAMLGLTSALD |
| | exonuclease | GAFCVDSITALKALERASSPSEHGPRKSYSLGSIYTRLYGQSPPDSHTAEG |
| | activity | DVLALLSICQWRPQALLRWVDAHARPFGTIRPMYGVTASARTKPRPSAV |
| | for | TTTAHLATTRNTSPSLGESRGTKDLPPVKDPGALSREGLLAPLGLLAILTL |
| | SSDNA | AVATLYGLSLATPGE |
| | and | |
| | dsDNA | |

TABLE 3 provides illustrative repeat sequences for use in guide nucleic acids that are useful in the compositions, systems and methods described herein.

TABLE 3
EXEMPLARY REPEAT SEQUENCES

| SEQ ID | |
|---|---|
| NO: | Repeat Sequence (5′-> 3′) |
| 24 | AUUGCUCCUUACGAGGAGAC |
| 38 | CUUUCAAGACUAAUAGAUUGCUCCUUACGAGGAGAC |

TABLE 4 provides illustrative spacer sequences for use in guide nucleic acids that are useful in the compositions, systems and methods described herein

TABLE 4
EXEMPLARY SPACER SEQUENCES

| SEQ ID NO: | Spacer Sequences (5′->3′) |
|---|---|
| 25 | AUAUAAGUGGAGGCGUCG |
| 26 | GCCUUAACAAGCUGCUCU |
| 27 | CUCACGUCAUCCAGCAGAGA |
| 39 | GAGCUGGUGGCGUAGGC |
| 40 | GAGCUGAUGGCGUAGGC |
| 41 | GAGAGAGGGACUUUCCCGUU |
| 55 | GAGCAACGGCGGAAGGU |

TABLE 5 provides illustrative crRNA sequences that are useful in the compositions, systems and methods described herein.

TABLE 5
EXEMPLARY CRRNA SEQUENCES

| SEQ | |
|---|---|
| ID | |
| NO: | crRNA Sequence (5′->3′) |
| 28 | AUUGCUCCUUACGAGGAGACAUAUAAGUGGAGGCGUCG |
| 29 | AUUGCUCCUUACGAGGAGACGCCUUAACAAGCUGCUCU |
| 30 | AUUGCUCCUUACGAGGAGACCUCACGUCAUCCAGCAGAGA |
| 34 | CUUUCAAGACUAAUAGAUUGCUCCUUACGAGGAGACGAGCU |
| | GGUGGCGUAmGmGmC |
| 35 | CUUUCAAGACUAAUAGAUUGCUCCUUACGAGGAGACGAGCU |
| | GAUGGCGUAmGmGmC |
| 42 | CUUUCAAGACUAAUAGAUUGCUCCUUACGAGGAGACGAGAG |
| | AGGGACUUUCCCGUU |
| 126 | AUAGAUUGCUCCUUACGAGGAGACGUAUUUGUGAAGUCUUA |
| 127 | AUAGAUUGCUCCUUACGAGGAGACUAUUUGUGAAGUCUUAC |
| 128 | AUAGAUUGCUCCUUACGAGGAGACCUUUGCACUUUCCUUAG |
| 129 | AUAGAUUGCUCCUUACGAGGAGACAGUCACUCUAAGUUACU |
| 130 | AUAGAUUGCUCCUUACGAGGAGACAAUAAAGCAUAGUGCAA |
| 131 | AUAGAUUGCUCCUUACGAGGAGACAAAUAAAGCAUAGUGCA |
| 132 | AUAGAUUGCUCCUUACGAGGAGACUGAGAUCAACAGCACAG |
| 133 | AUAGAUUGCUCCUUACGAGGAGACAUGAGAUCAACAGCACA |
| 134 | AUAGAUUGCUCCUUACGAGGAGACCAUUUUAGUCUGUCUUC |
| 135 | AUAGAUUGCUCCUUACGAGGAGACAAAGGCAGAAGAAAUAA |
| 136 | AUAGAUUGCUCCUUACGAGGAGACGGCUCUGAUUCCUACAG |
| 137 | AUAGAUUGCUCCUUACGAGGAGACCUGUAGGAAUCAGAGCC |
| 138 | AUAGAUUGCUCCUUACGAGGAGACGACUUAGAUUAUGCAUU |
| 139 | AUAGAUUGCUCCUUACGAGGAGACUUUUCUUCAGUAUUUAA |
| 140 | AUAGAUUGCUCCUUACGAGGAGACUCUUCAGUAUUUAACAA |

m = a 2′ O-methyl modification of the subsequent indicated nucleotide

TABLE 6 provides illustrative target nucleic acids that are useful in the compositions, systems and methods described herein.

TABLE 6
EXEMPLARY TARGET NUCLEIC ACIDS
Exemplary target nucleic acids

AAVS1, ABCA4, ABCB11, ABCC8, ABCD1, ABCG5, ABCG8, ACAD9, ACADM, ACADVL, ACAT1,
ACTA1, ACOX1, ACSF3, ADA, ADAMTS2, ADGRG1, AGA, AGL, AGPS, AGXT, AHI1, AIRE,
ALDH3A2, ALDOB, ALG6, ALK, ALKBH5, ALMS1, ALPL, AMRC9, AMT, ANAPC10, ANAPC11,
ANGPTL3, ANGPTL4, APC, Apo(a), APOCIII, APOEε4, APOL1, APP, AQP2, AR, ARFRP1, ARG1,
ARH, ARL13B, ARL6, ARSA, ARSB, ASL, ASNS, ASPA, ASS1, ATM, ATP6V1B1, ATP7A, ATP7B, ATRX,
ATXN1, ATXN10, ATXN2, ATXN3, ATXN7, ATXN8OS, AXIN1, AXIN2, B2M, BACE-1, BAK1, BAP1,
BARD1, BAX2, BBS1, BBS10, BBS12, BBS2, BCKDHA, BCKDHB, BCL2L2, BCS1L, BEST1, Betaglobin
gene, BLM, BMPR1A, BRAF, BRAFV600E, BRCA1, BRCA2, BRIP1, BSND, C9orf72, C282Y, CA4,
CACNA1A, CAH1, CAPN3, CASR, CBS, CCNB1 CC2D2A, CCR5, CD1, CD2, CD3, CD3D, CD3Z, CD4,
CD5, CD6, CD7, CD8A, CD8B, CD9, CD14, CD18, CD19, CD21, CD22, CD23, CD27, CD28, CD30,
CD33, CD34, CD36, CD38, CD40, CD40L, CD44, CD46, CD47, CD48, CD52, CD55, CD57, CD58,
CD59, CD68, CD69, CD72, CD73, CD74, CD79A, CD80, CD81, CD83, CD84, CD86, CD90, CD93,
CD96, CD99, CD100, CD123, CD160, CD163, CD164, CD164L2, CD166, CD200, CD204, CD207,
CD209, CD226, CD244, CD247, CD274, CD276, CD300, CD320, CDC73, CDH1, CDH23, CDK11,
CDK4, CDKN1A, CDKN1B, CDKN1C, CDKN2A, CDKN2B, CEBPA, CELA3B, CEP290, CERKL, CFB,
CFTR, CHCHD10, CHEK2, CHM, CHRNE, CIDEB, CIITA, CLN3, CLN5, CLN6, CLN8, CLRN1, CLTA,
CMT1A, CNBP, CNGB1, CNGB3, COL1A1, COL1A2, COL27A1, COL4A3, COL4A4, COL4A5,
COL6A1, COL6A2, COL6A3, COL7A1, CPS1, CPT1A, CPT2, CRB1, CREBBP, CRX, CRYAA, CTNNA1,
CTNNB1, CTNND2, CTNS, CTSK, CXCL12, CYBA, CYBB, CYP11B1, CYP11B2, CYP17A1, CYP19A1,
CYP21A2, CYP27A1, DBT, DCC, DCLRE1C, DERL2, DFNA36, DFNB31, DGAT2, DHCR7, DHDDS,
DICER1, DIS3L2, DLD, DMD, DMPK, DNAH5, DNAI1, DNAI2, DNM2, DNMT1, DPC4, DUX4, DYSF,
EDA, EDN3, EDNRB, EGFR, EIF2B5, EMC2, EMC3, EMD, EMX1, EN1, EPCAM, ERCC6, ERCC8,
ESCO2, ETFA, ETFDH, ETHE1, EVC, EVC2, EYS, F5, F9, FX1, FAH, FAM161A, FANCA, FANCB,
FANCC, FANCD1, FANCD2, FANCE, FANCF, FANCG, FANCI, FANCJ, FANCL, FANCM, FANCN,
FANCP, FANCS, FBN1, FGF14, FGFR2, FGFR3, FGA, FGB, FGG, FH, FHL1, FIX, FKRP, FKTN,
FLCN, FMR1, FOXP3, FSCN2, FSHD1, FUS, FUT8, FVIII, FXI, FXII, FXN, G6PC, GAA, GALC,
GALK1, GALT, GAMT, GATA2, GATA-4, GBA, GBE1, GCDH, GCGR, GDNF, GFAP, GFM1, GHR,
GJB1, GJB2, GLA, GLB1, GLDC, GLE1, GNE, GNPTAB, GNPTG, GNS, GPAM, GPC3, GPR98,
GREM1, GRHPR, GRIN2B, H2AFX, H2AX, HADHA, HAX1, HBA1, HBA2, HBB, HBV cccDNA, HER2,
HEXA, HEXB, HFE, HGSNAT, HLCS, HMGCL, HAO1, HOGA1, HOXB13, HPRPF3, HPRT1, HPS1,
HPS3, HRAS, HRD1, HSD17B4, HSD17B13, HSD3B2, HTT, HUS1, HYAL1, HYLS1, IDS, IDUA,
IFITM5, IFN, IFN-γ, IKBKAP, IL2RG, IL7R, IMPDH1, INPP5E, IRF4, ITGB2, ITPR1, IVD, JAG1,
JAK1, JAK3, KCNC3, KCND3, KCNJ11, KLKB1, KLHL7, KRAS, LAMA1, LAMA2, LAMA3, LAMB3,
LAMC2, LCA5, LDHA, LDLR, LDLRAP1, LHX3, LIFR, LIPA, LMNA, LMOD3, LOR, LOXHD1, LPA,
LPL, LRAT, LRP6, LRPPRC, LRRK2, MADR2, MAN2B1, MAPT, MARC1, MAX, MCM6, MCOLN1,
MECP2, MED17, MEFV, MEN1, MERTK, MESP2, MET, METex14, MFN2, MFSD8, MIA3, MITF,
MKL2, MKS1, MLC1, MLH1, MLH3, MMAA, MMAB, MMACHC, MMADHC, MMD, MPI, MPL,
MPV17, MSH2, MSH3, MSH6, MTHFD1L, MTHFR, MTM1, MTRR, MTTP, MUT, MUTYH, MYC,
MYH7, MYO7A, NAGLU, NAGS, NBN, NDRG1, NDUFAF5, NDUFS6, NEB, NF1, NF2, NKX2-5, NOG,
NOTCH1, NOTCH2, NPC1, NPC2, NPHP1, NPHS1, NPHS2, NRAS, NR2E3, NTHL1, NTRK, NTRK1,
OAT, OCT4, OFD1, OPA3, OTC, PAH, PALB2, PAQR8, PAX3, PC, PCCA, PCCB, PCDH15, PCSK9,
PD1, PDCD1, PDE6B, PDGFRA, PDHA1, PDHB, PEX1, PEX10, PEX12, PEX13, PEX14, PEX16,
PEX19, PEX2, PEX26, PEX3, PEX5, PEX6, PEX7, PFKM, PHGDH, PHOX2B, PKD1, PKD2, PKHD1,
PKK, PLEKHG4, PMM2, PMP22, PMS1, PMS2, PNPLA3, POLD1, POLE, POMGNT1, POT1,
POU5F1, PPM1A, PPP2R2B, PPT1, PRCD, PRKAG2, PRKAR1A, PRKCG, PRNP, PROM1, PROP1,
PRPF31, PRPF8, PRPH2, PRPS1, PSAP, PSD3, PSD95, PSEN1, PSEN2, PSRC1, PTCH1, PTEN, PTS,
PUS1, PYGM, RAB23, RAD50, RAD51C, RAD51D, RAG1, RAG2, RAPSN, RARS2, RB1, RDH12,
RECQL4, RET, RHO, RICTOR, RMRP, ROS1, RP1, RP2, RPE65, RPGR, RPGRIP1L, RPL32P3, RS1,
RTCA, RTEL1, RUNX1, SACS, SAMHD1, SCN1A, SCN2A, SDHA, SDHAF2, SDHB, SDHC, SDHD,
SEL1L, SEPSECS, SERPINA1, SERPINC1, SERPING1, SGCA, SGCB, SGCG, SGSH, SIRT1, SLC12A3,
SLC12A6, SLC17A5, SLC22A5, SLC25A13, SLC25A15, SLC26A2, SLC26A4, SLC35A3, SLC35B4,
SLC37A4, SLC39A4, SLC4A11, SLC6A8, SLC7A7, SMAD3, SMAD4, SMARCA4, SMARCAL1,
SMARCB1, SMARCE1, SMN1, SMPD1, SNAI2, SNCA, SNRNP200, SOD1, SOX10, SPARA7, SPTBN2,
STAR, STAT3, STK11, SUFU, SUMF1, SYNE1, SYNE2, SYS1, TARDBP, TAT, TBK1, TBP, TCIRG1,
TCTN3, TECPR2, TERC, TERT, TFR2, TGFBR2, TGM1, TH, TLE3, TMEM127, TMEM138, TMEM216,
TMEM43, TMEM67, TMPRSS6, TNNI2, TNNT1, TNNT3, TOP1, TOPORS, TP53, TPM2, TPM3, TPP1,
TRAC, TRMU, TSC1, TSC2, TSFM, TSPAN14, TTBK2, TTC8, TTPA, TTR, TULP1, TYMP, UBE2G2,
UBE2J1, UBE3A, USH1C, USH1G, USH2A, VEGF, VHL, VPS13A, VPS13B, VPS35, VPS45, VRK1,
VSX2, VWF, WAS, WDR19, WDR48, WFS1, WNT10A, WRN, WS2B, WS2C, WT1, XPA, XPC, XPF,
XRCC3, YAP1, ZAC1, ZEB1, ZFYVE26, and ZNF423.

TABLE 6.1 provides illustrative diseases and syndromes for compositions, systems and methods described herein.

TABLE 6.1
DISEASES AND SYNDROMES
Exemplary Diseases and Syndromes

11-hydroxylase deficiency; 17,20-desmolase deficiency; 17-hydroxylase deficiency; 3-
hydroxyisobutyrate aciduria; 3-hydroxysteroid dehydrogenase deficiency; 46,XY gonadal dysgenesis;
AAA syndrome; ABCA3 deficiency; ABCC8-associated hyperinsulinism; aceruloplasminemia;
acromegaly; achondrogenesis type 2; acral peeling skin syndrome; acrodermatitis enteropathica; acute
bacterial infection; adrenocortical micronodular hyperplasia; adrenoleukodystrophies;
adrenomyeloneuropathies; Aicardi-Goutieres syndrome; AIDS; Alagille disease (also called Alagille
Syndrome); Alexander Disease; Alpers syndrome; alpha-1 antitrypsin deficiency (AATD); alpha-
mannosidosis; Alstrom syndrome; Alzheimer's disease; amebic dysentery; amelogenesis imperfecta;
amish type microcephaly; amyotrophic lateral sclerosis (ALS); anaplastic large cell lymphoma; anauxetic
dysplasia; androgen insensitivity syndrome; angiopathic thrombosis; antiphospholipid syndrome;
Antley-Bixler syndrome; APECED; Apert syndrome; aplasia of lacrimal and salivary glands; arginase-1
deficiency; argininosuccinic aciduria; argininemia; arrhythmogenic right ventricular dysplasia; Arts
syndrome; ARVD2; arylsulfatase deficiency type metachromatic leukodystrophy; ataxia telangiectasia;
atherosclerotic cardiovascular disease; autoimmune lymphoproliferative syndrome; autoimmune
polyglandular syndrome type 1; autosomal dominant anhidrotic ectodermal dysplasia; autosomal
dominant deafness; autosomal dominant polycystic kidney disease; autosomal recessive microtia;
autosomal recessive renal glucosuria; autosomal visceral heterotaxy; babesiosis; bacterial vaginosis;
balantidial dysentery; Bardet-Biedl syndrome; Bartter syndrome; basal cell nevus syndrome; Batten
disease; benign recurrent intrahepatic cholestasis; beta-mannosidosis; β-thalassemia; Bethlem myopathy;
Blackfan-Diamond anemia; bleeding disorder (coagulation); blepharophimosis; Byler disease; C
syndrome; CADASIL; calcific aortic stenosis; calcification of joints and arteries; carbamoyl phosphate
synthetase I deficiency; carcinoid syndrome diarrhea; cardiofaciocutaneous syndrome; cardiovascular
disease (CVD); Carney triad; carnitine palmitoyltransferase deficiencies; cartilage-hair hypoplasia; cblC
type of combined methylmalonic aciduria; CD18 deficiency; CD3Z-associated primary T-cell
immunodeficiency; CD40L deficiency; CDAGS syndrome; CDG1A; CDG1B; CDG1M; CDG2C;
CEDNIK syndrome; central core disease; centronuclear myopathy; cerebral capillary malformation;
cerebrooculofacioskeletal syndrome type 4; cerebrooculogacioskeletal syndrome; cerebrotendinous
xanthomatosis; Chaga's Disease; Charcot Marie Tooth Disease; chemotherapy; cherubism; CHILD
syndrome; chronic granulomatous disease; chronic recurrent multifocal osteomyelitis; cirrhosis; citrin
deficiency; citrullinemia type I; citrullinemia type II; classic hemochromatosis; CNPPB syndrome;
cobalamin C disease; Cockayne syndrome; coenzyme Q10 deficiency; Coffin-Lowry syndrome; Cohen
syndrome; combined deficiency of coagulation factors V; common variable immune deficiency 3;
complement hyperactivation; complete androgen insentivity; cone rod dystrophies; conformational
diseases; congenital adrenal hyperplasia; congenital bile adid synthesis defect type 1; congenital bile adid
synthesis defect type 2; congenital defect in bile acid synthesis type; congenital erythropoietic porphyria;
congenital generalized osteosclerosis; congenital hyperplasia (CAH); congenital muscular dystrophy 1A
(MDC1A); Cornelia de Lange syndrome; coronary heart disease; Cousin syndrome; Cowden disease;
COX deficiency; Cri du chat syndrome; Crigler-Najjar disease; Crigler-Najjar syndrome type 1; Crisponi
syndrome; Crouzon syndrome; Currarino syndrome; Curth-Macklin type ichthyosis hystrix; cutaneous
T-cell lymphoma; cutis laxa; cystic fibrosis; cystinosis; d-2-hydroxyglutaric aciduria; DDP syndrome;
Dejerine-Sottas disease; Denys-Drash syndrome; Dercum disease; desmin cardiomyopathy; desmin
myopathy; DGUOK-associated mitochondrial DNA depletion; diabetes Type I; diabetes Type II;
disorders of glutamate metabolism; distal spinal muscular atrophy type 5; DNA repair diseases; dominant
optic atrophy; Doyne honeycomb retinal dystrophy; Dravet Syndrome; Duchenne muscular dystrophy;
dyskeratosis congenita; Ehlers-Danlos syndrome type 4; Ehlers-Danlos syndromes; Elejalde disease;
Ellis-van Creveld disease; Emery-Dreifuss muscular dystrophies; encephalomyopathic mtDNA depletion
syndrome; encephalitis; enzymatic diseases; EPCAM-associated congenital tufting enteropathy;
epidermolysis bullosa with pyloric atresia; epilepsy; fabry disease; facioscapulohumeral muscular
dystrophy; Factor V Leiden thrombophilia; Faisalabad histiocytosis; familial atypical mycobacteriosis;
familial capillary malformation-arteriovenous; Familial Creutzfeld-Jakob disease; familial esophageal
achalasia; familial glomuvenous malformation; familial hemophagocytic lymphohistiocytosis; familial
mediterranean fever; familial megacalyces; familial schwannomatosis; familial spina bifida; familial
splenic asplenia/hypoplasia; familial thrombotic thrombocytopenia purpura; Fanconi disease (Fanconi
anemia); Feingold syndrome; FENIB; fibrodysplasia ossificans progressiva; FKTN; Fragile X syndrome;
Francois-Neetens fleck corneal dystrophy; Frasier syndrome; Friedreich's ataxia; FTDP-17; Fuchs
corneal dystrophy; fucosidosis; G6PD deficiency; galactosialidosis; Galloway syndrome; Gardner
syndrome; Gaucher disease; Gitelman syndrome; glaucoma; GLUT1 deficiency; GM2- Gangliosidoses
(e.g., Tay Sachs Disease, Sandhoff Disease) glycogen storage disease type 1b; glycogen storage disease
type 2; glycogen storage disease type 3; glycogen storage disease type 4; glycogen storage disease type
9a; glycogen storage diseases; GM1-gangliosidosis; Greenberg syndrome; Greig cephalopolysyndactyly
syndrome; hair genetic diseases; hairy cell leukemia; HANAC syndrome; harlequin type ichtyosis
congenita; HDR syndrome; hearing loss; heart failure; hemochromatosis type 3; hemochromatosis type
4; hemolytic anemia; hemolytic uremic syndrome; hemophilia A; hemophilia B; hepatis C infection;
hereditary angioedema type 3; hereditary angioedemas; hereditary hemorrhagic telangiectasia; hereditary
hypofibrinogenemia; hereditary intraosseous vascular malformation; hereditary leiomyomatosis and
renal cell cancer; hereditary neuralgic amyotrophy; hereditary orotic aciduria; hereditary sensory and
autonomic neuropathy type; Hermansky-Pudlak disease; HHH syndrome; HHT2; hidrotic ectodermal
dysplasia type 1; hidrotic ectodermal dysplasias; histiocytic sarcoma; HNF4A-associated
hyperinsulinism; HNPCC; homozygous familial hypercholesterolemia; hormone refractory prostate
cancer; human immunodeficiency with microcephaly; Human monkeypox (MPX); human papilloma
virus (HPV) infection; Huntington's disease; hyper-IgD syndrome; hyperinsulinism-hyperammonemia
syndrome; hypercholesterolemia; hypertrophy of the retinal pigment epithelium; hypochondrogenesis;
hypohidrotic ectodermal dysplasia; hypertension; hypotension; ICF syndrome; idiopathic congenital
intestinal pseudo-obstruction; immunodeficiency 13; immunodeficiency 17; immunodeficiency 25;
immunodeficiency with hyper-IgM type 1; immunodeficiency with hyper-IgM type 3; immunodeficiency
with hyper-IgM type 4; immunodeficiency with hyper-IgM type 5; immunoglobulin alpha deficiency;
inborn errors of thyroid metabolism; infantile myofibromatosis; infantile visceral myopathy; infantile X-
linked spinal muscular atrophy; influenza A; influenza B; intradialytic hypotension; intrahepatic
cholestasis of pregnancy; invasive aspergillosis; invasive mucormycosis; IPEX syndrome; IRAK4
deficiency; isolated congenital asplenia; Jeune syndrome; Johanson-Blizzard syndrome; Joubert
syndrome; JP-HHT syndrome; juvenile hemochromatosis; juvenile hyalin fibromatosis; juvenile
nephronophthisis; Kabuki mask syndrome; Kallmann syndromes; Kartagener syndrome; KCNJ11-
associated hyperinsulinism; Kearns-Sayre syndrome; Kostmann disease; Kozlowski type of
spondylometaphyseal dysplasia; Krabbe disease; LADD syndrome; late infantile-onset neuronal ceroid
lipofuscinosis; LCK deficiency; LDHCP syndrome; Leber Congenital Amaurosis Teyp 10; Legius
syndrome; Leigh syndrome; lethal congenital contracture syndrome 2; lethal congenital contracture
syndromes; lethal contractural syndrome type 3; lethal neonatal CPT deficiency type 2; lethal
osteosclerotic bone dysplasia; leukocyte adhesion deficiency; Li Fraumeni syndrome; LIG4 syndrome;
limb girdle muscular dystrophies (LGMD1B, LGMD2A, LGMD2B); lipodystrophy; lissencephaly type
1; lissencephaly type 3; Loeys-Dietz syndrome; low phospholipid-associated cholelithiasis; Lynch
Syndrome; lysinuric protein intolerance; a lysosomal storage disease (e.g., Hunter syndrome, Hurler
syndrome); macular dystrophy; Maffucci syndrome; Majeed syndrome; malaria; mannose-binding
protein deficiency; mantle cell lymphoma; Marfan disease; Marshall syndrome; MASA syndrome;
mastocytosis; MCAD deficiency; McCune-Albright syndrome; MCKD2; Meckel syndrome; MECP2
Duplication Syndrome; Meesmann corneal dystrophy; megacystis-microcolon-intestinal hypoperistalsis;
megaloblastic anemia type 1; MEHMO; MELAS; Melnick-Needles syndrome; MEN2s; meningitis;
Menkes disease; metachromatic leukodystrophies; methymalonic acidemia due to transcobalamin
receptor defect; methylmalonic acidurias; methylvalonic aciduria; microcoria-congenital nephrosis
syndrome; microvillous atrophy; migraine; mitochondrial neurogastrointestinal encephalomyopathy;
monilethrix; monosomy X; mosaic trisomy 9 syndrome; Mowat-Wilson syndrome; mucolipidosis type
2; mucolipidosis type Ma; mucolipidosis type IV; mucopolysaccharidoses; mucopolysaccharidosis type
3A; mucopolysaccharidosis type 3C; mucopolysaccharidosis type 4B; multiminicore disease; multiple
acyl-CoA dehydrogenation deficiency; multiple cutaneous and mucosal venous malformations; multiple
endocrine neoplasia type 1; multiple myeloma; multiple sclerosis; multiple sulfatase deficiency; mycosis
fungoides; myotonic dystrophy; NAIC; nail-patella syndrome; nemaline myopathies; neonatal diabetes
mellitus; neonatal surfactant deficiency; nephronophtisis; Netherton disease; neurofibromatoses;
neurofibromatosis type 1; Niemann-Pick disease type A; Niemann-Pick disease type B; Niemann-Pick
disease type C; NKX2E; non-alcoholic fatty liver disease (NAFLD); non-alcoholic steatohepatitis
(NASH); Noonan syndrome; North American Indian childhood cirrhosis; NROB1 duplication-associated
DSD; ocular genetic diseases; oculo-auricular syndrome; OLEDAID; oligomeganephronia;
oligomeganephronic renal hypolasia; Ollier disease; Opitz-Kaveggia syndrome; ornithine
transcarbamylase deficiency (OTCD); orofaciodigital syndrome type 1; orofaciodigital syndrome type 2;
osseous Paget disease; osteogenesis imperfecta; otopalatodigital syndrome type 2; orthostatic
hypotension; overactive bladder; OXPHOS diseases; palmoplantar hyperkeratosis; panlobar
nephroblastomatosis; Parkes-Weber syndrome; Parkinson's disease; partial deletion of 21q22.2-q22.3;
Pearson syndrome; Pelizaeus-Merzbacher disease; Pendred syndrome; pentalogy of Cantrell;
peroxisomal acyl-CoA-oxidase deficiency; Peutz-Jeghers syndrome; Pfeiffer syndrome; Pierson
syndrome; pigmented nodular adrenocortical disease; pipecolic acidemia; Pitt-Hopkins syndrome;
plasmalogens deficiency; platelet glycoprotein IV deficiency; pleuropulmonary blastoma and cystic
nephroma; pneumonia; polycystic kidney disease; polycystic ovarian disease; polycystic
lipomembranous osteodysplasia; Pompe disease; including infantile onset Pompe disease (IOPD) and
late onset Pompe disease (LOPD); porphyrias; post-herpetic neuralgia; PRKAG2 cardiac syndrome;
premature ovarian failure; primary erythermalgia; primary hemochromatoses; primary hyperoxaluria;
progressive familial intrahepatic cholestasis; propionic acidemia; prostate cancer; protein-losing
enteropathy; pulmonary arterial hypertension; pyruvate decarboxylase deficiency; RAPADILINO
syndrome; renal cystinosis; restless leg syndrome; retinitis pigmentosa; Rett Syndrome; rhabdoid tumor
predisposition syndrome; Rieger syndrome; ring chromosome 4; Roberts syndrome; Robinow-Sorauf
syndrome; Rothmund-Thomson syndrome; severe combined immunodeficiency disorder (SCID);
Saethre-Chotzen syndrome; Sandhoff disease; SC phocomelia syndrome; SCAS; Schinzel phocomelia
syndrome; schizophrenia; severe hypertriglyceridemia; short rib-polydactyly syndrome type 1; short rib-
polydactyly syndrome type 4; short-rib polydactyly syndrome type 2; short-rib polydactyly syndrome
type 3; Shwachman disease; Shwachman-Diamond disease; sickle cell anemia; Silver-Russell syndrome;
Simpson-Golabi-Behmel syndrome; skin infection; Smith-Lemli-Opitz syndrome; SPG7-associated
hereditary spastic paraplegia; spherocytosis; spinocerebellar ataxia; spinal muscular atrophy; split-
hand/foot malformation with long bone deficiencies; spondylocostal dysostosis; sporadic amyotrophic
lateral sclerosis; sporadic visceral myopathy with inclusion bodies; storage diseases; Stargardt macular
dystrophy; STRA6-associated syndrome; stroke; tardive dyskinesia; Tay-Sachs disease; thanatophoric
dysplasia; thromboembolism; thrombosis; thrombophilia due to antithrombin III deficiency; thyroid
metabolism diseases; Tourette syndrome; transcarbamylase deficiency; transthyretin-associated
amyloidosis; trisomy 13; trisomy 22; trisomy 2p syndrome; tuberous sclerosis; tufting enteropathy;
ullrich congenital muscular dystrophy (UCMD); urea cycle diseases; Usher Syndrome; Van Den Ende-
Gupta syndrome; Van der Woude syndrome; variegated mosaic aneuploidy syndrome; VLCAD
deficiency; von Hippel-Lindau disease; von Willebrand disease; Waardenburg syndrome; WAGR
syndrome; Walker-Warburg syndrome; Werner syndrome; Wilson's disease; Wiskott-Aldrich
Syndrome; Wolcott-Rallison syndrome; Wolfram syndrome; X-linked agammaglobulinemia; X-linked
chronic idiopathic intestinal pseudo-obstruction; X-linked cleft palate with ankyloglossia; X-linked
dominant chondrodysplasia punctata; X-linked ectodermal dysplasia; X-linked Emery-Dreifuss muscular
dystrophy; X-linked lissencephaly; X-linked lymphoproliferative disease; X-linked visceral heterotaxy;
xanthinuria type 1; xanthinuria type 2; xeroderma pigmentosum; XPV; and Zellweger disease.

### Examples

The following examples are included for illustrative purposes only and are not intended to limit the scope of the invention.

### Example 1: Indel Activity of CasPhi.12 Variants

Variants of CasPhi.12 were generated and tested to identify variants with increased binding affinity and greater genomic editing efficiency. Briefly, plasmid constructs encoding CasPhi.12 variants were generated by mutating nucleotides that encode single amino acids of interest within regions that interact with guide nucleic acid or the target nucleic acid from the wild-type residue to arginine. Generated variants had a single amino acid alteration—an arginine (R)—at amino acid positions: 23, 24, 25, 26, 28, 29, 51, 52, 53, 54, 55, 56, 57, 125, 126, 127, 128, 129, 130, 131, 316, 511, 512, 513, 514, 515, 516, 517, 540, 541, 542, 543, 544, 545, 546, 590, 591, 592, 593, 594, 595, 596, 602, 603, 604, 605, 606, 607, or 608 (positions as identified with respect to SEQ ID NO: 1). Wild-type CasPhi.12 (WT) (SEQ ID NO: 1) and a non-targeting control (NTC) were included as controls.

HEK293T cells were prepared, seeded and plated for plasmid lipofection. Plasmid preparations of the various constructs containing a guide targeting either B2M or FUT8 were incubated in reduced serum media (Opti-MEM) and lipid reagent. The mixture containing the CasPhi.12 variants plasmid constructs and guides targeting B2M or FUT8 were delivered by lipofection to HEK293T cells. 20 μL of the lipid:DNA mix and HEK293T cells were incubated for 3 days. The cells were lysed with QuickExtract (QE) solution and suspended for next-generation sequencing (NGS).

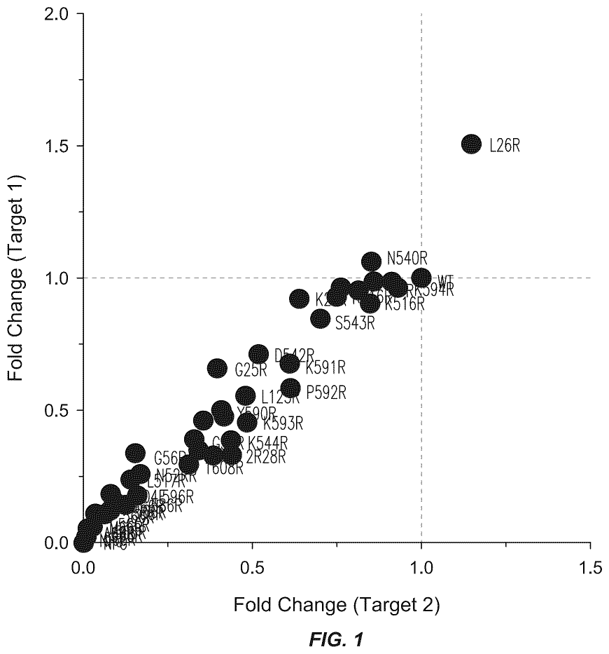

Indels were detected by NGS at the targeted locis. Indel percentage was calculated as the fraction of sequencing reads containing insertions or deletions relative to an unedited reference sequence. Raw indel data is shown in TABLE 7, normalized data is shown in TABLE 8. To demonstrate relative nuclease activity, the mean of replicate values were plotted in relation to the two target loci and normalized to the wild type. Results can be seen in FIG. 1. Indel activity was highest in the compositions that contained the L26R mutation, which is projected to be in the region of the effector protein with T-strand and NT-strand PAM interacting domains (i.e., TPID, NPID). Without being bound by theory, it is contemplated that the L26R mutation improves the binding efficiency/strength of the CasPhi.12 variant with the target nucleic acid, e.g., the genomic DNA.

TABLE 7
RAW INDEL DATA

| | PL7889 | | PL7895 | |
|---|---|---|---|---|
| Mutation | FUT8 | | B2M | |
| T23R | 40.876993166 | 33.650773361 | 42.672132675 | 35.340833885 |
| A24R | 2.0717985355 | 1.8742526626 | 0.4884547069 | 0.5820182424 |
| G25R | 26.235242676 | 23.579793742 | 17.132684051 | 16.681480488 |
| L26R | 61.012715713 | 52.934638952 | 49.325521188 | 48.84152257 |
| L28R | 18.7297172 | 16.082247984 | 13.460261235 | 16.844182045 |
| K29R | 37.365774093 | 32.226375069 | 26.529411765 | 28.1023859 |
| P51R | 20.974950629 | 15.231072159 | 18.999797119 | 16.588401185 |
| N52R | 13.952373342 | 11.004470155 | 14.98967247 | 17.678765246 |
| F53R | 7.4508560558 | 3.7136329018 | 4.2908633217 | 3.3490401186 |
| Q54R | 10.056249143 | 9.4506364176 | 7.6043237808 | 6.5556894353 |
| G55R | 14.580841579 | 14.807440398 | 13.845069181 | 14.219509269 |
| G56R | 12.748262165 | 12.714336295 | 7.1302428256 | 6.0666392055 |
| P57R | 1.6268035247 | 1.427402026 | 1.1138014528 | 0.9756036626 |
| L125R | 22.820124834 | 19.15660432 | 20.540080799 | 20.479229975 |
| I126R | 2.5208217657 | 2.5047470122 | 1.3509364446 | 1.0429735518 |
| I127R | 1.0343822844 | 0.8854057611 | 0.6996770721 | 0.9276388107 |
| K128R | 25.039228872 | 0 | 18.465077373 | 19.070725503 |
| N129R | 8.4337349398 | 11.325185861 | 7.2006472492 | 7.1492134573 |
| A130R | 1.2349066959 | 1.6122961104 | 0.6531842733 | 0.9591373439 |
| V131R | 4.7246257382 | 6.2485207101 | 4.6503044842 | 4.91959273 |
| T316R | 4.1080912215 | 4.0438607493 | 1.2746585736 | 1.8757964976 |
| Q511R | 41.933318416 | 30.916100187 | 30.75384923 | 34.433360204 |
| D512R | 27.614669314 | 26.222434403 | 20.570071259 | 23.714054837 |
| Y513R | 2.8526315789 | 2.2555410691 | 1.0457860972 | 1.2256332438 |
| K514R | 14.638888889 | 11.833137995 | 12 | 17.191358441 |
| P515R | 4.1509707331 | 4.3051913147 | 1.8143009605 | 3.8539001687 |
| K516R | 37.71281362 | 30.952255481 | 35.123797831 | 37.170249684 |
| L517R | 8.6568827791 | 9.1801985877 | 5.8177417853 | 6.1249518703 |
| N540R | 39.758416859 | 40.639304834 | 32.567012326 | 40.215294835 |
| K541R | 35.733512786 | 34.531846199 | 26.854219949 | 37.137085408 |
| L542R | 4.3135783946 | 3.2927431777 | 1.5424872607 | 2.3479778877 |
| S543R | 35.335650663 | 28.62076775 | 28.263608871 | 31.639555365 |
| K544R | 15.513361463 | 13.775012768 | 15.221751727 | 22.075256556 |
| S545R | 36.643797098 | 35.476798102 | 34.195879309 | 35.381935484 |
| R546R | 39.511115794 | 41.47524189 | 44.830917874 | 42.650810577 |
| Y590R | 17.935610181 | 19.827667477 | 16.536385632 | 18.375897231 |
| K591R | 26.49896623 | 24.826515262 | 27.340341134 | 24.878955495 |
| P592R | 22.166951656 | 21.95414915 | 25.254738687 | 27.131135213 |
| K593R | 18.929530895 | 15.483476132 | 21.108892282 | 20.287286144 |
| K594R | 37.837453822 | 35.152935923 | 39.927949565 | 39.558390986 |
| E595R | 0.6606606607 | 0.6674045008 | 0.3588248486 | 0.5730879145 |
| N596R | 7.9125392607 | 5.7279106944 | 5.614973262 | 7.8704643675 |
| A602R | 5.4086407661 | 5.4720236628 | 6.0059788024 | 4.7688306566 |
| I603R | 0.6626332469 | 0.2962231548 | 0.1692198076 | 0.1931174677 |
| H604R | 7.4059637498 | 6.4590818363 | 3.2286383145 | 3.590690372 |
| K605R | 42.978896104 | 31.594433113 | 36.626815532 | 36.827641895 |
| A606R | 6.1785747468 | 2.6755995562 | 2.9095074456 | 3.4120481928 |
| L607R | 1.0147234381 | 0.9888298846 | 0.3773219814 | 0.3559746007 |
| T608R | 11.564927858 | 10.79544249 | 12.954186414 | 13.533553201 |
| WT | 47.319725296 | 28.283171945 | 44.574162679 | 40.941168563 |
| NTC | 0.010448229 | 0.0080469944 | 0.0091675834 | 0.0229042602 |

TABLE 8
NORMALIZED INDEL DATA

| Mutations | PL7889 (Fut8) | PL7895 (B2M) |
|---|---|---|
| N129R | 0.1678346281 | 0.2613613863 |
| P515R | 0.06629475 | 0.1118539953 |
| S545R | 0.8137756116 | 0.953976127 |
| N596R | 0.1577244167 | 0.1804292322 |
| T23R | 0.9124323574 | 0.9858170176 |
| F53R | 0.0893555958 | 0.1476784254 |
| K128R | 0.4390152383 | 0.3312067311 |
| A130R | 0.0188575628 | 0.0376614128 |
| K516R | 0.8455444154 | 0.9082681098 |
| A602R | 0.1260211633 | 0.1439241327 |
| A24R | 0.0125201515 | 0.0521964444 |
| Q54R | 0.1656141897 | 0.2580275868 |
| WT | 1.0001793128 | 1.0000383233 |
| V131R | 0.1119286224 | 0.1451474398 |
| L517R | 0.1396806276 | 0.2359402297 |
| Y590R | 0.4083307937 | 0.4995142547 |
| I603R | 0.0042378629 | 0.0126832857 |
| G25R | 0.3954873045 | 0.6589290532 |
| G55R | 0.3282406836 | 0.3887338886 |
| NTC | 0.0003751093 | 0.0002446458 |
| T316R | 0.0368474277 | 0.1078300525 |
| N540R | 0.8512550545 | 1.0634619272 |
| K591R | 0.6107520074 | 0.6789084853 |
| H604R | 0.0797582303 | 0.1834000739 |
| L26R | 1.1481525586 | 1.5072401411 |
| G56R | 0.1543494974 | 0.3368068579 |
| Q511R | 0.7624235022 | 0.9636166482 |
| K541R | 0.74843632 | 0.9294359654 |
| P592R | 0.6127002795 | 0.5836124445 |
| K605R | 0.8591164611 | 0.9864196986 |
| L28R | 0.3544379331 | 0.4604757299 |
| P57R | 0.0244374867 | 0.0403995443 |
| D512R | 0.5179429953 | 0.7121310015 |
| L542R | 0.0455025164 | 0.1006127192 |
| K593R | 0.4841658295 | 0.4551985056 |
| A606R | 0.0739363233 | 0.1171187077 |
| K29R | 0.6389683937 | 0.9205310736 |
| L125R | 0.4797580208 | 0.5552477401 |
| Y513R | 0.0265663081 | 0.0675684213 |
| S543R | 0.7006218039 | 0.8459843705 |
| K594R | 0.9296648018 | 0.9654813458 |
| L607R | 0.0085765682 | 0.0265020281 |
| P51R | 0.4162362375 | 0.4789156453 |
| I126R | 0.0279989473 | 0.0664757775 |
| K514R | 0.341419397 | 0.3501590858 |
| K544R | 0.4362223191 | 0.3874123575 |
| E595R | 0.0108995645 | 0.0175669995 |
| T608R | 0.3097981242 | 0.2957720946 |
| N52R | 0.3820869908 | 0.3301169775 |
| I127R | 0.0190329343 | 0.0253940218 |

### Example 2: Indel Activity of CasPhi.12 Variants

Variants of CasPhi.12 were generated and tested to identify variants with increased binding affinity and greater genomic editing efficiency. Briefly, plasmid constructs encoding CasPhi.12 variants were generated by mutating nucleotides that encode single amino acids of interest within regions that interact with guide nucleic acid or the target nucleic acid from the wild-type residue to arginine. Generated variants had a single amino acid mutation—an arginine (R)—at amino acid positions: 13, 14, 15, 16, 17, 18, 19, 20, 21, 22, 30, 31, 32, 33, 34, 35, 36, 37, 38, 39, 40, 41, 42, 43, 44, 108, 109, 110, 111, 112, 113, 114, 115, 116, 117, 118, 119, 120, 121, 122, 123, 124, 132, 133, 134, 135, 136, 137, 138, 139, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, or 210 (positions as identified with respect to SEQ ID NO: 1). Wild-type CasPhi.12 (WT) (SEQ ID NO: 1) was included as a baseline control.

HEK293T cells were prepared, seeded and plated for plasmid lipofection. Plasmid preparations of the various constructs containing a guide targeting FUT8 were incubated in reduced serum media (Opti-MEM) and lipid reagent. The mixture containing the CasPhi.12 variants plasmid constructs and guides targeting FUT8 were delivered by lipofection to HEK293T cells. 20 μL of the lipid:DNA mix and HEK293T cells were incubated for 3 days. The cells were lysed with QuickExtract (QE) solution and suspended for next-generation sequencing (NGS).

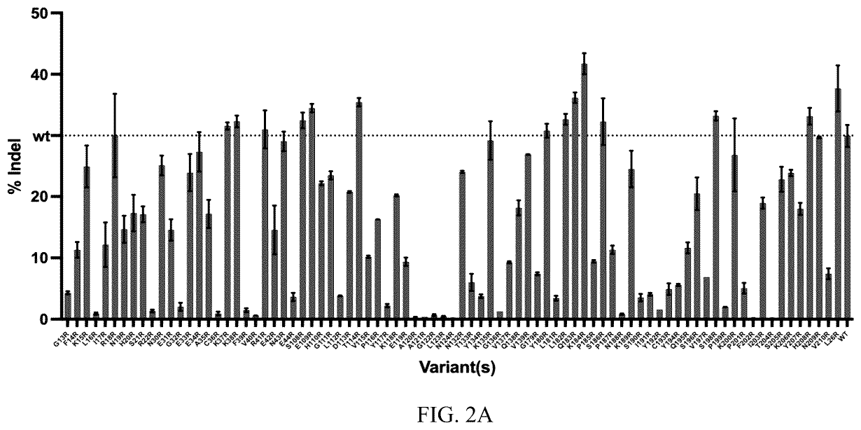

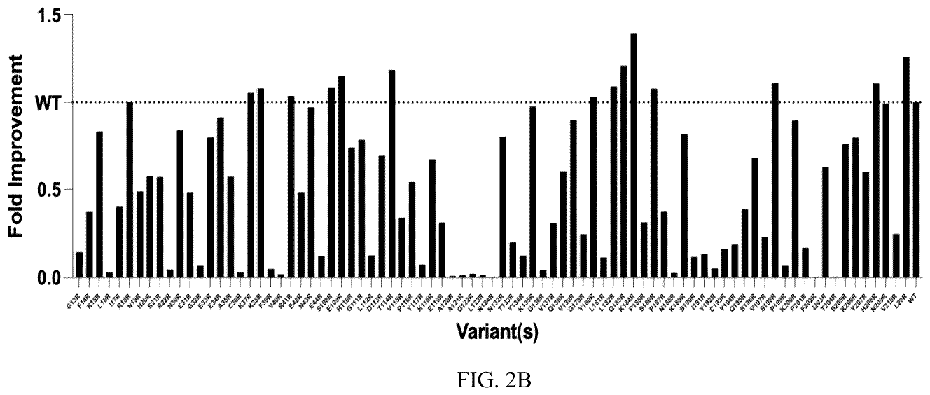

Indels were detected by NGS at the targeted loci. Indel percentage was calculated as the fraction of sequencing reads containing insertions or deletions relative to an unedited reference sequence. Raw indel data is shown in TABLE 9 and FIG. 2A, normalized data against the WT baseline is shown in TABLE 10 and FIG. 2B. To demonstrate relative nuclease activity, the mean of replicate values were normalized to the wild type. Indel activity was highest for the compositions that contained the E109R, H208R, K184R, K38R, L182R, Q183R, S108R, S198R, and the T114R mutation.

TABLE 9
RAW INDEL DATA

| Mutation | Rep 1 | Rep 2 |
|---|---|---|
| G13R | 4.5003309067 | 4.1178947368 |
| F14R | 12.206251426 | 10.393336418 |
| K15R | 27.370356749 | 22.535314891 |
| L16R | 1.0535099614 | 0.802580258 |
| I17R | 14.719523935 | 9.5904330155 |
| R18R | 34.806727129 | 25.182765593 |
| N19R | 16.213097413 | 13.097593142 |
| H20R | 19.400196657 | 15.205479452 |
| S21R | 18.029395754 | 16.194899658 |
| R22R | 1.5044870667 | 1.1980500702 |
| N30R | 26.281142394 | 24.000960999 |
| E31R | 15.780542986 | 13.319206902 |
| G32R | 2.5 | 1.5377024916 |
| E33R | 21.770462633 | 26.105442177 |
| E34R | 25.064671101 | 29.625677959 |
| A35R | 15.547151196 | 18.783196523 |
| C36R | 0.696969697 | 1.1538461538 |
| K37R | 31.155411752 | 31.969653318 |
| K38R | 31.628910464 | 32.978345758 |
| F39R | 1.250899928 | 1.6980606848 |
| V40R | 0.6051257712 | 0.5850757721 |
| R41R | 28.826509426 | 33.197420823 |
| E42R | 11.733734487 | 17.358958462 |
| N43R | 27.927927928 | 30.18134715 |
| E44R | 4.1102077687 | 3.1665758967 |
| S108R | 33.388189739 | 31.579476861 |
| E109R | 34.958382878 | 33.974801519 |
| H110R | 21.974554707 | 22.439066701 |
| G111R | 23.984652817 | 23.048262716 |
| L112R | 3.7715086034 | 3.8577358183 |
| D113R | 20.668558255 | 20.897323387 |
| T114R | 35.923423423 | 34.967500275 |
| V115R | 10.044712488 | 10.327837787 |
| P116R | 16.302521008 | 16.266944734 |
| Y117R | 2.0328599276 | 2.40853098 |
| K118R | 20.304849885 | 20.082081895 |
| E119R | 9.8519562919 | 8.8972538284 |
| A120R | 0.355073818 | 0.1931034483 |
| A121R | 0.3860091382 | 0.3927986907 |
| G122R | 0.5516867143 | 0.7775524003 |
| L123R | 0.5246589717 | 0.4078111521 |
| N124R | 0.102541566 | 0.1495827429 |
| N132R | 23.942687747 | 24.2152872 |
| T133R | 5.033492823 | 6.9926526476 |
| Y134R | 3.541472507 | 3.9736914223 |
| K135R | 31.407035176 | 26.978154071 |
| G136R | 1.2723762724 | 1.2704005935 |
| V137R | 9.1770084912 | 9.3946376204 |
| Q138R | 19.021393773 | 17.279305355 |
| V139R | 26.953579359 | 26.877682403 |
| G179R | 7.2240685674 | 7.587201901 |
| Y180R | 31.608056794 | 29.996979156 |
| L181R | 3.7063059898 | 3.1621926853 |
| L182R | 33.270998909 | 32.022787451 |
| Q183R | 36.78236193 | 35.55438226 |
| K184R | 40.511697907 | 42.926622965 |
| P185R | 9.5792946999 | 9.2819310584 |
| S186R | 34.958563536 | 29.583292286 |
| P187R | 11.816121722 | 10.857672694 |
| N188R | 0.9019700926 | 0.7430664574 |
| K189R | 26.65433621 | 22.426631625 |
| S190R | 3.9630413002 | 3.0875918435 |
| I191R | 4.2350247997 | 3.8957915832 |
| Y192R | 1.5611092092 | 1.5761376155 |
| C193R | 5.5651643097 | 4.2355155825 |
| Y194R | 5.4571293226 | 5.7247150997 |
| Q195R | 10.986594051 | 12.267358626 |
| S196R | 18.595825427 | 22.389335156 |
| V197R | 6.8554396423 | 6.8561382948 |
| S198R | 33.756652153 | 32.667512154 |
| P199R | 1.9747615837 | 2.0315423684 |
| K200R | 22.593167702 | 31.040652023 |
| P201R | 5.6712672522 | 4.4456404736 |
| F202R | 0.1279426817 | 0.1712946268 |
| I203R | 19.580975077 | 18.28257717 |
| T204R | 0.1446712747 | 0.0895977063 |
| S205R | 24.3180736 | 21.372446307 |
| K206R | 24.267158153 | 23.54387292 |
| Y207R | 17.295995945 | 18.674256335 |
| H208R | 34.12089598 | 32.185903984 |
| N209R | 29.793089389 | 29.590754877 |
| V210R | 8.0384900074 | 6.7905547017 |
| L26R | 40.346760527 | 35.032894737 |
| WT | 28.693106202 | 31.212074063 |

TABLE 10
NORMALIZED DATA

| Mutation | Normalized to WT |
|---|---|
| G13R | 0.1438644472 |
| F14R | 0.3772559859 |
| K15R | 0.8330777308 |
| L16R | 0.03098380159 |
| I17R | 0.4058072581 |
| R18R | 1.001407432 |
| N19R | 0.4892847401 |
| H20R | 0.5776741837 |
| S21R | 0.5713077777 |
| R22R | 0.04511357991 |
| N30R | 0.8393615238 |
| E31R | 0.4857634976 |
| G32R | 0.06740155816 |
| E33R | 0.7991947374 |
| E34R | 0.9129485767 |
| A35R | 0.5730781139 |
| C36R | 0.0308957563 |
| K37R | 1.053749689 |
| K38R | 1.078491976 |
| F39R | 0.04922713862 |
| V40R | 0.01986809051 |
| R41R | 1.035368393 |
| E42R | 0.4856456958 |
| N43R | 0.97002087 |
| E44R | 0.121471693 |
| S108R | 1.084508323 |
| E109R | 1.150704899 |
| H110R | 0.7413986772 |
| G111R | 0.7851226776 |
| L112R | 0.127355337 |
| D113R | 0.693861223 |
| T114R | 1.183385533 |
| V115R | 0.3400799428 |
| P116R | 0.543683628 |
| Y117R | 0.07414034793 |
| K118R | 0.6741809573 |
| E119R | 0.312981449 |
| A120R | 0.009150748965 |
| A121R | 0.01300067583 |
| G122R | 0.02218905124 |
| L123R | 0.01556576776 |
| N124R | 0.004208722982 |
| N132R | 0.8039033475 |
| T133R | 0.2007530136 |
| Y134R | 0.125450986 |
| K135R | 0.9746267182 |
| G136R | 0.04244669417 |
| V137R | 0.3100173646 |
| Q138R | 0.6059692829 |
| V139R | 0.8986077919 |
| G179R | 0.2472452366 |
| Y180R | 1.028375771 |
| L181R | 0.1146561724 |
| L182R | 1.089952256 |
| Q183R | 1.207520683 |
| K184R | 1.392839826 |
| P185R | 0.3148513313 |
| S186R | 1.077400244 |
| P187R | 0.3784947198 |
| N188R | 0.02746067273 |
| K189R | 0.8193109113 |
| S190R | 0.1176965517 |
| I191R | 0.1357281014 |
| Y192R | 0.05237020923 |
| C193R | 0.1636032118 |
| Y194R | 0.1866590564 |
| Q195R | 0.388179329 |
| S196R | 0.684167219 |
| V197R | 0.2288880173 |
| S198R | 1.108821708 |
| P199R | 0.06687742086 |
| K200R | 0.8953118827 |
| P201R | 0.1688820179 |
| F202R | 0.004995182506 |
| I203R | 0.6320580638 |
| T204R | 0.003910663151 |
| S205R | 0.7627140041 |
| K206R | 0.798111797 |
| Y207R | 0.6004531181 |
| H208R | 1.10686254 |
| N209R | 0.9912973136 |
| V210R | 0.2475419428 |
| L26R | 1.258316141 |
| WT | 1 |

### Example 3: Indel Activity of CasPhi.12 Variants with Multiple Mutations

CasPhi.12 Variants with combinations of mutations are generated and tested to identify variants with increased binding affinity and greater genomic editing efficiency using the methods described herein. Briefly, plasmid constructs encoding CasPhi.12 variants are generated by co-mutating top-performing variants generated in Example 1 and Example 2. Generated variants can have two or more amino acid mutations—an arginine (R)—as described herein, including at amino acid positions: 26, 109, 208, 184, 38, 182, 183, 108, 198, and/or 114 (positions as identified with respect to SEQ ID NO: 1). TABLE 11 provides exemplary CasPhi.12 variants having such mutations.

The generated variants are assayed for increased binding strength and/or indel formation efficiency using guide nucleic acids or target nucleic acids described herein.

TABLE 11
EXEMPLARY CASPHI.12 VARIANTS
WITH MULTIPLE MUTATIONS

| Mutation I | Mutation 2 | Mutation 3 | Mutation 4 |
|---|---|---|---|
| L26R | K184R | | |
| L26R | Q183R | | |
| L26R | T114R | | |
| L26R | E109R | | |
| L26R | S198R | | |
| L26R | H208R | | |
| L26R | L182R | | |
| L26R | S108R | | |
| L26R | K38R | | |
| K184R | Q183R | | |
| K184R | T114R | | |
| K184R | E109R | | |
| K184R | S198R | | |
| K184R | H208R | | |
| K184R | L182R | | |
| K184R | S108R | | |
| K184R | K38R | | |
| L26R | K184R | H208R | |
| L26R | Q183R | K184R | |
| L26R | Q183R | H208R | |
| L26R | Q183R | K184R | T114R |

### Example 4: CasPhi.12 L26R Variant Efficiently and Selectively Induces Cells Death in Pancreatic Cells Expressing Mutant Kras in a Dose-Dependent Manner

CasPhi.12 L26R Variant (L26R Variant), Casphi.12 WT, and Cas9 knockout of the oncogene KRAS in the human pancreatic adenocarcinoma cell lines BxPC3 (KRAS-WT) and AsPC-1 (KRAS-G12D) were assessed by transfection of Cas-RNPs.

KRAS (Kirsten rat sarcoma 2 viral oncogene homolog) is a proto-oncogene and one of the most common driver oncogenes, which is mutated in over 90% of pancreatic cancers and over 30% of lung and colon cancers. It functions as a GTPase and is attached to the inner surface of the cell membrane. The most common mutations include:
KRAS p.G12C-c.34G→T (12th a.a. is mutated from Gly to Cys; 34th nucleotide is mutated from G to T;
KRAS p.G12D-c.35G→A (12th a.a. is mutated from Gly to Asp; 35th nucleotide is mutated from G to A); and
KRAS p.G12V-C.35G→T (12th a.a. is mutated from Gly to Val; 35th nucleotide is mutated from G to T).

Selective knockdown of the S-G12D mutant has been shown to lead to tumor cell death (see WO 2022/140572). The Panc08.13 cell line is a homozygous KRAS-G12D mutant.

BxPC-3 and AsPC-1 cells were seeded in T-75 flasks and cells were grown to approximately 70-80% confluence. Cells were then trypsinized and resuspended in R Buffer (provided in the Neon™ 10 μl kit) at a concentration of 1×10⁷ cells/ml.

To form L26R Variant and CasPhi.12 WT RNPs, L26R Variant or CasPhi.12 WT were mixed with wildtype KRAS-targeting guide RNA or G12D mutant KRAS targeting guide RNA. Cas9 RNPs were similarly formed, which serve as controls. Guide RNA sequences are indicated in TABLE 12.

TABLE 12
KRAS GUIDE RNA SEQUENCES

| | | | SEQ |
|---|---|---|---|
| | | | ID |
| KRAS | Cas | Guide RNA Sequence | NO: |
| WT | CasPhi.12 WT | CUUUCAAGACUAAUAGAUUGCUCCUU | 34 |
| | L26R Variant | ACGAGGAGACGAGCUGGUGGCGUAmG | |
| | | mGmC | |
| G12D | CasPhi.12 WT | CUUUCAAGACUAAUAGAUUGCUCCUU | 35 |
| | L26R Variant | ACGAGGAGACGAGCUGAUGGCGUAmG | |
| | | mGmC | |
| WT | Cas9 | GUAGUUGGAGCUGGUGGCGU | 36 |
| G12D | Cas9 | GUAGUUGGAGCUGAUGGCGU | 37 |

m = a 2′ O-methyl modification of the subsequent indicated nucleotide

As a baseline determination of KRAS G12D knockout, 2 μg of L26R Variant or CasPhi.12 WT was mixed with 500 pmol guide RNA and 2 μg of Cas9 was mixed with 200 pmol guide RNA. The resulting RNP complexes were each added to 1×10⁷ cells and diluted with R Buffer (Neon™).

Electroporation was performed according to Neon™ Transfection System manufacturer's instructions. Following electroporation, cells were incubated at 37° C. and 5% CO₂ for approximately 72 hours before performing next generation sequencing (NGS) analysis. DNA was extracted from cells and barcoded for sequencing. Indel formation as indicated by sequencing results was quantified and analyzed.

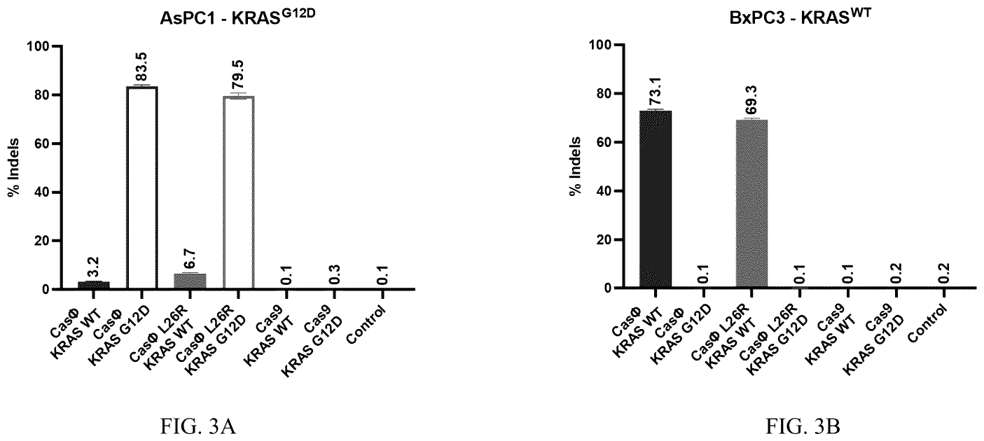

Exemplary results of this baseline determination are shown in FIG. 3A (AsPC-1 cells) and FIG. 3B (BxPC-3 cells). While a marginal difference is seen in indel production between the L26R Variant and the CasPhi.12 WT, both still demonstrate high knock-out of KRAS G12D. The L26R Variant also demonstrated slightly increased non-target editing of KRAS WT compared to the controls.

To evaluate dose response, calculated concentrations of guide RNA and Cas proteins were mixed according to TABLE 13 to form RNP complexes and added to 1×10⁷ cells. Electroporation, incubation, DNA extraction, and sequencing were performed as described above.

TABLE 13
CONCENTRATION CALCULATIONS FOR DOSE RESPONSE

| Guide RNA | CasPhi | Mix | R buffer |
|---|---|---|---|
| dilution (pmol) | dilution (μg) | volume (μl) | volume (μl) |
| 500 | 5 | 12 | 0 |
| 250 | 2.5 | 12 from 5 ug | 12 |
| 125 | 1.25 | 12 from 2.5 ug | 12 |
| 62.5 | 0.625 | 12 from 1.25 | 12 |
| 31.25 | 0.3125 | 12 from 0.625 | 12 |
| 15.625 | 0.15625 | 12 from 0.3125 | 12 |

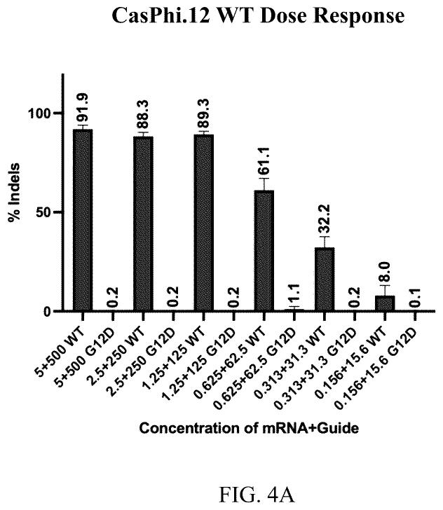

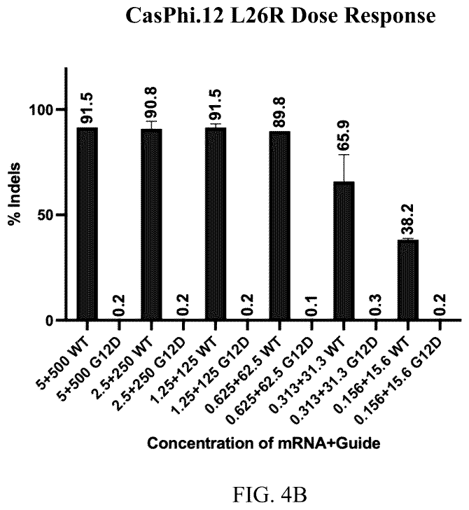

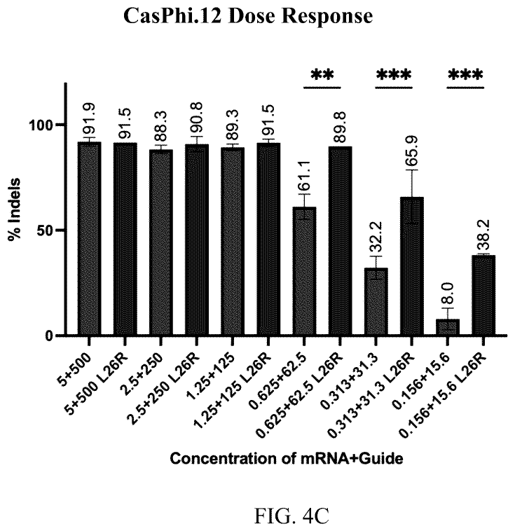

Results for dose response characterizations are shown in FIGS. 4A, 4B, and 4C. FIG. 4A demonstrates that CasPhi.12 WT has excellent specificity, but on target cutting efficiency reduces significantly with dose, especially below 0.625 ug nuclease and 62.5 pmol guide RNA. FIG. 4B demonstrates that the L26R Variant retains the excellent specificity of the WT version, and furthermore, on target cutting efficiency is much higher at low doses when compared to WT, especially at and below 1.25 ug nuclease and 125 pmol guide RNA.

FIG. 4C is a combined plot and statistical significance calculated by ONE-WAY ANOVA. This experiment demonstrated that both WT and L26R CasPhi.12 have high editing efficiency at the higher concentration—5 ug, 2.5 ug, 1.25 ug. However, as the concentration reduces—0.6 ug, 0.3 ug, 0.15 ug—the engineered L26R Variant demonstrates higher target cutting efficiency than WT CasPhi.12.

Without being bound by theory, it is contemplated that CasPhi.12 Variants work well with guide nucleic acids specific for KRAS mutants and induce cell death in only those cells that contain a mutant allele. These results show that use of CasPhi.12 Variants may be particularly relevant to treatment of various types of cancers. Furthermore, these results indicate that lower doses of disclosed effector protein may be used compared to wild-type CasPhi.12.

### Example 5: CasPhi.12 Fusion with Exonucleases can Modify Nuclease Activity

CasPhi.12 fusions with various exonucleases were tested for their ability to generate indels in HEK293T cells. The fusion protein comprised a fusion partner either on the N terminus, C terminus or both. The fusion partner was linked to the CasPhi.12 effector protein by XTEN80 linker. About 17 fusion proteins were generated and tested, including the CasPhi.12 fusions with one or two exonucleases, which included: CasPhi.12-TREX1, TREX1-CasPhi.12, CasPhi.12-sbcB, sbcB-CasPhi.12, exo5-CasPhi.12-recJ, exo5-CasPhi.12-sbcB, recJ-CasPhi.12-exo5, sbcB-CasPhi.12-exo5, CasPhi.12-FenA, FenA-CasPhi.12, CasPhi.12-nurA, nurA-CasPhi.12, CasPhi.12-exo5, exo5-CasPhi.12, CasPhi.12-dam1, CasPhi.12-recJ and recJ-CasPhi.12. Briefly, a first plasmid encoding a fusion protein and a second plasmid encoding a crRNA were delivered by lipofection to HEK293T cells. Two target nucleic acids (Target A and Target B) were tested for the indel activity. The sequence of the crRNAs included a nucleotide sequence of either SEQ ID NO: 29 for Target A or SEQ ID NO: 42 for Target B. The crRNA spacer was designed to hybridize to a target sequence adjacent to a PAM of NTTN (SEQ ID NO: 14). For lipofections, 15 ng or 150 ng of the nuclease mutant and 150 ng of the guide RNA encoding plasmid were delivered to ˜30,000 HEK293T cells in 200 μl using TransIT-293 lipofection reagent. Lipofected cells were grown for −72 hrs at 37° C. to allow for indel formation. Indels were detected by next generation sequencing of PCR amplicons at the targeted loci and indel percentage was calculated as the fraction of sequencing reads containing insertions or deletions relative to an unedited reference sequence. Sequencing libraries with less than 20% of reads aligning to the reference sequence were excluded from the analysis for quality control purposes.

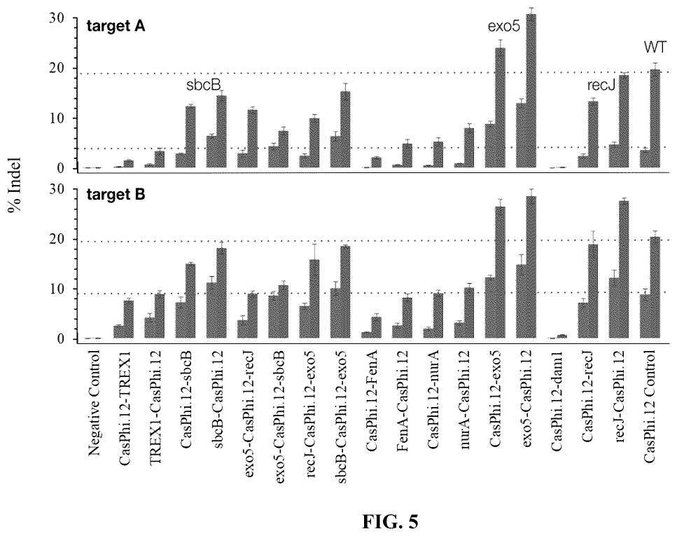

FIG. 5 show indel activity for each of the fusion proteins relative to the CasPhi.12 effector protein. An analysis of FIG. 5 indicates that fusion partners (e.g., exonucleases) can modify nuclease activity of the CasPhi.12 effector protein. In particular, the CasPhi.12-exo5 and exo5-CasPhi.12 fusion proteins showed higher indel activity relative to the CasPhi.12 effector protein alone in both targets (Target A and Target B). Also, the recJ-CasPhi.12 fusion protein showed higher indel activity than the CasPhi.12 effector protein against target B.

### Example 6. CasPhi.12 Fusion Containing a Single Fusion Partner can Modify Nuclease Activity of Corresponding CasPhi.12 Effector Protein

Effector proteins, CasPhi.12 effector protein, exo5-CasPhi.12 fusion protein and sbcB-CasPhi.12 fusion protein, were tested for the ability to produce indels in HEK293T cells. The exo5-CasPhi.12 fusion protein comprised an exonuclease sbcB linked to the effector protein on N-terminus by an XTEN80 linker. Similarly, the sbcB-CasPhi.12 fusion protein comprised an exonuclease sbcB linked to the effector protein on N-terminus by an XTEN80 linker. Briefly, a first plasmid encoding the effector protein and a second plasmid encoding a single guide RNA (crRNA) were delivered by lipofection to HEK293T cells. The sequence of the crRNA included a nucleotide sequence of SEQ ID NO: 42. The crRNA spacer was designed to hybridize to a target sequence adjacent to a PAM of GTTC (SEQ ID NO: 58). For lipofections, 15 ng or 150 ng of the effector protein in combination with 150 ng of the guide RNA encoding plasmid were delivered to ˜30,000 HEK293T cells in 200 μl using TransIT-293 lipofection reagent. Lipofected cells were grown for −72 hrs at 37° C. to allow for indel formation. Indels were detected by next generation sequencing of PCR amplicons at the targeted loci and indel percentage was calculated as the fraction of sequencing reads containing insertions or deletions relative to an unedited reference sequence. Sequencing libraries with less than 20% of reads aligning to the reference sequence were excluded from the analysis for quality control purposes.

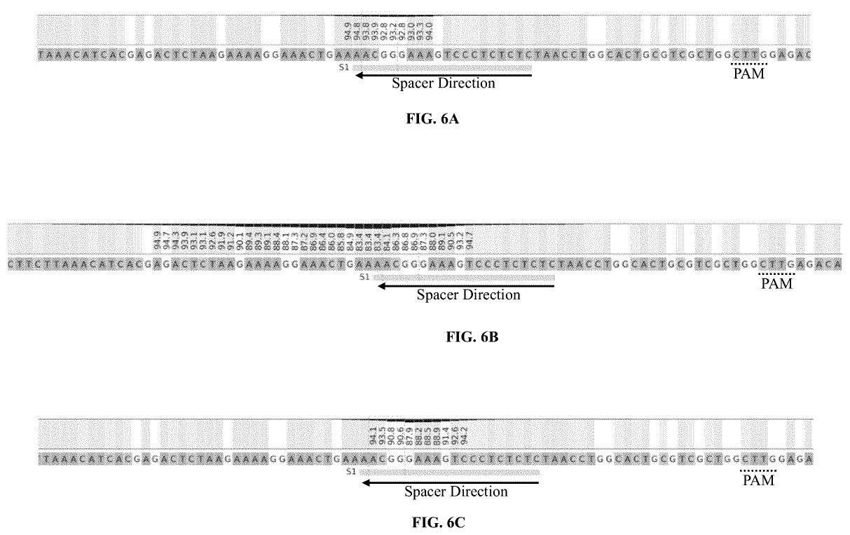

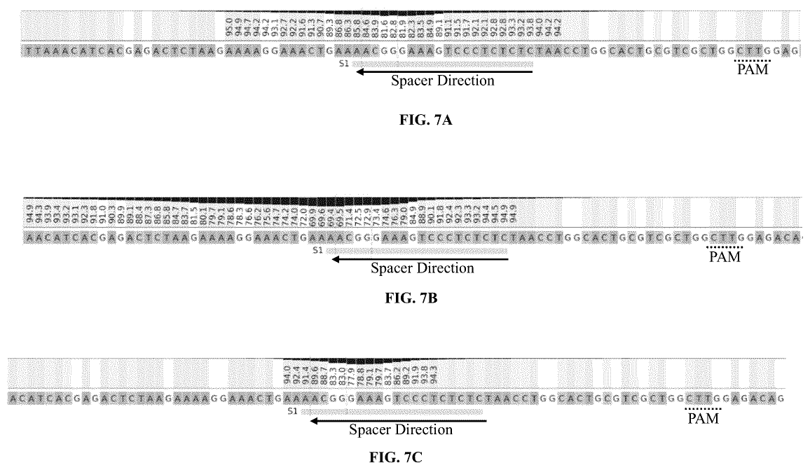

FIGS. 6A-6C and 7A-7C show exemplary indels produced in a target nucleic acid having a nucleotide sequence of GTTCGGTCGCTGCGTCACGGTCCAATCTCTCTCCCTGAAAGGGCAAAA GTCAAAGGAAAAGAATCTCAGAGCACTACAAATTCTTC (SEQ ID NO: 53). FIGS. 6A-6C show indel activity window at 15 ng dose for the CasPhi.12 effector protein, the exo5-CasPhi.12 fusion protein, and the sbcB-CasPhi.12 fusion protein, respectively. FIG. 7A-7C show indel activity window at 150 ng dose for the CasPhi.12 effector protein, the exo5-CasPhi.12 fusion protein, and the sbcB-CasPhi.12 fusion protein, respectively. A comparative analysis of FIGS. 6A-6C and FIGS. 7A-7C indicates that indel activity window is narrower at 15 ng dose relative to 150 ng dose of effector protein. Moreover, a comparative analysis of FIG. 6A and FIG. 6B, and FIG. 7A and FIG. 7B indicates that the exo5-CasPhi.12 fusion protein has broader indel activity window relative to the CasPhi.12 protein at both a 15 ng and 150 ng dose. Similarly, a comparative analysis of FIG. 6A and FIG. 6C, and FIG. 7A and FIG. 7C indicates that the sbcB-CasPhi.12 fusion protein has narrower indel activity window relative to the CasPhi.12 effector protein at both a 15 ng and 150 ng dose.

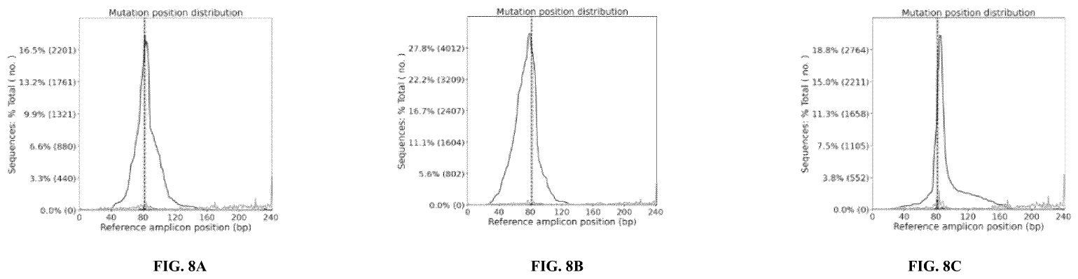

FIGS. 8A-8C summarizes nuclease activity at 150 ng dose of the CasPhi.12 protein, the exo5-CasPhi.12 fusion protein, and the sbcB-CasPhi.12 fusion protein, respectively, by assessing amplicon sizes that span across an indel activity window. FIG. 8A shows that the majority of amplicons having indels are distributed over about 50 nucleotides (55 bp to 105 bp) for the CasPhi.12 protein. FIG. 8B shows that the majority of amplicons having indels are distributed over about 60 nucleotides (40-100 bp) for the exo5-CasPhi.12 fusion protein. Thus, similar to the results shown in FIG. 7A and FIG. 7B, the indel activity window (cut site) is increased. In contrast, FIG. 8C shows that the majority of amplicons having indels are distributed over about 20 nucleotides (75-95 bp) for the sbcB-CasPhi.12 fusion protein. Thus, similar to the results shown in FIG. 7A and FIG. 7C, the indel activity window (cut site) is reduced.

### Example 7. CasPhi.12 Fusion Containing Two Fusion Partners can Modify Nuclease Activity of Corresponding CasPhi.12 Effector Protein

Effector proteins, CasPhi.12 effector protein, sbcB-CasPhi.12-exo5 fusion protein and recJ-CasPhi.12-exo5 fusion protein, were tested for the ability to produce indels in HEK293T cells. The sbcB-CasPhi.12-exo5 fusion protein comprised sbcB and exo5 fusion partners linked to the CasPhi.12 effector protein on N-terminus and C-terminus, respectively. Similarly, the recJ-CasPhi.12-exo5 fusion protein comprised recJ and exo5 fusion partners linked to the CasPhi.12 effector protein on N-terminus and C-terminus, respectively. The fusion partners were linked to the effector protein by an XTEN80 linker. Briefly, a first plasmid encoding the effector protein and a second plasmid encoding a crRNA were delivered by lipofection to HEK293T cells. The sequence of the crRNA included a nucleotide sequence of SEQ ID NO: 42. The crRNA spacer was designed to hybridize to a target sequence adjacent to a PAM of GTTC. For lipofections, 15 ng or 150 ng of the effector protein in combination with 150 ng of the guide RNA encoding plasmid were delivered to ˜30,000 HEK293T cells in 200 μl using TransIT-293 lipofection reagent. Lipofected cells were grown for ˜72 hrs at 37° C. to allow for indel formation. Indels were detected by next generation sequencing of PCR amplicons at the targeted loci and indel percentage was calculated as the fraction of sequencing reads containing insertions or deletions relative to an unedited reference sequence. Sequencing libraries with less than 20% of reads aligning to the reference sequence were excluded from the analysis for quality control purposes.

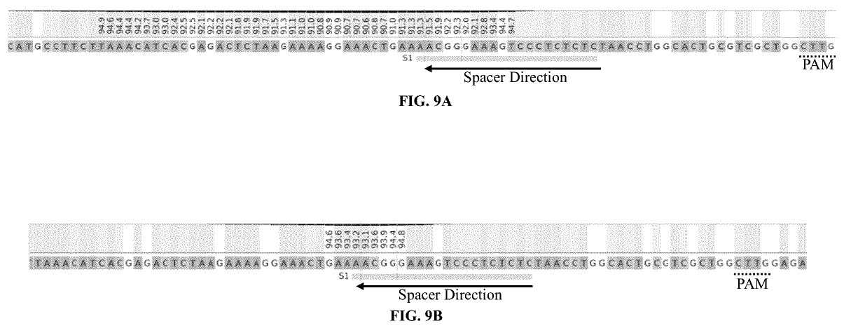

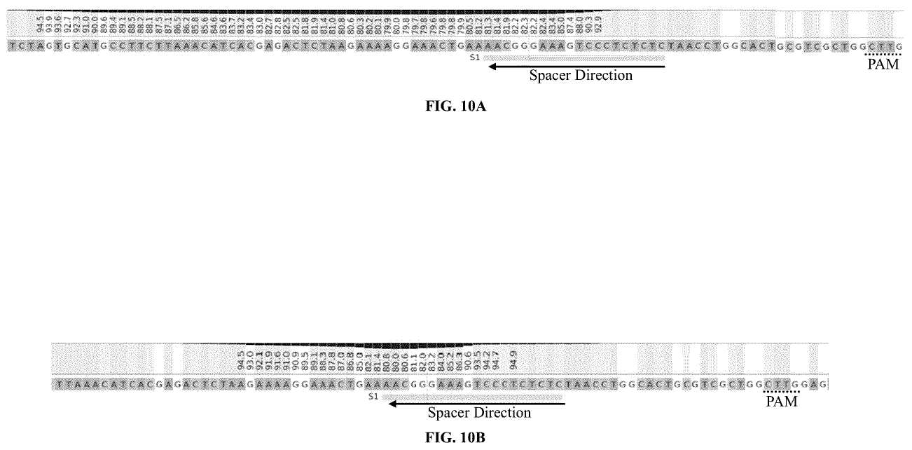

FIGS. 6A, 7A, 10A-10B and 11A-11B show exemplary indel produced in a target nucleic acid having a nucleotide sequence of GTTCGGTCGCTGCGTCACGGTCCAATCTCTCTCCCTGAAAGGG CAAAAGTCAAAGGAAAAGAATCTCAGAGCACTACAAATTCTTC (SEQ ID NO: 53). FIGS. 6A, 9A and 9B show indel activity window at 15 ng dose for the CasPhi.12 effector protein, the sbcB-CasPhi.12-exo5 fusion protein, and the recJ-CasPhi.12-exo5 fusion protein, respectively. FIGS. 7A, 10A and 10B show indel activity window at 150 ng dose for the CasPhi.12 effector protein, the sbcB-CasPhi.12-exo5 fusion protein, and the recJ-CasPhi.12-exo5 fusion protein, respectively. A comparative analysis of FIG. 6A to FIG. 7A, FIG. 9A to 10A, and FIG. 9B to FIG. 10B indicates that indel activity window is narrower at 15 ng dose relative to 150 ng dose of effector protein. Moreover, a comparative analysis of FIG. 6A to FIG. 9A, and FIG. 7A and FIG. 10A indicates that the sbcB-CasPhi.12-exo5 fusion protein has broader indel activity window relative to the CasPhi.12 effector protein at both a 15 ng and 150 ng dose. In contrast, a comparative analysis of FIG. 6A to FIG. 9B, and FIG. 7A to FIG. 10B indicates that the recJ-CasPhi.12-exo5 fusion protein has narrower indel activity window relative to the CasPhi.12 protein at both a 15 ng and 150 ng dose.

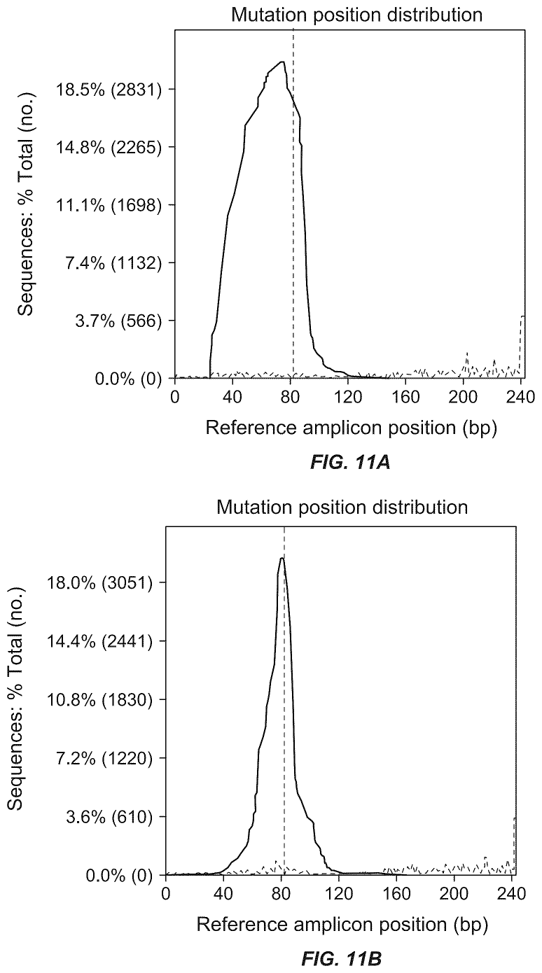

FIGS. 8A, 11A, and 11B show nuclease activity for all the target nucleic acids tested at 150 ng dose of the CasPhi.12 effector protein, the sbcB-CasPhi.12-exo5 fusion protein, and the recJ-CasPhi.12-exo5 fusion protein, respectively. An analysis of FIGS. 8A, 11A, and 11B suggests that both, the sbcB-CasPhi.12-exo5 fusion protein and the recJ-CasPhi.12-exo5 fusion protein, have higher nuclease activity relative to the CasPhi.12 protein. The analysis further indicates that the sbcB-CasPhi.12-exo5 fusion protein has higher indel activity window relative to the CasPhi.12 protein, and the indel activity window expands on the 3′ side of the spacer. Also, the analysis indicates that the recJ-CasPhi.12-exo5 fusion protein has narrower indel activity window relative to the CasPhi.12 protein. In conclusion, fusion partner can be used to modify the indel activity window of the CasPhi.12 effector protein.

### Example 8. In Vivo Editing of PCSK9 in a Mammal Using AAV Vector Encoding CasPhi.12 and L26R Variant Thereof

This example demonstrates that genome editing can be performed with an AAV8 vector encoding an effector protein and a guide RNA having a repeat sequence of SEQ ID NO: 24 and a spacer sequence of SEQ ID NO: 55. The effector protein was CasPhi.12 protein (SEQ ID NO: 1) or CasPhi.12 L26R variant protein (SEQ ID NO: 2). An AAV8 vector was constructed to contain a transgene between its ITRs. The AAV8 vector was expressed with supporting plasmids to produce an adeno-associated virus.

8-week old male mice were administered 2e+13 (vg/kg) of AAV virus by tail vein injection. Each dosing group had three animals. Negative control groups were injected with vehicle only. A positive control group was injected with SaCas9 system. Liver and serum from each mice were harvested 4 weeks post injection.

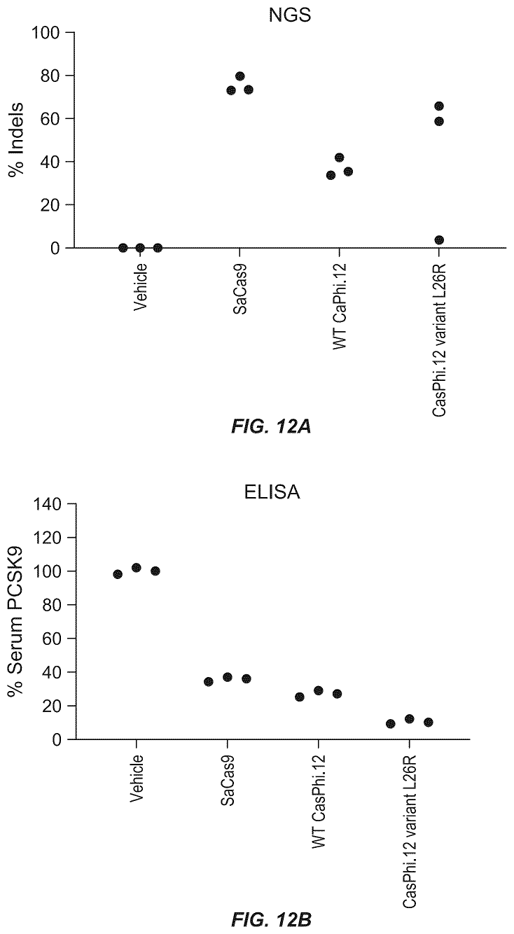

Genomic DNA was extracted from the liver and the frequency of indels was determined using NGS. FIG. 12A illustrates the frequency of CasPhi.12 system induced indels in mice liver transfected with AAV plasmid. The results depicted in FIG. 12A demonstrate that both CasPhi.12 systems introduced at least 30% indel mutations in PCSK9 gene. FIG. 12B illustrates that effect of CasPhi.12 system on the % serum of PCSK9 protein. In particular, the results depicted in FIG. 12B demonstrate that a group treated with the CasPhi.12 system targeting PCSK9 showed significant reduction in PCSK9 protein serum concentration. This study demonstrates that CasPhi.12 system can be used for in vivo genome editing in mice liver.

### Example 9: Cis Cleavage Assay for CasPhi.12 Variants

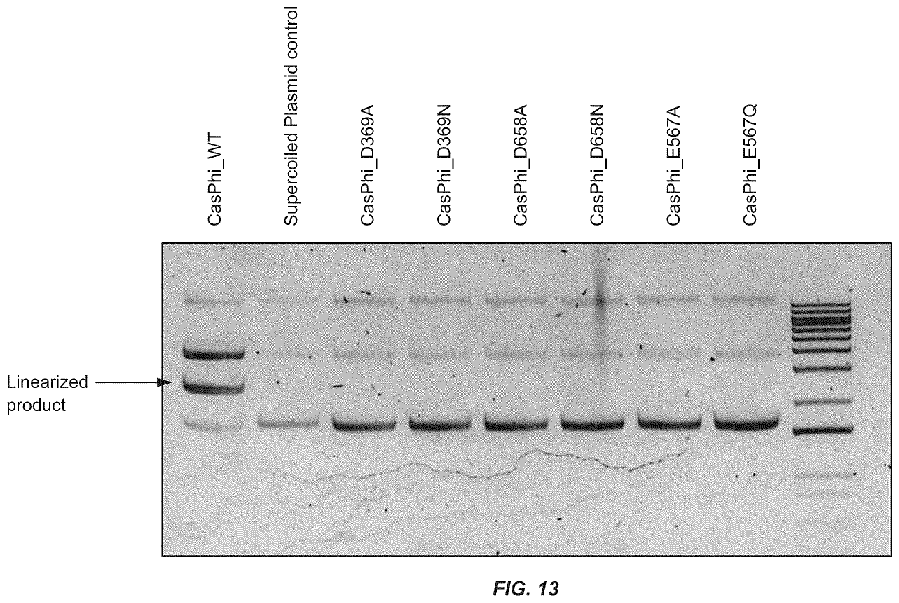

This experiment was performed to determine if variants of CasPhi.12 can perform cis cleavage. Briefly, effector protein, CasPhi.12, was mutated to generate nuclease dead variants using the following substitutions: D369A, D369N, D658A, D658N, E567A, and E567Q. The variants were then complexed with crRNA having nucleotide sequence of CUUUCAAGACUAAUAGAUUGCUCCUUACGAGGAGACUAUUAAAUACUCGUAUUGCU (SEQ ID NO: 56) for 20 minutes at room temperature to form RNP complexes. The RNP complexes were added to an IVE reaction mix. Cis cleavage assay was carried out with 5 μl of RNP for at least 30 minutes at 37° C. for identifying catalytically dead variants. A plasmid containing TTTG PAM was used as target nucleotide. Wildtype CasPhi.12 (CasPhi_WT) with supercoiled plasmid DNA was included as a positive control. Cis cleavage by each complex was assessed by gel electrophoresis. The results are shown in FIG. 13. An absence of a linearized product indicates a lack of cleavage activity. These results indicate that none of the variants assayed showed detectable cis cleavage activity.

### Example 10: Binding Affinity of CasPhi.12 Variants

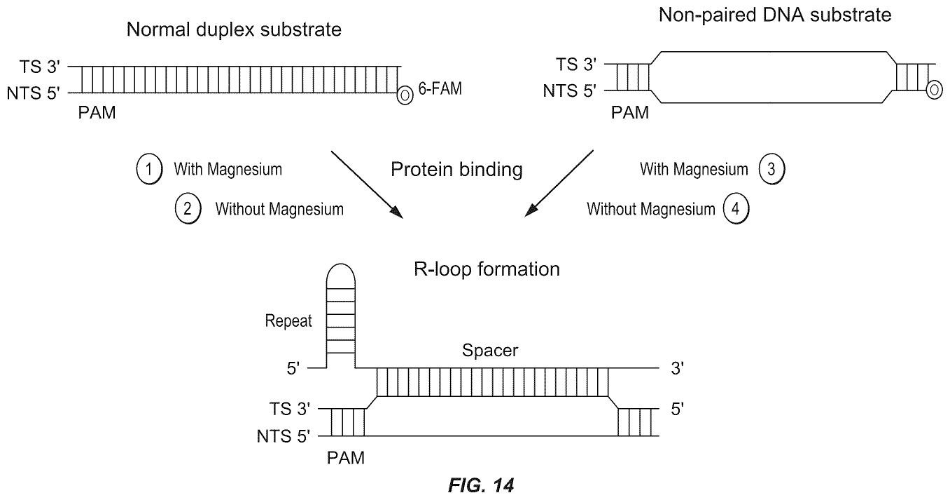

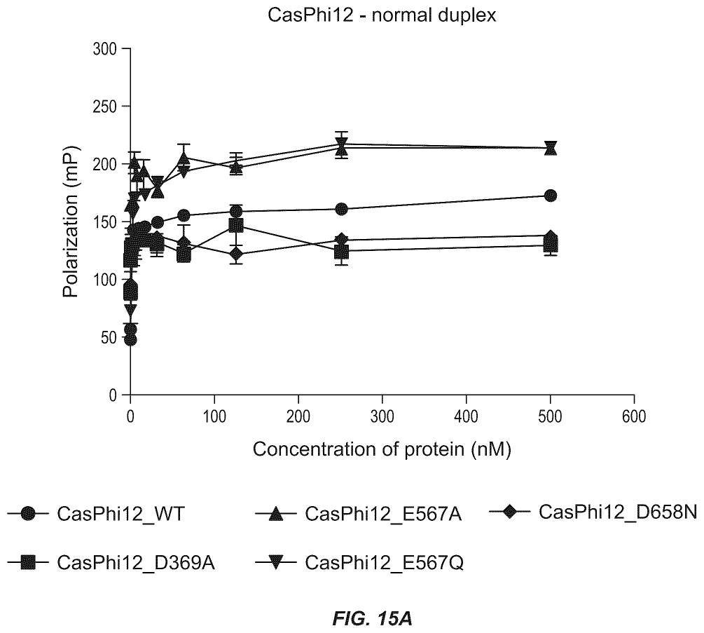

Fluorescence polarization assays were performed to assess the DNA binding affinity of the RNP complexes comprising CasPhi.12 variants, D369A, D658N, E567A, and E567Q, and crRNA (SEQ ID NO: 56). Reactions were carried out in the presence of magnesium. Two different DNA substrates were tested with magnesium: double stranded duplex DNA (“normal duplex substrate”) and non-paired DNA substrate. These conditions are represented in FIG. 14. Without being bound by theory, non-paired DNA substrate may help mimic an R loop making it easier for an RNP to bind linear DNA. The assay generates different kinds of emissions depending on whether or not a RNP:DNA complex is formed. In the absence of a complex, emissions are depolarized emissions. In the presence of a complex, there are polarized emissions.

The RNP complexes were serially diluted ranging from 500 nM-0.5 nM. Reactions were carried out with the fluorescence polarization assay buffer (20 mM HEPES pH 7.5, 0.2 mg/mL BSA, 1 mM TCEP, 100 mM NaCl, 5 mM Magnesium acetate/EDTA). A 60 bp linear DNA labelled with 6-FAM at the 3′ end was used at a concentration of 1 nM for all experiments. DNA only wells and buffer only wells served as controls. The reactions were carried out in black 384-well flat bottom plates and incubated at 37° C. for 30 minutes and read using the Biotek-Synergy H2 plate reader using the fluorescence polarization filter. The reads were adjusted to extended gain and the well height was calibrated before each run. The graphs were interpreted on PRISM using a combination of their K_{D} values and the maximum polarization value that was recorded at saturating concentrations—referred to as the plateau values.

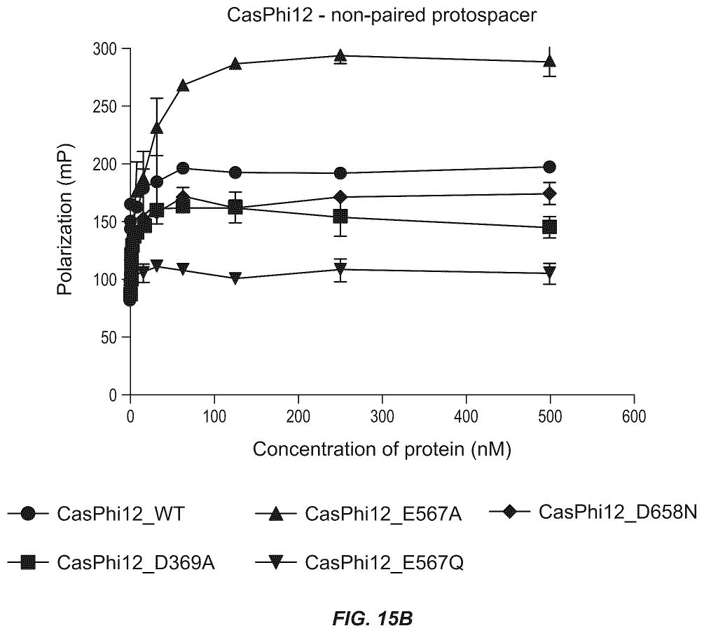

FIGS. 15A-15B shows results of fluorescence polarization assay for the RNPs comprising variants of CasPhi.12, relative to corresponding wildtype effector protein. K_{D} values (nM) for the RNPs comprising variants of CasPhi.12 were calculated based on results shown in FIGS. 15A-15B. K_{D} values (nM) for the variants are recited in TABLE 14.

TABLE 4
K_{D} VALUES FOR CASPHI.12 AND VARIANTS THEREOF

| With Magnesium | Non-paired DNA | Normal DNA |
|---|---|---|
| WT | Cleavage | Cleavage |
| D658N | 5.67 | 0.47 |
| E567Q | 1.33 | 0.46 |
| E567A | 0.49 | 0.73 |
| D369A | 2.23 | Unstable |

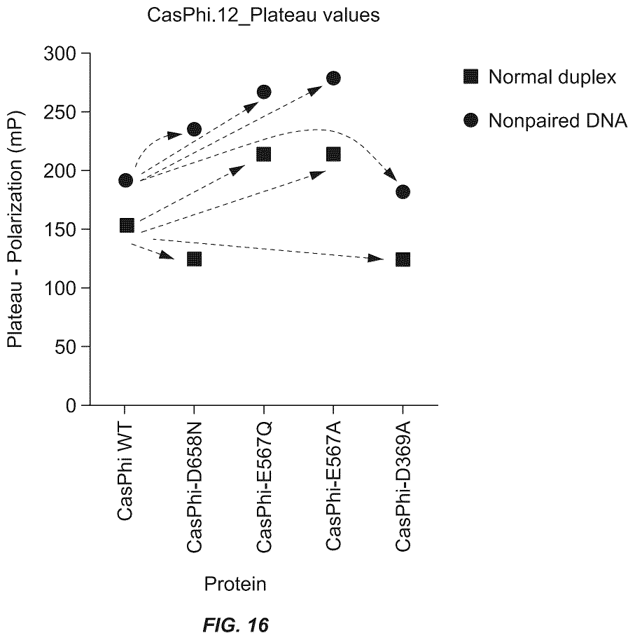

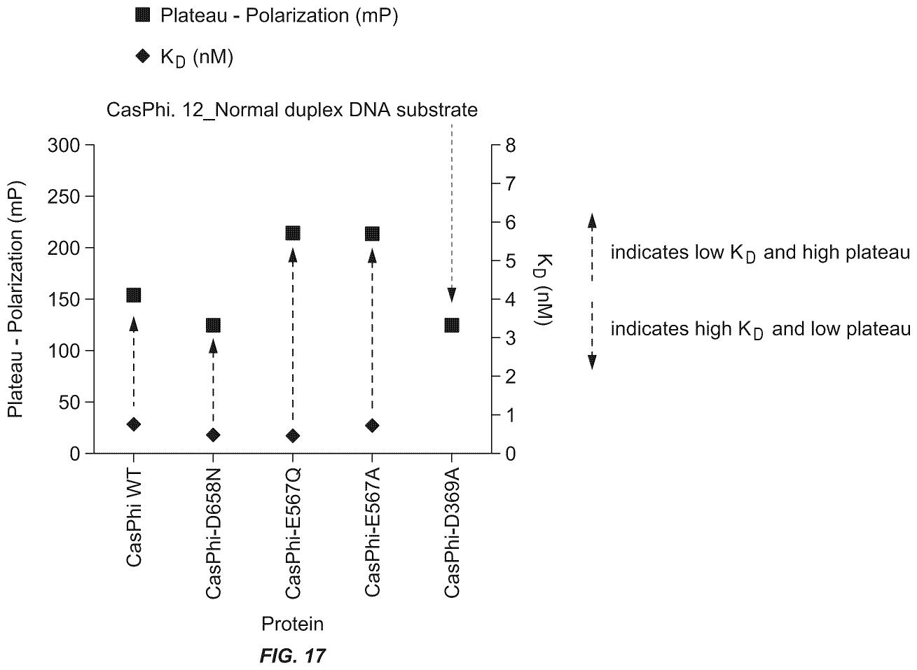

As shown in FIG. 16, the plateau amplitude was interpreted as a measure of stability of binding. FIG. 17 shows both K_{D} and plateau polarization. An analysis of FIG. 17 indicates that E567A and E567Q binds more effectively to duplex DNA relative to the corresponding wildtype.

### Example 11: CasPhi.12 Variants Engineering and Indel Activity

CasPhi.12 variants were engineered and tested for their ability to produce indels in a mammalian cell line (HEK293T cells). In total 345 plasmids were constructed and tested, each plasmid encoding for a CasPhi.12 variant engineered by an arginine scan. The plasmids encoding the variant effector proteins were tested in triplicates having been complexed with plasmids encoding guide nucleic acids having a repeat sequence of AUUGCUCCUUACGAGGAGAC (SEQ ID NO: 24). Likewise, plasmids encoding the wild-type control (CasPhi.12) were complexed with guide nucleic acids. Each experimental variant was tested in triplicate within the transfection plate, experimental controls (CasPhi.12 wild-type) included 6 replicates in each transfection plate. 300 ng of plasmid DNA was delivered by lipofection to the mammalian cells and incubated at 37° C. with 5% CO₂ for 72 hours before harvesting.

Data was filtered to remove samples with less than 1000 reads, normalized to the actual variant effector protein dose (by dividing the indel % by the experimental variant effector protein dose delivered and calculating the average normalized indel % for all triplicates), and normalized to the actual control (wild-type) effector protein dose by (dividing the indel % by the wildtype effector protein dose delivered and calculating the average normalized indel % for all 6 replicates for each plate individually). Then fold change was calculated by dividing the average normalized indel % of experimental samples by normalized indel % of control samples for the variant's corresponding plate.

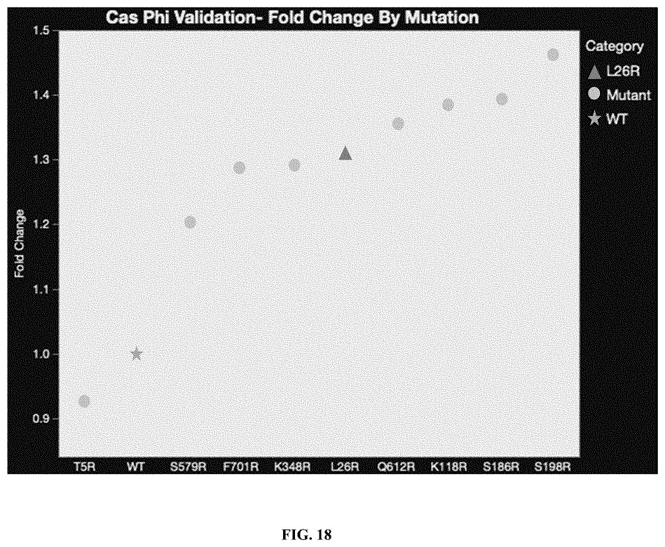

Results demonstrate that CasPhi.12 variants L26R, K118R, S186R, S198R, K348R, Q612R, F701R, T5R, and S579R were the strongest performers (FIG. 18).

### Example 12: Indel Activity of CasPhi.12 Variants with Double Mutations

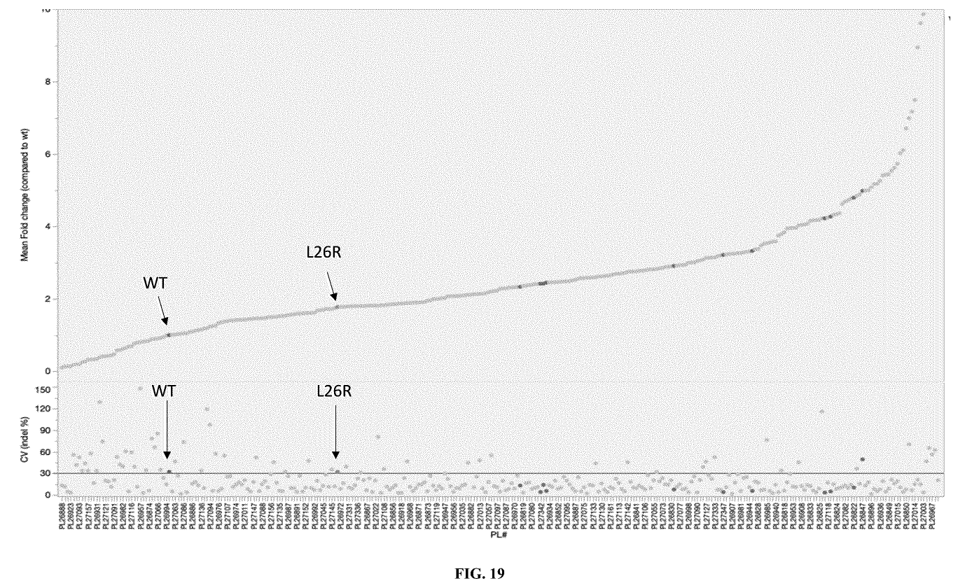

Engineered variants of CasPhi.12 double mutated based on the results of Example 11 were tested for their ability to produce indels in a mammalian cell line (e.g., HEK293T cells). Briefly, plasmids encoding the effector proteins and guides nucleic acids were delivered by lipofection to the mammalian cells as described in Example 11. This was performed with guide nucleic acid targeting loci adjacent to a PAM of NTTN (SEQ ID NO: 14). Indels in the loci were detected by next generation sequencing of PCR amplicons at the targeted loci and % indel was calculated as the fraction of sequencing reads containing insertions or deletions relative to an unedited reference sequence. FIG. 19 shows the activity of the engineered variants relative to that of wildtype as fold change. TABLE 15 lists variants that demonstrated increased indel production relative to wildtype CasPhi.12 (with highest fold change at FIG. 19 to the right).

TABLE 15
EXEMPLARY DOUBLE MUTATIONS
WITH INCREASED POTENCY

| Plasmid | Mutation 1 | Mutation 2 |
|---|---|---|
| PL27326 | L26R | K99R |
| PL27347 | L26R | P707R |
| PL27335 | L26R | L149R |
| PL27324 | L26R | N30R |
| PL27344 | L26R | N355R |
| PL27342 | L26R | K281R |
| PL27327 | L26R | S108R |
| PL27343 | L26R | K348R |
| PL26847 | T5R | V139R |
| PL26822 | I2R | V139R |
| PL26942 | K99R | S186R |

### Example 13: Potency Screening of CasPhi.12 Variants with Double Mutations

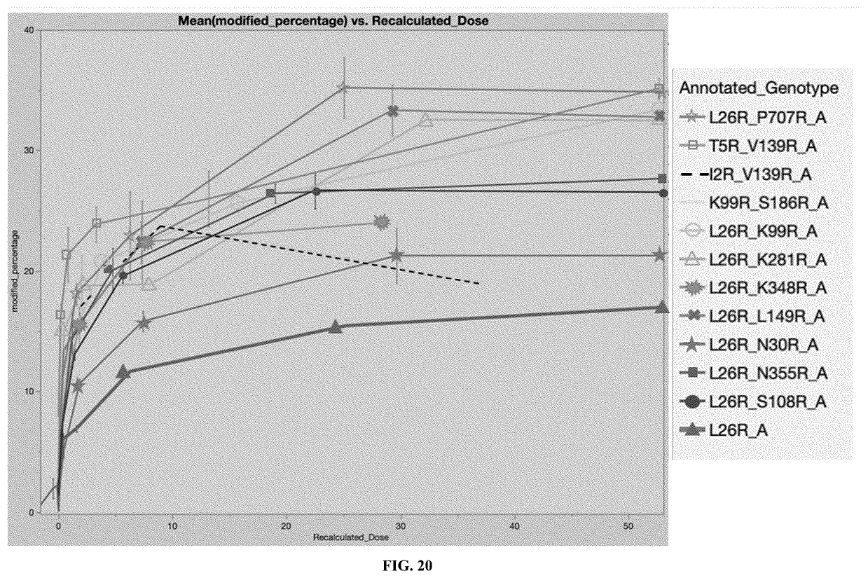

Double mutated engineered variants tested in Example 12 were tested in a dose titration experiment. Briefly, various doses of plasmids encoding the effector proteins and guides nucleic acids having a repeat sequence of 5′-AUUGCUCCUUACGAGGAGAC-3′ (SEQ ID NO: 24) were delivered by lipofection to HEK293T cells. Plasmids containing the encoded effector proteins were remeasured after transfection via QuantIT kit, and then dose was recalculated to account for any potential false positives in dataset. FIG. 20 provides the recalculated dose plotted against indel percent, demonstrating the potency of the double mutants. Variants with TSR, V139R and L26R, P707R mutations continue to appear more potent relative to CasPhi.L26R.

### Example 14: CasPhi.12 Variants Engineering and Indel Activity

CasPhi.12 variants were engineered by rational design and tested for their ability to produce indels in a mammalian cell line (HEK293T cells). Briefly, plasmids encoding the effector proteins and guide nucleic acids having a repeat sequence of 5′-AUUGCUCCUUACGAGGAGAC-3′ (SEQ ID NO: 24) were delivered by lipofection to the mammalian cells. This was performed with guide nucleic acids targeting loci adjacent to a CasPhi.12 PAM (NTTN). Indels in the loci were detected by next generation sequencing of PCR amplicons at the targeted loci and indel percentage was calculated as the fraction of sequencing reads containing insertions or deletions relative to an unedited reference sequence.

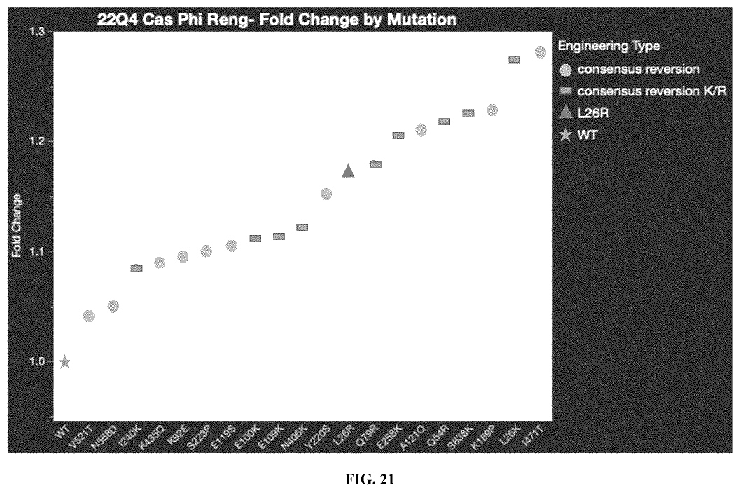

Results are demonstrated in TABLE 16 and FIG. 21. CasPhi.12 variants having the highest indels were found to be I471T, L26K, K189P, S638K, Q54R, A121Q, E258K, Q79R, Y220S, N406K, E119S, S223P, K92E, K435Q, N568D, and V521T.

TABLE 16
MUTATION AND RAW FOLD CHANGE AND INDEL DATA

| Mutation | Average Fold Change | Average Indel Percent |
|---|---|---|
| WT | 1 | 2.99131786 |
| Q54R | 1.217481236 | 3.5352771 |
| Q79R | 1.180018586 | 3.45935732 |
| T11R | 1.273070033 | 3.68843785 |
| E100K | 1.112073769 | 3.25540575 |
| I59K | 0.922422245 | 2.7046407 |
| S223P | 1.100412508 | 3.3009322 |
| P187I | 0.899900114 | 2.70321794 |
| K99S | 0.867819881 | 2.6020639 |
| K189P | 1.230786036 | 3.69464763 |
| Y220S | 1.152728157 | 3.45984038 |
| E101K | 0.991768734 | 2.97708762 |
| L26K | 1.274930417 | 3.8246202 |
| P116G | 1.010050931 | 3.03149281 |
| V84Y | 0.893609402 | 2.68115186 |
| T87G | 1.712800821 | 5.14335832 |
| G276V | 0.853685124 | 2.56259316 |
| C285V | 0.897452543 | 2.69519881 |
| E258K | 1.204876494 | 3.61306407 |
| P273A | 0.851760713 | 2.55373602 |
| A121Q | 1.211446548 | 3.64187634 |
| P89T | 0.759960457 | 2.28138115 |
| E119S | 1.108044239 | 3.32629247 |
| N147K | 1.021803044 | 3.06998605 |
| L107F | 0.861048091 | 2.58523016 |
| Y231K | 0.818853815 | 2.4594515 |
| E109K | 1.113457133 | 3.34041286 |
| I240K | 1.085766123 | 3.25913399 |
| S77V | 0.74646196 | 2.23933962 |
| H110T | 1.032992725 | 3.10081177 |
| E68P | 0.706065635 | 2.12142424 |
| Y231G | 0.751347784 | 2.25328784 |
| P94E | 0.895540887 | 2.68456365 |
| K90E | 0.859535265 | 2.58163139 |
| K92E | 1.096112544 | 3.28500933 |
| S186G | 1.00295987 | 3.00827936 |
| T295N | 0.908234286 | 2.72622376 |
| K184P | 0.978632394 | 2.93888174 |
| P304E | 0.919457505 | 2.75853099 |
| F312L | 0.806835991 | 2.41618597 |
| Y301L | 0.943533214 | 2.83155524 |
| K480L | 0.996917543 | 2.9948535 |
| N340S | 0.972020143 | 2.91481364 |
| C357L | 0.945475403 | 2.83842052 |
| R329T | 0.777901577 | 2.33721977 |
| W599F | 0.996979815 | 2.99202939 |
| L607F | 0.750430566 | 2.25109207 |
| R617Y | 0.860267627 | 2.58590891 |
| D501K | 0.99116147 | 2.97360705 |
| L620E | 0.964187536 | 2.8965944 |
| M334E | 0.752654092 | 2.25491899 |
| V483G | 0.991493918 | 2.9779046 |
| G497K | 0.981654357 | 2.94853339 |
| P399F | 0.881963045 | 2.64633187 |
| K639E | 0.885277701 | 2.65643691 |
| S526N | 1.367179326 | 4.10448663 |
| T400L | 0.838073818 | 2.513949 |
| I653A | 0.786809905 | 2.35822219 |
| E529K | 0.8655367 | 2.60016453 |
| R531E | 0.943210077 | 2.82866375 |
| E529L | 0.958212456 | 2.8749734 |
| C405L | 1.0278196 | 3.08261565 |
| N406K | 1.121731938 | 3.36874901 |
| V521T | 1.041533322 | 3.13020206 |
| K634G | 0.982107934 | 2.94604191 |
| M624A | 0.991982923 | 2.97571848 |
| V370L | 0.874289672 | 2.62220368 |
| D523K | 0.913124823 | 2.73865062 |
| S638K | 1.225218345 | 3.67999805 |
| C363V | 0.9561343 | 2.87014099 |
| M503K | 0.933302126 | 2.79785603 |
| A366V | 0.931760258 | 2.79433556 |
| F509A | 0.755665211 | 2.26767261 |
| K392A | 0.911767816 | 2.74113797 |
| E536A | 0.965434552 | 2.9026338 |
| D549L | 0.911127159 | 2.73329291 |
| I471T | 1.281172693 | 3.84160182 |
| G577H | 1.113276682 | 3.34330907 |
| F445S | 0.903545369 | 2.71080385 |
| N568D | 1.050947292 | 3.15788895 |
| K407E | 0.797520971 | 2.39229917 |
| K435Q | 1.091170629 | 3.27846952 |

### Example 15: Potency Screening of CasPhi.12 Variants

CasPhi.12 and engineered variants from Example 14 were tested in a dose titration experiment. Tested mutants are set forth in TABLE 17. Briefly, varying doses of plasmids encoding the effector proteins and guide nucleic acids having a repeat sequence of 5′-AUUGCUCCUUACGAGGAGAC-3′ (SEQ ID NO: 24) were delivered by lipofection to the mammalian cells. This was performed with guide nucleic acids targeting loci adjacent to a CasPhi12's PAM. Indels in the loci were detected by next generation sequencing of PCR amplicons at the targeted loci and indel percentage was calculated as the fraction of sequencing reads containing insertions or deletions relative to an unedited reference sequence.

Results demonstrate that CasPhi.12 variants A121Q, L26K, and 1471T showed better indel activity or close to the L26R Variant (TABLE 17).

TABLE 17
MUTATION, DOSE AND RAW INDEL
DATA OF POTENCY ASSAY

| Mutation | Dose | Average Indel Percent |
|---|---|---|
| A121Q | 1.15 | 1.968963446 |
| A121Q | 2.3 | 2.344603371 |
| A121Q | 4.6 | 3.590591356 |
| A121Q | 9.375 | 6.053025396 |
| A121Q | 18.75 | 6.027300554 |
| A121Q | 37.5 | 11.83926509 |
| A121Q | 75 | 12.89928821 |
| A121Q | 150 | 7.230144136 |
| E119S | 1.15 | 0.470644815 |
| E119S | 2.3 | 0.582587924 |
| E119S | 4.6 | 1.080769692 |
| E119S | 9.375 | 1.795653219 |
| E119S | 18.75 | 2.917481985 |
| E119S | 37.5 | 6.834740652 |
| E119S | 75 | 7.956903607 |
| E119S | 150 | 10.80289632 |
| E258K | 1.15 | 0.877032758 |
| E258K | 2.3 | 1.337995647 |
| E258K | 4.6 | 1.969563187 |
| E258K | 9.375 | 2.545820375 |
| E258K | 18.75 | 4.335265326 |
| E258K | 37.5 | 10.13442723 |
| E258K | 75 | 9.576394437 |
| E258K | 150 | 9.329053296 |
| I240K | 1.15 | 0.466670471 |
| I240K | 2.3 | 0.92992489 |
| I240K | 4.6 | 1.783943308 |
| I240K | 9.375 | 2.06957855 |
| I240K | 18.75 | 4.70943762 |
| I240K | 37.5 | 4.454481877 |
| I240K | 75 | 6.417903832 |
| I240K | 150 | 8.858150537 |
| I471T | 1.15 | 1.68692559 |
| I471T | 2.3 | 2.775842023 |
| I471T | 4.6 | 3.569315397 |
| I471T | 9.375 | 4.763189153 |
| I471T | 18.75 | 5.855023345 |
| I471T | 37.5 | 8.437983013 |
| I471T | 75 | 9.23343022 |
| I471T | 150 | 14.9865208 |
| K189P | 1.15 | 0.87612497 |
| K189P | 2.3 | 1.206035016 |
| K189P | 4.6 | 2.0962779 |
| K189P | 9.375 | 2.87526053 |
| K189P | 18.75 | 5.511960549 |
| K189P | 37.5 | 7.410887281 |
| K189P | 75 | 9.844950211 |
| K189P | 150 | 13.11380879 |
| K435Q | 1.15 | 0.555564425 |
| K435Q | 2.3 | 1.002254784 |
| K435Q | 4.6 | 1.651015461 |
| K435Q | 9.375 | 1.9540815 |
| K435Q | 18.75 | 3.745215527 |
| K435Q | 37.5 | 3.580189404 |
| K435Q | 75 | 8.355095122 |
| K435Q | 150 | 9.515037132 |
| L26K | 1.15 | 1.727246189 |
| L26K | 2.3 | 2.931248775 |
| L26K | 4.6 | 4.293001985 |
| L26K | 9.375 | 5.347712645 |
| L26K | 18.75 | 7.986249423 |
| L26K | 37.5 | 10.7479327 |
| L26K | 75 | 13.55824048 |
| L26K | 150 | 13.88834294 |
| N147K | 1.15 | 1.318186364 |
| N147K | 2.3 | 1.700387565 |
| N147K | 4.6 | 2.762247374 |
| N147K | 9.375 | 3.953555319 |
| N147K | 18.75 | 7.199764622 |
| N147K | 37.5 | 8.493246535 |
| N147K | 75 | 8.909627356 |
| N147K | 150 | 13.18498619 |
| N406K | 1.15 | 0.035323967 |
| N406K | 2.3 | 0.982592178 |
| N406K | 4.6 | 1.813351602 |
| N406K | 9.375 | 3.935477456 |
| N406K | 18.75 | 5.663257183 |
| N406K | 37.5 | 7.059157851 |
| N406K | 75 | 7.778378974 |
| N406K | 150 | 13.67843955 |
| Q54R | 1.15 | 0.449317058 |
| Q54R | 2.3 | 1.000667111 |
| Q54R | 4.6 | 2.013357935 |
| Q54R | 9.375 | 3.186292654 |
| Q54R | 18.75 | 3.923783369 |
| Q54R | 37.5 | 7.738681102 |
| Q54R | 75 | 8.158505667 |
| Q54R | 150 | 11.70332469 |
| S223P | 1.15 | 0.131782609 |
| S223P | 2.3 | 0.837403111 |
| S223P | 4.6 | 2.468454479 |
| S223P | 9.375 | 3.122385243 |
| S223P | 18.75 | 5.967776274 |
| S223P | 37.5 | 6.306133784 |
| S223P | 75 | 6.693507801 |
| S223P | 150 | 11.77809082 |
| S638K | 1.15 | 0.955784084 |
| S638K | 2.3 | 1.761952954 |
| S638K | 4.6 | 2.29560362 |
| S638K | 9.375 | 2.937868357 |
| S638K | 18.75 | 2.957254604 |
| S638K | 37.5 | 4.889101169 |
| S638K | 75 | 7.028343452 |
| S638K | 150 | 9.269143332 |
| V521T | 1.15 | 0.6314048 |
| V521T | 2.3 | 0.956702392 |
| V521T | 4.6 | 1.736793055 |
| V521T | 9.375 | 2.535246769 |
| V521T | 18.75 | 4.514496868 |
| V521T | 37.5 | 4.456335157 |
| V521T | 75 | 7.87654374 |
| V521T | 150 | 9.451403661 |
| WT | 1.15 | 0.545313155 |
| WT | 2.3 | 0.943677675 |
| WT | 4.6 | 1.580152574 |
| WT | 9.375 | 2.539016166 |
| WT | 18.75 | 4.265859454 |
| WT | 37.5 | 5.760735486 |
| WT | 75 | 7.208800959 |
| WT | 150 | 8.568746121 |
| Y220S | 1.15 | 1.070756284 |
| Y220S | 2.3 | 1.128614402 |
| Y220S | 4.6 | 2.798698408 |
| Y220S | 9.375 | 3.8740505 |
| Y220S | 18.75 | 6.409898274 |
| Y220S | 37.5 | 9.271693969 |
| Y220S | 75 | 9.132443855 |
| Y220S | 150 | 11.61565298 |

### Example 16: CasPhi.12 Variants Engineering and Indel Activity

CasPhi.12 and engineered variants thereof were tested for their ability to produce indels in a mammalian cell line (HEK293T cells). Briefly, 15 ng plasmid encoding effector proteins and guide nucleic acids having a sequence of 5′-AUUGCUCCUUACGAGGAGACGCCUUAACAAGCUGCUCU-3′(SEQ ID NO: 29) were delivered by lipofection to the mammalian cells. This was performed with guide RNAs targeting loci adjacent to a CasPhi.12 PAM. Indels in the loci were detected by next generation sequencing of PCR amplicons at the targeted loci and indel percentage was calculated as the fraction of sequencing reads containing insertions or deletions relative to an unedited reference sequence. Results are presented as mean fold change relative to indels generated with wildtype CasPhi.12 (SEQ ID NO: 1) in TABLE 18 below.

TABLE 18
VARIANT MEAN FOLD CHANGE
RELATIVE TO SEQ ID NO: 1

| Description | Mean Fold Change to WT |
|---|---|
| L26R, A673G | 3.11733539 |
| L26R, Q674R | 2.953257165 |
| S579R, L26K | 2.882393457 |
| F701R, E258K | 2.692477551 |
| T5R, L26K | 2.653730513 |
| L26R, K435Q | 2.653321586 |
| L26R, G685R | 2.614565181 |
| L26R, Q674K | 2.566579769 |
| L26R, P699R | 2.549090458 |
| L26R, T70E | 2.511755344 |
| L26R, Q232R | 2.435417978 |
| L26R, T252R | 2.316627455 |
| L26R, P679R | 2.305408759 |
| L26R, E83K | 2.291962533 |
| L26R, E73P | 2.28666561 |
| L26R, K248E | 2.285055309 |
| L26R | 2.278446422 |
| T5R, S223P | 2.230381351 |
| S579R, S223P | 2.218563275 |
| L26R, S223P | 2.19607818 |
| T5R, A121Q | 2.167072598 |
| L26R, A696R | 2.144561241 |
| S198R, I471T | 2.097749483 |
| L26R, N153R | 2.090687002 |
| L26R, E682R | 2.079506039 |
| L26R, D703R | 2.035653506 |
| Q612R, L26K | 1.998708371 |
| L26R, I471T | 1.990256661 |
| K348R, L26K | 1.961982845 |
| S579R, I471T | 1.941257676 |
| L26R, V228R | 1.918570245 |
| T5R, S638K | 1.917688113 |
| S579R, K189P | 1.905198048 |
| S579R, E258K | 1.903955388 |
| L26R, K260R | 1.899633546 |
| L26R, S638K | 1.894016501 |
| S579R, Y220S | 1.890295928 |
| T5R, I471T | 1.880336505 |
| L26R, F233R | 1.835862734 |
| L26R, V521T | 1.824379936 |
| F701R, A121Q | 1.82427261 |
| L26R, G361R | 1.812009584 |
| S198R, E258K | 1.806297913 |
| L26R, S472R | 1.795831632 |
| T5R, Y220S | 1.795746639 |
| L26R, A150K | 1.750680132 |
| L26R, S684R | 1.749145135 |
| L26R, E157R | 1.727470888 |
| L26R, K248R | 1.722683942 |
| F701R, L26K | 1.699743448 |
| S198R, N406K | 1.679898805 |
| S198R, Y220S | 1.673037467 |
| S198R, S638K | 1.64765257 |
| S198R, V521T | 1.647071553 |
| S579R, A121Q | 1.632517972 |
| K348R, Y220S | 1.625280249 |
| S198R, K189P | 1.611402453 |
| L26R, E242R | 1.599354511 |
| L26R, K678R | 1.598048698 |
| T5R, N406K | 1.593001639 |
| L26R, I158K | 1.591996336 |
| T5R, V521T | 1.584129708 |
| L26R, N259R | 1.559293551 |
| L26R, K257R | 1.552100135 |
| L26R, K256R | 1.552100135 |
| T5R, K189P | 1.476348249 |
| L26R, C405R | 1.446360226 |
| S579R, V521T | 1.397112204 |
| S579R, N406K | 1.394790397 |
| T5R, K92E | 1.378668882 |
| T5R, E258K | 1.347658267 |
| L26R, I97R | 1.343965755 |
| S579R, S638K | 1.334430286 |
| T5R, K435Q | 1.288692878 |
| F701R, S638K | 1.276631645 |
| L26R, L236R | 1.274327294 |
| F701R, I471T | 1.216824097 |
| Q612R, S223P | 1.207567256 |
| F701R, S223P | 1.183624155 |
| S198R, E119S | 1.144995508 |
| S579R, K92E | 1.135753612 |
| L26R, E715R | 1.130486484 |
| Q612R, I471T | 1.130168321 |
| F701R, Y220S | 1.120466313 |
| S198R, S223P | 1.119463414 |
| L26R, K266R | 1.111463077 |
| S198R, K92E | 1.099073399 |
| L26R, I268R | 1.079018004 |
| L26R, G244R | 1.061187211 |
| F701R, K189P | 1.053038183 |
| Q612R, E119S | 1.047068553 |
| F701R, N406K | 1.040572504 |
| L26R, E109K | 1.03409299 |
| F701R, K435Q | 1.016541831 |
| CasPhi.12 WT | 1 |
| L26R, Y220S | 0.996571759 |
| L26R, G57R | 0.992006397 |
| L26R, A716R | 0.964182232 |
| S579R, E119S | 0.962943905 |
| K348R, V521T | 0.941678966 |
| Q612R, S638K | 0.891799493 |
| K118R, K189P | 0.862773847 |
| F701R, V521T | 0.849823891 |
| K118R, K92E | 0.835177763 |
| Q612R, A121Q | 0.830640146 |
| F701R, E119S | 0.805287112 |
| L26R, P239R | 0.792924619 |
| L26R, Y231R | 0.772053746 |
| K348R, S223P | 0.760388575 |
| L26R, C279R | 0.758710955 |
| K348R, E119S | 0.756104818 |
| L26R, E100K | 0.755381659 |
| K348R, N406K | 0.744773406 |
| Q612R, E258K | 0.736106841 |
| L26R, S255R | 0.717073928 |
| F701R, K92E | 0.7165933 |
| Q612R, N406K | 0.71648782 |
| K348R, K189P | 0.691835156 |
| S186R, E258K | 0.690038456 |
| Q612R, Y220S | 0.688872411 |
| L26R, N568D | 0.665687091 |
| L26R, G241R | 0.630454779 |
| F701R, N568D | 0.625920655 |
| Q612R, K435Q | 0.623735079 |
| T5R, N568D | 0.586190352 |
| Q612R, V521T | 0.548695816 |
| S186R, V521T | 0.540059693 |
| K348R, A121Q | 0.539923339 |
| L26R, D234R | 0.514928206 |
| K348R, K92E | 0.512793993 |
| S579R, N568D | 0.48837324 |
| L26R, A59R | 0.473724067 |
| Q612R, K92E | 0.472560163 |
| L26R, H297R | 0.458553639 |
| L26R, E258R | 0.443211882 |
| K348R, K435Q | 0.436021979 |
| L26R, P94K | 0.380129252 |
| K348R, I471T | 0.376607598 |
| L26R, E119S | 0.374260586 |
| S579R, K435Q | 0.370671665 |
| L26R, Y709R | 0.352941041 |
| S198R, N568D | 0.316753581 |
| S186R, N568D | 0.222272124 |
| L26R, G497R | 0.211450566 |
| Q612R, N568D | 0.207916011 |
| K118R, N568D | 0.203867088 |
| L26R, L254R | 0.185076735 |
| K348R, E258K | 0.166136945 |
| L26R, P96K | 0.15508531 |
| L26R, Y245R | 0.13562419 |
| L26R, I238R | 0.130864714 |
| L26R, P243R | 0.1290174 |
| L26R, L16F | 0.068931814 |
| L26R, P230R | 0.06877497 |
| L26R, P199R | 0.066994434 |
| L26R, G585R | 0.049913234 |
| K348R, N568D | 0.034148842 |
| L26R, A673R | 0.022461934 |
| L26R, V246R | 0.016114335 |
| L26R, S229R | 0.012467481 |
| L26R, S265R | 0.012451569 |
| L26R, C50P | 0.011878756 |
| L26R, F253R | 0.010914596 |
| L26R, L264R | 0.006817561 |
| L26R, I227R | 0.005457392 |
| L26R, P247R | 0 |
| L26R, Q250R | 0 |

### Example 17: Determining Analog Incubation Conditions for Assessing Translocation Rates Across Three Different Target Nucleic Acids (B2M, TRAC, CIITA) after Multiplex Editing with L26R Variant of CasPhi.12

Described herein is the method for measuring and evaluating L26R variant of CasPhi.12 translocations (chromosomal rearrangements) rates in comparison to Cas9. The guide nucleic acids were engineered to target B2M gene, TRAC gene, or CIITA gene. Briefly, T cells were thawed and activated with CD3/CD28 dyna beads for 72 hours. Followingly, 3e5 activated T cells were electroporated with mRNA encoding effector protein (5 μg) and guide nucleic acids (500 μM) for editing single gene or all three genes simultaneously. The electroporated T cells were cultured for 3 days in a 48 well plate. The electroporated cells were then evaluated for editing efficiency by FACS and NGS analysis, translocations/chromosomal rearrangement assessment was determined by dGH assay, and cell viability was assessed by counting trypan blue stained T cells by Countess cell counter. For dGH assay, the electroporated cells were treated with an analog 3 days post-electroporation for 17 hours. Followingly, the cells were treated with colcemid for 4 hours. Alternatively, the electroporated cells were also treated with an analog 3 days post-electroporation for 14 hours followed by colcemid treatment for 4 hours (the results are not shown).

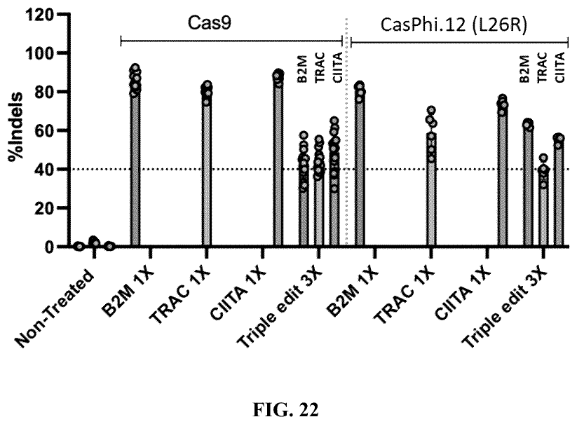

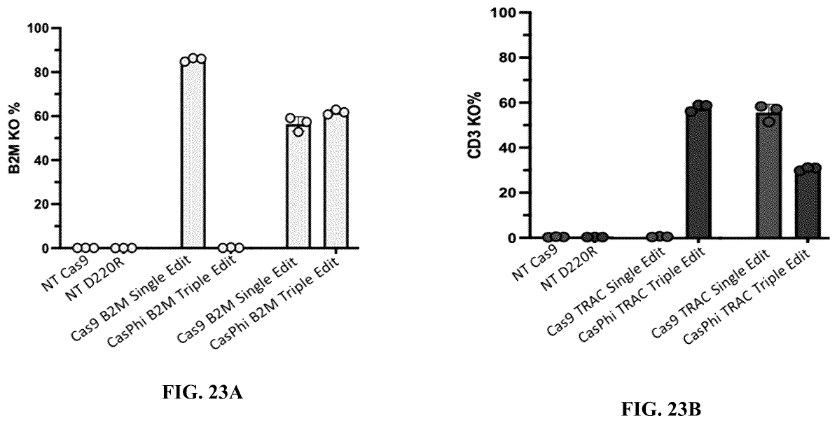

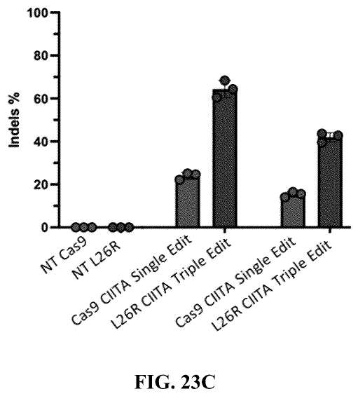

FIG. 22 shows results of NGS study. Similarly, FIGS. 23A-23C shows results of FACS. An analysis of FIG. 22 and FIGS. 23A-23C indicated that editing efficiency for Cas9 and L26R variant of CasPhi.12 effector protein was similar for both single gene editing and simultaneous triple gene editing. The analysis further indicates that fewer T cells were observed to be consistently triple gene edited by Cas9 effector protein relative to L26R variant of CasPhi.12 effector protein.

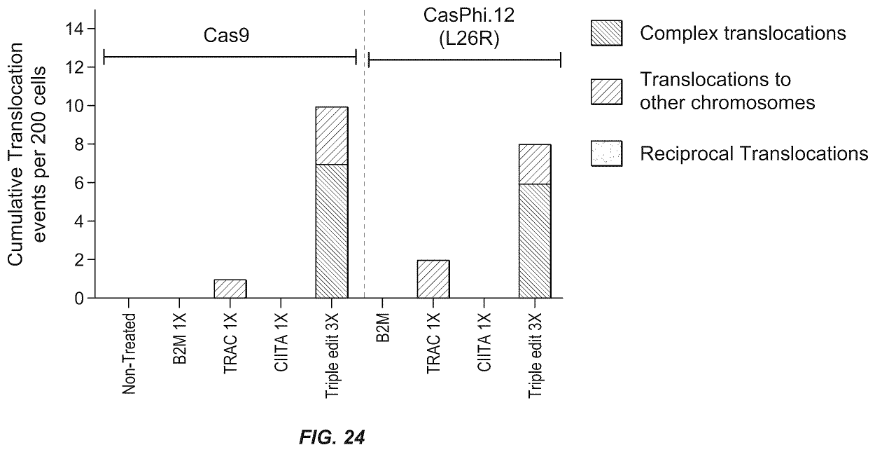

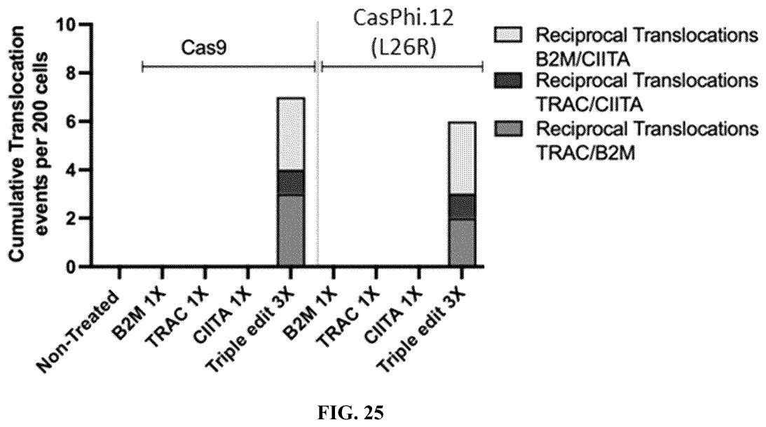

FIG. 24 shows results of translocation rates based on dGH assay. Similarly, FIG. 25 shows results of reciprocal translocations rates based on dGH assay. An analysis of FIG. 24 and FIG. 25 indicated that primary human T cells that were triple edited with L26R variant of CasPhi.12 showed fewer translocations relative to Cas9 edited T cells.

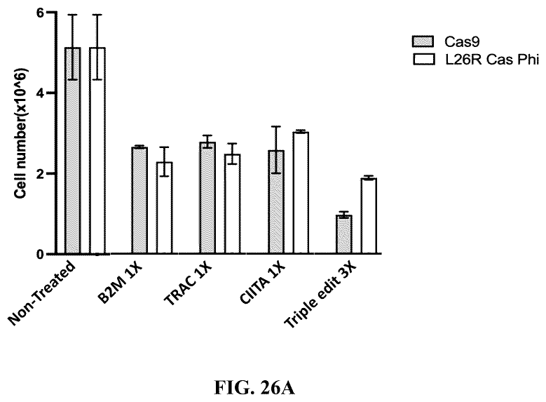

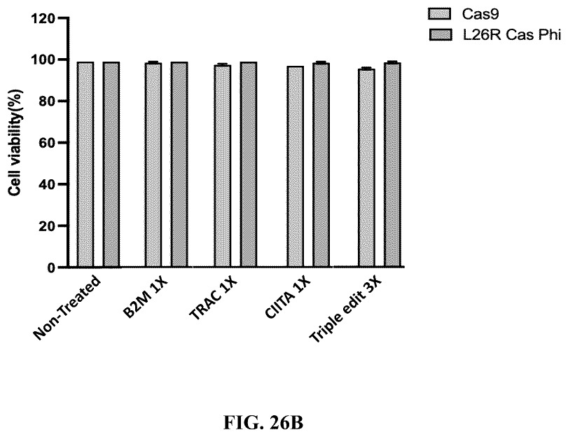

FIG. 26A and FIG. 26B show results of cell counts and cell viability, respectively.

### Example 18: Optimizing Dose of mRNA Encoding Effector Protein for Assessing Translocation Rates Across Three Different Target Nucleic Acids (B2M, TRAC, CIITA) after Multiplex Editing L26R Variant of CasPhi.12

Described herein is a method for measuring and evaluating L26R variant of CasPhi.12 translocations (chromosomal rearrangements) rates in comparison to Cas9. The guide nucleic acids were engineered to target B2M gene, TRAC gene, or CIITA gene. Briefly, T cells were thawed and activated with CD3/CD28 dyna beads for 72 hours. Followingly, 3e5 activated T cells were electroporated with mRNA encoding effector protein (10 μg) and guide nucleic acids (500 μM) for editing single gene or all three genes simultaneously. The electroporated T cells were cultured for 3 days in a 48 well plate. The electroporated cells were then evaluated for editing efficiency by NGS analysis. For dGH assay, the electroporated cells were treated with an analog 3 days post-electroporation for 17 hours. Followingly, the cells were treated with colcemid for 3 hours.

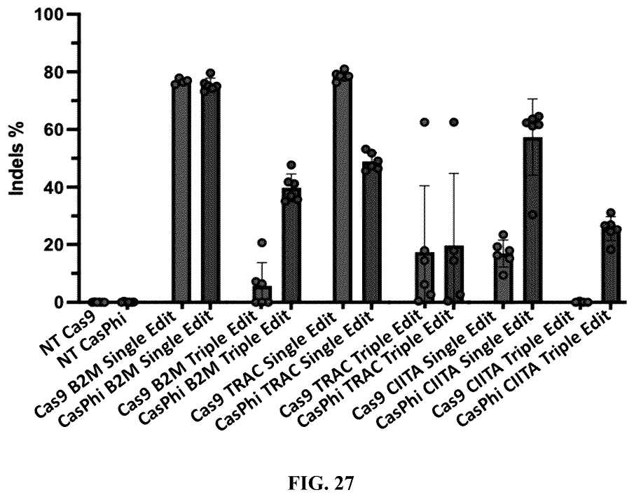

FIG. 27 shows results of an NGS study. An analysis of FIG. 27 indicated had relatively low editing activity relative to the single gene editing control.

### Example 19: Dose Titration of mRNA Encoding Effector Protein for Assessing Translocation Rates Across Three Different Target Nucleic Acids (B2M, TRAC, CIITA) after Multiplex Editing with L26R Variant of CasPhi.12

Described herein is a method for measuring and evaluating L26R variant of CasPhi.12 translocations (chromosomal rearrangements) rates in comparison to Cas9. The guide nucleic acids were engineered to target B2M gene, TRAC gene, or CIITA gene. Briefly, T cells were thawed and activated with CD3/CD28 dyna beads for 72 hours. Followingly, 3e5 activated T cells were electroporated with mRNA encoding effector protein (6 μg or 9 μg) and guide nucleic acids (500 μM) for editing single gene or all three genes simultaneously. The electroporated T cells were cultured for 3 days in a 48 well plate. The electroporated cells were then evaluated for editing efficiency by FACS and NGS analysis, translocations/chromosomal rearrangement assessment was determined by dGH assay, and cell viability was assessed by counting trypan blue stained T cells by Countess cell counter. For dGH assay, the electroporated cells were treated with an analog 3 days post-electroporation for 17 hours. Followingly, the cells were treated with colcemid for 3 hours.

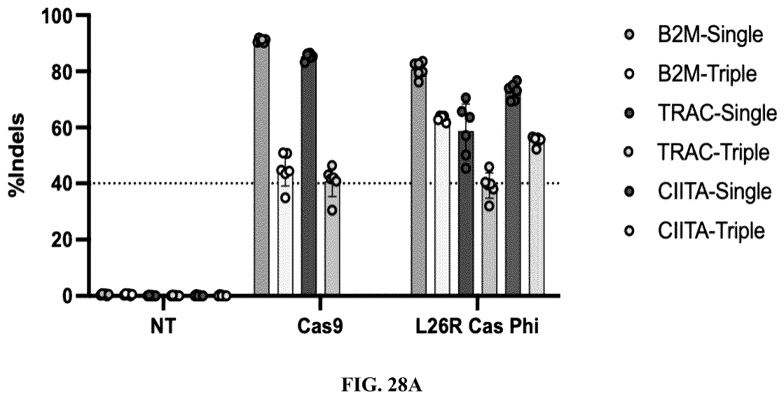

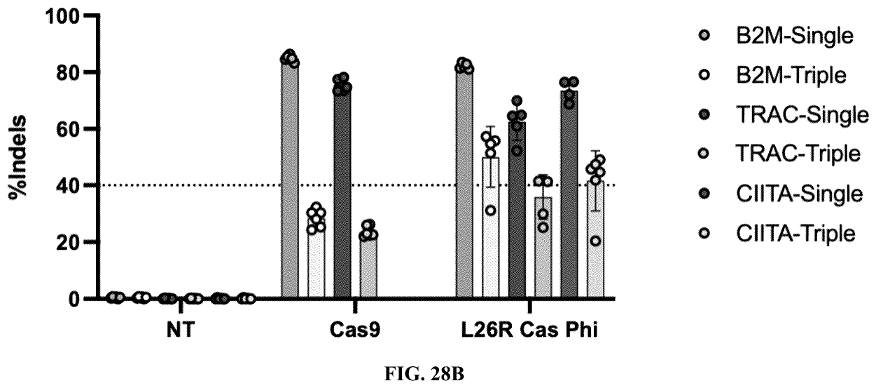

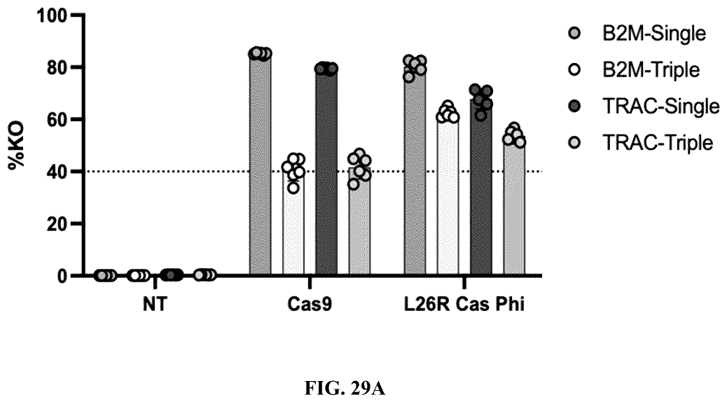

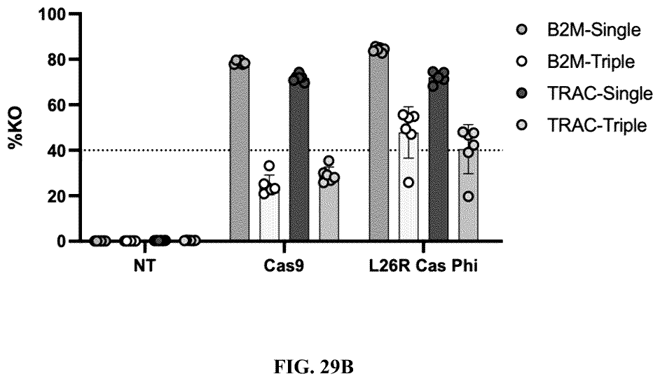

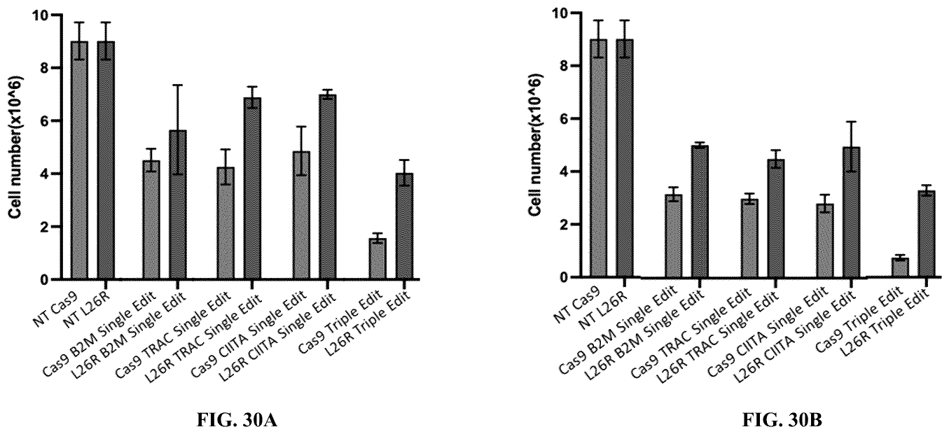

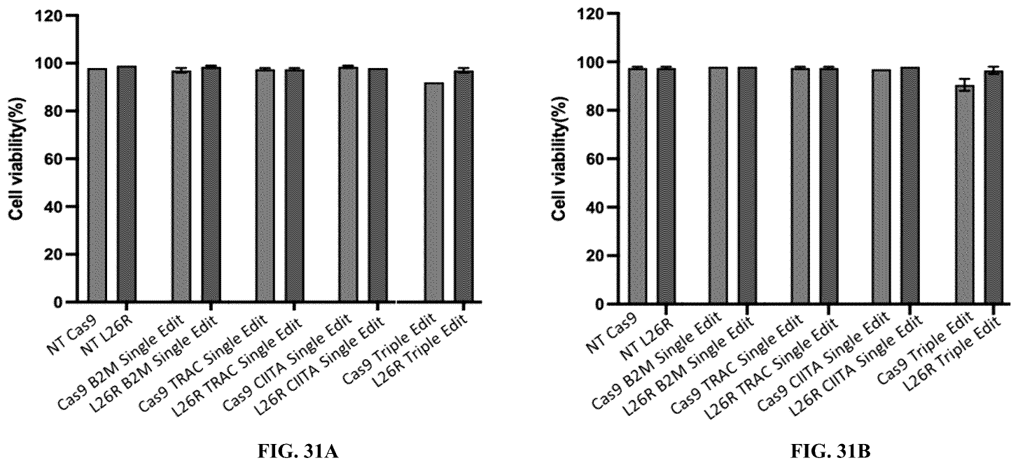

FIG. 28A and FIG. 28B shows results of an NGS study at 6 μg or 9 μg, respectively. Similarly, FIG. 29A and FIG. 29B shows results of FACS study at 6 μg or 9 μg, respectively. FIG. 30A and FIG. 30B shows results of cell counts at 6 μg or 9 μg, respectively. FIG. 31A and FIG. 31B shows results of cell viability at 6 μg or 9 μg, respectively.

### Example 20: Effector Protein Mediated Integration of a Bidirectional AAV Reporter in Eukaryotic Cells

### Example 20.1: Dose Optimization of Effector Protein Mediated Integration

Effector protein systems (e.g., CasPhi.12 L26R) were tested for their ability to generate indel in primary human hepatocytes within or adjacent to intron 1 of human albumin gene. In brief, the effector protein system (e.g., CasPhi.12 L26R) was tested for its ability to facilitate homology-independent targeted insertion (HITI) of a donor nucleic acid encoding a luciferase protein (e.g., nano luciferase reporter, nanoLuc, nLuc, nLuc AAV reporter) into the intron 1 of human albumin gene in primary human hepatocyte cells. 20,000 cells were seeded in a low adhesion 96-well plate and transfected using lipofectamine MessengerMax. Primary human hepatocyte cells were transfected with different concentrations of RNA (e.g., 25 ng, 100 ng, 400 ng) (1:1 mRNA to gRNA ratio). The gRNA used are provided in TABLE 19.

TABLE 19
GUIDE SEQUENCES USED FOR TESTING EFFECTOR PROTEIN
MEDIATED INTEGRATION OF A BIDIRECTIONAL AAV
REPORTER

| SEQ | Type | | |
|---|---|---|---|
| ID | of | Ref. | |
| NO: | gRNA | No. | Sequence |
| 126 | crRNA | R10006 | AUAGAUUGCUCCUUACGAGGAGACGUAUUUGUG |
| | | | AAGUCUUA |
| 127 | crRNA | R10007 | AUAGAUUGCUCCUUACGAGGAGACUAUUUGUGA |
| | | | AGUCUUAC |
| 128 | crRNA | R10039 | AUAGAUUGCUCCUUACGAGGAGACCUUUGCACU |
| | | | UUCCUUAG |
| 129 | crRNA | R10044 | AUAGAUUGCUCCUUACGAGGAGACAGUCACUCU |
| | | | AAGUUACU |
| 130 | crRNA | R10059 | AUAGAUUGCUCCUUACGAGGAGACAAUAAAGCA |
| | | | UAGUGCAA |
| 131 | crRNA | R10060 | AUAGAUUGCUCCUUACGAGGAGACAAAUAAAGC |
| | | | AUAGUGCA |
| 132 | crRNA | R10067 | AUAGAUUGCUCCUUACGAGGAGACUGAGAUCAA |
| | | | CAGCACAG |
| 133 | crRNA | R10068 | AUAGAUUGCUCCUUACGAGGAGACAUGAGAUCA |
| | | | ACAGCACA |
| 134 | crRNA | R10081 | AUAGAUUGCUCCUUACGAGGAGACCAUUUUAGU |
| | | | CUGUCUUC |
| 135 | crRNA | R10128 | AUAGAUUGCUCCUUACGAGGAGACAAAGGCAGA |
| | | | AGAAAUAA |
| 136 | crRNA | R10131 | AUAGAUUGCUCCUUACGAGGAGACGGCUCUGAU |
| | | | UCCUACAG |
| 137 | crRNA | R10139 | AUAGAUUGCUCCUUACGAGGAGACCUGUAGGAA |
| | | | UCAGAGCC |
| 138 | crRNA | R10144 | AUAGAUUGCUCCUUACGAGGAGACGACUUAGAU |
| | | | UAUGCAUU |
| 139 | crRNA | R10162 | AUAGAUUGCUCCUUACGAGGAGACUUUUCUUCA |
| | | | GUAUUUAA |
| 140 | crRNA | R10163 | AUAGAUUGCUCCUUACGAGGAGACUCUUCAGUA |
| | | | UUUAACAA |

The gRNA used for CasPhi.12 L26R system included SEQ ID NOS: 126-140. One or more SpyCas9 gRNA sequences were used as positive controls. The transfection was carried in conjunction with AAV transduction such that the different concentrations of RNA were paired with different concentrations of viral particles or MOI (multiplicity of infection) (e.g., 2.5×10³, 1×10⁴, 4×10⁴), where AAV carrying reporter AAV vector was added to the media. The reporter AAV vector was packaged with the donor nucleic acid encoding a luciferase protein (e.g., nLuc). The donor nucleic acid was co-delivered to the cells with the nuclease mRNA and gRNA. The composition comprising the effector protein system was incubated for 2 hours while rocking the low adhesion 96-well plate before being transferred to a Collagen I coated 96-well plate for culture. 48 hours post-transfer, the cells were harvested and were subject to DNA extraction. Indels were quantified by NGS and integration assessed by luciferase assay.

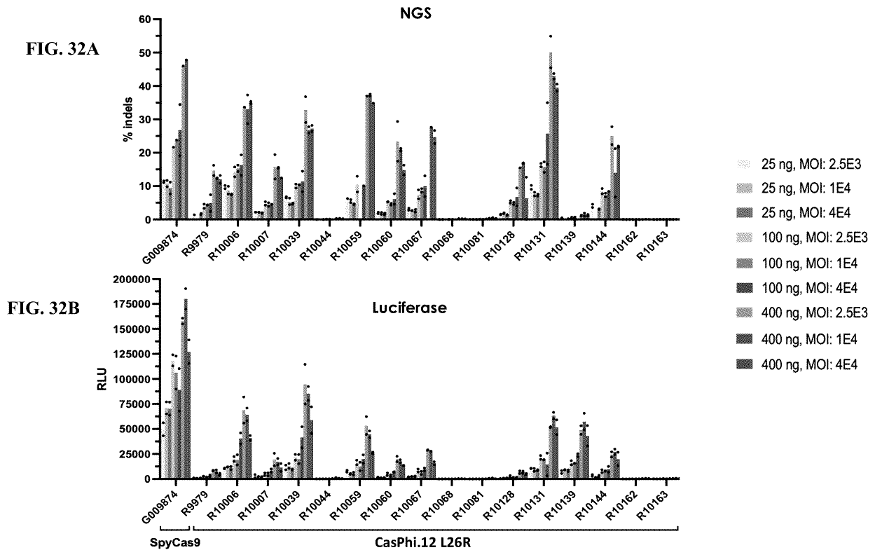

FIG. 32A shows % indel activity of the effector protein system (e.g., CasPhi.12 L26R) within or adjacent to intron 1 of human albumin gene as related to different concentrations of RNA and MOI. As shown in FIG. 32A, the effector protein system (e.g., CasPhi.12 L26R) may be used to generate indels within intron 1 of human albumin gene, as compared to the positive control.

FIG. 32B shows relative light units (RLU) as a measure of integration activity of the effector protein system (e.g., CasPhi.12 L26R) within or adjacent to intron 1 of human albumin gene as related to different concentrations of RNA and MOI. As shown in FIG. 32B, the effector protein system (e.g., CasPhi.12 L26R) may be used to insert a nucleotide sequence encoding a donor nucleic acid encoding a luciferase protein (e.g., nLuc) within intron 1 of human albumin gene, as compared to the positive control. In particular, CasPhi.12 L26R can achieve 50%-70% integration. Also as shown in FIG. 32B, the highest levels of integration were achieved upon pairing the effector protein system with 400 ng of RNA and MOI of 1×10⁴.

### Example 20.2: Dose Optimization of Effector Protein Mediated Integration

Similar to Example 20.1, an effector protein (e.g., CasPhi.12 L26R) system capable of indel activity within or adjacent to intron 1 of human albumin gene was tested for its ability to facilitate homology-independent targeted insertion (HITI) of a donor nucleic acid encoding a luciferase protein (e.g., nLuc, nLuc AAV reporter) into the intron 1 of human albumin gene in primary human hepatocyte cells. Briefly, 20,000 cells were seeded in a low adhesion 96-well plate and transfected using lipofectamine MessengerMax. Primary human hepatocyte cells were transfected with 400 ng of RNA. The gRNA used are provided in TABLE 19.

The gRNA used for CasPhi.12 L26R system included SEQ ID NOS: 126-140. One or more SpyCas9 gRNA sequences were used as positive controls. The transfection was carried in conjunction with AAV transduction with MOI of 1×10⁴, where AAV carrying reporter AAV vector was added to the media. The reporter AAV vector was packaged with the donor nucleic acid encoding a luciferase protein (e.g., nLuc). The donor nucleic acid was co-delivered to the cells with the nuclease mRNA and gRNA. The composition comprising the effector protein system was incubated for 2 hours while rocking the low adhesion 96-well plate before being transferred to a Collagen I coated 96-well plate for culture. 48 hours post-transfer, the cells were harvested and were subject to DNA extraction. Indels were quantified by NGS and integration assessed by luciferase assay and reverse transcription droplet digital PCR (RT-ddPCR) as measured via RT-ddPCR.

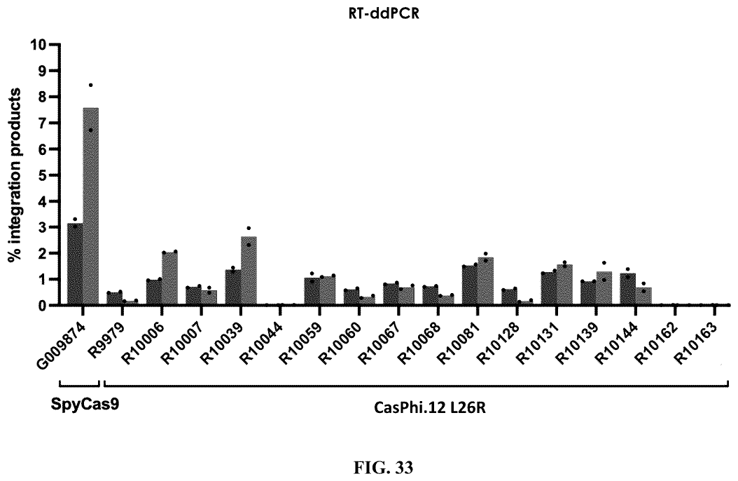

FIG. 33 shows % integration products as a measure of integration activity of the effector protein system (e.g., CasPhi.12 L26R) within or adjacent to intron 1 of human albumin gene. As shown in FIG. 33, the effector protein system (e.g., CasPhi.12 L26R) may be used to insert a nucleotide sequence encoding a donor nucleic acid encoding a luciferase protein (e.g., nLuc) within intron 1 of human albumin gene, as compared to the positive control. For example, CasPhi.12 L26R variant can achieve 3% integration, respectively.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

1. A composition comprising an engineered polypeptide or a nucleic acid encoding the engineered polypeptide, wherein the engineered polypeptide comprises one or more amino acid substitutions relative to SEQ ID NO: 1, wherein the one or more amino acid substitutions are selected from: I2R, T5R, K15R, R18R, H20R S21R, L26R, L26K, N30R, E33R, E34R, A35R, K37R, K38R, R41R, N43R, Q54R Q79R, K92E, K99R, S108R, E109R, H110R, G111R, D113R, T114R, P116R, K118R, E119S, A121Q, N132R, K135R, Q138R V139R, L149R, Y180R, L182R, Q183R, K184R, S186R, K189R, K189P, S196R, S198R, K200R, 1203R S205R, K206R, Y207R, H208R, N209R, Y220S, S223P, E258K, K281R, K348R, N355R, N406K, K4350, 1471T, V521T, N568D, S579R, Q612R, S638K, F701R, and P707R, and a combination thereof; and wherein other than the one or more amino acid substitutions, the engineered polypeptide comprises an amino acid sequence that is at least 90% identical to the amino acid sequence of SEQ ID NO: 1.

2. The composition of claim 1, wherein other than the one or more amino acid substitutions, the amino acid sequence of the engineered polypeptide is at least 95% identical to the amino acid sequence of SEQ ID NO: 1.

3.-7. (canceled)

8. The composition of claim 1, wherein the one or more amino acid substitutions comprise L26R or L26K.

9. The composition of claim 1, wherein the one or more amino acid substitutions comprises L26R and I471T.

10. The composition of claim 1, wherein the one or more amino acid substitutions are selected from: I471T, L26K, K189P, S638K, Q54R A121Q, E258K, Q79R Y220S, N406K, E119S, S223P, K92E, K435Q, N568D, and V521T, and a combination thereof.

11. The composition of claim 1, wherein the engineered polypeptide comprises a combination of amino acid substitutions as recited in TABLE 1.1.

12. The composition of claim 1, wherein the engineered polypeptide comprises a combination of amino acid substitutions as recited TABLE 1.2.

13. The composition of claim 1, wherein the engineered polypeptide comprises a combination of amino acid substitutions as recited TABLE 1.3.

14.-16. (canceled)

17. The composition of claim 1, wherein the engineered polypeptide further comprises an amino acid substitution selected from: D369A, D369N, D658A, D658N, E567A, E567Q, and a combination thereof.

18. The composition of claim 17, wherein the engineered polypeptide is fused to a fusion partner.

19. The composition of claim 18, wherein the fusion partner is selected from an exonuclease, a reverse transcriptase, a deaminase, a transcriptional activator, a transcriptional repressor, or a functional domain thereof.

20. The composition of claim 18, wherein the fusion partner comprises a methyltransferase or functional domain thereof.

21. (canceled)

22. The composition of claim 1, wherein the engineered polypeptide recognizes a protospacer adjacent motif (PAM) sequence adjacent to a target sequence in a target nucleic acid, and wherein the PAM sequence comprises 5′-NTTN-3′.

23. The composition of claim 1, comprising an engineered guide nucleic acid or a nucleic acid encoding an engineered guide nucleic acid.

24. The composition of claim 23, wherein the engineered guide nucleic acid comprises a repeat sequence that is at least 90% identical to any one of the nucleotide sequences set forth in TABLE 3.

25.-26. (canceled)

27. A method of modifying a target nucleic acid, the method comprising contacting the target nucleic acid with the composition of claim 1.

28. (canceled)

29. A method of modifying a target nucleic acid in a human subject, comprising administering the composition of claim 1 to the human subject.

30.-37. (canceled)

38. A cell comprising or modified by the composition of claim 1.

39.-49. (canceled)

50. A messenger RNA encoding an engineered polypeptide, wherein the engineered polypeptide comprises one or more amino acid substitutions relative to SEQ ID NO: 1, wherein the one or more amino acid substitutions are selected from: I2R, T5R, K15R, R18R, H20R, S21R, L26R, L26K, N30R, E33R, E34R, A35R, K37R, K38R, R41R, N43R, Q54R, Q79R, K92E, K99R, S108R, E109R, H110R, G111R, D113R, T114R, P116R, K118R, E119S, A121Q, N132R, K135R, Q138R, V139R, L149R, Y180R, L182R, Q183R, K184R, S186R, K189R, K189P, S196R, S198R, K200R, 1203R, S205R, K206R, Y207R, H208R, N209R, Y220S, S223P, E258K, K281R, K348R, N355R, N406K, K435Q, 1471T, V521T, N568D, S579R, Q612R, S638K, F701R, and P707R, and a combination thereof; and wherein the engineered polypeptide comprises an amino acid sequence that is at least 90% identical to the amino acid sequence of SEQ ID NO: 1.

51. A lipid nanoparticle comprising the messenger RNA of claim 50.
